(19) United States
(12) Patent Application Publication
(10) Pub. No.: US 2025/0346577 A1
(43) Pub. Date: Nov. 13, 2025

(54) PROCESSES FOR THE PREPARATION OF SUBSTITUTED 3-((3-AMINOPHENYL)AMINO)PIPERIDINE-2,6-DIONE COMPOUNDS

(71) Applicant: Bristol-Myers Squibb Company, Princeton, NJ (US)

(72) Inventors: Gregory Beutner, Green Book, NJ (US); Ronald Carrasquillo, Glen Gardner, NJ (US); William P. Gallagher, Clarksburg, NJ (US); Maryll Geherty, Pennington, NJ (US); Jianxin Han, Green Book, NJ (US); Kelvin Jones, Somerset, NJ (US); Damian Reyes Luyanda, Jersey City, NJ (US); C. Derek Ma, East Hanover, NJ (US); Christina B. Madsen-Duggan, Scotch Plains, NJ (US); David Primer, Golden, CO (US); Scott A. Savage, Yardley, PA (US); John Fitzgerald Traverse, Holliston, MA (US); Christopher R. Wilbert, Robbinsville, NJ (US)

(21) Appl. No.: 19/201,806

(22) Filed: May 7, 2025

Related U.S. Application Data
(60) Provisional application No. 63/643,784, filed on May 7, 2024.

(51) Int. Cl. C07D 401/14

(52) U.S. Cl. CPC C07D 401/14

(57) ABSTRACT

Provided herein are processes for preparing 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3- ylamino)phenyl)acetamide or a stereoisomer or mixture of stereoisomers thereof.

### RELATED APPLICATIONS

This application claims the benefit of U.S. Provisional Application No. 63/643,784, filed May 7, 2024, the disclosure of which is incorporated herein by reference in its entirety.

### FIELD

Provided herein are processes for preparing 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or mixture of stereoisomers thereof.

### BACKGROUND

2-((R)-4-(2-(4-(3-(4-Cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or mixture of stereoisomers thereof, pharmaceutically acceptable salt or tautomer thereof has been reported for treating, preventing, managing, and/or ameliorating an androgen receptor mediated disease, or one or more symptoms or causes thereof. Exemplary formulations of the compound, methods of use and methods of synthesis of the compound are disclosed in U.S. Pat. Nos. 11,149,007 and 11,873,283; and International Application Publicaiton No. WO2024/102706, the disclosures of each of which are incorporated herein by reference in their entireties.

There is a need for a more efficient, commercially viable, safe, less toxic, and environmentally friendly process for synthesis of 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or mixture of stereoisomers thereof.

### BRIEF SUMMARY

Provided herein are efficient processes for preparing 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or mixture of stereoisomers thereof. In one embodiment, provided herein is a process for preparing 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide as a diastereomeric mixture (Compound 1). In one embodiment, provided herein is a process for preparing 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(((R)-2,6-dioxopiperidin-3-yl)amino)phenyl) acetamide (Compound 1-1).

In one embodiment, provided herein is a process for preparing Compound 1 or a stereoisomer or mixture of stereoisomers thereof.

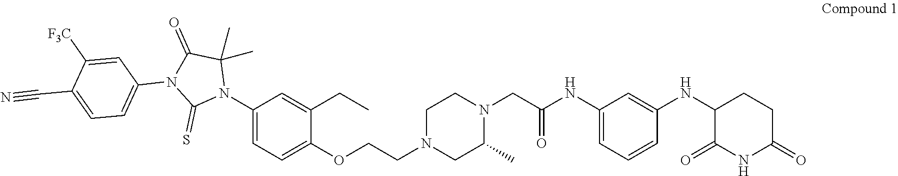 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1]

comprising contacting an acid salt of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid with an acid salt of 3-((3-aminophenyl)amino) piperidine-2,6-dione in the presence of a base in a solvent under conditions suitable to provide Compound 1 or a stereoisomer or mixture of stereoisomers thereof.

In one embodiment, provided herein is a process for preparing Compound 1, comprising a step of contacting Compound W

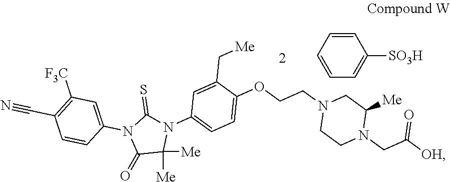

with Compound J

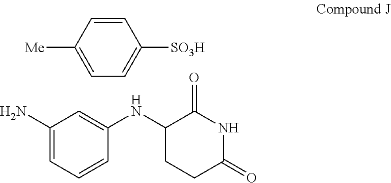

in the presence of a base in a solvent under conditions suitable to provide Compound 1 or a stereoisomer or mixture of stereoisomers thereof.

In one embodiment, provided herein is a process for preparing Compound W, comprising an acid mediated deprotection of the tert-butyl ester from Compound U

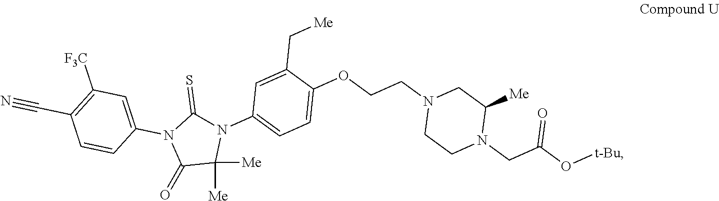 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

under conditions suitable to provide Compound W.

In one embodiment, provided herein is a process for preparing Compound U comprising contacting Compound P

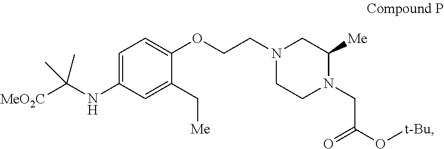 [SMILES: CCc1cc(NC(C)(C)C(=O)OC)ccc1OCCN1CCN(CC(=O)OC(C)(C)C)[C@H](C)C1]

with Compound S

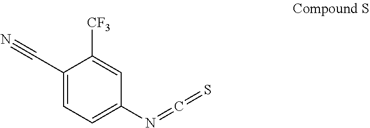 [SMILES: N#Cc1ccc(N=C=S)cc1C(F)(F)F]

under conditions suitable to provide Compound U.

In one embodiment, provided herein is a process for preparing Compound P, comprising contacting Compound N

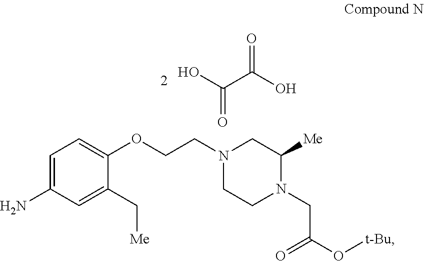

with methyl 2-bromo-2-methylpropionate under conditions suitable to provide Compound P.

In one embodiment, provided herein is a process for preparing Compound P, comprising contacting Compound N1

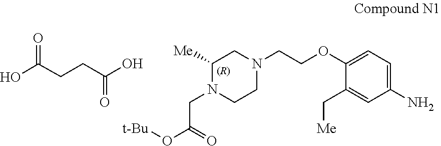

with methyl 2-bromo-2-methylpropionate under conditions suitable to provide Compound P.

In one embodiment, provided herein is a process for preparing Compound N1, comprising contacting Compound T

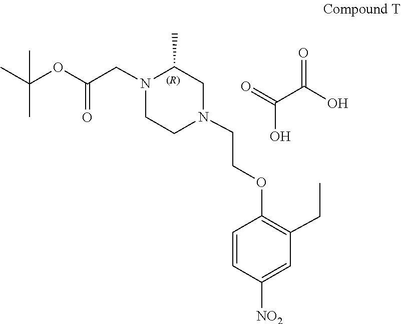

with platinum catalyst under and succinic acid conditions suitable to provide Compound N1.

In one embodiment, provided herein is a process for preparing Compound J, comprising contacting Compound H

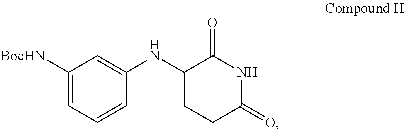 [SMILES: CC(C)(C)OC(=O)Nc1cccc(NC2CCC(=O)NC2=O)c1]

with p-toluenesulfonic acid monohydrate under conditions suitable to provide Compound J.

In one embodiment, provided herein is a process for preparing Compound H, comprising contacting an acid salt of 3-aminopiperidine-2,6-dione

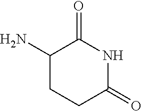

with tert-butyl (3-bromophenyl) carbamate

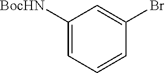 [SMILES: CC(C)(C)OC(=O)Nc1cccc(Br)c1]

under conditions suitable to provide Compound H.

In one embodiment, provided herein is a process for preparing Compound 1 comprising
a) contacting Compound N

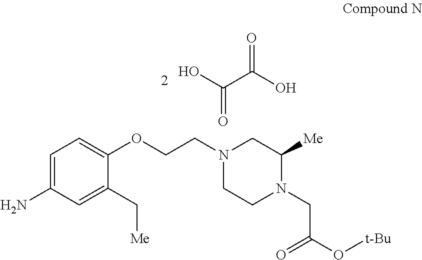

with methyl 2-bromo-2-methylpropionate under conditions suitable to provide Compound P

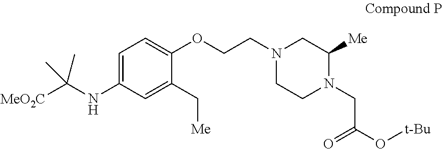 [SMILES: CCc1cc(NC(C)(C)C(=O)OC)ccc1OCCN1CCN(CC(=O)OC(C)(C)C)[C@H](C)C1]

b) contacting Compound P with Compound S

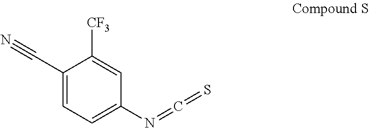 [SMILES: N#Cc1ccc(N=C=S)cc1C(F)(F)F]

under conditions suitable to provide Compound U,

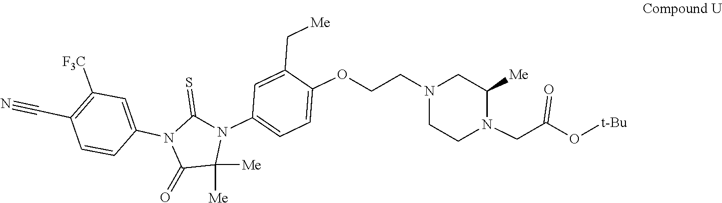 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

c) contacting Compound U with an acid to deprotect the tert-butyl ester group under conditions suitable to provide Compound W

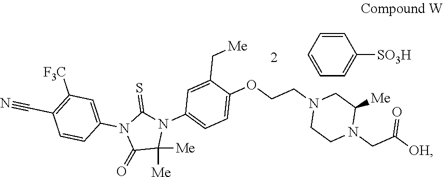

and d) contacting Compound W with Compound J

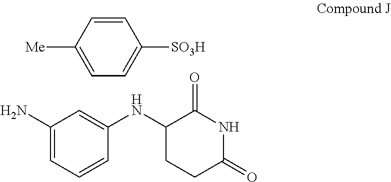

under conditions suitable to provide Compound 1.

In one embodiment, provided herein is a process for preparing Compound 1 comprising
a) contacting Compound N1

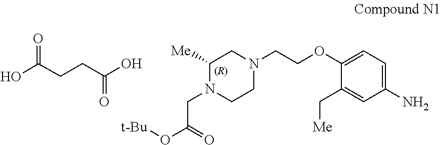

with methyl 2-bromo-2-methylpropionate under conditions suitable to provide Compound P

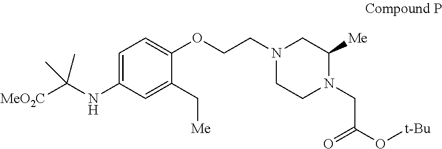 [SMILES: CCc1cc(NC(C)(C)C(=O)OC)ccc1OCCN1CCN(CC(=O)OC(C)(C)C)[C@H](C)C1]

b) contacting Compound P with Compound S

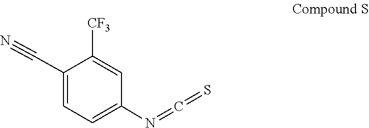 [SMILES: N#Cc1ccc(N=C=S)cc1C(F)(F)F]

under conditions suitable to provide Compound U,

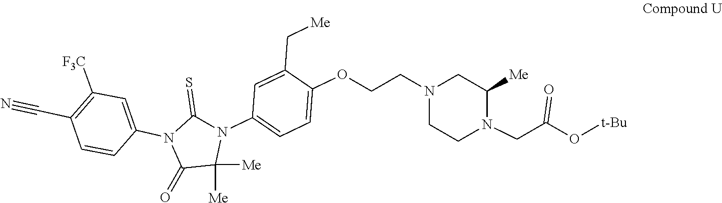 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

c) contacting Compound U with an acid to deprotect the tert-butyl ester group under conditions suitable to provide Compound W

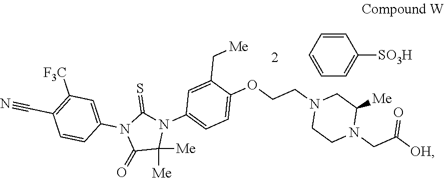

and d) contacting Compound W with Compound J

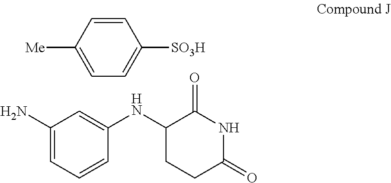

under conditions suitable to provide Compound 1.

In one embodiment, provided herein is a process for preparing Compound 1-1

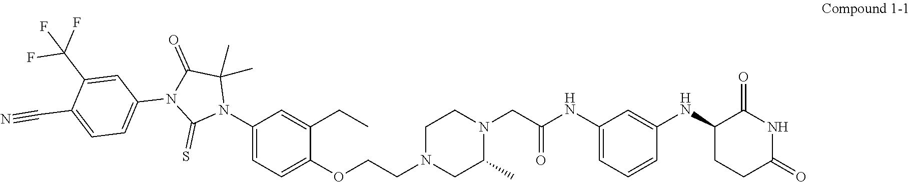 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

comprising
a) contacting an acid salt of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid with an acid salt of (R)-3-((3-aminophenyl)amino) piperidine-2,6-dione in the presence of a base in a solvent and benzenesulfonic acid (BSA) under conditions suitable to provide BSA salt of Compound 1-1;
b) contacting the BSA salt of Compound 1-1 with a buffer in a solvent to obtain a solution comprising Compound 1-1; and
c) precipitating Compound 1-1 from the solution.

In one embodiment, provided herein is a process for preparing Compound 1-1 comprising
a) contacting Compound N

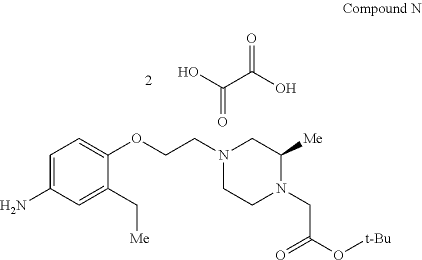

with methyl 2-bromo-2-methylpropionate under conditions suitable to provide Compound P

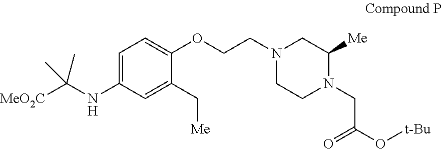 [SMILES: CCc1cc(NC(C)(C)C(=O)OC)ccc1OCCN1CCN(CC(=O)OC(C)(C)C)[C@H](C)C1]

b) contacting Compound P with Compound S

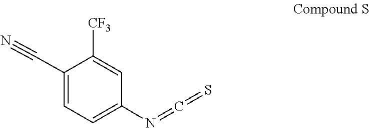 [SMILES: N#Cc1ccc(N=C=S)cc1C(F)(F)F]

under conditions suitable to provide Compound U,

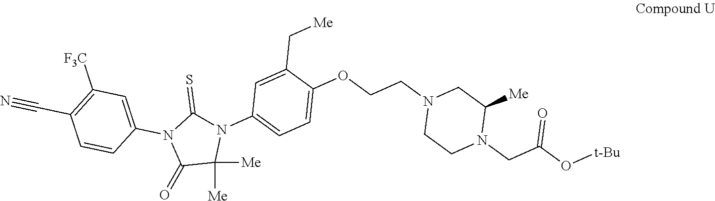 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

c) contacting Compound U with an acid to deprotect the tert-butyl ester group under conditions suitable to provide Compound W

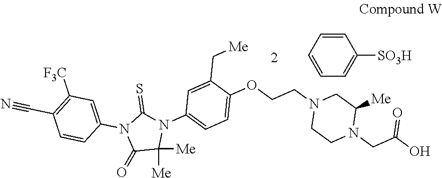

d) contacting Compound W with Compound (R)-J

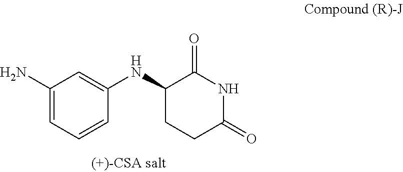 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1

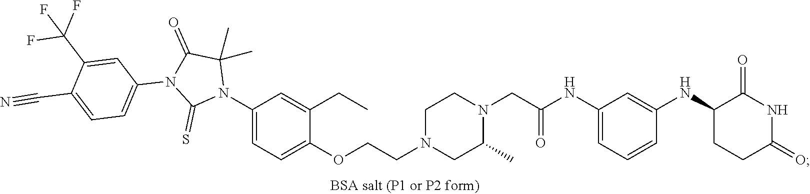 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

e) contacting the BSA salt of Compound 1-1 with a buffer in a solvent to obtain a solution comprising Compound 1-1; and
f) contacting the solution with water/acetone mixture to obtain Compound 1-1.

In certain embodiments, the processes for preparing Compound 1 or further comprises
a) contacting an acid salt of 3-aminopiperidine-2,6-dione

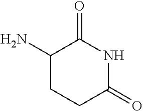

with tert-butyl (3-bromophenyl) carbamate

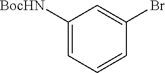 [SMILES: CC(C)(C)OC(=O)Nc1cccc(Br)c1]

under conditions suitable to provide Compound H,

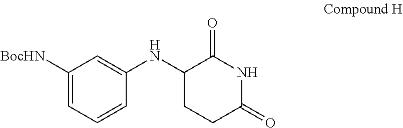 [SMILES: CC(C)(C)OC(=O)Nc1cccc(NC2CCC(=O)NC2=O)c1]

b) contacting Compound H with with p-toluenesulfonic acid monohydrate under conditions suitable to provide Compound J

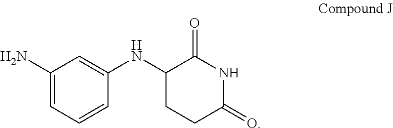 [SMILES: Nc1cccc(NC2CCC(=O)NC2=O)c1]

In one embodiment, provided herein is a process for preparing Compound viii

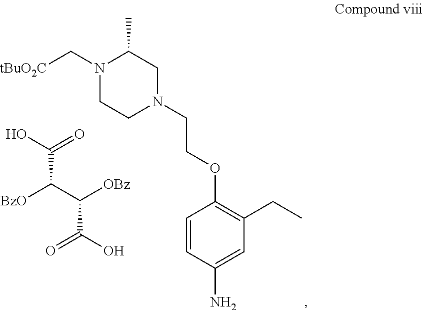

wherein the process comprises contacting Compound vii

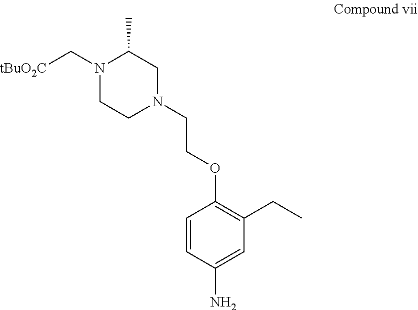 [SMILES: CCc1cc(N)ccc1OCCN1CCN(CC(=O)OC(C)(C)C)[C@H](C)C1]

with (+)-dibenzoyl-D-tartaric acid to provide Compound viii.

Further provided herein are chemical intermediates useful in the processes provided herein.

2-((R)-4-(2-(4-(3-(4-Cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or a mixture of stereoisomers thereof are useful in methods of treating an androgen receptor (AR) mediated disease. Also disclosed are 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or a mixture of stereoisomers thereof for use in methods of treating an androgen receptor (AR) mediated disease. In certain embodiments, the AR mediated disease is prostate cancer. In some such embodiments, the prostate cancer is castration resistant prostate cancer (CRPC).

These and other aspects of the subject matter described herein will become evident upon reference to the following detailed description.

### BRIEF DESCRIPTION OF DRAWINGS

FIG. 1 provides a ¹H NMR spectrum of tert-butyl (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetate.

FIG. 2 depicts a ¹H NMR spectrum of benzene sulfonate salt of tert-butyl (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetate.

FIG. 3 depicts a 14C NMR spectrum of benzene sulfonate salt of tert-butyl (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetate.

### DETAILED DESCRIPTION

### Definitions

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In general, the technical teaching of one embodiment can be combined with that disclosed in other embodiments provided herein.

The use of the word “a” or “an” when used.in conjunction with the term “comprising” in the claims and/or the specification can mean “one”, but it is also consistent with the meaning of “one or more”, “at least one” and “one or more than one.”

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive. E.g., “treating, preventing or managing” or similar listings means: “treating; preventing; managing; treating and preventing; treating and managing; preventing and managing; treating, preventing and managing”.

The term “Compound 1” refers to “2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide” having the structure:

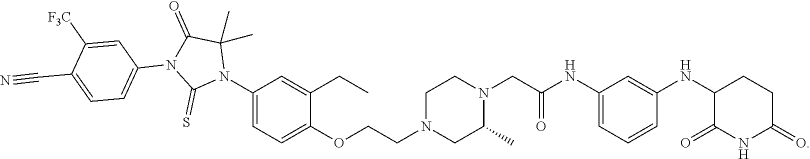 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1]

The term “Compound 1-1” refers to “2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(((R)-2,6-dioxopiperidin-3-yl)amino)phenyl) acetamide” having the structure:

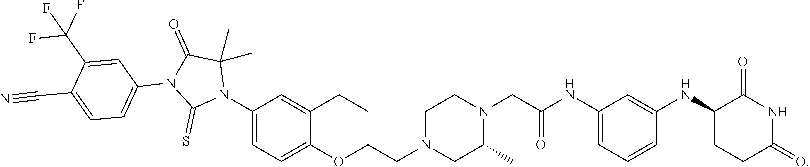 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

Unless specifically stated otherwise, where a compound may assume alternative tautomeric, regioisomeric and/or stereoisomeric forms, all alternative isomers are intended to be encompassed within the scope of the claimed subject matter. For example, where a compound can have one of two tautomeric forms, it is intended that both tautomers be encompassed herein.

As used herein and unless otherwise indicated, the term “stereoisomer” or “stereoisomerically pure” means one stereoisomer of 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide that is substantially free of other stereoisomers of that compound. For example, a stereoisomerically pure compound will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. 2-((R)-4-(2-(4-(3-(4-Cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide can occur as racemate, individual diastereomers, and mixtures thereof. All such isomeric forms are included within the embodiments disclosed herein, including mixtures thereof.

It should also be noted the compounds herein can contain unnatural proportions of atomic isotopes at one or more of the atoms. For example, the compounds may be radiolabeled with radioactive isotopes, such as for example deuterium (²H), tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds described herein, whether radioactive or not, are intended to be encompassed within the scope of the embodiments provided herein. In some embodiments, there are provided isotopologues of the compounds described herein, for example, the isotopologues are deuterium, carbon-13, and/or nitrogen-15 enriched Compound 1. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound referred to herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof, may otherwise vary independently from the selection of the pharmaceutically acceptable salt of the respective compound.

As used herein and unless otherwise indicated, the term “process(es) provided herein” refers to the methods disclosed herein which are useful for preparing 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide or a stereoisomer or mixture of stereoisomers thereof. Modifications to the methods disclosed herein (e.g., starting materials, reagents, protecting groups, solvents, temperatures, reaction times, purification) are also encompassed by the present embodiments.

As used herein and unless otherwise indicated, the term “adding” or the like means contacting one reactant, reagent, solvent, catalyst, or the like with another reactant, reagent, solvent, catalyst, or the like. Reactants, reagents, solvents, catalysts, or the like can be added individually, simultaneously, or separately and can be added in any order. They can be added in the presence or absence of heat and can optionally be added under an inert atmosphere.

As used herein and unless otherwise indicated, a reaction that is “substantially complete” or is driven to “substantial completion” means that the reaction contains more than about 80% by percent yield, in one embodiment, more than about 90% by percent yield, in another embodiment, more than about 95% by percent yield, and in yet another embodiment, more than about 97% by percent yield of the desired product.

The term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

The term “androgen receptor” or “AR” or “NR3C4” as used herein refers to a nuclear hormone receptor activated by binding of the androgenic hormones, including testosterone or dihydrotestosterone. The term “androgen receptor” may refer to the nucleotide sequence or protein sequence of human androgen receptor (e.g., Entrez 367, Uniprot P10275, RefSeq NM_000044, or RefSeq NP_000035).

The term “AR-full length” (AR-FL) as used herein refers to AR protein that contains all four functional domains, including the N-terminal transactivation domain (NTD, exon 1), the DNA-binding domain (DBD, exons 2-3), the hinge domain (exon 4), and the C-terminal ligand binding domain (LBD, exons 4-8).

The term “castration resistant prostate cancer” (CRPC) refers to advanced prostate cancer that is worsening or progressing while the patient remains on androgen deprivation therapy or other therapies to reduce testosterone, or prostate cancer which is considered hormone refractory, hormone naive, androgen independent or chemical or surgical castration resistant. Castration resistant prostate cancer (CRPC) is an advanced prostate cancer that developed despite ongoing ADT and/or surgical castration. Castration resistant prostate cancer is defined as prostate cancer that continues to progress or worsen or adversely affect the health of the patient despite prior surgical castration, continued treatment with gonadotropin releasing hormone agonists (e.g., leuprolide) or antagonists (e.g., degarelix or abarelix), antiandrogens (e.g., bicalutamide, flutamide, enzalutamide, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®), cabozantinib (Cometriq®, also known as XL184)) or other prostate cancer therapies (e.g., vaccines (sipuleucel-T (Provenge®), GVAX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

As used herein, and unless otherwise specified, the expression “unit dose” refers to a physically discrete unit of a formulation appropriate for a subject to be treated (e.g., for a single dose); each unit containing a predetermined quantity of an active agent selected to produce a desired therapeutic effect (it being understood that multiple doses may be required to achieve a desired or optimum effect), optionally together with a pharmaceutically acceptable carrier, which may be provided in a predetermined amount. The unit dose may be, for example, a volume of liquid (e.g. an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, ½ normal saline, sucrose, glucose, bulking agents, buffers, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms “bulking agent”, and “buffer” are used in accordance with the plain and ordinary meaning within the art.

As used herein, and unless otherwise specified, the term “about,” when used in connection with doses, amounts, or weight percent of ingredients of a composition or a dosage form, means dose, amount, or weight percent that is recognized by those of ordinary skill in the art to provide a pharmacological effect equivalent to that obtained from the specified dose, amount, or weight percent is encompassed. Specifically, the term “about” contemplates a dose, amount, or weight percent within 30%, 25%, 20%, 15%, 10%, or 5% of the specified dose, amount, or weight percent is encompassed.

As used herein, “administer” or “administration” refers to the act of physically delivering a substance as it exists outside the body into a subject. Administration includes all forms known in the art for delivering therapeutic agents, including but not limited to topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

By “co-administer” it is meant that compounds, compositions or agents described herein are administered at the same time, just prior to, or just after the administration of one or more additional compounds, compositions or agents, including for example an anti-cancer agent. Co-administration is meant to include simultaneous or sequential administration of compounds, compositions or agents individually or in combination (more than one compound or agent). Co-administration includes administering two compounds, compositions or agents simultaneously, approximately simultaneously (e.g., within about 1, 5, 10, 15, 20, or 30 minutes of each other), or sequentially in any order. Thus, co-administration can include administering one active agent (e.g. a compound described herein) within 0.5, 1, 2, 4, 6, 8, 10, 12, 16, 20, or 24 hours of a second active agent. Co-administration can also be accomplished by co-formulation, e.g., preparing a single dosage form including both active agents. The active agents can be formulated separately. In such instances, the active agents are admixed and included together in the final form of the dosage unit. Alternatively, co-administration as described herein can include administering two separate unit dosage forms of at least two separate active agents (e.g., a compound and a second active agent described herein).

An “effective amount” is an amount sufficient to achieve the effect for which it is administered (e.g., treat a disease or reduce one or more symptoms of a disease or condition). Thus, administration of an “amount” of a compound described herein to a subject refers to administration of “an amount effective,” to achieve the desired therapeutic result. A “therapeutically effective amount” of a compound described herein for purposes herein is thus determined by such considerations as are known in the art. The term “therapeutically effective amount” of a composition described herein refers to the amount of the composition that, when administered, is sufficient to treat one or more of the symptoms of a disease described herein. Administration of a compound described herein can be determined according to factors such as, for example, the disease state, age, sex, and weight of the individual. A therapeutically effective amount also refers to any toxic or detrimental effects of a compound provided herein are outweighed by the therapeutically beneficial effects.

The term “relapsed” refers to a situation where patients who have had a remission of leukemia after therapy have a return of leukemia cells in the marrow and a decrease in normal blood cells.

The term “refractory or resistant” refers to a circumstance where patients, even after intensive treatment, have residual leukemia cells in their marrow.

The term “drug resistance” refers to the condition when a disease does not respond to the treatment of a certain drug or drugs. Drug resistance can be either intrinsic, which means the disease has never been responsive to the particular drug or drugs, or it can be acquired, which means the disease ceases responding to particular a drug or drugs that the disease had previously responded to. In certain embodiments, drug resistance is intrinsic. In certain embodiments, the drug resistance is acquired.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11:942-944).

If there is a discrepancy between a depicted structure and a name given that structure, the depicted structure is to be accorded more weight. Furthermore, if the stereochemistry of a structure or a portion thereof is not indicated, e.g., with bold or dashed lines, the structure or portion thereof is to be interpreted as encompassing all stereoisomers of it.

### Processes

In one embodiment, provided herein is a process for preparing Compound 1 or a stereoisomer or mixture of stereoisomers thereof.

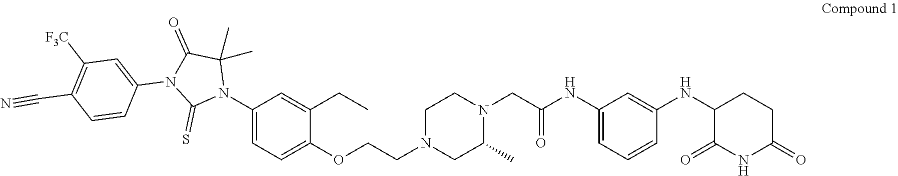 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1]

comprising contacting an acid salt of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid with an acid salt of 3-((3-aminophenyl)amino) piperidine-2,6-dione in the presence of a base in a solvent under conditions suitable to provide Compound 1 or a stereoisomer or mixture of stereoisomers thereof. Exemplary bases for use in the process include N-methyl imidazole, triethyl amine and pyridine. In one embodiment, the base is N-methyl imidazole. Exemplary solvents for use in the process include THE, DMF, EtOAc, MeCN, DMAc, DMSO or a mixture thereof, such as a mixture of EtOAc/THF. In one embodiment, the solvent comprises THF.

In one embodiment, provided herein is a process for preparing Compound 1, comprising a step of contacting Compound W

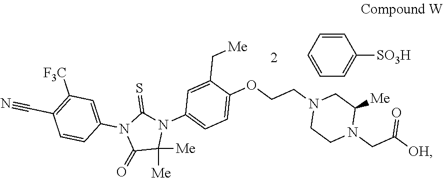

with Compound J

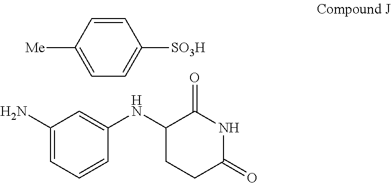

in the presence of a base in a solvent under conditions suitable to provide Compound 1 or a stereoisomer or mixture of stereoisomers thereof. Exemplary bases for use in the process include N-methyl imidazole, triethyl amine and pyridine. In one embodiment, the base is N-methyl imidazole. Exemplary solvents for use in the process include THE, DMF, EtOAc, MeCN, DMAc, DMSO and a mixture thereof, such as a mixture of EtOAc/THF. In one embodiment, the solvent comprises THF.

In one aspect of the process, Compound W is contacted with Compound J in the presence of a coupling agent. Exemplary coupling agents for use in the process include propanephosphonic acid anhydride (T3P), Hexafluorophosphate Azabenzotriazole Tetramethyl Uronium (HATU), 1-Ethyl-3-(3-dimethylaminopropyl) carbodiimide (EDC), (1-Cyano-2-ethoxy-2-oxoethylidenaminooxy) dimethylamino-morpholino-carbenium hexafluorophosphate (COMU) and (benzotriazol-1-yloxy)tripyrrolidinophosphonium hexafluorophosphate (PyBop). In one embodiment, the coupling agent is propanephosphonic acid anhydride (T3P).

In one aspect of the process, 1 equivalent of Compound W is contacted with about 1.08 to 1.5 equivalents of Compound J in the presence of a base in a solvent under conditions suitable to provide Compound 1. In one aspect of the process, 1 equivalent of Compound W is contacted with about 1.08 equivalents of Compound J in the presence of a base in a solvent under conditions suitable to provide Compound 1.

In one aspect, the process comprises contacting Compound W, Compound J and N-methyl imidazole in THF in the presence of T3P under conditions suitable to provide Compound 1. In one aspect, the process comprises contacting 1 equivalent of Compound W, about 1.08 equivalents Compound J and N-methyl imidazole in THF in the presence of T3P under conditions suitable to provide Compound 1.

In one aspect, the process comprises contacting Compound W, Compound J and N-methyl imidazole in THF in the presence of T3P at about 25° C. under conditions suitable to provide Compound 1. In one aspect, the process comprises contacting Compound W, Compound J and N-methyl imidazole in THF in the presence of T3P at about 25° C. for about 15 minutes to about 48 hours under conditions suitable to provide Compound 1. In one aspect, the process comprises contacting Compound W, Compound J and N-methyl imidazole in THF in the presence of T3P at about 25° C. for about 8 hours under conditions suitable to provide Compound 1.

In one embodiment, provided herein is a process for preparing Compound W, comprising an acid mediated deprotection of the tert-butyl ester from Compound U

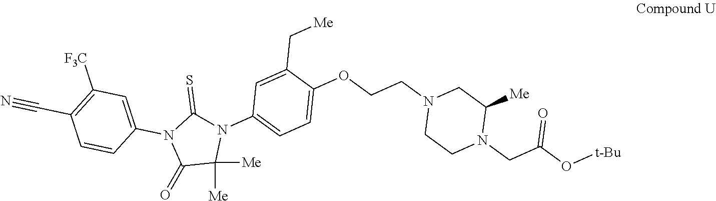 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

under conditions suitable to provide Compound W. In one aspect, the process comprises contacting Compound U with BSA in a solvent under conditions suitable to provide Compound W. In one embodiment, the solvent comprises acetonitrile. In one embodiment, the process comprises contacting Compound U with BSA in acetonitrile at about 50° C. under conditions suitable to provide Compound W. In one embodiment, the process comprises contacting Compound U with BSA in acetonitrile at about 50° C. for about 30 hours under conditions suitable to provide Compound W.

In one embodiment, provided herein is a process for preparing Compound U comprising contacting Compound P

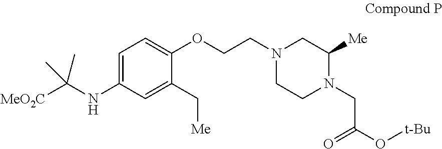 [SMILES: CCc1cc(NC(C)(C)C(=O)OC)ccc1OCCN1CCN(CC(=O)OC(C)(C)C)[C@H](C)C1]

with Compound S

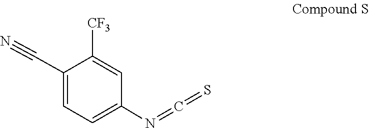 [SMILES: N#Cc1ccc(N=C=S)cc1C(F)(F)F]

in a solvent under conditions suitable to provide Compound U. In one aspect, the process comprises contacting Compound P with Compound S in a solvent under conditions suitable to provide Compound U. In one embodiment, the solvent comprises 2-methyltetrahydrofuran. In one embodiment, the process comprises contacting Compound P with Compound S in 2-methyltetrahydrofuran at about 10-30° C. under conditions suitable to provide Compound U. In one embodiment, the process comprises contacting Compound P with Compound S in 2-methyltetrahydrofuran at about 15-20° C. under conditions suitable to provide Compound U. In one embodiment, the process comprises contacting Compound P with Compound S in 2-methyltetrahydrofuran at about 10-30° C. for about 4-30 hours under conditions suitable to provide Compound U. In one embodiment, the process comprises contacting Compound P with Compound S in 2-methyltetrahydrofuran at about 15-20° C. for about 4-30 hours under conditions suitable to provide Compound U. In one embodiment, the process comprises contacting Compound P with Compound S in 2-methyltetrahydrofuran at about 15-20° C. for about 10-12 hours under conditions suitable to provide Compound U.

In one embodiment, provided herein is a process for preparing Compound P, comprising contacting Compound N

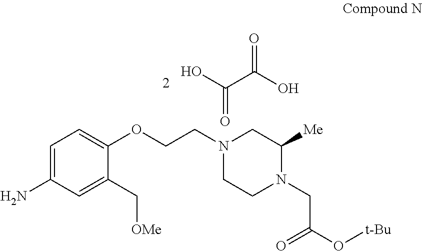

with methyl 2-bromo-2-methylpropionate in a solvent in the presence of a base under conditions suitable to provide Compound P. Exemplary bases for use in the process include K₂CO₃, TEA, DIEA, DBU, Na₂CO₃, Cs₂CO₃, Na₃PO₄ and K₃PO₄. In one embodiment, the base is K₂CO₃. Exemplary solvents for use in the process include NMP, CH₃CN, MeTHF, DMF, DMSO and DMAc. In one embodiment, the solvent comprises NMP. In certain embodiments, the process comprises contacting Compound N with methyl 2-bromo-2-methylpropionate in a solvent in the presence of a base and a catalyst under conditions suitable to provide Compound P. In one embodiment, the catalyst is tetrabutylammonium iodide or tetrabutylammonium bromide. In one embodiment, the process comprises contacting Compound N with methyl 2-bromo-2-methylpropionate in NMP in the presence of K₂CO₃ at about 55-65° C. under conditions suitable to provide Compound P. In one embodiment, the process comprises contacting Compound N with methyl 2-bromo-2-methylpropionate in NMP in the presence of K₂CO₃ at about 55-65° C. for at least about 38 hours under conditions suitable to provide Compound P.

In one embodiment, provided herein is a process for preparing Compound N, comprising contacting Compound L

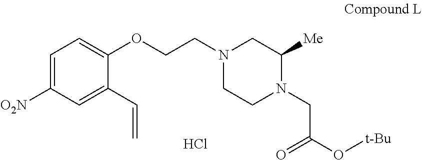 [SMILES: C=Cc1cc([N+](=O)[O-])ccc1OCCN1CCN(CC(=O)OC(C)(C)C)[C@H](C)C1 ; Cl]

with a palladium catalyst in a solvent under conditions suitable to provide Compound N. In one embodiment, the solvent comprises 2-methyltetrahydrofuran. In certain embodiments, the process comprises contacting Compound L with a palladium catalyst in 2-methyltetrahydrofuran at about 20-25° C. In one embodiment, the process further comprises contacting with oxalic acid under conditions suitable to provide Compound N.

In one embodiment, provided herein is a process for preparing Compound L via a Suzuki coupling reaction. In one embodiment, the process comprises contacting Compound K

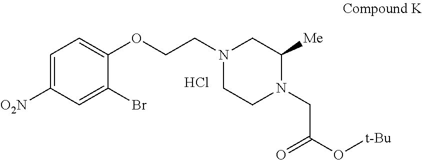 [SMILES: C[C@@H]1CN(CCOc2ccc([N+](=O)[O-])cc2Br)CCN1CC(=O)OC(C)(C)C ; Cl]

with vinyl dioxaborolane in the presence of a palladium catalyst and a base in a solvent under conditions suitable to provide Compound L. Exemplary solvents for use in this process include dimethylformamide, isopropanol, dioxane, toluene, dimethylacetamide, tetrahydrofuran, acetonitrile, isopropyl acetate, dimethyl sulfoxide, acetone, methanol, methyl t-butyl ether and a combination thereof, with or without the presence of water. In one embodiment, the solvent comprises a mixture of tetrahydrofuran and water. Exemplary palladium catalysts for use in this process include dichloro [1,1′-bis(diphenylphosphino)-ferrocene]palladium (II) dichloro-methane), palladium (dba) 2/tri-o-tolylphosphine, dichloro [1,1′-bis(ditert-butylphosphino) ferrocene]palladium, dichlorobis (p-dimethylamino phenylditbutylphosphine) palladium (II), tetrakis (triphenylphosphine) palladium (0), dichloro (2 diphenylphosphino ethyl trimethyl-ammonium) palladium, or palladium (II) acetate/4,5-bis(diphenylphosphino)-9,9-dimethylxanthene. In one embodiment, the catalyst is Pd (DPPF) C12 (dichloro [1,1′-bis(diphenylphosphino)-ferrocene]palladium (II) dichloro-methane). Exemplary bases suitable for use in the process include triethylamine, sodium carbonate, diisopropylethyl amine, piperidine, pyridine, cesium carbonate, potassium carbonate, potassium phosphate, or sodium hydroxide. In one embodiment, the base is potassium phosphate.

In certain embodiments, the process comprises contacting Compound K with 4,4,5,5-tetramethyl-2-vinyl-1,3,2-dioxaborolane in the presence of Pd (DPPF) Cl₂ catalyst and in tetrahydrofuran and water at about 70-80° C. under conditions suitable to provide Compound L.

In one embodiment, provided herein is a process for preparing Compound K comprising contacting Compound E

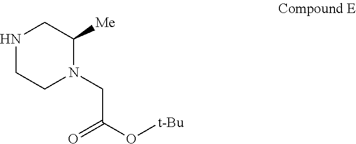 [SMILES: C[C@@H]1CNCCN1CC(=O)OC(C)(C)C]

with Compound I

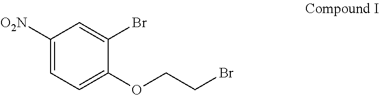 [SMILES: O=[N+]([O-])c1ccc(OCCBr)c(Br)c1]

in the presence of a base in a solvent under conditions suitable to provide Compound K. In one embodiment, the solvent comprises a mixture of tetrahydrofuran and water. In one embodiment, the base is potassium carbonate. In certain embodiments, the process comprises contacting Compound E with Compound I in the presence of potassium carbonate in a mixture of tetrahydrofuran and water at about 75-85° C. under conditions suitable to provide Compound K.

In one embodiment, provided herein is a process for preparing Compound E comprising contacting Compound AA

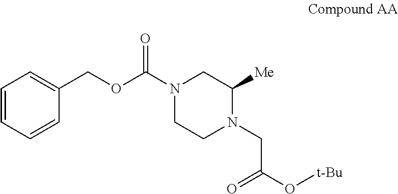 [SMILES: C[C@@H]1CN(C(=O)OCc2ccccc2)CCN1CC(=O)OC(C)(C)C]

with a palladium catalyst in a solvent under conditions suitable to provide Compound E. In one embodiment, the solvent comprises 2-methyltetrahydrofuran. In certain embodiments, the process comprises contacting Compound AA with a palladium catalyst in 2-methyltetrahydrofuran at about 25-35° C. under conditions suitable to provide Compound E.

In one embodiment, provided herein is a process for preparing Compound AA comprising contacting Compound A′

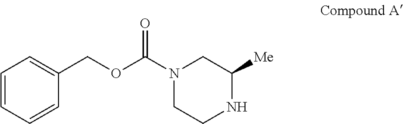 [SMILES: C[C@@H]1CN(C(=O)OCc2ccccc2)CCN1]

with tert-butyl 2-bromoacetate in a solvent under conditions suitable to provide Compound AA. In one embodiment, the solvent comprises 2-methyltetrahydrofuran and water. In certain embodiments, the process comprises contacting Compound A′ with tert-butyl 2-bromoacetate and a base in a solvent comprising 2-methyltetrahydrofuran and water under conditions suitable to provide Compound AA. In certain embodiments, the process comprises contacting Compound A′ with tert-butyl 2-bromoacetate and a potassium carbonate in a solvent comprising 2-methyltetrahydrofuran and water at about 45-50° C. under conditions suitable to provide Compound AA.

In one embodiment, provided herein is a process for preparing Compound J, comprising contacting Compound H

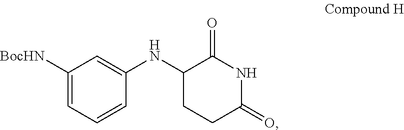 [SMILES: CC(C)(C)OC(=O)Nc1cccc(NC2CCC(=O)NC2=O)c1]

with p-toluenesulfonic acid monohydrate under conditions suitable to provide Compound J. In one aspect, the process comprises contacting Compound H with p-toluenesulfonic acid monohydrate in acetonitrile at 70 to 75° C. to provide Compound J. In one aspect, the process comprises contacting Compound H with p-toluenesulfonic acid monohydrate in acetonitrile/water mixture at 60 to 70° C. to provide Compound J.

In one embodiment, provided herein is a process for preparing Compound H, comprising contacting an acid salt of 3-aminopiperidine-2,6-dione

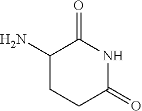

with tert-butyl (3-bromophenyl) carbamate

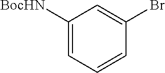 [SMILES: CC(C)(C)OC(=O)Nc1cccc(Br)c1]

under conditions suitable to provide Compound H. In one embodiment, the process comprises contacting a hydrochloride salt of 3-aminopiperidine-2,6-dione with tert-butyl (3-bromophenyl) carbamate in a solvent in the presence of a base and a catalyst under conditions suitable to provide Compound H. In one embodiment, the base is KOtBu. In one embodiment, the solvent comprises DMAC and tert-amyl alcohol. In one embodiment, the solvent comprises NMP and tert-amyl alcohol. In one embodiment, the solvent comprises tert-amyl alcohol. In one embodiment, the catalyst comprises allylpalladium chloride dimer, AgOTf and tBuXPhos. In one embodiment, the process further comprises preparing the catalyst by contacting allylpalladium chloride dimer, AgOTf and tBuXPhos under conditions suitable to provide the catalyst.

In one embodiment, provided herein is a process for preparing Compound H, comprising contacting 3-bromopiperidine-2,6-dione (Compound D)

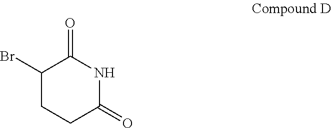

with tert-butyl (3-aminophenyl) carbamate

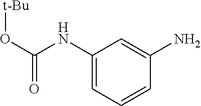 [SMILES: CC(C)(C)OC(=O)Nc1cccc(N)c1]

under conditions suitable to provide Compound H. In one embodiment, the process comprises contacting Compound A with tert-butyl (3-aminophenyl) carbamate in a solvent in the presence of a base under conditions suitable to provide Compound H. In one embodiment, the base is 2,6-lutidine. In one embodiment, the solvent comprises 2-methyltetrahydrofuran.

In one embodiment, provided herein is a process for preparing Compound D, comprising contacting 6,7-dihydro-5H-oxazolo[3,2-a]pyridine-2,3,5-trione (Compound C)

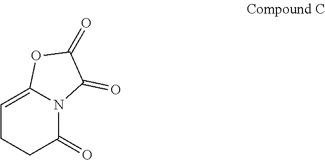 [SMILES: O=C1OC2=CCCC(=O)N2C1=O]

with n-bromosuccinimide (NBS) under conditions suitable to provide Compound D. In one embodiment, the process comprises contacting Compound C with n-bromosuccinimide in a solvent comprising isopropyl acetate and water.

In one embodiment, provided herein is a process for preparing Compound C, comprising contacting piperidine-2,6-dione (Compound A)

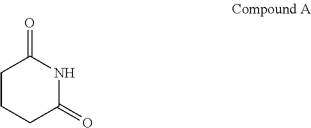

with oxalyl chloride under conditions suitable to provide Compound C. In one embodiment, the process comprises contacting Compound A with oxalyl chloride in isopropyl acetate.

In one embodiment, provided herein is a process for preparing Compound 1 comprising
a) contacting Compound N

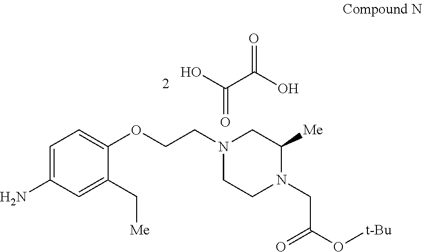

with methyl 2-bromo-2-methylpropionate under conditions suitable to provide Compound P

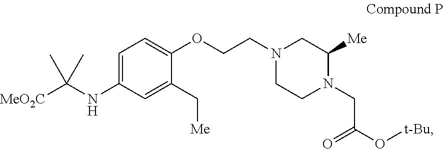 [SMILES: CCc1cc(NC(C)(C)C(=O)OC)ccc1OCCN1CCN(CC(=O)OC(C)(C)C)[C@H](C)C1]

b) contacting Compound P with Compound S

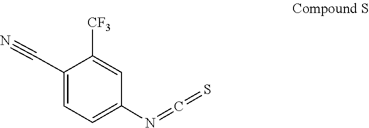 [SMILES: N#Cc1ccc(N=C=S)cc1C(F)(F)F]

under conditions suitable to provide Compound U

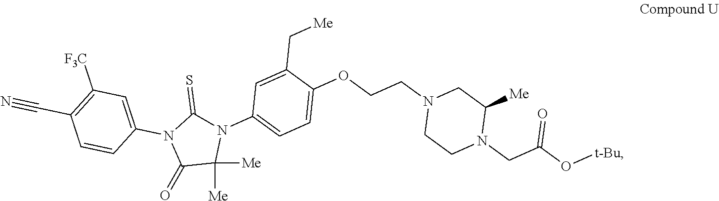 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

c) contacting Compound U with an acid to deprotect the tert-butyl ester group under conditions suitable to provide Compound W

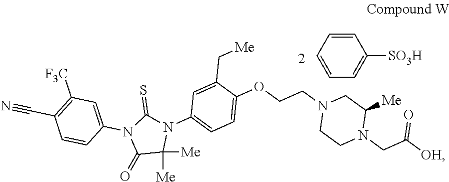

and d) contacting Compound W with Compound J

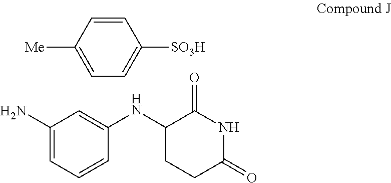

under conditions suitable to provide Compound 1.

In certain embodiments, the process for preparing Compound 1 further comprises
i) contacting an acid salt of 3-aminopiperidine-2,6-dione

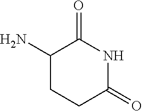

with tert-butyl (3-bromophenyl) carbamate

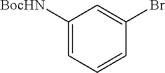 [SMILES: CC(C)(C)OC(=O)Nc1cccc(Br)c1]

under conditions suitable to provide Compound H

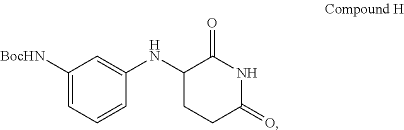 [SMILES: CC(C)(C)OC(=O)Nc1cccc(NC2CCC(=O)NC2=O)c1]

and ii) contacting Compound H with p-toluenesulfonic acid monohydrate under conditions suitable to provide Compound J

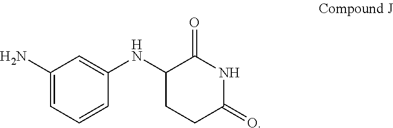 [SMILES: Nc1cccc(NC2CCC(=O)NC2=O)c1]

In certain embodiments, the process for preparing Compound 1 further comprises
i) contacting piperidine-2,6-dione (Compound A)

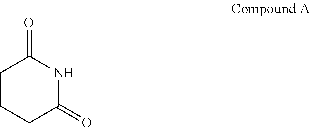

with oxalyl chloride under conditions suitable to provide Compound C

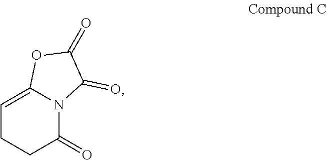 [SMILES: O=C1OC2=CCCC(=O)N2C1=O]

ii) contacting Compound C with n-bromosuccinimide (NBS) under conditions suitable to provide Compound D

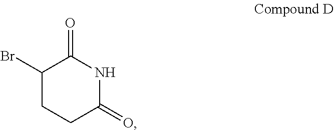

iii) contacting Compound D with tert-butyl (3-aminophenyl) carbamate

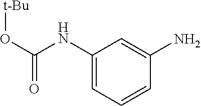 [SMILES: CC(C)(C)OC(=O)Nc1cccc(N)c1]

under conditions suitable to provide Compound H

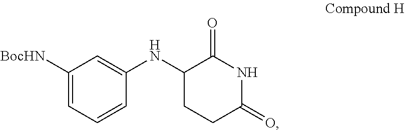 [SMILES: CC(C)(C)OC(=O)Nc1cccc(NC2CCC(=O)NC2=O)c1]

and
iv) contacting Compound H with p-toluenesulfonic acid monohydrate under conditions suitable to provide Compound J.

In certain embodiments, the process for preparing Compound 1 further comprises
1) contacting Compound A′

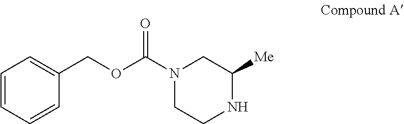 [SMILES: C[C@@H]1CN(C(=O)OCc2ccccc2)CCN1]

with tert-butyl 2-bromoacetate in a solvent under conditions suitable to provide Compound AA

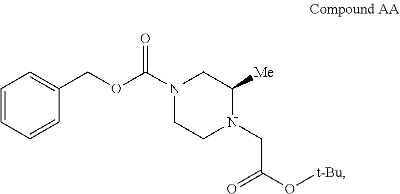 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

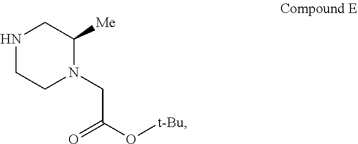 [SMILES: C[C@@H]1CNCCN1CC(=O)OC(C)(C)C]

3) contacting Compound E with Compound I

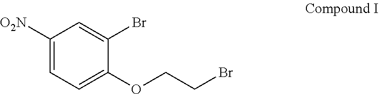 [SMILES: O=[N+]([O-])c1ccc(OCCBr)c(Br)c1]

in the presence of a base in a solvent under conditions suitable to provide Compound K

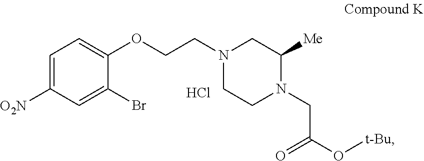 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

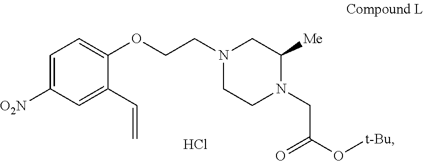 [SMILES: C=Cc1cc([N+](=O)[O-])ccc1OCCN1CCN(CC(=O)OC(C)(C)C)[C@H](C)C1 ; Cl]

and 5) contacting Compound L with a palladium catalyst in a solvent under conditions suitable to provide Compound N.

In one embodiment, provided herein is a process for preparing Compound 1 comprising
1) contacting Compound A′

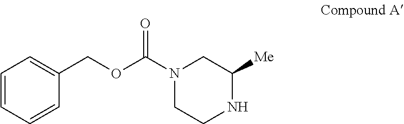 [SMILES: C[C@@H]1CN(C(=O)OCc2ccccc2)CCN1]

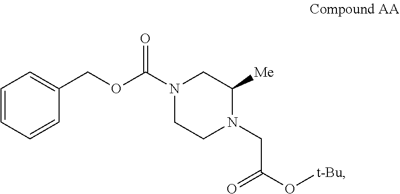 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

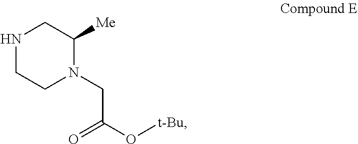 [SMILES: C[C@@H]1CNCCN1CC(=O)OC(C)(C)C]

3) contacting Compound E with Compound I

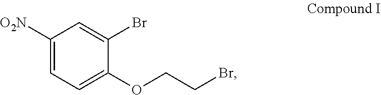 [SMILES: O=[N+]([O-])c1ccc(OCCBr)c(Br)c1]

in the presence of a base in a solvent under conditions suitable to provide Compound K

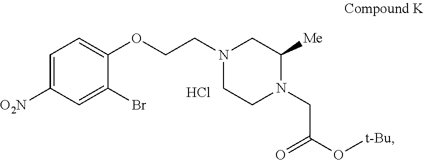 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

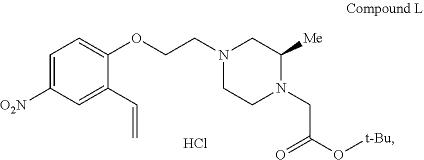 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

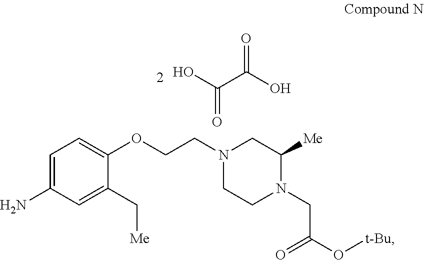

6) contacting Compound N with methyl 2-bromo-2-methylpropionate under conditions suitable to provide Compound P

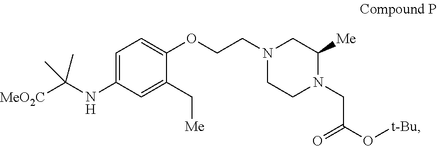 [SMILES: CCc1cc(NC(C)(C)C(=O)OC)ccc1OCCN1CCN(CC(=O)OC(C)(C)C)[C@H](C)C1]

7) contacting Compound P with Compound S

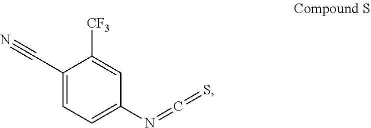 [SMILES: N#Cc1ccc(N=C=S)cc1C(F)(F)F]

under conditions suitable to provide Compound U

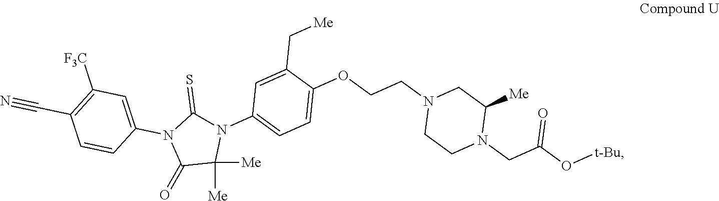 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

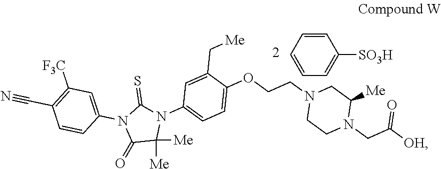

and 9) contacting Compound J

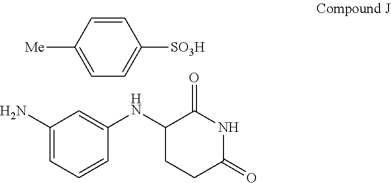

under conditions suitable to provide Compound 1,
wherein Compound J is prepared by
i) contacting an acid salt of 3-aminopiperidine-2,6-dione

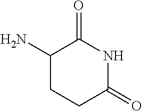

with tert-butyl (3-bromophenyl) carbamate

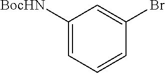 [SMILES: CC(C)(C)OC(=O)Nc1cccc(Br)c1]

under conditions suitable to provide Compound H

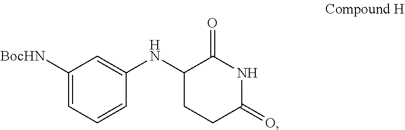 [SMILES: CC(C)(C)OC(=O)Nc1cccc(NC2CCC(=O)NC2=O)c1]

and ii) contacting Compound HI with p-toluenesulfonic acid monohydrate under conditions suitable to provide Compound J.

In one embodiment, provided herein is a process for preparing Compound 1 comprising
1) contacting Compound A′

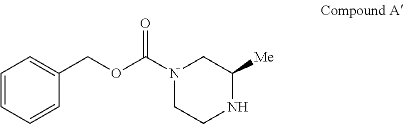 [SMILES: C[C@@H]1CN(C(=O)OCc2ccccc2)CCN1]

with tert-butyl 2-bromoacetate in a solvent under conditions suitable to provide Compound AA

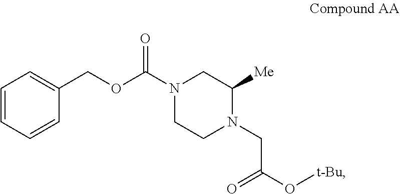 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

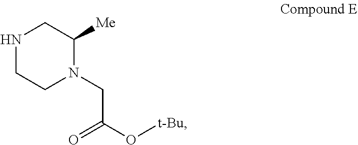 [SMILES: C[C@@H]1CNCCN1CC(=O)OC(C)(C)C]

3) contacting Compound E with Compound I

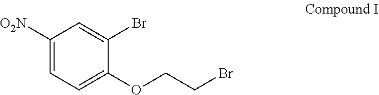 [SMILES: O=[N+]([O-])c1ccc(OCCBr)c(Br)c1]

in the presence of a base in a solvent under conditions suitable to provide Compound K

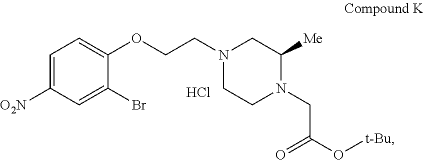 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

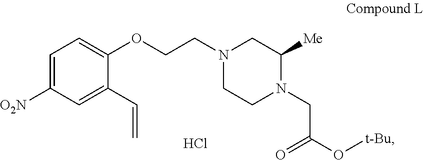 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

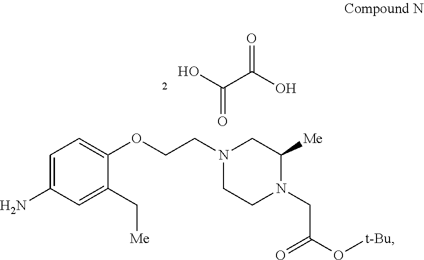

6) contacting Compound N with methyl 2-bromo-2-methylpropionate under conditions suitable to provide Compound P

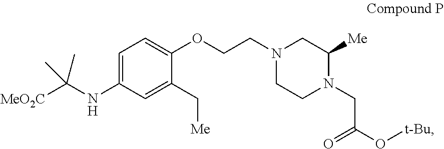 [SMILES: CCc1cc(NC(C)(C)C(=O)OC)ccc1OCCN1CCN(CC(=O)OC(C)(C)C)[C@H](C)C1]

7) contacting Compound P with Compound S

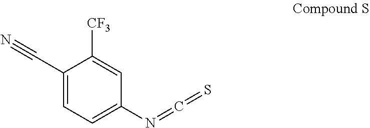 [SMILES: N#Cc1ccc(N=C=S)cc1C(F)(F)F]

under conditions suitable to provide Compound U

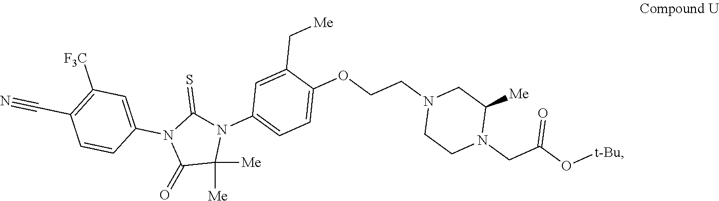 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

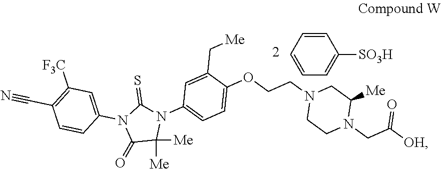

and 9) contacting Compound W with Compound J

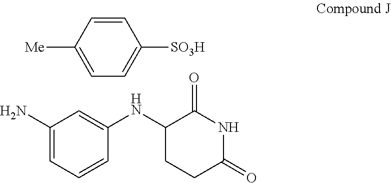

under conditions suitable to provide Compound 1,
wherein Compound J is prepared by
i) contacting piperidine-2,6-dione (Compound A)

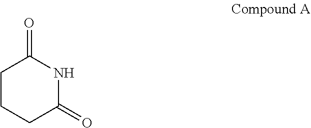

with oxalyl chloride under conditions suitable to provide Compound C

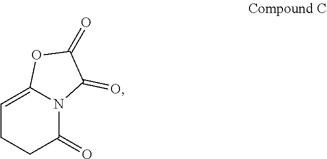 [SMILES: O=C1OC2=CCCC(=O)N2C1=O]

ii) contacting Compound C with n-bromosuccinimide (NBS) under conditions suitable to provide Compound D

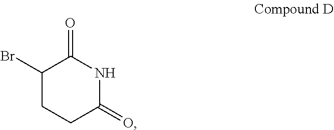

iii) contacting Compound D with tert-butyl (3-aminophenyl) carbamate

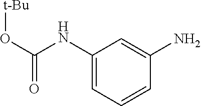 [SMILES: CC(C)(C)OC(=O)Nc1cccc(N)c1]

under conditions suitable to provide Compound H

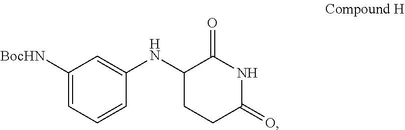 [SMILES: CC(C)(C)OC(=O)Nc1cccc(NC2CCC(=O)NC2=O)c1]

and iv) contacting Compound H with p-toluenesulfonic acid monohydrate under conditions suitable to provide Compound J.

In one embodiment, provided herein is a process for preparing Compound 1 comprising:
a) contacting 2-hydroxy-5-nitroacetophenone, Compound i

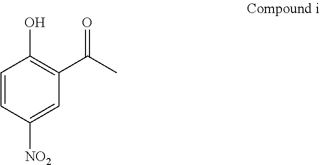

with trifluoroacetic acid and triethylsilane in a solvent under conditions suitable to provide Compound ii

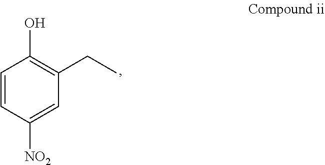

b) contacting Compound ii with ethylene carbonate in a solvent under conditions suitable to provide Compound iii

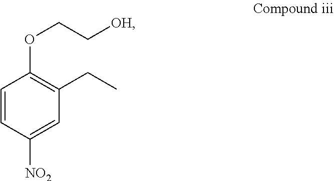

c) contacting Compound iii with 4-toluenesulfonyl chloride (TsCl) and triethylamine (TEA) in a solvent under conditions suitable to provide Compound iv in solution

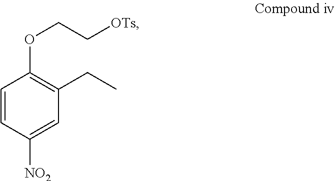 [SMILES: CCc1cc([N+](=O)[O-])ccc1OCCOS(=O)(=O)c1ccc(C)cc1]

d) contacting Compound iv in solution with Compound E in the presence of a base in a solvent, followed by oxalic acid under conditions suitable to provide Compound T

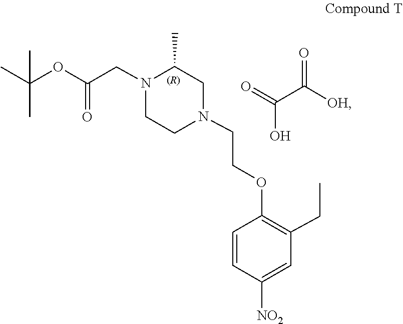

e) contacting Compound T with a platinum catalyst in a solvent under conditions suitable to provide Compound N1

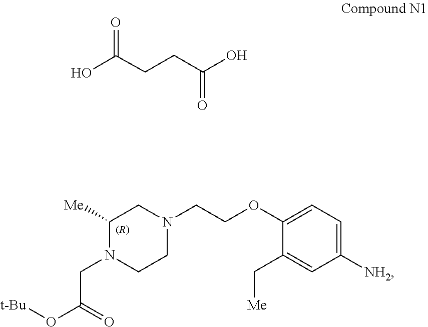

f) contacting Compound N1 with methyl 2-bromo-2-methylpropionate under conditions suitable to provide Compound P

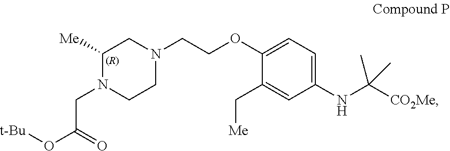

g) contacting Compound P with Compound S

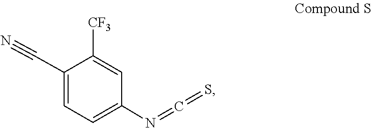 [SMILES: N#Cc1ccc(N=C=S)cc1C(F)(F)F]

h) followed by oxalic acid in a solvent under conditions suitable to provide Compound U

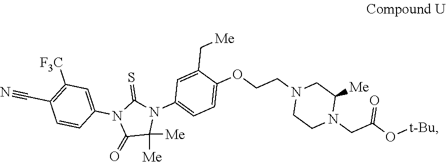 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

i) contacting Compound U with potassium carbonate followed by p-tosyl sulfonic acid in a solvent under conditions suitable to provide Compound W

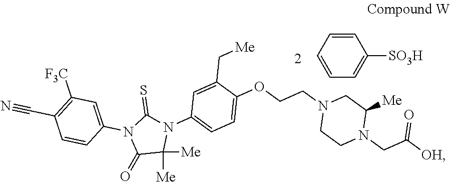

j) contacting Compound W with Compound J

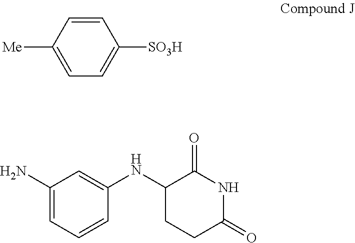 [SMILES: Cc1ccc(S(=O)(=O)O)cc1 ; Nc1cccc(NC2CCC(=O)NC2=O)c1]

followed by butyl acetate under conditions suitable to provide Compound 1, butyl acetate solvate

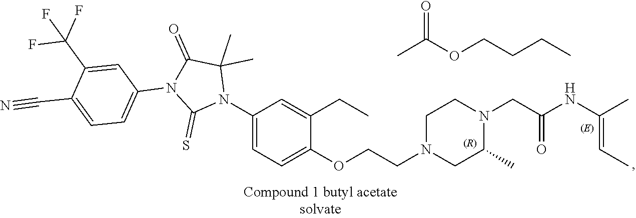

k) dissolving Compound 1, butyl acetate solvate to obtain a solution, and
l) spray drying the solution to obtain Compound 1, free base

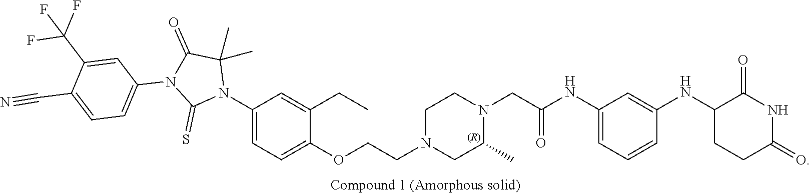

In one embodiment, the process provided herein comprises steps a)-k) as described above, followed by precipitation of Compound 1, free base.

In one embodiment, Compound E is prepared by
contacting Compound ix with a)ethyl trifluoroacetate in 2 methyltetrahydrofuran in presence of DIEPA; b) tert-butyl bromoacetate; c) tetramethylammonium hydroxide (MeqNOH) in methyl tetrahydrofuran; under conditions suitable to provide Compound E

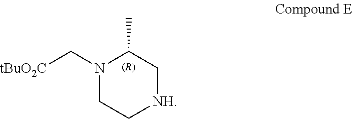

In one embodiment, provided herein is a process for preparing Compound T comprising with contacting Compound iv with Compound E in presence of a base in a solvent, followed by oxalic acid in methyl tetrahydrofuran under conditions suitable to provide Compound Y

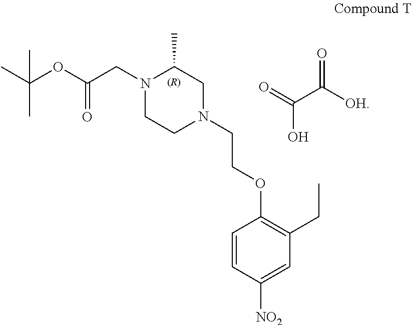

In one embodiment, provided herein is a process for preparing Compound N1 comprising contacting Compound T

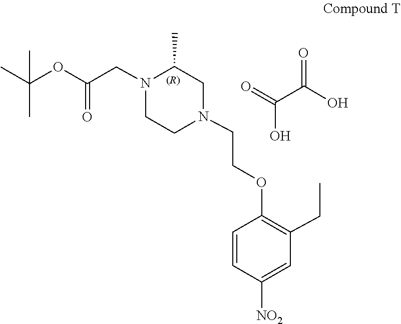

with a platinum catalyst in a solvent under conditions suitable to provide Compound N1

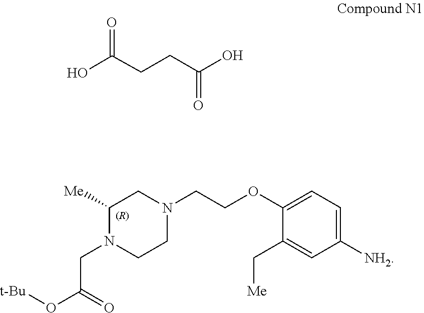

In one embodiment, the solvent comprises methyl tert-butyl ether (MTBE).

In certain embodiments, the process comprises contacting Compound T with a platinum catalyst in MTBE and sodium carbonate at about 20-25° C. In one embodiment, the process comprises contacting with succinic acid under conditions suitable to provide Compound N1.

In one embodiment, provided herein is a process for preparing Compound P, comprising contacting Compound N1 with methyl 2-bromo-2-methylpropionate in a solvent in the presence of a base under conditions suitable to provide Compound P. Exemplary bases for use in the process include K₂CO₃, TEA, DIEA, DBU, Na₂CO₃, Cs₂CO₃, Na₃PO₄, K₂HPO₄ and K₃PO₄. In one embodiment, the base is K₂HPO₄. Exemplary solvents for use in the process include NMP, CH₃CN, MeTHF, DMF, DMSO and DMAc. In one embodiment, the solvent comprises NMP. In one embodiment, the process comprises contacting Compound N1 with methyl 2-bromo-2-methylpropionate in NMP in the presence of K₂HPO₄ at about 75° C. under conditions suitable to provide Compound P. In one embodiment, the process comprises contacting Compound N1 with methyl 2-bromo-2-methylpropionate in NMP in the presence of K₂HPO₄ at about 75° C. for about 22 hours under conditions suitable to provide Compound P.

In one embodiment, provided herein is a process for preparing Compound 1-1

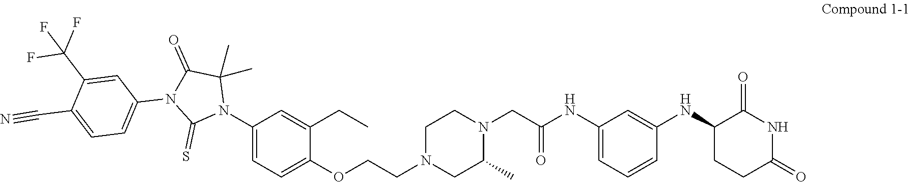 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

comprising
a) contacting Compound W

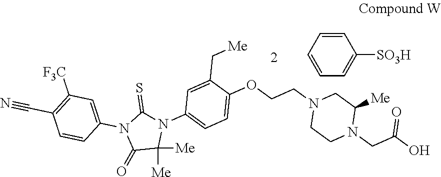

with Compound (R)-J

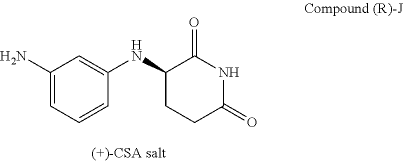 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1,
b) contacting the BSA salt of Compound 1-1 with a buffer in a solvent to obtain a solution comprising Compound 1-1, and
c) contacting the solution with water/acetone mixture to obtain Compound 1-1.

Exemplary bases for use in the process include N-methyl imidazole, triethyl amine and pyridine. In one embodiment, the base is N-methyl imidazole. Exemplary solvents for use in the process include THE, DMF, EtOAc, MeCN, DMAc, DMSO and a mixture thereof, such as a mixture of EtOAc/THF. In one embodiment, the solvent comprises THF.

In one aspect of the process, Compound W is contacted with Compound (R)-J in the presence of a coupling agent. Exemplary coupling agents for use in the process include propanephosphonic acid anhydride (T3P), Hexafluorophosphate Azabenzotriazole Tetramethyl Uronium (HATU), 1-Ethyl-3-(3-dimethylaminopropyl) carbodiimide (EDC), (1-Cyano-2-ethoxy-2-oxoethylidenaminooxy) dimethylamino-morpholino-carbenium hexafluorophosphate (COMU) and (benzotriazol-1-yloxy)tripyrrolidinophosphonium hexafluorophosphate (PyBop). In one embodiment, the coupling agent is propanephosphonic acid anhydride (T3P).

In one aspect of the process, 1 equivalent of Compound W is contacted with about 1.08 to 1.5 equivalents of Compound (R)-J in the presence of a base in a solvent under conditions suitable to provide Compound 1. In one aspect of the process, 1 equivalent of Compound W is contacted with about 1.08 equivalents of Compound (R)-J in the presence of a base in a solvent under conditions suitable to provide Compound 1-1.

In one aspect, the process comprises contacting Compound W, Compound (R)-J and N-methyl imidazole in THF in the presence of T3P under conditions suitable to provide Compound 1-1. In one aspect, the process comprises contacting 1 equivalent of Compound W, about 1.08 equivalents Compound (R)-J and N-methyl imidazole in THF in the presence of T3P under conditions suitable to provide Compound 1-1.

In one aspect of the process, the solvent in step b) is ethyl acetate. In one aspect of the process, the buffer in step b) is a potassium phosphate buffer.

In one embodiment, provided herein is a process for Compound (R)-J, comprising contacting Compound J

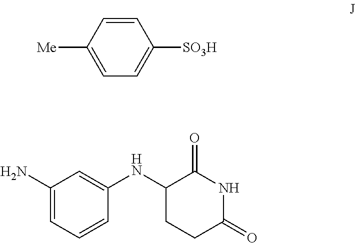 [SMILES: Cc1ccc(S(=O)(=O)O)cc1 ; Nc1cccc(NC2CCC(=O)NC2=O)c1]

with (+) camphor-10-sulphonic acid ((+) CSA) and a base under conditions suitable to provide Compound (R)-J. In one aspect, the process comprises contacting Compound J with (+) camphor-10-sulphonic acid ((+) CSA) and potassium carbonate in THF to provide Compound (R)-J.

In one embodiment, provided herein is a process for Compound(S)-J, comprising contacting Compound J

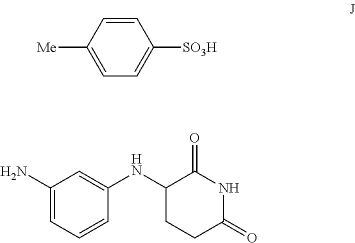 [SMILES: Cc1ccc(S(=O)(=O)O)cc1 ; Nc1cccc(NC2CCC(=O)NC2=O)c1]

with (−) camphor-10-sulphonic acid ((−) CSA) and a base under conditions suitable to provide Compound(S)-J

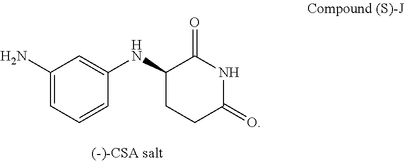 [SMILES: Nc1cccc(N[C@@H]2CCC(=O)NC2=O)c1]

In one aspect, the process comprises contacting Compound J with (−) camphor-10-sulphonic acid ((−) CSA) and potassium carbonate in THF to provide Compound(S)-J.

In one embodiment, provided herein is a process for preparing Compound 1-1 comprising
1) contacting Compound A′

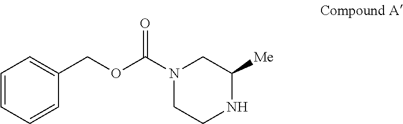 [SMILES: C[C@@H]1CN(C(=O)OCc2ccccc2)CCN1]

with tert-butyl 2-bromoacetate in a solvent under conditions suitable to provide Compound AA

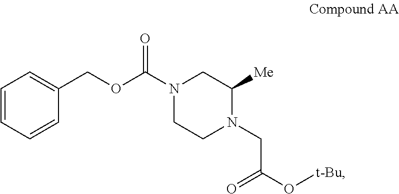 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

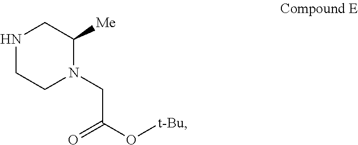 [SMILES: C[C@@H]1CNCCN1CC(=O)OC(C)(C)C]

3) contacting Compound E with Compound I

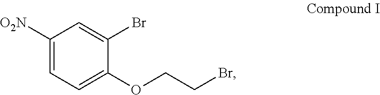 [SMILES: O=[N+]([O-])c1ccc(OCCBr)c(Br)c1]

in the presence of a base in a solvent under conditions suitable to provide Compound K

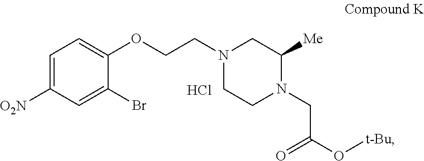 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

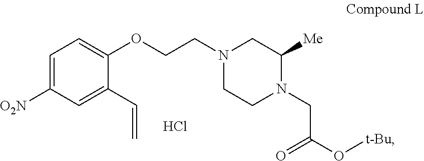 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

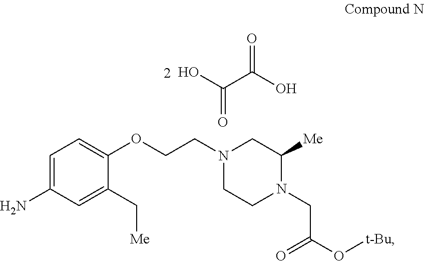

6) contacting Compound N with methyl 2-bromo-2-methylpropionate under conditions suitable to provide Compound P

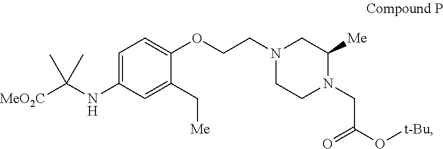 [SMILES: CCc1cc(NC(C)(C)C(=O)OC)ccc1OCCN1CCN(CC(=O)OC(C)(C)C)[C@H](C)C1]

7) contacting Compound P with Compound S

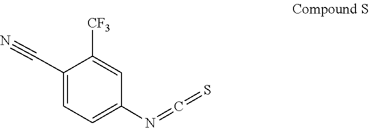 [SMILES: N#Cc1ccc(N=C=S)cc1C(F)(F)F]

under conditions suitable to provide Compound U

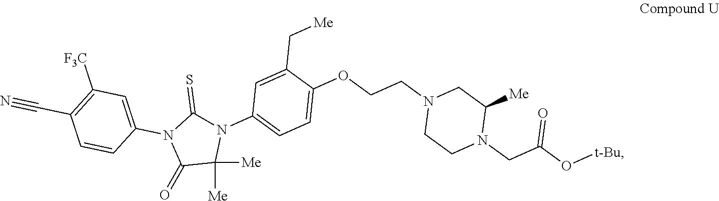 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

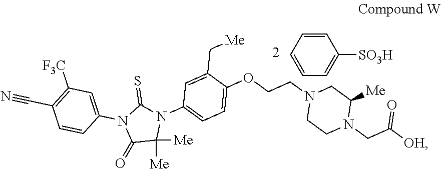

9) contacting Compound W with Compound (R)-J

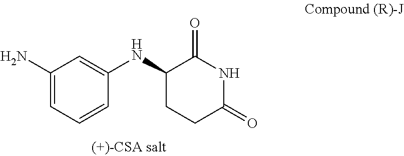 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1

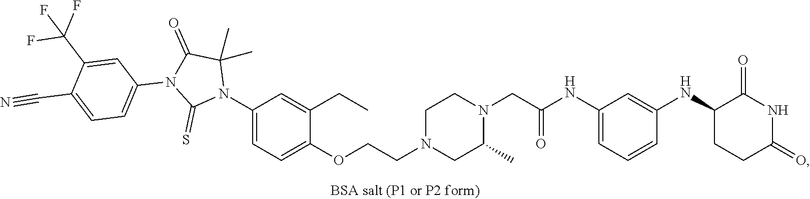 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

10) contacting the BSA salt of Compound 1-1 with a buffer in a solvent to obtain a solution comprising Compound 1-1, and
11) contacting the solution with water/acetone mixture to obtain Compound 1 1.

In certain embodiments, the process for preparing Compound 1-1 further comprises
i) contacting an acid salt of 3-aminopiperidine-2,6-dione

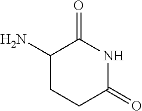

with tert-butyl (3-bromophenyl) carbamate

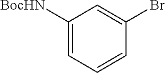 [SMILES: CC(C)(C)OC(=O)Nc1cccc(Br)c1]

under conditions suitable to provide Compound H

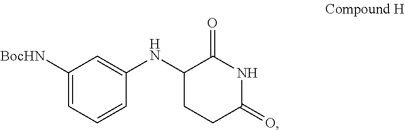 [SMILES: CC(C)(C)OC(=O)Nc1cccc(NC2CCC(=O)NC2=O)c1]

ii) contacting Compound H with p-toluenesulfonic acid monohydrate under conditions suitable to provide Compound J

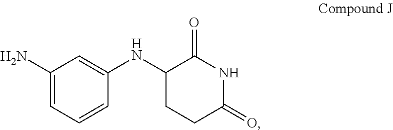 [SMILES: Nc1cccc(NC2CCC(=O)NC2=O)c1]

and iii) contacting Compound J with (+) camphor-10-sulphonic acid ((+) CSA) and a base under conditions suitable to provide Compound (R)-J

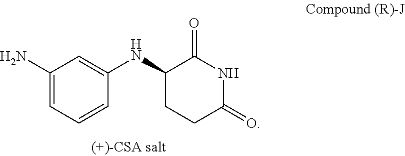 [SMILES: Nc1cccc(N[C@@H]2CCC(=O)NC2=O)c1]

In certain embodiments, the process for preparing Compound 1 further comprises
i) contacting piperidine-2,6-dione (Compound A)

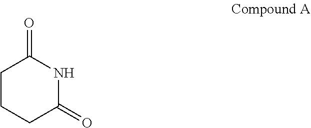

with oxalyl chloride under conditions suitable to provide Compound C

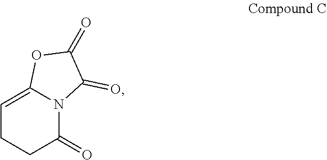 [SMILES: O=C1OC2=CCCC(=O)N2C1=O]

ii) contacting Compound C with n-bromosuccinimide (NBS) under conditions suitable to provide Compound D

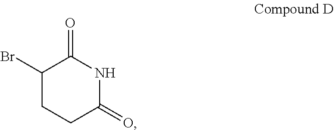

iii) contacting Compound D with tert-butyl (3-aminophenyl) carbamate

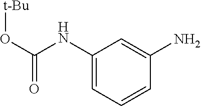 [SMILES: CC(C)(C)OC(=O)Nc1cccc(N)c1]

under conditions suitable to provide Compound H

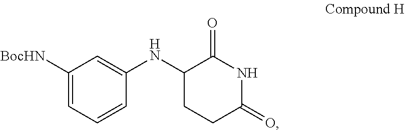 [SMILES: CC(C)(C)OC(=O)Nc1cccc(NC2CCC(=O)NC2=O)c1]

iv) contacting Compound H with p-toluenesulfonic acid monohydrate under conditions suitable to provide Compound J

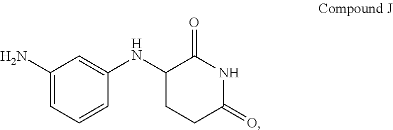 [SMILES: Nc1cccc(NC2CCC(=O)NC2=O)c1]

and v) contacting Compound J with (+) camphor-10-sulphonic acid ((+) CSA) and a base under conditions suitable to provide Compound (R)-J

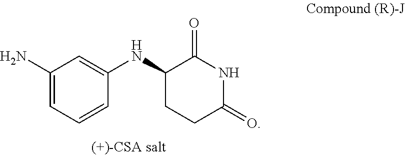 [SMILES: Nc1cccc(N[C@@H]2CCC(=O)NC2=O)c1]

In one embodiment, provided herein is a process for preparing Compound 1-1 comprising
1) contacting Compound A′

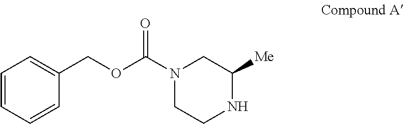 [SMILES: C[C@@H]1CN(C(=O)OCc2ccccc2)CCN1]

with tert-butyl 2-bromoacetate in a solvent under conditions suitable to provide Compound AA

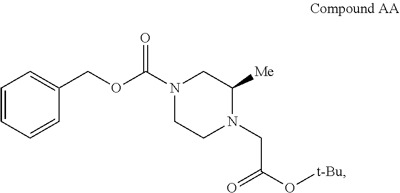 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

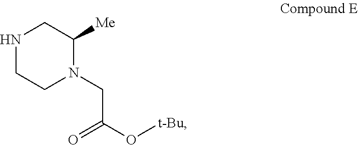 [SMILES: C[C@@H]1CNCCN1CC(=O)OC(C)(C)C]

3) contacting Compound E with Compound I

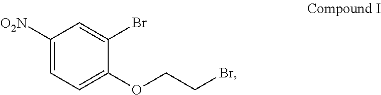 [SMILES: O=[N+]([O-])c1ccc(OCCBr)c(Br)c1]

in the presence of a base in a solvent under conditions suitable to provide Compound K

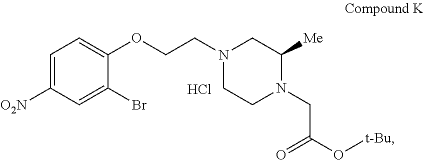 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

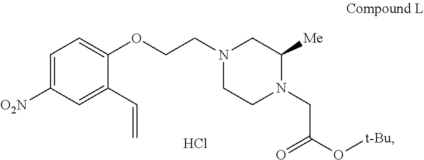 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

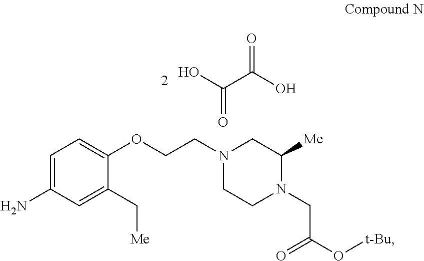

6) contacting Compound N with methyl 2-bromo-2-methylpropionate under conditions suitable to provide Compound P

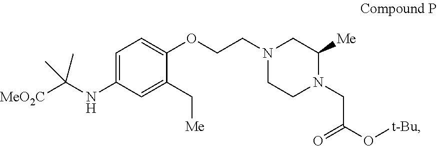 [SMILES: CCc1cc(NC(C)(C)C(=O)OC)ccc1OCCN1CCN(CC(=O)OC(C)(C)C)[C@H](C)C1]

7) contacting Compound P with Compound S

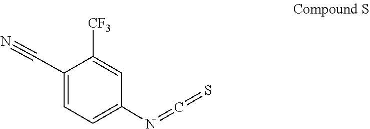 [SMILES: N#Cc1ccc(N=C=S)cc1C(F)(F)F]

under conditions suitable to provide Compound U

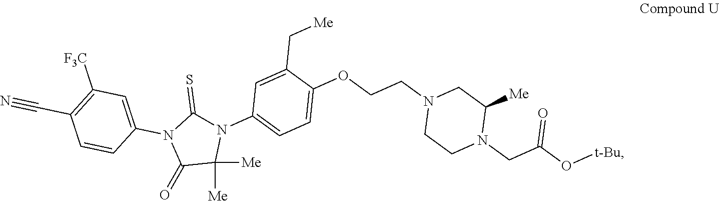 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

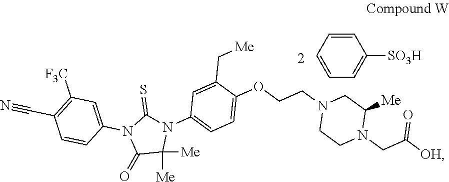

9) contacting Compound W with Compound (R)-J

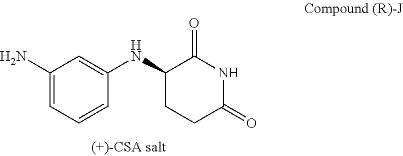 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1

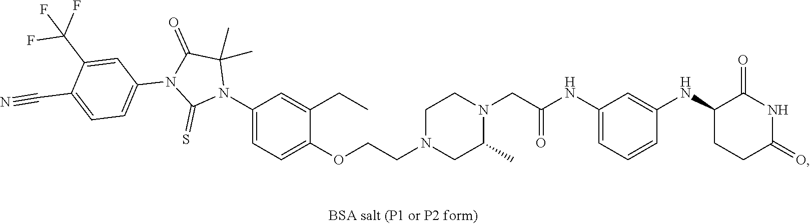 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

10) contacting the BSA salt of Compound 1-1 with a buffer in a solvent to obtain a solution comprising Compound 1-1, and
11) contacting the solution with water/acetone mixture to obtain Compound 1 1, wherein Compound (R)-J is prepared by
i) contacting an acid salt of 3-aminopiperidine-2,6-dione

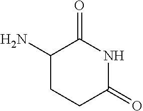

with tert-butyl (3-bromophenyl) carbamate

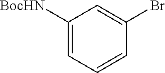 [SMILES: CC(C)(C)OC(=O)Nc1cccc(Br)c1]

under conditions suitable to provide Compound H

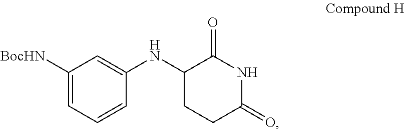 [SMILES: CC(C)(C)OC(=O)Nc1cccc(NC2CCC(=O)NC2=O)c1]

ii) contacting Compound H with p-toluenesulfonic acid monohydrate under conditions suitable to provide Compound J

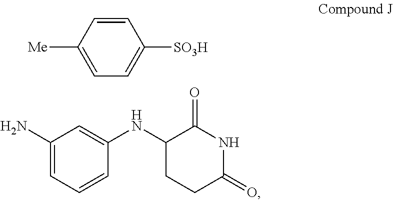 [SMILES: Cc1ccc(S(=O)(=O)O)cc1 ; Nc1cccc(NC2CCC(=O)NC2=O)c1]

and iii) contacting Compound J with (+) camphor-10-sulphonic acid ((+) CSA) and a base under conditions suitable to provide Compound (R)-J

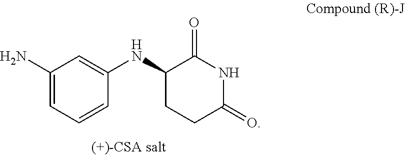 [SMILES: Nc1cccc(N[C@@H]2CCC(=O)NC2=O)c1]

In one embodiment, provided herein is a process for preparing Compound 1 comprising
1) contacting Compound A′

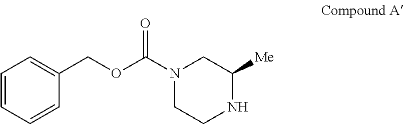 [SMILES: C[C@@H]1CN(C(=O)OCc2ccccc2)CCN1]

with tert-butyl 2-bromoacetate in a solvent under conditions suitable to provide Compound AA

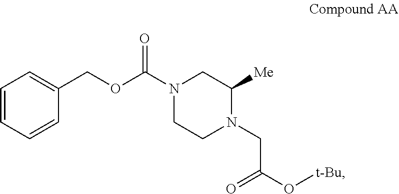 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

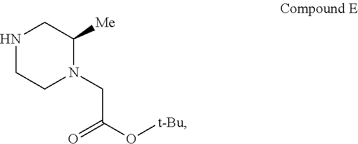 [SMILES: C[C@@H]1CNCCN1CC(=O)OC(C)(C)C]

3) contacting Compound E with Compound I

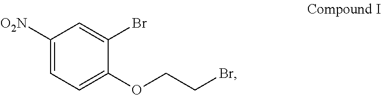 [SMILES: O=[N+]([O-])c1ccc(OCCBr)c(Br)c1]

in the presence of a base in a solvent under conditions suitable to provide Compound K

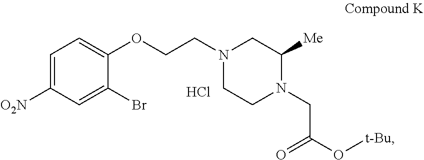 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

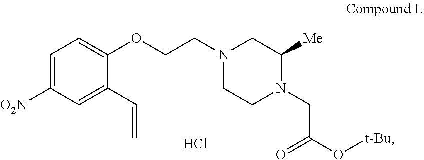 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

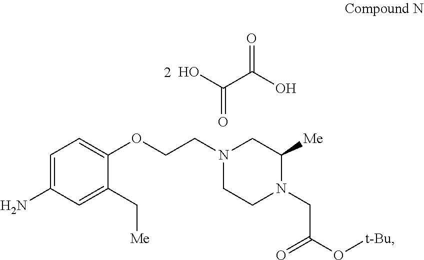

6) contacting Compound N with methyl 2-bromo-2-methylpropionate under conditions suitable to provide Compound P

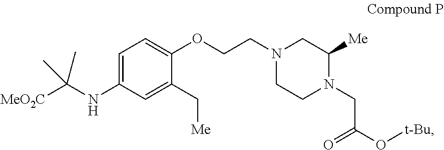 [SMILES: CCc1cc(NC(C)(C)C(=O)OC)ccc1OCCN1CCN(CC(=O)OC(C)(C)C)[C@H](C)C1]

7) contacting Compound P with Compound S

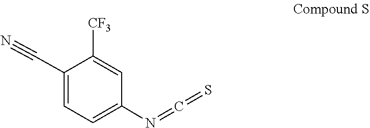 [SMILES: N#Cc1ccc(N=C=S)cc1C(F)(F)F]

under conditions suitable to provide Compound U

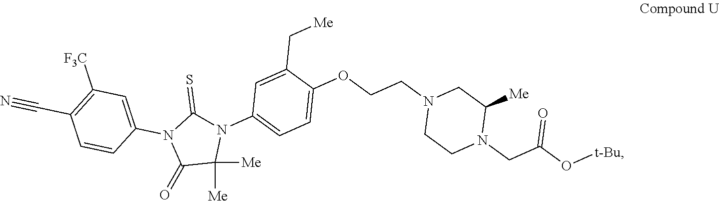 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

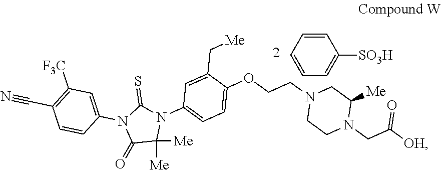

9) contacting Compound W with Compound (R)-J

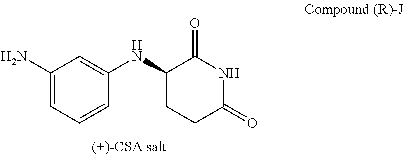 [SMILES: Nc1cccc(N[C@@H]2CCC(=O)NC2=O)c1]

under conditions suitable to provide suitable to provide BSA salt of Compound 1-1

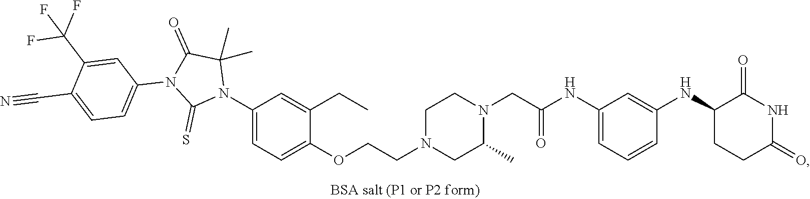 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

10) contacting the BSA salt of Compound 1-1 with a buffer in a solvent to obtain a solution comprising Compound 1-1, and
11) contacting the solution with water/acetone mixture to obtain Compound 1 1, wherein Compound (R)-J is prepared by
i) contacting piperidine-2,6-dione (Compound A)

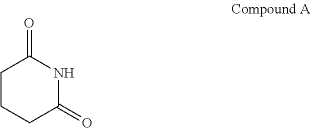

with oxalyl chloride under conditions suitable to provide Compound C

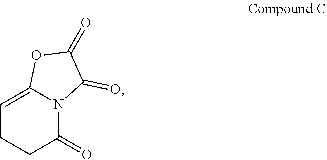 [SMILES: O=C1OC2=CCCC(=O)N2C1=O]

ii) contacting Compound C with n-bromosuccinimide (NBS) under conditions suitable to provide Compound D

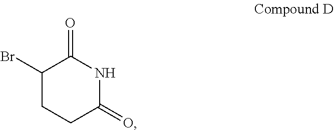

iii) contacting Compound D with tert-butyl (3-aminophenyl) carbamate

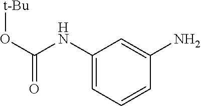 [SMILES: CC(C)(C)OC(=O)Nc1cccc(N)c1]

under conditions suitable to provide Compound H

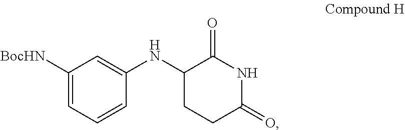 [SMILES: CC(C)(C)OC(=O)Nc1cccc(NC2CCC(=O)NC2=O)c1]

iv) contacting Compound H with p-toluenesulfonic acid monohydrate under conditions suitable to provide Compound J

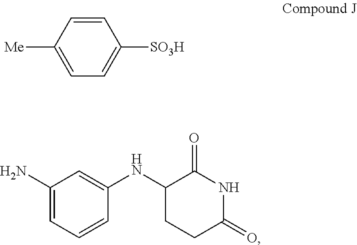 [SMILES: Cc1ccc(S(=O)(=O)O)cc1 ; Nc1cccc(NC2CCC(=O)NC2=O)c1]

and v) contacting Compound J with (+) camphor-10-sulphonic acid ((+) CSA) and a base under conditions suitable to provide Compound (R)-J

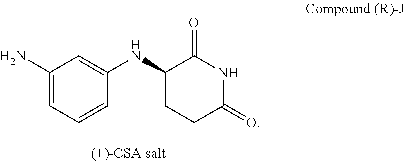 [SMILES: Nc1cccc(N[C@@H]2CCC(=O)NC2=O)c1]

In one embodiment, provided herein is a process for preparing Compound 1-1

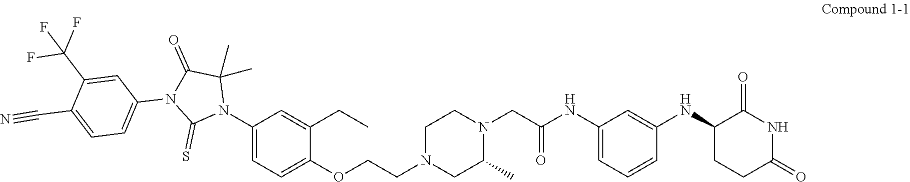 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

comprising
a) contacting Compound W

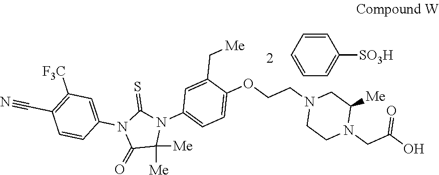

with Compound (R)-J

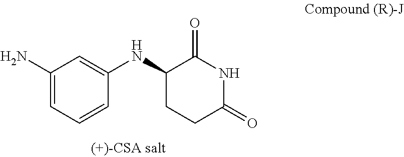 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1,
b) contacting the BSA salt of Compound 1-1 with a buffer in a solvent to obtain a solution comprising Compound 1-1, and
c) contacting the solution with water/acetone mixture to obtain Compound 1 1.

In one embodiment, provided herein is a process for preparing Compound viii

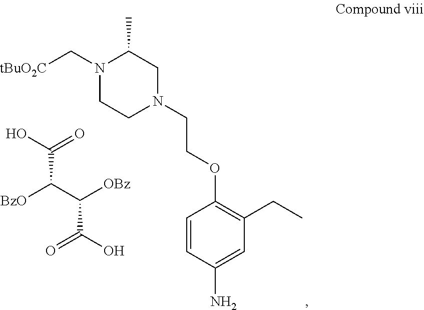

wherein the process comprises contacting Compound vii

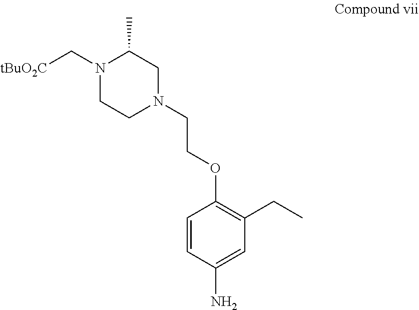 [SMILES: CCc1cc(N)ccc1OCCN1CCN(CC(=O)OC(C)(C)C)[C@H](C)C1]

with (+)-dibenzoyl-D-tartaric acid to provide Compound viii.

In one embodiment, provided herein is a process for preparing Compound vii

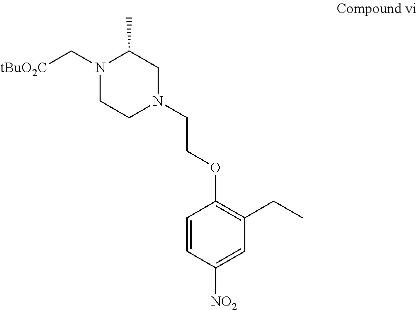 [SMILES: CCc1cc([N+](=O)[O-])ccc1OCCN1CCN(CC(=O)OC(C)(C)C)[C@H](C)C1]

wherein the process comprises contacting Compound vi

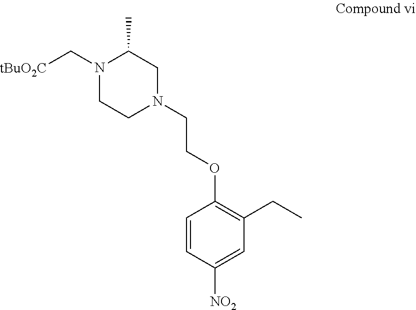 [SMILES: CCc1cc([N+](=O)[O-])ccc1OCCN1CCN(CC(=O)OC(C)(C)C)[C@H](C)C1]

with a palladium catalyst in a solvent under conditions suitable to provide Compound vii. In one embodiment, the solvent comprises 2-methyltetrahydrofuran and ethanol. In certain embodiments, the process comprises contacting Compound vi with a palladium catalyst in a solvent comprising 2-methyltetrahydrofuran and ethanol mixture at about 20° C. to provide Compound vii.

In one embodiment, provided herein is a process for preparing Compound vi

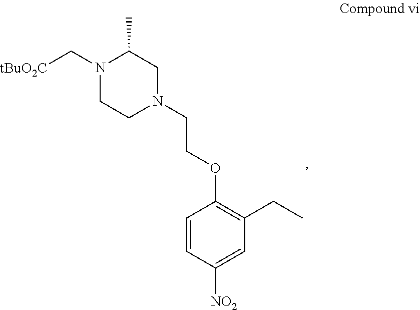 [SMILES: CCc1cc([N+](=O)[O-])ccc1OCCN1CCN(CC(=O)OC(C)(C)C)[C@H](C)C1]

wherein the process comprises contacting Compound v

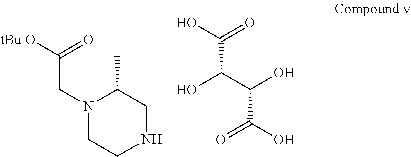

with Compound iv

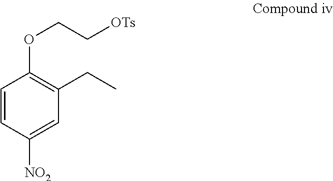 [SMILES: CCc1cc([N+](=O)[O-])ccc1OCCOS(=O)(=O)c1ccc(C)cc1]

in a solvent under conditions suitable to provide Compound vi. In one embodiment, the solvent comprises 2-methyltetrahydrofuran and water. In certain embodiments, the process comprises contacting Compound v with Compound iv in 2-methyltetrahydrofuran and water mixture in presence of sodium carbonate at about 71° C. to provide Compound vi.

In one embodiment, provided herein is a process for preparing Compound v

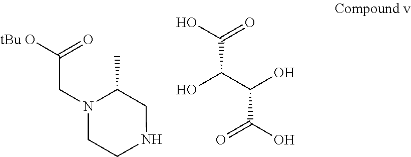

wherein the process comprises contacting (R)-(−)-2-methylpiperazine, Compound ix

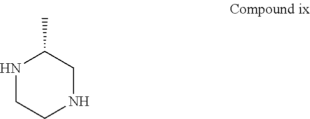

with a)ethyl trifluoroacetate in a solvent in presence of a base; b) tert-butyl bromoacetate; c) 25% tetramethylammonium hydroxide (Me₄NOH) in methanol; and d) D-tartaric acid under conditions suitable to provide Compound v. In one embodiment, the solvent comprises N-methyl-2-pyrrolidone. In certain embodiments, the process comprises contacting Compound ix with a)ethyl trifluoroacetate in 2-methyltetrahydrofuran in presence of DIEPA; b) tert-butyl bromoacetate; c) 25% tetramethylammonium hydroxide (Me₄NOH) in methanol; and d) D-tartaric acid under conditions suitable to provide Compound v.

In one embodiment, provided herein is a process for preparing Compound iv

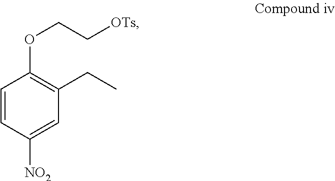 [SMILES: CCc1cc([N+](=O)[O-])ccc1OCCOS(=O)(=O)c1ccc(C)cc1]

wherein the process comprises contacting Compound iii

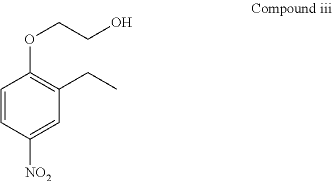

with 4-toluenesulfonyl chloride (TsC1) and triethylamine (TEA) in a solvent under conditions suitable to provide Compound iv. In one embodiment, the solvent comprises 2-methyltetrahydrofuran. In certain embodiments, the process comprises contacting Compound iii with TsCl and TEA in 2-methyltetrahydrofuran at about 20° C. to provide Compound iv.

In one embodiment, provided herein is a process for preparing Compound iii

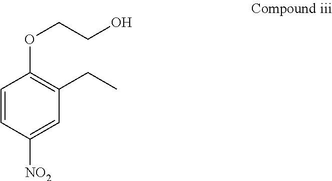

wherein the process comprises contacting Compound ii

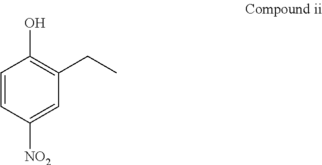

with sodium carbonate and ethylene carbonate in a solvent under conditions suitable to provide Compound iii. In one embodiment, the solvent comprises N-methyl-2-pyrrolidone. In certain embodiments, the process comprises contacting Compound ii with sodium carbonate and ethylene carbonate in N-methyl-2-pyrrolidone at about 100° C. to provide Compound iii.

In one embodiment, provided herein is a process for preparing Compound ii

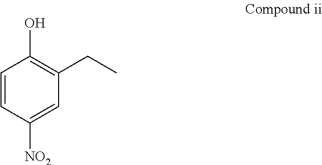

wherein the process comprises contacting 2-hydroxy-5-nitroacetophenone, Compound i

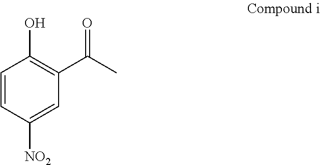

with trifluoroacetic acid and triethylsilane in a solvent under conditions suitable to provide Compound ii. In one embodiment, the solvent comprises toluene and heptane. In certain embodiments, the process comprises contacting Compound i with trifluoroacetic acid and triethylsilane in a mixture of toluene and heptane to provide Compound ii.

In one embodiment, provided herein is a process for preparing Compound viii, wherein the process comprises contacting 2-hydroxy-5-nitroacetophenone, Compound i

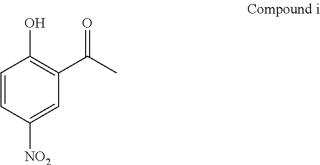

with trifluoroacetic acid and triethylsilane in a solvent under conditions suitable to provide Compound ii

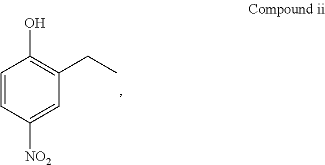

contacting Compound ii with sodium carbonate and ethylene carbonate in a solvent under conditions suitable to provide Compound iii

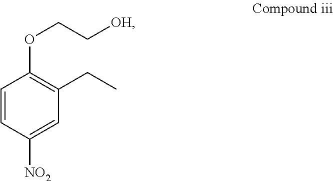

contacting Compound iii with 4-toluenesulfonyl chloride (TsCl) and triethylamine (TEA) in a solvent under conditions suitable to provide Compound iv

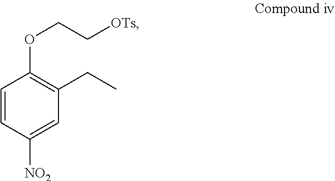 [SMILES: CCc1cc([N+](=O)[O-])ccc1OCCOS(=O)(=O)c1ccc(C)cc1]

contacting Compound iv with Compound v

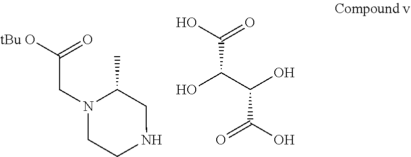

in a solvent under conditions suitable to provide Compound vi

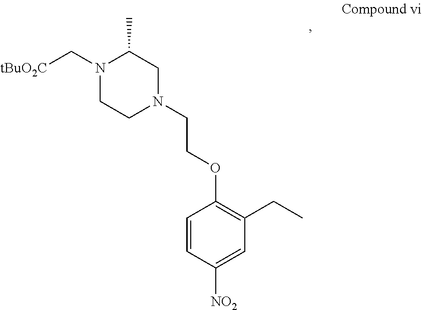 [SMILES: CCc1cc([N+](=O)[O-])ccc1OCCN1CCN(CC(=O)OC(C)(C)C)[C@H](C)C1]

contacting Compound vi with a palladium catalyst in a solvent under conditions suitable to provide Compound vii

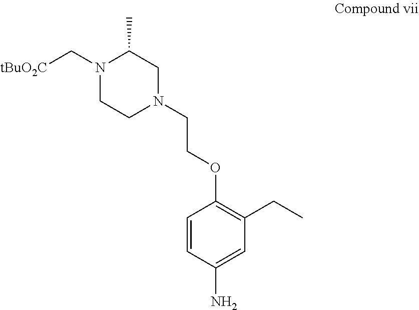 [SMILES: CCc1cc(N)ccc1OCCN1CCN(CC(=O)OC(C)(C)C)[C@H](C)C1]

contacting Compound vii with (+)-dibenzoyl-D-tartaric acid to provide Compound viii

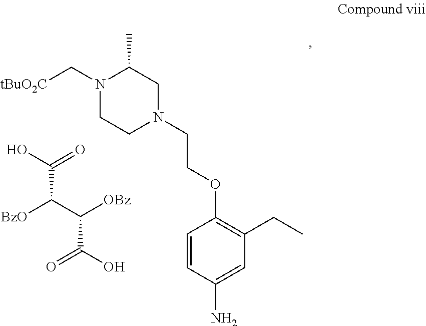

wherein Compound v is prepared by contacting (R)-(−)-2-methylpiperazine, Compound ix

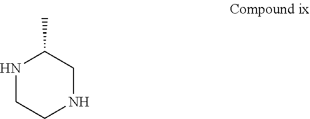

with a)ethyl trifluoroacetate in a solvent in presence of a base; b) tert-butyl bromoacetate; c) 25% tetramethylammonium hydroxide (Me₄NOH) in methanol; and d) D-tartaric acid under conditions suitable to provide Compound v.

In one embodiment, the processes herein provide an improvement in yield of Compound 1 as compared to the processes known in the art. In one embodiment, the improvement in the processes provided herein as compared to the processes known in the art includes improved efficiency. In one embodiment, the processes provided herein result in improved yields.

In certain embodiments, provided herein are chemical intermediates useful in the methods provided herein. In certeain embodiments, provided herein are chemical intermediates useful in the synthesis of Compound 1.

In one embodiment, provided herein is Compound K

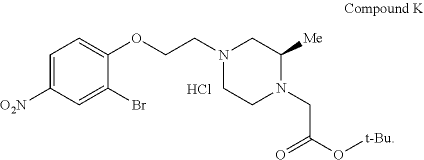 [SMILES: C[C@@H]1CN(CCOc2ccc([N+](=O)[O-])cc2Br)CCN1CC(=O)OC(C)(C)C ; Cl]

In one embodiment, provided herein is Compound L

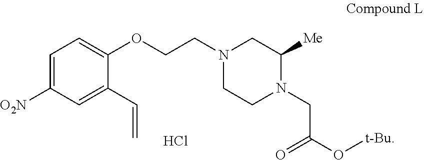 [SMILES: C=Cc1cc([N+](=O)[O-])ccc1OCCN1CCN(CC(=O)OC(C)(C)C)[C@H](C)C1 ; Cl]

In one embodiment, provided herein is Compound N

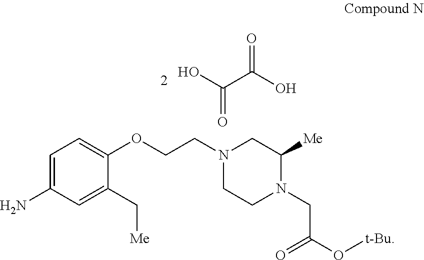

In one embodiment, provided herein is Compound P

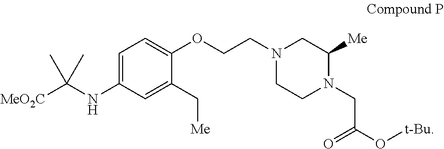 [SMILES: CCc1cc(NC(C)(C)C(=O)OC)ccc1OCCN1CCN(CC(=O)OC(C)(C)C)[C@H](C)C1]

In one embodiment, provided herein is Compound U

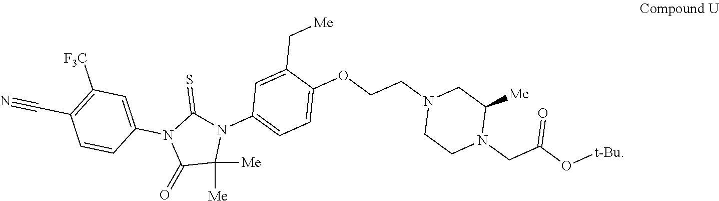 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

In one embodiment, provided herein is Compound W

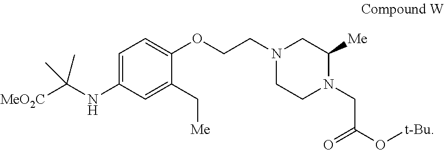 [SMILES: CCc1cc(NC(C)(C)C(=O)OC)ccc1OCCN1CCN(CC(=O)OC(C)(C)C)[C@H](C)C1]

### Synthetic Schemes

In certain embodiments, Compound 1 is prepared as outlined in schemes 1 or 2 shown below, as well as in the examples set forth in the Example section. It should be noted that one skilled in the art can modify the procedures set forth in the illustrative schemes and examples to arrive at the desired product.

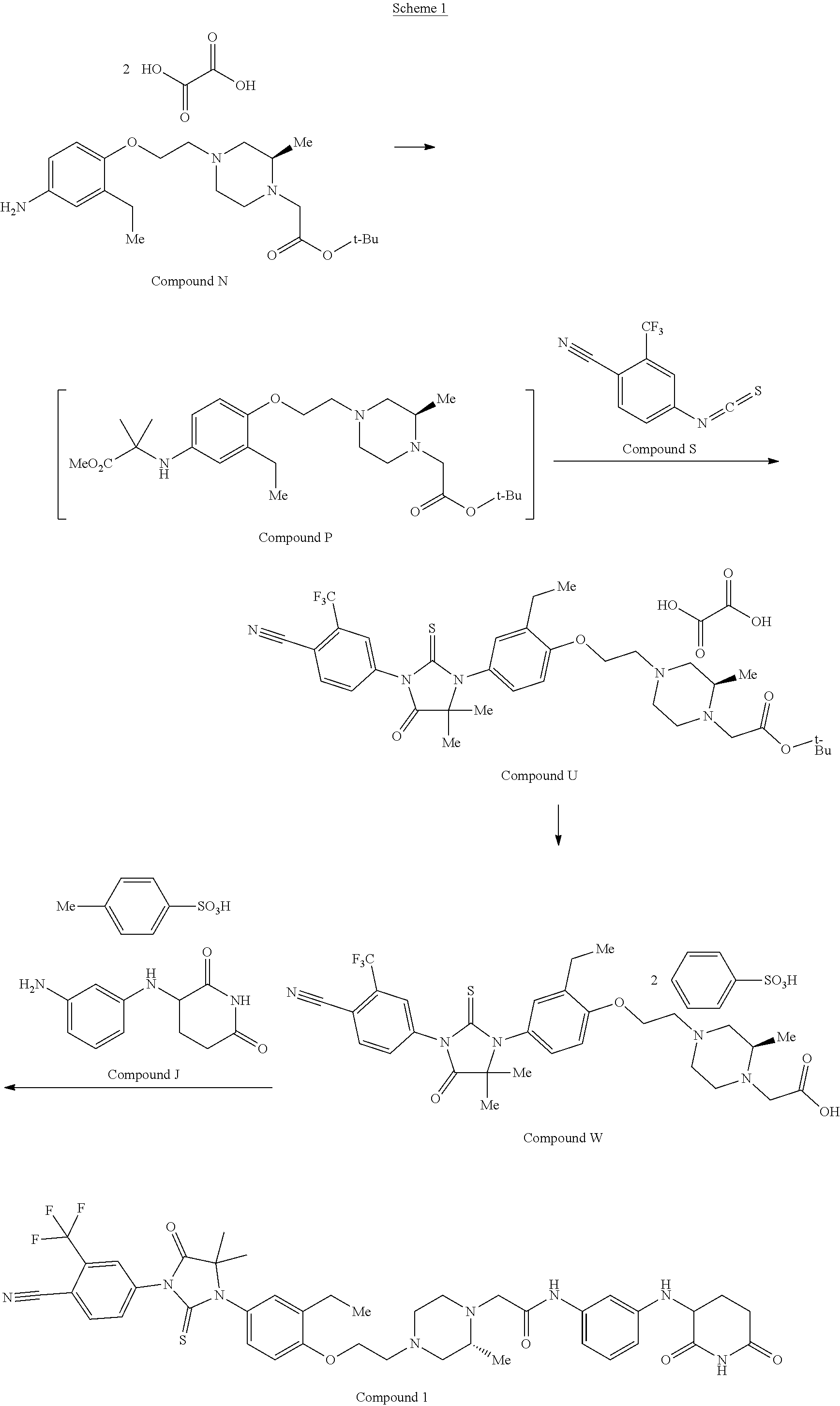

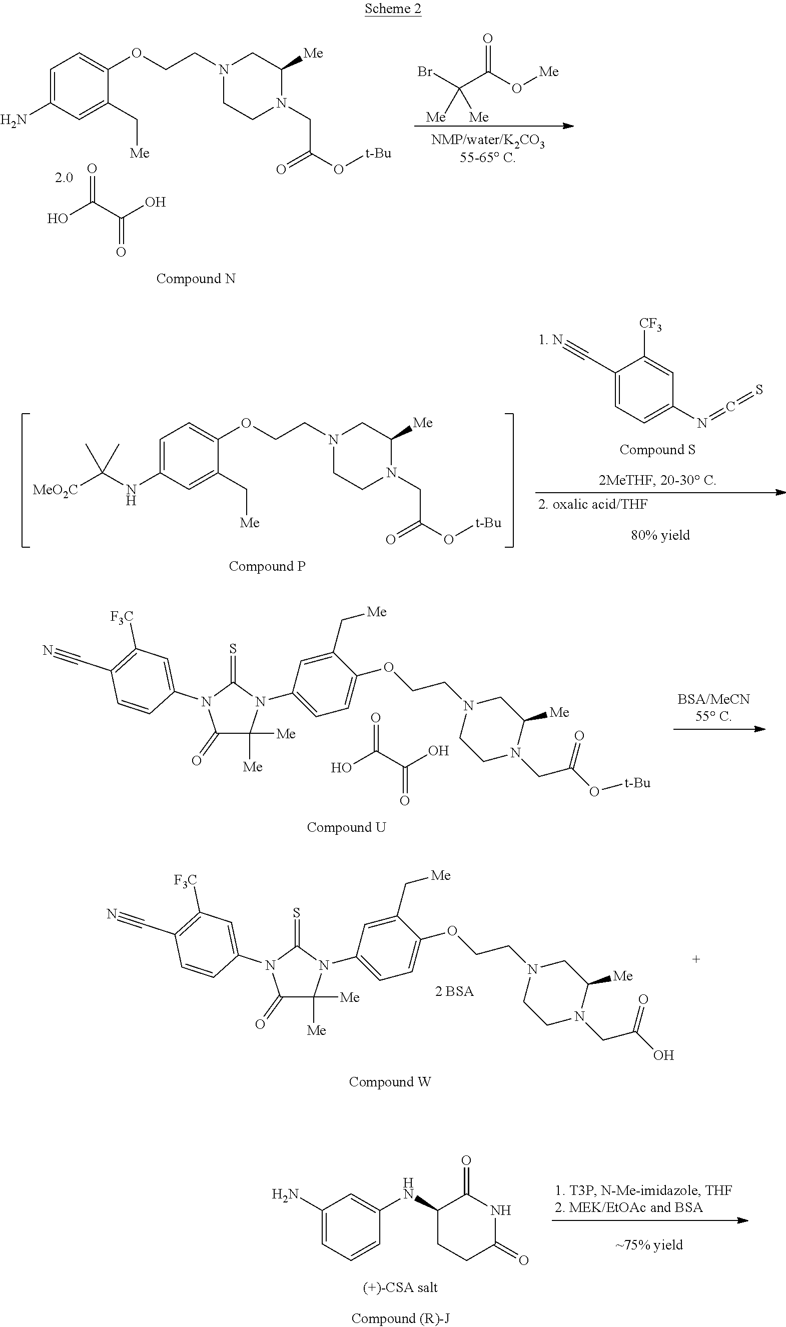

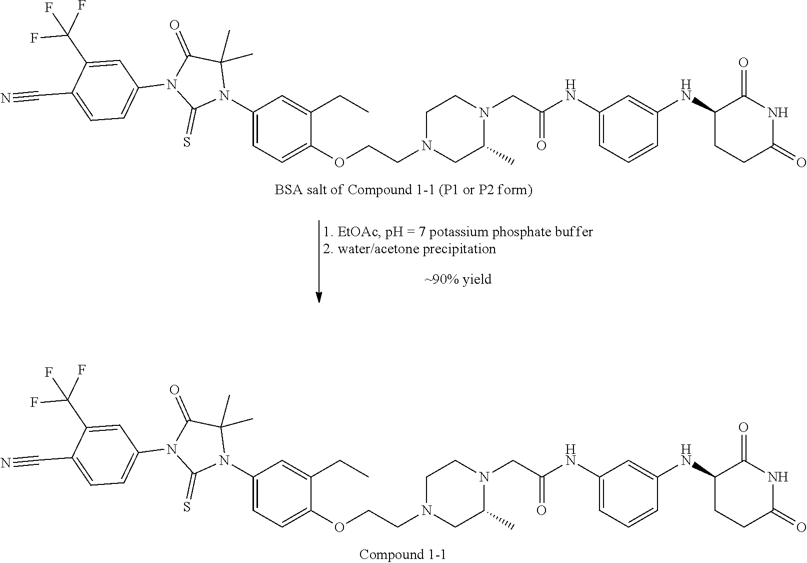 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1 ; CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

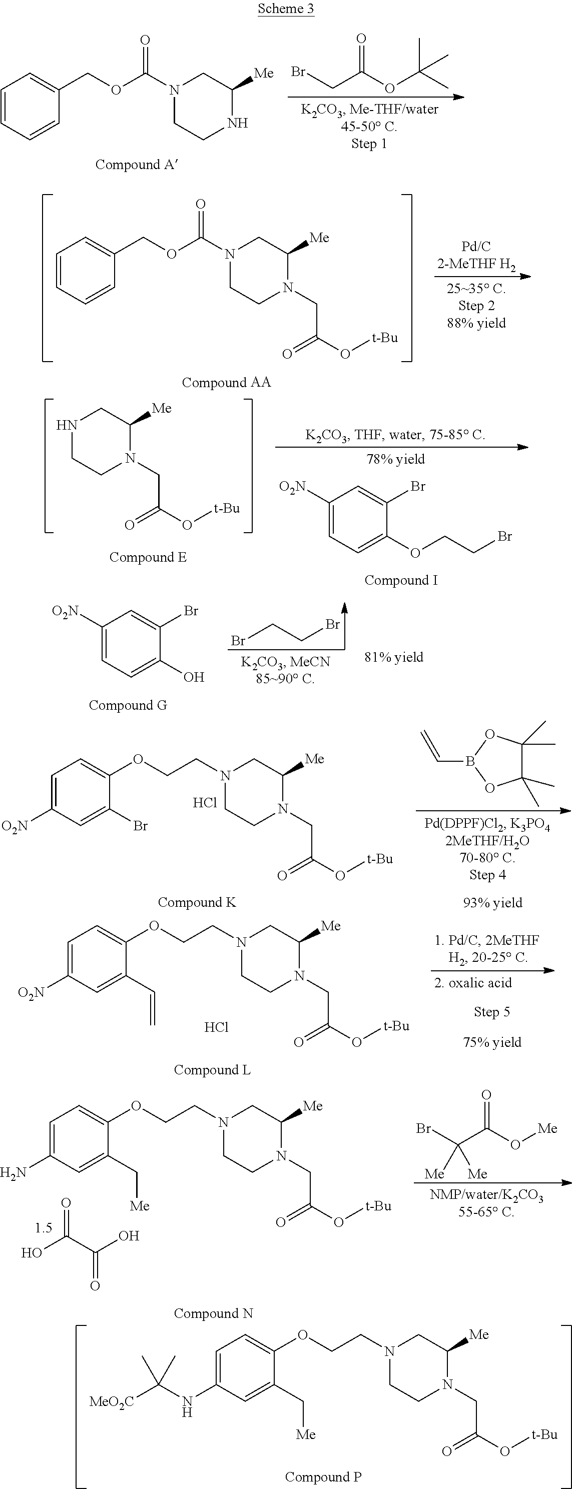

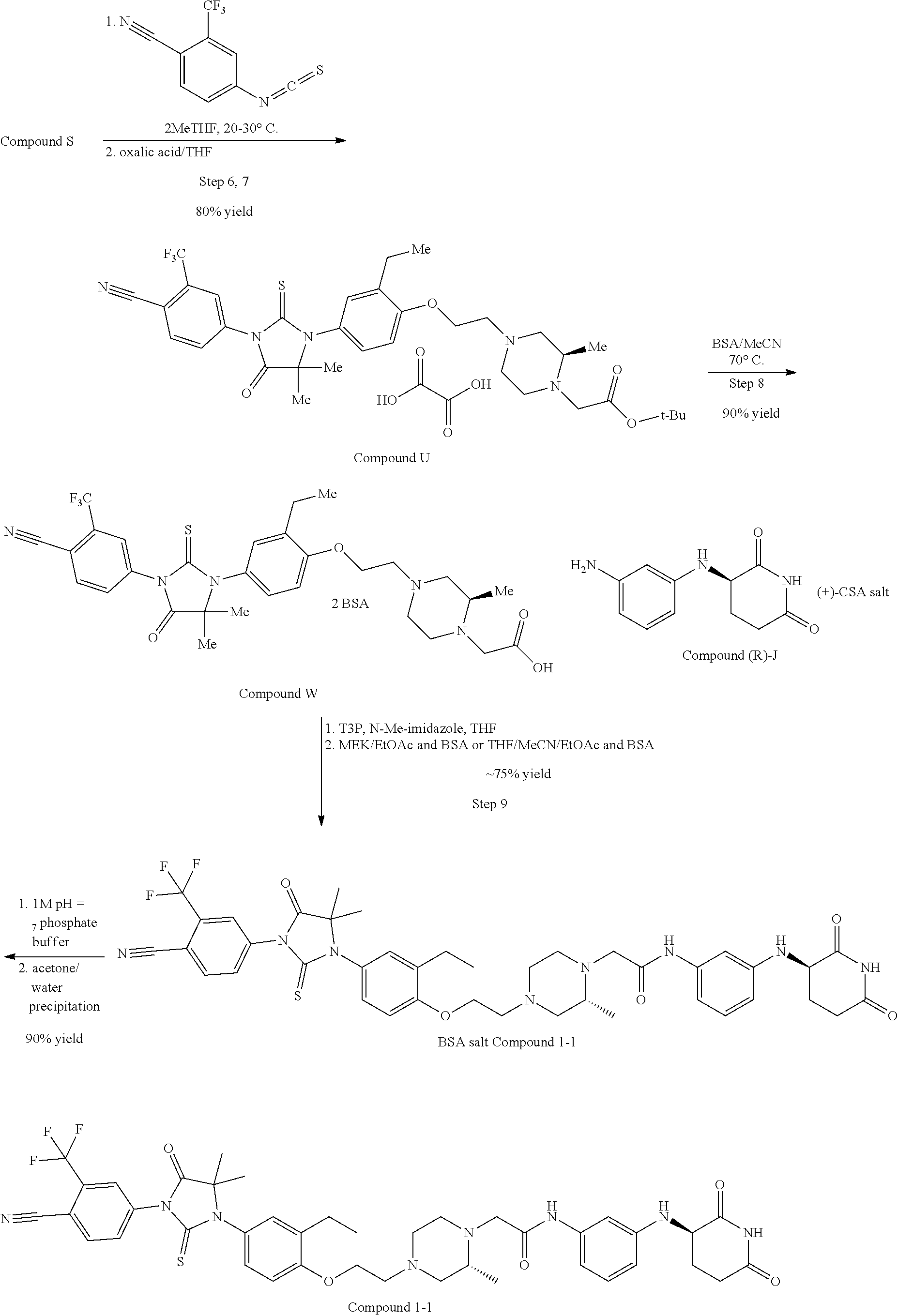

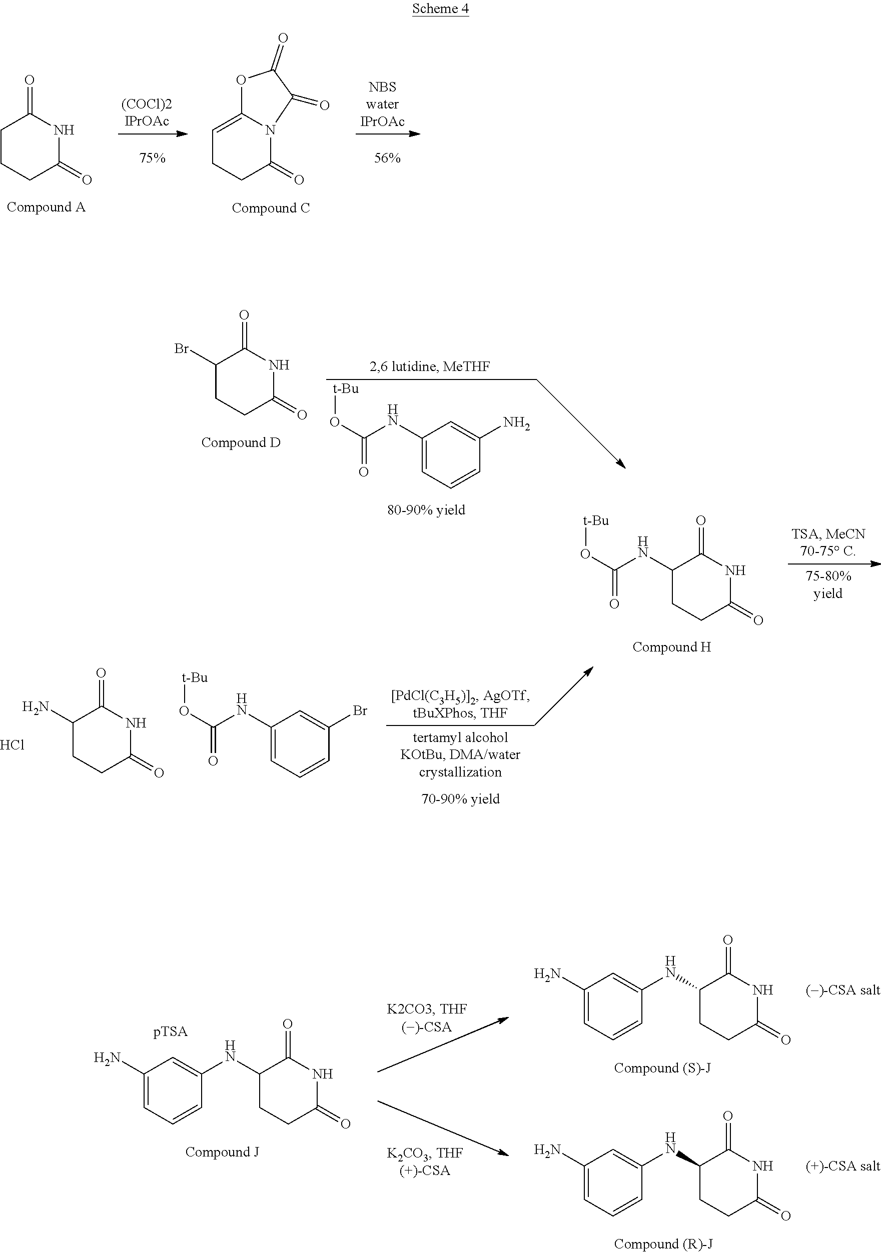

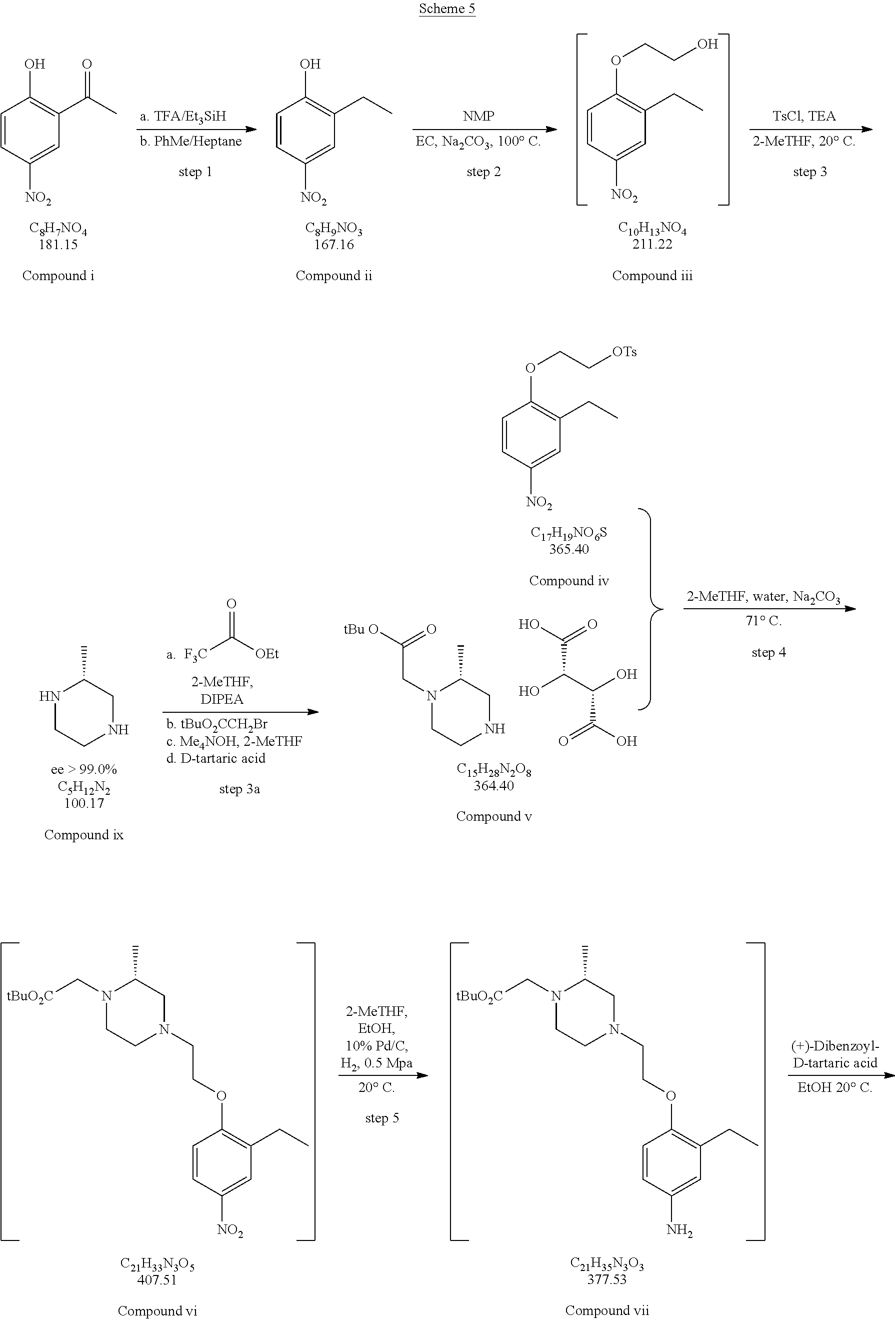

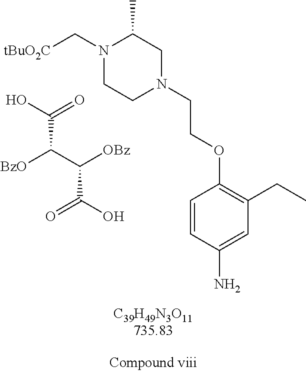

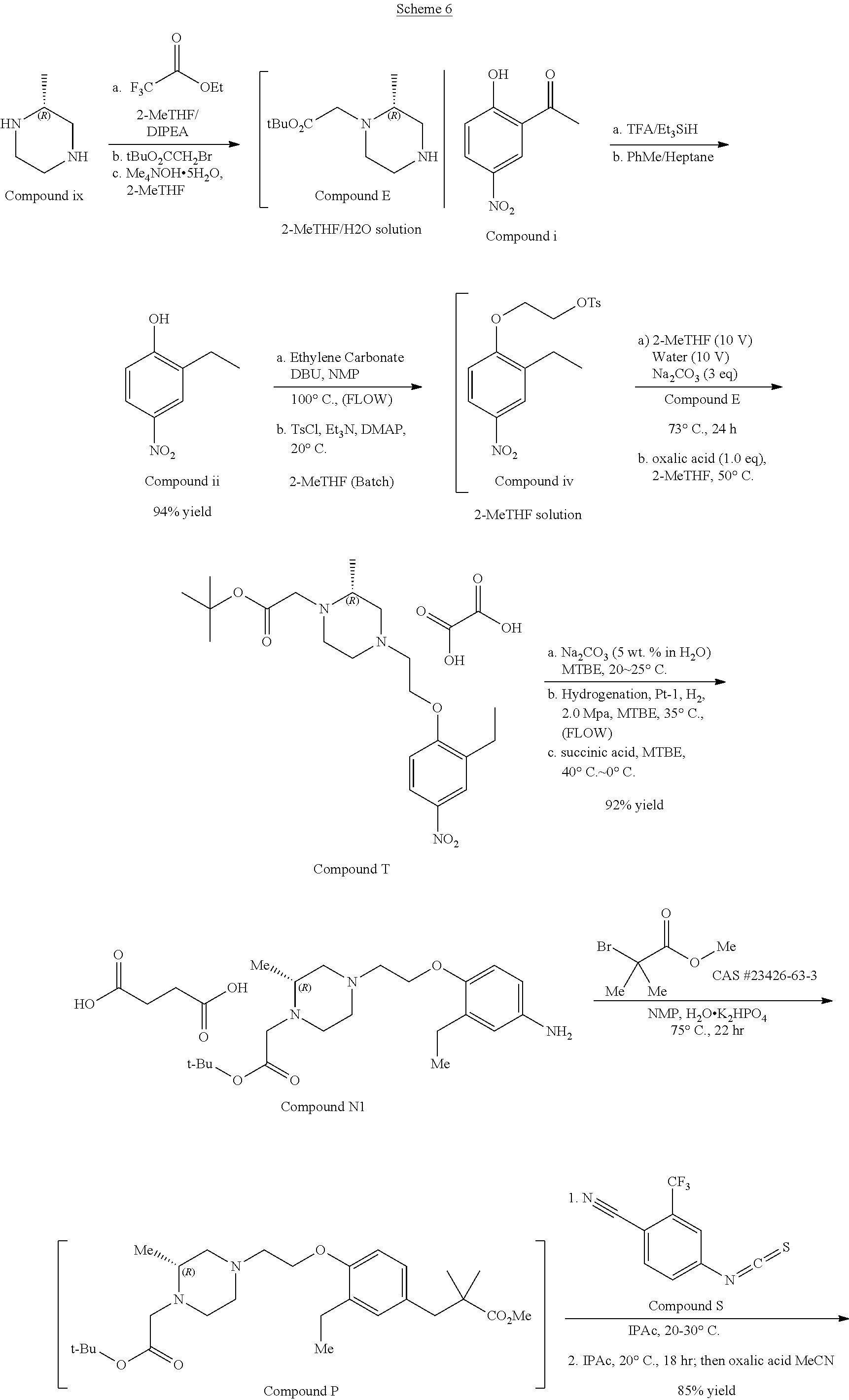

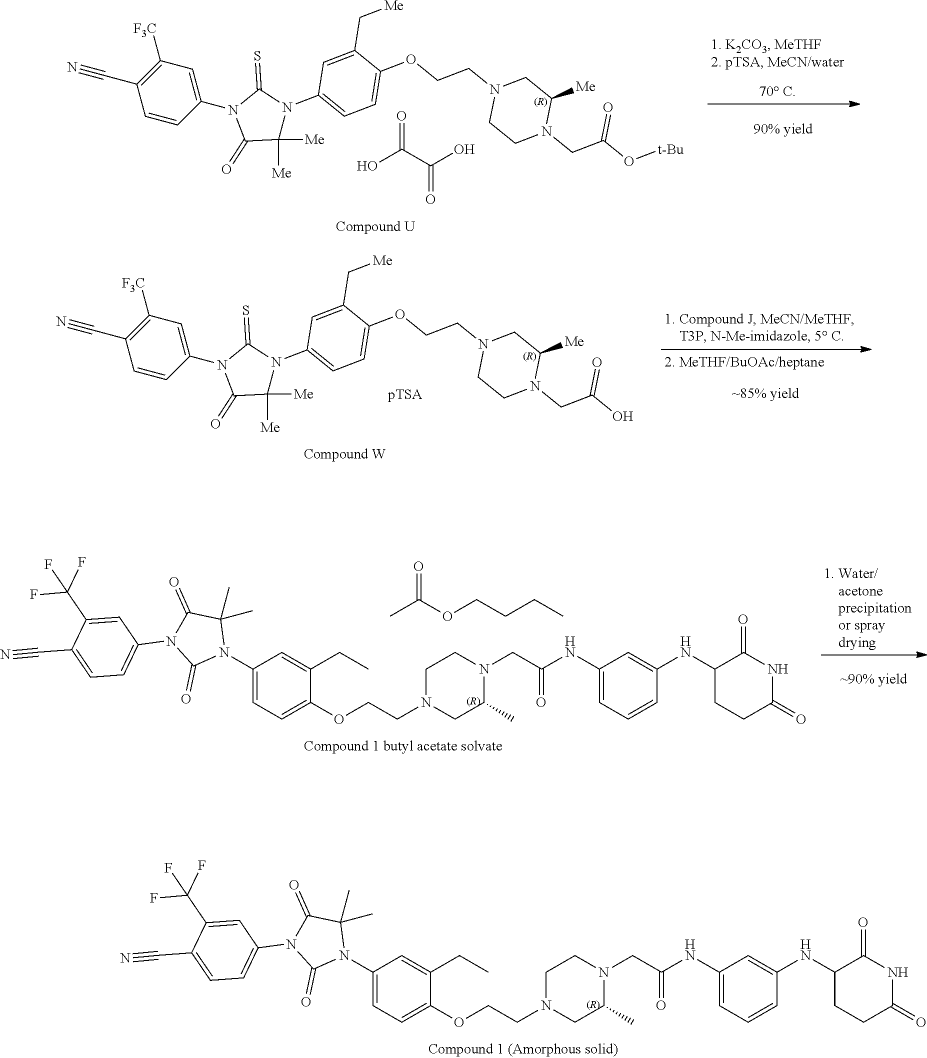

### Methods of Use

Compound 1 and Compound 1-1 described herein have utility in methods of treating an androgen receptor (AR) mediated disease. Described herein are Compound 1 and Compound 1-1 for use in methods of treating an androgen receptor mediated disease. Exemplary methods of treating an androgen receptor mediated disease comprising administering Compound 1 and/or Compound 1-1 are described in U.S. Pat. Nos. 11,149,007 and 11,873,283; and International Application No. PCT/US2023/078908.

In the following, embodiments are provided herein that include all possible combinations of the particular embodiments set forth herein.

In some embodiments, the AR mediated disease is an AR wild-type mediated disease. In other embodiments, the AR mediated disease is the result of AR amplification.

In certain embodiments, the AR mediated disease is prostate cancer. In some such embodiments, the prostate cancer is castration resistant prostate cancer (CRPC). In some such embodiments, the prostate cancer is metastatic castration resistant prostate cancer (mCRPC). In still another embodiment, the prostate cancer is non-metastatic CRPC (nmCRPC). In some embodiments, the prostate cancer is hormone refractory. In some embodiments, the prostate cancer is resistant to treatment with an AR antagonist. For example, the prostate cancer is resistant to treatment with one or more of enzalutamide, bicalutamide, abiraterone, ARN-509, ODM-201, EPI-001, EPI-506, AZD-3514, galeterone, ASC-J9, flutamide, hydroxyflutamide, nilutamide, cyproterone acetate, ketoconazole, or spironolactone.

Compound 1 and Compound 1-1 described herein have utility in methods of reducing AR levels relative to a baseline level, the methods comprise administering to a subject an effective amount of Compound 1 and/or Compound 1-1. In certain embodiments, the methods comprise administering to a subject an effective amount of Compound 1 and/or Compound 1-1 in combination with one or more second agents selected from a PI3K inhibitor, an AKT inhibitor, a BET inhibitor, JAK inhibitor, and an EZH2 inhibitor. In one embodiment, Compound 1 and Compound 1-1 described herein have utility in a method of reducing levels of wild-type AR within a tumor, the method comprising administering a therapeutically effective amount of Compound 1 and/or Compound 1-1 to reduce the level of wild-type AR within the tumor. In one embodiment, Compound 1 and Compound 1-1 described herein have utility in a method of reducing levels of wild-type AR within a tumor, the method comprising administering a therapeutically effective amount of Compound 1 and/or Compound 1-1 in combination with one or more second agents selected from a PI3K inhibitor, an AKT inhibitor, a BET inhibitor, JAK inhibitor, and an EZH2 inhibitor, to reduce the level of wild-type AR within the tumor. In one embodiment, Compound 1 and Compound 1-1 described herein have utility in methods of reducing levels of AR-full length (AR-FL) within a tumor, the method comprising administering a therapeutically effective amount of Compound 1 and/or Compound 1-1 to reduce the level of AR-full length (AR-FL) within the tumor. In one embodiment, Compound 1 and Compound 1-1 described herein have utility in methods of reducing levels of AR-full length (AR-FL) within a tumor, the method comprising administering a therapeutically effective amount of Compound 1 and/or Compound 1-1 in combination with one or more second agents selected from a PI3K inhibitor, an AKT inhibitor, a BET inhibitor, JAK inhibitor, and an EZH2 inhibitor, to reduce the level of AR-full length (AR-FL) within the tumor. In some embodiments, the AR levels are reduced compared to the AR levels prior to Compound 1 and/or Compound 1-1 administration. In some embodiments, the AR levels are reduced by 20%, 30%, 40%, 50%, 60%, 70%, 80%, 90%, 95%, or 99% compared to the AR levels prior to administration of Compound 1 and/or Compound 1-1.

Also provided herein are Compound 1 and Compound 1-1 for use in methods of treating any disease provided herein.

### Pharmaceutical Compositions and Routes of Administration

Compound 1 and Compound 1-1 prepared by the processes provided herein are useful for the preparation of pharmaceutical compositions, comprising an effective amount of Compound 1 and/or Compound 1-1 and a pharmaceutically acceptable excipient, carrier or vehicle. In some embodiments, the pharmaceutical composition described herein are suitable for oral, parenteral, mucosal, transdermal or topical administration.

### ENUMERATED EMBODIMENTS

Embodiment 1. A process for preparing Compound 1 or a stereoisomer or mixture of stereoisomers thereof.

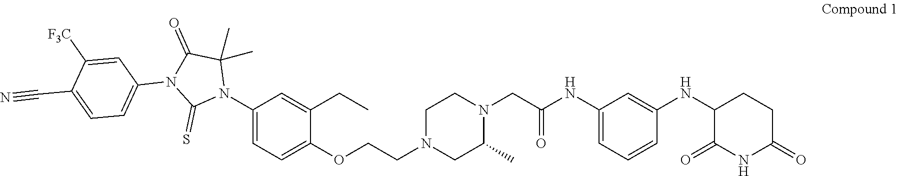 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1]

comprising contacting an acid salt of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid with an acid salt of 3-((3-aminophenyl)amino) piperidine-2,6-dione in the presence of a base in a solvent under conditions suitable to provide Compound 1 or a stereoisomer or mixture of stereoisomers thereof.

Embodiment 2. The process of embodiment 1, wherein the base is N-methyl imidazole, triethyl amine or pyridine.

Embodiment 3. The process of embodiment 1, wherein the base is N-methyl imidazole.

Embodiment 4. The process of any one of embodiments 1 to 3, wherein the solvent comprises THF, DMF, EtOAc, MeCN, DMAc, DMSO or a mixture thereof.

Embodiment 5. The process of any one of embodiments 1 to 3, wherein the solvent comprises a mixture of EtOAc/THF.

Embodiment 6. The process of any one of embodiments 1 to 3, wherein the solvent comprises THF.

Embodiment 7. The process of any one of embodiments 1 to 6, wherein the process comprises contacting Compound W

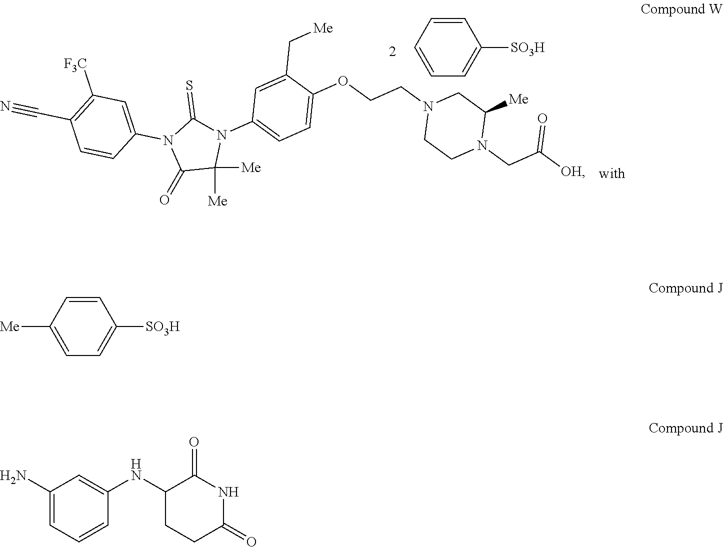

in the presence of a base in a solvent under conditions suitable to provide Compound 1 or a stereoisomer or mixture of stereoisomers thereof.

Embodiment 8. The process of embodiment 7, wherein the solvent comprises THF.

Embodiment 9. The process of embodiment 7 or 8, wherein Compound W is contacted with Compound J in the presence of a coupling agent.

Embodiment 10. The process of embodiment 9, wherein the coupling agent is propanephosphonic acid anhydride, hexafluorophosphate azabenzotriazole tetramethyl uronium, 1-ethyl-3-(3-dimethylaminopropyl) carbodiimide, (1-cyano-2-ethoxy-2-oxoethylidenaminooxy) dimethylamino-morpholino-carbenium hexafluorophosphate or (benzotriazol-1-yloxy)tripyrrolidinophosphonium hexafluorophosphate.

Embodiment 11. The process of embodiment 10, wherein the coupling agent is propanephosphonic acid anhydride.

Embodiment 12. The process of any one of embodiments 1 to 11, wherein the process further comprises contacting Compound U

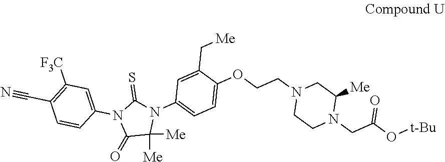 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

with BSA in a solvent under conditions suitable to provide Compound W.

Embodiment 13. The process of embodiment 12, wherein the solvent comprises acetonitrile.

Embodiment 14. The process of embodiment 12, wherein the process further comprises contacting Compound P

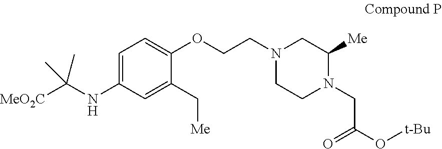 [SMILES: CCc1cc(NC(C)(C)C(=O)OC)ccc1OCCN1CCN(CC(=O)OC(C)(C)C)[C@H](C)C1]

with Compound S

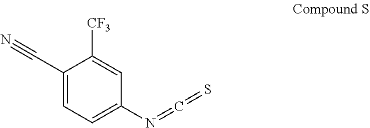 [SMILES: N#Cc1ccc(N=C=S)cc1C(F)(F)F]

in a solvent under conditions suitable to provide Compound U.

Embodiment 15. The process of embodiment 14, wherein the solvent comprises 2-methyltetrahydrofuran, IPrOAc or MeCN.

Embodiment 16. The process of embodiment 14 or 15, wherein the process further comprises contacting Compound N

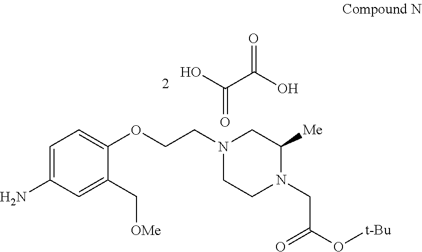

with methyl 2-bromo-2-methylpropionate in a solvent in the presence of a base under conditions suitable to provide Compound P.

Embodiment 17. The process of embodiment 16, wherein the base comprises K₂CO₃, TEA, DIEA, DBU, Na₂CO₃, Cs₂CO₃, Na₃PO₄, K₃PO₄, K₂HPO₄ or Na₂HPO₄.

Embodiment 18. The process of embodiment 16 or 17, wherein the base is K₂CO₃.

Embodiment 19. The process of any one of embodiments 16 to 18, wherein the solvent comprises NMP, CH₃CN, MeTHF, DMF, DMSO, DMAc or MeOH.

Embodiment 20. The process of any one of embodiments 1 to 19, wherein the solvent comprises NMP.

Embodiment 21. The process of embodiment 16, wherein the process further comprises contacting Compound L

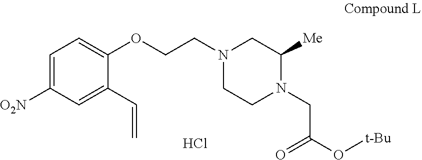 [SMILES: C=Cc1cc([N+](=O)[O-])ccc1OCCN1CCN(CC(=O)OC(C)(C)C)[C@H](C)C1 ; Cl]

with a palladium catalyst in a solvent under conditions suitable to provide Compound N.

Embodiment 22. The process of embodiment 21, wherein the solvent comprises 2-methyltetrahydrofuran.

Embodiment 23. The process of embodiment 21 or 22, wherein the process further comprises contacting Compound L with with a palladium catalyst and oxalic acid under conditions suitable to provide Compound N.

Embodiment 24. The process of any one of embodiments 21 to 23, wherein the process further comprises contacting Compound K

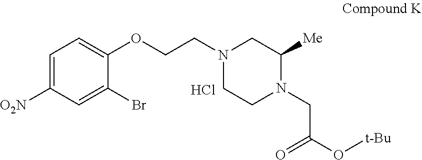 [SMILES: C[C@@H]1CN(CCOc2ccc([N+](=O)[O-])cc2Br)CCN1CC(=O)OC(C)(C)C ; Cl]

with vinyl dioxaborolane in the presence of a palladium catalyst and a base in a solvent under conditions suitable to provide Compound L.

Embodiment 25. The process of embodiment 24, wherein the solvent comprises a mixture of tetrahydrofuran and water.

Embodiment 26. The process of embodiment 24 or 25, wherein the catalyst is Pd (DPPF) C12 (dichloro [1,1′-bis(diphenylphosphino)-ferrocene]palladium (II) dichloro-methane).

Embodiment 27. The process of any one of embodiments 24 to 26, wherein the base is potassium phosphate.

Embodiment 28. The process of any one of embodiments 24 to 27, wherein the process further comprises contacting Compound E

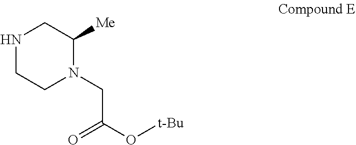 [SMILES: C[C@@H]1CNCCN1CC(=O)OC(C)(C)C]

with Compound I

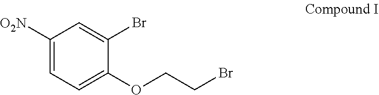 [SMILES: O=[N+]([O-])c1ccc(OCCBr)c(Br)c1]

in the presence of a base in a solvent under conditions suitable to provide Compound K.

Embodiment 29. The process of embodiment 28, wherein the solvent comprises a mixture of tetrahydrofuran and water.

Embodiment 30. The process of embodiment 28 or 29, wherein the base is potassium carbonate.

Embodiment 31. The process of any one of embodiments 28 to 30, wherein the process further comprises contacting Compound AA

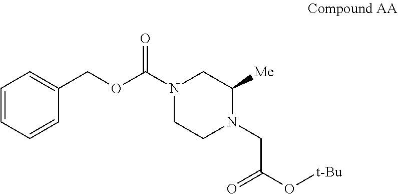 [SMILES: C[C@@H]1CN(C(=O)OCc2ccccc2)CCN1CC(=O)OC(C)(C)C]

with a palladium catalyst in a solvent under conditions suitable to provide Compound E.

Embodiment 32. The process of embodiment 31, wherein the solvent comprises 2-methyltetrahydrofuran.

Embodiment 33. The process of embodiment 31 or 32, wherein the process further comprises contacting Compound A′

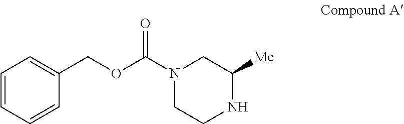 [SMILES: C[C@@H]1CN(C(=O)OCc2ccccc2)CCN1]

with tert-butyl 2-bromoacetate in a solvent under conditions suitable to provide Compound AA.

Embodiment 34. The process of embodiment 33, wherein the solvent comprises 2-methyltetrahydrofuran and water.

Embodiment 35. The process of embodiment 7, wherein the process further comprises contacting Compound H

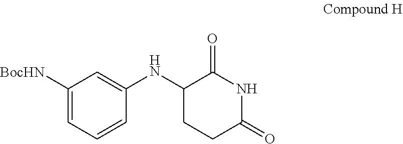 [SMILES: CC(C)(C)OC(=O)Nc1cccc(NC2CCC(=O)NC2=O)c1]

with p-toluenesulfonic acid monohydrate under conditions suitable to provide Compound J.

Embodiment 36. The process of embodiment 35, wherein the process comprises contacting Compound H with p-toluenesulfonic acid monohydrate in acetonitrile at 70 to 75° C. to provide Compound J.

Embodiment 37. The process of embodiment 35 or 36, wherein the process comprises contacting an acid salt of 3-aminopiperidine-2,6-dione

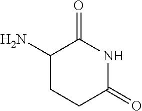

with tert-butyl (3-bromophenyl) carbamate

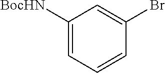 [SMILES: CC(C)(C)OC(=O)Nc1cccc(Br)c1]

under conditions suitable to provide Compound H.

Embodiment 38. The process of embodiment 37, wherein the process further comprises contacting a hydrochloride salt of 3-aminopiperidine-2,6-dione with tert-butyl (3-bromophenyl) carbamate in a solvent in the presence of a base and a catalyst under conditions suitable to provide Compound H.

Embodiment 39. The process of embodiment 37 or 38, wherein the process further comprises contacting 3-bromopiperidine-2,6-dione (Compound D)

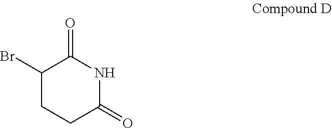

with tert-butyl (3-aminophenyl) carbamate

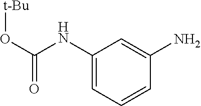 [SMILES: CC(C)(C)OC(=O)Nc1cccc(N)c1]

under conditions suitable to provide Compound H.

Embodiment 40. The process of embodiment 39, wherein the process comprises contacting 6,7-dihydro-5H-oxazolo[3,2-a]pyridine-2,3,5-trione (Compound C)

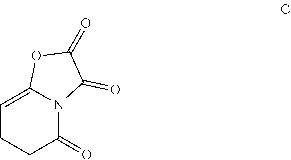 [SMILES: O=C1OC2=CCCC(=O)N2C1=O]

with n-bromosuccinimide (NBS) under conditions suitable to provide Compound D.

Embodiment 41. The process of embodiment 40, wherein the process comprises contacting piperidine-2,6-dione (Compound A)

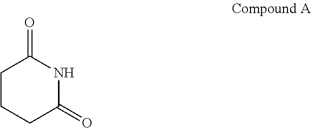

with oxalyl chloride under conditions suitable to provide Compound C.

Embodiment 42. The process of embodiment 1, wherein the process comprises
a) contacting Compound N

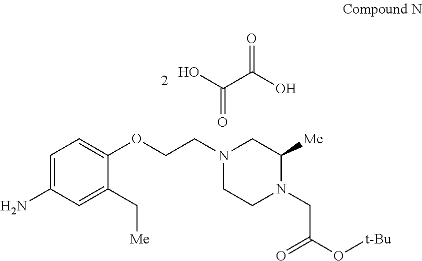

with methyl 2-bromo-2-methylpropionate under conditions suitable to provide Compound P

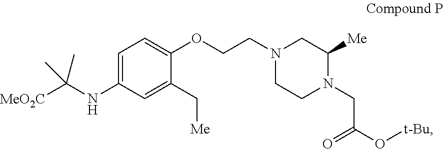 [SMILES: CCc1cc(NC(C)(C)C(=O)OC)ccc1OCCN1CCN(CC(=O)OC(C)(C)C)[C@H](C)C1]

b) contacting Compound P with Compound S

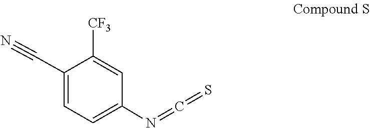 [SMILES: N#Cc1ccc(N=C=S)cc1C(F)(F)F]

under conditions suitable to provide Compound U

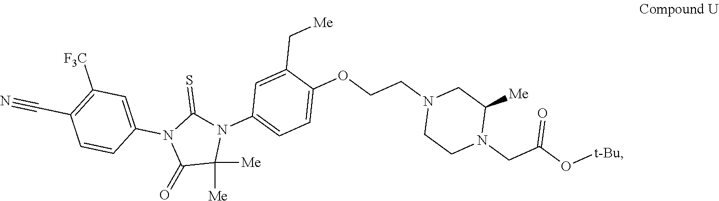 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

c) contacting Compound U with an acid under conditions suitable to provide Compound W

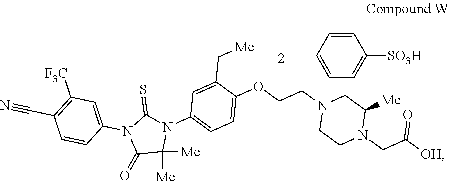

and d) contacting Compound W with Compound J

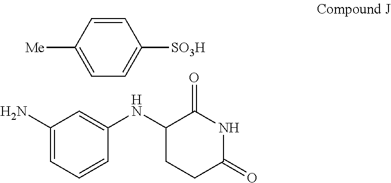

under conditions suitable to provide Compound 1.

Embodiment 43. The process of embodiment 42, wherein the process further comprises
i) contacting an acid salt of 3-aminopiperidine-2,6-dione

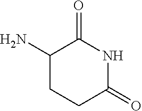

with tert-butyl (3-bromophenyl) carbamate

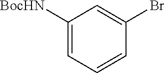 [SMILES: CC(C)(C)OC(=O)Nc1cccc(Br)c1]

under conditions suitable to provide Compound H

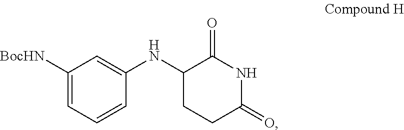 [SMILES: CC(C)(C)OC(=O)Nc1cccc(NC2CCC(=O)NC2=O)c1]

and ii) contacting Compound H with p-toluenesulfonic acid monohydrate under conditions suitable to provide Compound J

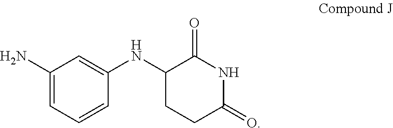 [SMILES: Nc1cccc(NC2CCC(=O)NC2=O)c1]

Embodiment 44. The process of embodiment 43 further comprising
i) contacting piperidine-2,6-dione (Compound A)

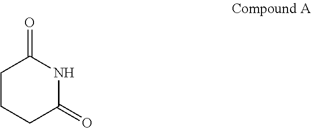

with oxalyl chloride under conditions suitable to provide Compound C

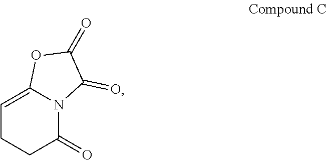 [SMILES: O=C1OC2=CCCC(=O)N2C1=O]

ii) contacting Compound C with n-bromosuccinimide (NBS) under conditions suitable to provide Compound D

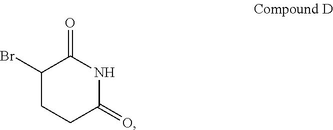

iii) contacting Compound D with tert-butyl (3-aminophenyl) carbamate

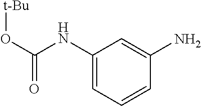 [SMILES: CC(C)(C)OC(=O)Nc1cccc(N)c1]

under conditions suitable to provide Compound H

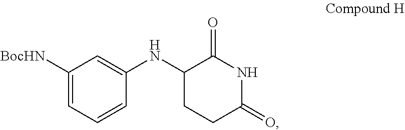 [SMILES: CC(C)(C)OC(=O)Nc1cccc(NC2CCC(=O)NC2=O)c1]

and
iv) contacting Compound H with p-toluenesulfonic acid monohydrate under conditions suitable to provide Compound J.

Embodiment 45. The process of embodiment 42, where Compound N is prepared by
1) contacting Compound A′

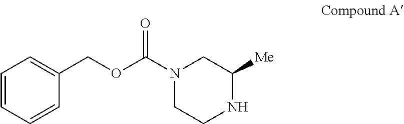 [SMILES: C[C@@H]1CN(C(=O)OCc2ccccc2)CCN1]

with tert-butyl 2-bromoacetate in a solvent under conditions suitable to provide Compound AA

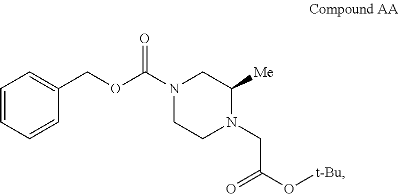 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

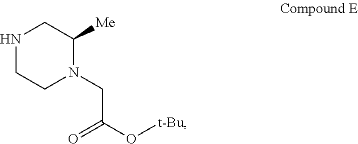 [SMILES: C[C@@H]1CNCCN1CC(=O)OC(C)(C)C]

3) contacting Compound E with Compound I

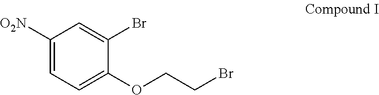 [SMILES: O=[N+]([O-])c1ccc(OCCBr)c(Br)c1]

in the presence of a base in a solvent under conditions suitable to provide Compound K

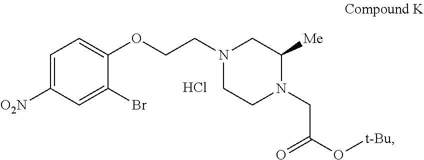 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

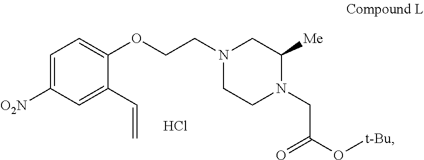 [SMILES: C=Cc1cc([N+](=O)[O-])ccc1OCCN1CCN(CC(=O)OC(C)(C)C)[C@H](C)C1 ; Cl]

and 5) contacting Compound L with a palladium catalyst in a solvent under conditions suitable to provide Compound N.

Embodiment 46. The process of embodiment 1, wherein the process comprises
1) contacting Compound A′

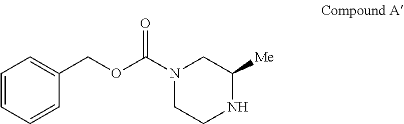 [SMILES: C[C@@H]1CN(C(=O)OCc2ccccc2)CCN1]

with tert-butyl 2-bromoacetate in a solvent under conditions suitable to provide Compound AA

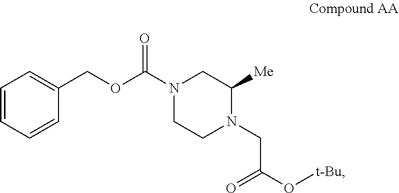 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

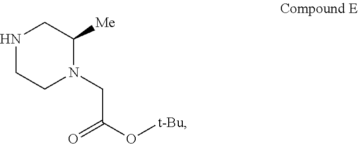 [SMILES: C[C@@H]1CNCCN1CC(=O)OC(C)(C)C]

3) contacting Compound E with Compound I

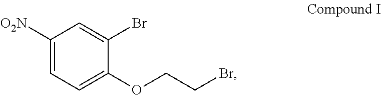 [SMILES: O=[N+]([O-])c1ccc(OCCBr)c(Br)c1]

in the presence of a base in a solvent under conditions suitable to provide Compound K

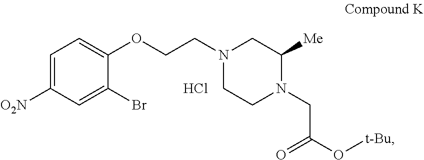 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

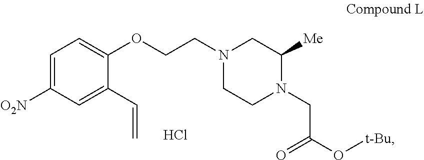 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

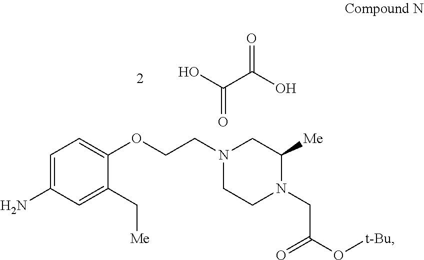

6) contacting Compound N with methyl 2-bromo-2-methylpropionate under conditions suitable to provide Compound P

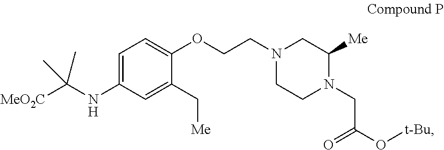 [SMILES: CCc1cc(NC(C)(C)C(=O)OC)ccc1OCCN1CCN(CC(=O)OC(C)(C)C)[C@H](C)C1]

7) contacting Compound P with Compound S

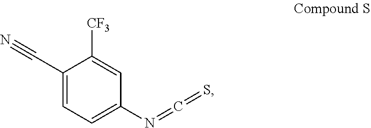 [SMILES: N#Cc1ccc(N=C=S)cc1C(F)(F)F]

under conditions suitable to provide Compound U

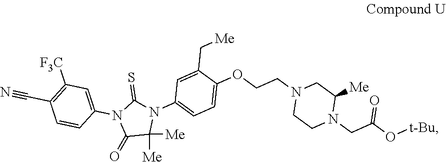 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

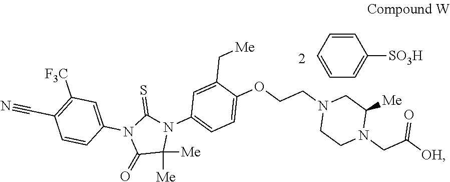

and 9) contacting Compound W with Compound J

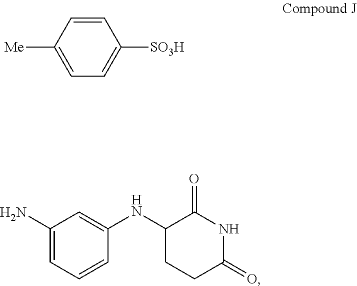 [SMILES: Cc1ccc(S(=O)(=O)O)cc1 ; Nc1cccc(NC2CCC(=O)NC2=O)c1]

under conditions suitable to provide Compound 1, wherein Compound J is prepared by
i) contacting an acid salt of 3-aminopiperidine-2,6-dione

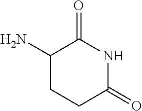

with tert-butyl (3-bromophenyl) carbamate

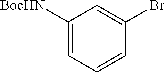 [SMILES: CC(C)(C)OC(=O)Nc1cccc(Br)c1]

under conditions suitable to provide Compound H

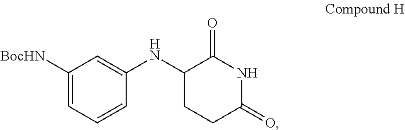 [SMILES: CC(C)(C)OC(=O)Nc1cccc(NC2CCC(=O)NC2=O)c1]

and ii) contacting Compound H with p-toluenesulfonic acid monohydrate under conditions suitable to provide Compound J.

Embodiment 47. The process of embodiment 1, wherein the process comprises 1) contacting Compound A′

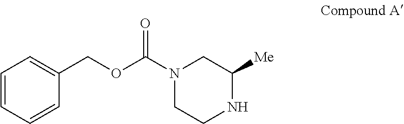 [SMILES: C[C@@H]1CN(C(=O)OCc2ccccc2)CCN1]

with tert-butyl 2-bromoacetate in a solvent under conditions suitable to provide Compound AA

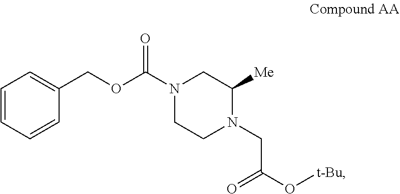 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

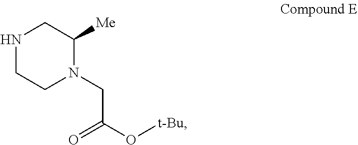 [SMILES: C[C@@H]1CNCCN1CC(=O)OC(C)(C)C]

3) contacting Compound E with Compound I

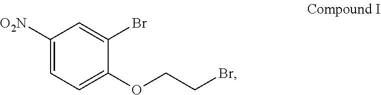 [SMILES: O=[N+]([O-])c1ccc(OCCBr)c(Br)c1]

in the presence of a base in a solvent under conditions suitable to provide Compound K

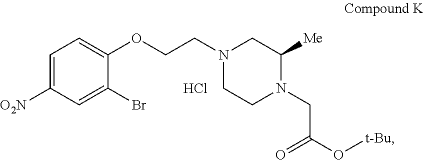 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

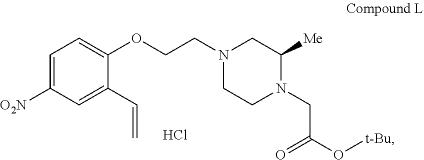 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

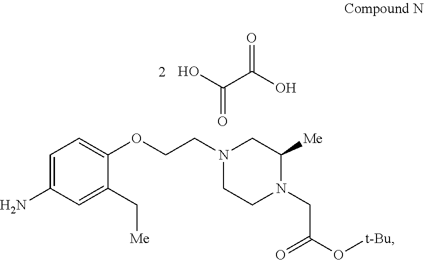

6) contacting Compound N with methyl 2-bromo-2-methylpropionate under conditions suitable to provide Compound P

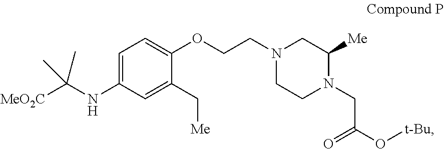 [SMILES: CCc1cc(NC(C)(C)C(=O)OC)ccc1OCCN1CCN(CC(=O)OC(C)(C)C)[C@H](C)C1]

7) contacting Compound P with Compound S

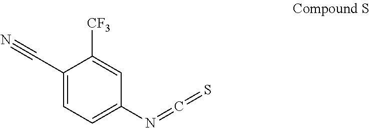 [SMILES: N#Cc1ccc(N=C=S)cc1C(F)(F)F]

under conditions suitable to provide Compound U

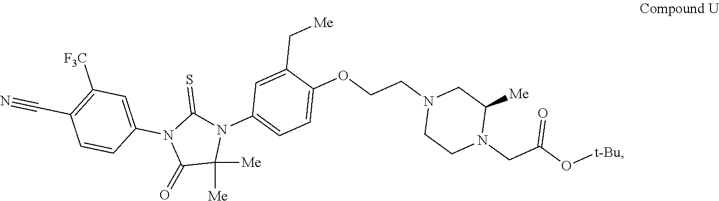 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

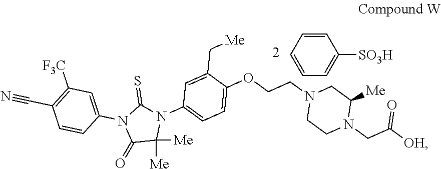

and 9) contacting Compound W with Compound J

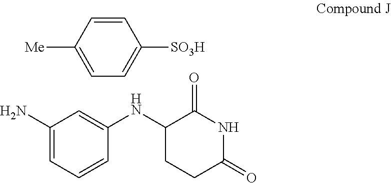

under conditions suitable to provide Compound 1,
wherein Compound J is prepared by
i) contacting piperidine-2,6-dione (Compound A)

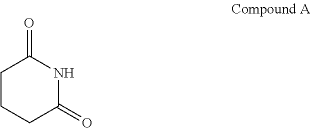

with oxalyl chloride under conditions suitable to provide Compound C

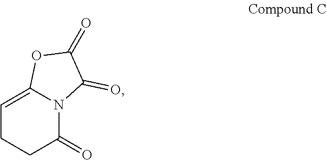 [SMILES: O=C1OC2=CCCC(=O)N2C1=O]

ii) contacting Compound C with n-bromosuccinimide (NBS) under conditions suitable to provide Compound D

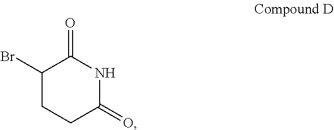

iii) contacting Compound D with tert-butyl (3-aminophenyl) carbamate

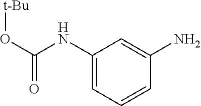 [SMILES: CC(C)(C)OC(=O)Nc1cccc(N)c1]

under conditions suitable to provide Compound H

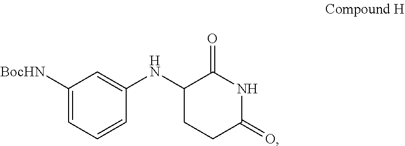 [SMILES: CC(C)(C)OC(=O)Nc1cccc(NC2CCC(=O)NC2=O)c1]

and iv) contacting Compound H with p-toluenesulfonic acid monohydrate under conditions suitable to provide Compound J.

Embodiment 48. A process for preparing Compound 1-1

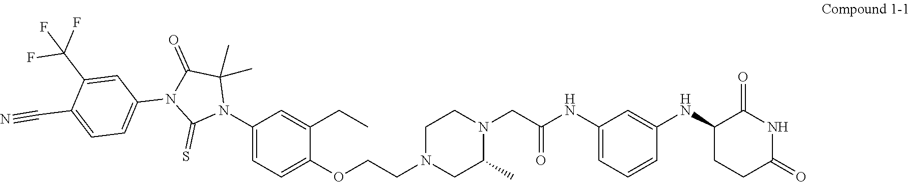 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

comprising
a) contacting Compound W

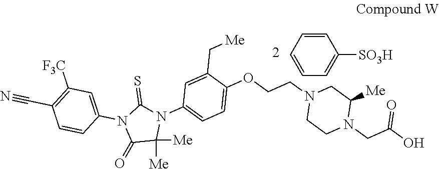

with Compound (R)-J

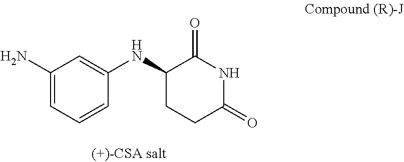 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1,
b) contacting the BSA salt of Compound 1-1 with a buffer in a solvent to obtain a solution comprising Compound 1-1, and
c) contacting the solution with water/acetone mixture to obtain Compound 1-1.

Embodiment 49. The process of embodiment 48, wherein the base is N-methyl imidazole.

Embodiment 50. The process of embodiment 48, wherein the solvent in step a) comprises THF.

Embodiment 51. The process of embodiment 48, wherein Compound Wis contacted with Compound (R)-J in the presence of a coupling agent.

Embodiment 52. The process of embodiment 51, wherein the coupling agent is propanephosphonic acid anhydride (T3P).

Embodiment 53. The process of embodiment 48, wherein the solvent in step b) is ethyl acetate.

Embodiment 54. The process of embodiment 48, wherein the buffer in step b) is a potassium phosphate buffer.

Embodiment 55. A process for preparing Compound (R)-J

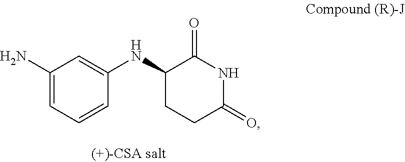 [SMILES: Nc1cccc(N[C@@H]2CCC(=O)NC2=O)c1]

comprising contacting Compound J

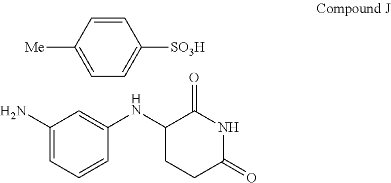

with (+) camphor-10-sulphonic acid ((+) CSA) and a base under conditions suitable to provide Compound (R)-J.

Embodiment 56. A process for preparing Compound(S)-J

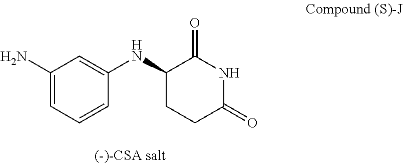 [SMILES: Nc1cccc(N[C@@H]2CCC(=O)NC2=O)c1]

comprising contacting Compound J

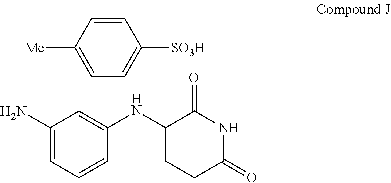

with (−) camphor-10-sulphonic acid ((−) CSA) and a base under conditions suitable to provide Compound(S)-J.

Embodiment 57. A process for preparing Compound 1-1 comprising
1) contacting Compound A′

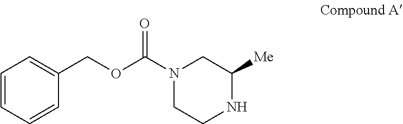 [SMILES: C[C@@H]1CN(C(=O)OCc2ccccc2)CCN1]

with tert-butyl 2-bromoacetate in a solvent under conditions suitable to provide Compound AA

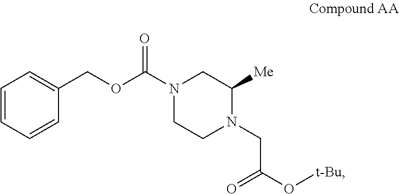 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

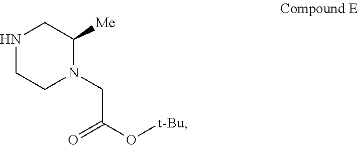 [SMILES: C[C@@H]1CNCCN1CC(=O)OC(C)(C)C]

3) contacting Compound E with Compound I

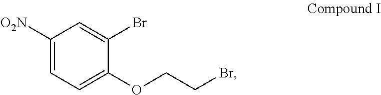 [SMILES: O=[N+]([O-])c1ccc(OCCBr)c(Br)c1]

in the presence of a base in a solvent under conditions suitable to provide Compound K

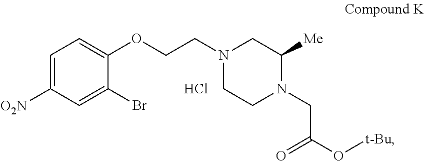 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

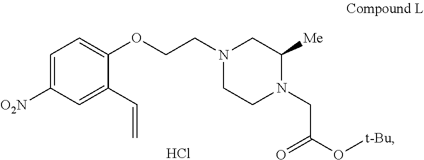 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

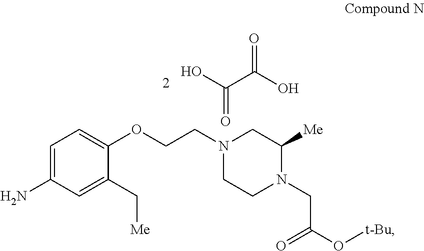

6) contacting Compound N with methyl 2-bromo-2-methylpropionate under conditions suitable to provide Compound P

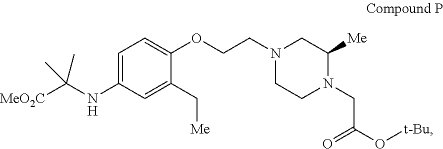 [SMILES: CCc1cc(NC(C)(C)C(=O)OC)ccc1OCCN1CCN(CC(=O)OC(C)(C)C)[C@H](C)C1]

7) contacting Compound P with Compound S

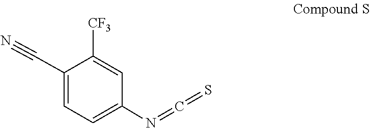 [SMILES: N#Cc1ccc(N=C=S)cc1C(F)(F)F]

under conditions suitable to provide Compound U

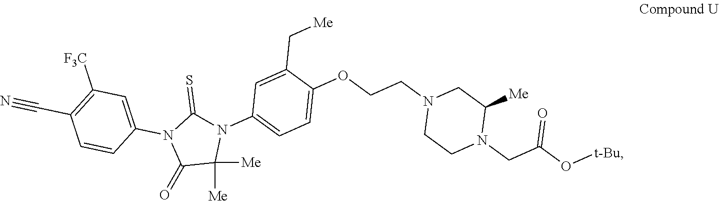 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

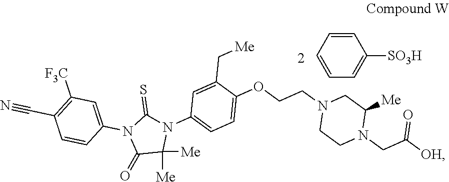

9) contacting Compound W with Compound (R)-J

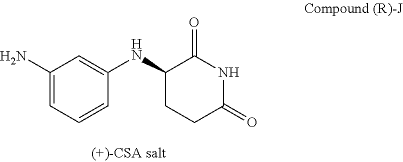 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1

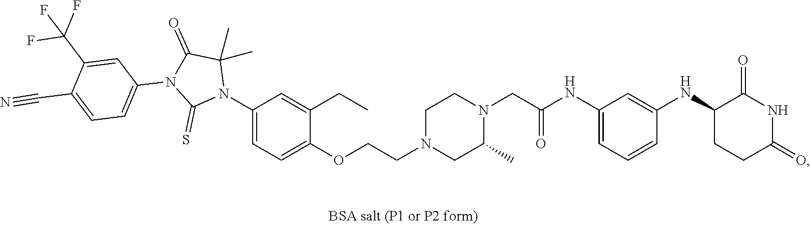 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

10) contacting the BSA salt of Compound 1-1 with a buffer in a solvent to obtain a solution comprising Compound 1-1, and
11) contacting the solution with water/acetone mixture to obtain Compound 1 1.

Embodiment 58. The process of embodiment 57, further comprising
i) contacting an acid salt of 3-aminopiperidine-2,6-dione

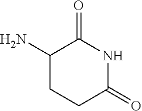

with tert-butyl (3-bromophenyl) carbamate

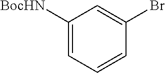 [SMILES: CC(C)(C)OC(=O)Nc1cccc(Br)c1]

under conditions suitable to provide Compound H

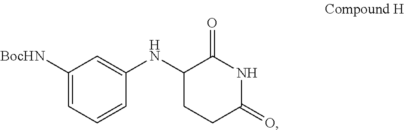 [SMILES: CC(C)(C)OC(=O)Nc1cccc(NC2CCC(=O)NC2=O)c1]

ii) contacting Compound HI with p-toluenesulfonic acid monohydrate under conditions suitable to provide Compound J

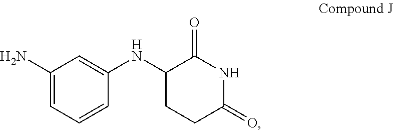 [SMILES: Nc1cccc(NC2CCC(=O)NC2=O)c1]

and iii) contacting Compound J with (+) camphor-10-sulphonic acid ((+) CSA) and a base under conditions suitable to provide Compound (R)-J

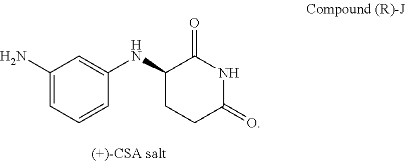 [SMILES: Nc1cccc(N[C@@H]2CCC(=O)NC2=O)c1]

Embodiment 59. The process of embodiment 57, further comprising
i) contacting piperidine-2,6-dione (Compound A)

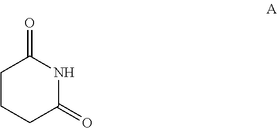

with oxalyl chloride under conditions suitable to provide Compound C

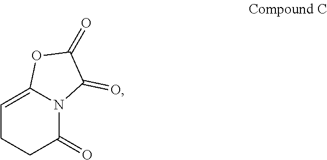 [SMILES: O=C1OC2=CCCC(=O)N2C1=O]

ii) contacting Compound C with n-bromosuccinimide (NBS) under conditions suitable to provide Compound D

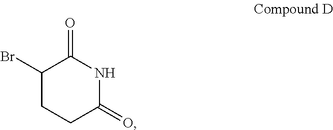

iii) contacting Compound D with tert-butyl (3-aminophenyl) carbamate

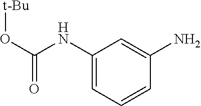 [SMILES: CC(C)(C)OC(=O)Nc1cccc(N)c1]

under conditions suitable to provide Compound H

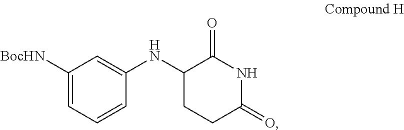 [SMILES: CC(C)(C)OC(=O)Nc1cccc(NC2CCC(=O)NC2=O)c1]

iv) contacting Compound H with p-toluenesulfonic acid monohydrate under conditions suitable to provide Compound J

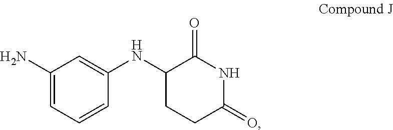 [SMILES: Nc1cccc(NC2CCC(=O)NC2=O)c1]

and v) contacting Compound J with (+) camphor-10-sulphonic acid ((+) CSA) and a base under conditions suitable to provide Compound (R)-J

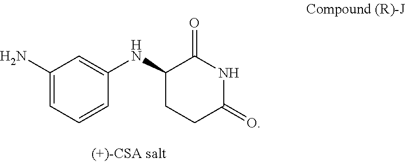 [SMILES: Nc1cccc(N[C@@H]2CCC(=O)NC2=O)c1]

Embodiment 60. A process for preparing Compound 1-1 comprising
1) contacting Compound A′

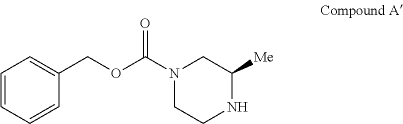 [SMILES: C[C@@H]1CN(C(=O)OCc2ccccc2)CCN1]

with tert-butyl 2-bromoacetate in a solvent under conditions suitable to provide Compound AA

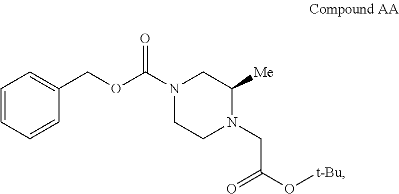 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

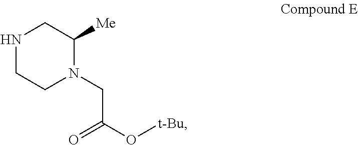 [SMILES: C[C@@H]1CNCCN1CC(=O)OC(C)(C)C]

3) contacting Compound E with Compound I

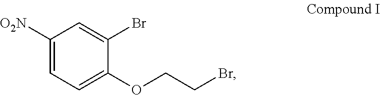 [SMILES: O=[N+]([O-])c1ccc(OCCBr)c(Br)c1]

in the presence of a base in a solvent under conditions suitable to provide Compound K

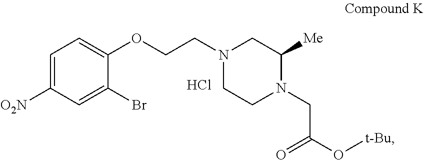 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

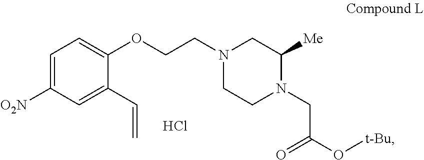 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

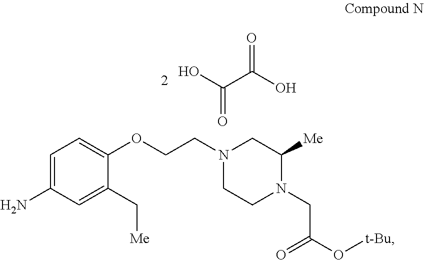

6) contacting Compound N with methyl 2-bromo-2-methylpropionate under conditions suitable to provide Compound P

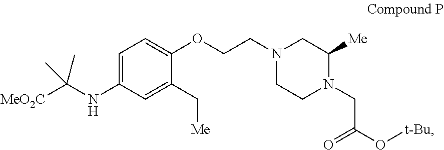 [SMILES: CCc1cc(NC(C)(C)C(=O)OC)ccc1OCCN1CCN(CC(=O)OC(C)(C)C)[C@H](C)C1]

7) contacting Compound P with Compound S

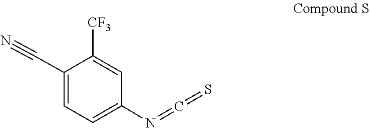 [SMILES: N#Cc1ccc(N=C=S)cc1C(F)(F)F]

under conditions suitable to provide Compound U

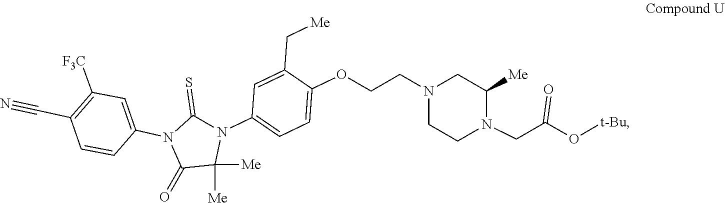 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

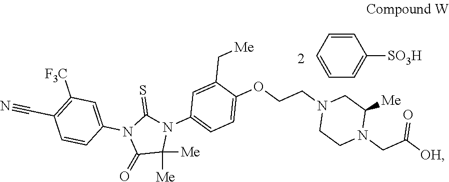

9) contacting Compound W with Compound (R)-J

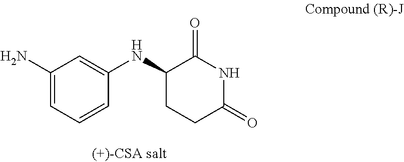 [SMILES: Nc1cccc(N[C@@H]2CCC(=O)NC2=O)c1]

in the presence of a base in a solvent and benzenesulfonic acid (BSA) under conditions suitable to provide BSA salt of Compound 1-1

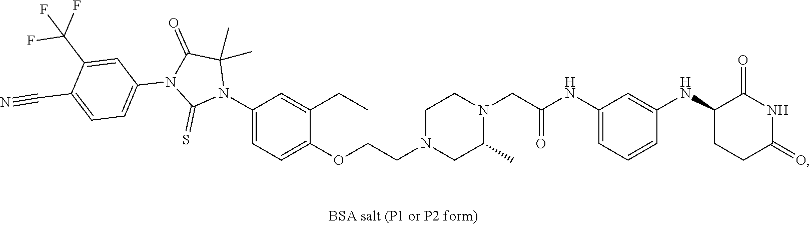 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

10) contacting the BSA salt of Compound 1-1 with a buffer in a solvent to obtain a solution comprising Compound 1-1, and
11) contacting the solution with water/acetone mixture to obtain Compound 1 1, wherein Compound (R)-J is prepared by
i) contacting an acid salt of 3-aminopiperidine-2,6-dione

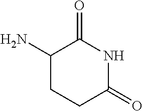

with tert-butyl (3-bromophenyl) carbamate

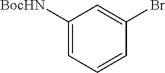 [SMILES: CC(C)(C)OC(=O)Nc1cccc(Br)c1]

under conditions suitable to provide Compound H

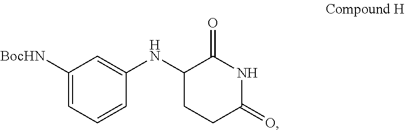 [SMILES: CC(C)(C)OC(=O)Nc1cccc(NC2CCC(=O)NC2=O)c1]

ii) contacting Compound H with p-toluenesulfonic acid monohydrate under conditions suitable to provide Compound J

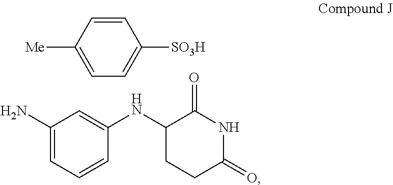

and iii) contacting Compound J with (+) camphor-10-sulphonic acid ((+) CSA) and a base under conditions suitable to provide Compound (R)-J

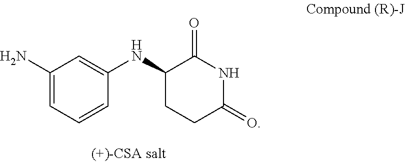 [SMILES: Nc1cccc(N[C@@H]2CCC(=O)NC2=O)c1]

Embodiment 61. A process for preparing Compound 1-1 comprising
1) contacting Compound A′

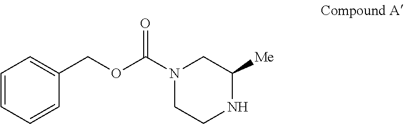 [SMILES: C[C@@H]1CN(C(=O)OCc2ccccc2)CCN1]

with tert-butyl 2-bromoacetate in a solvent under conditions suitable to provide Compound AA

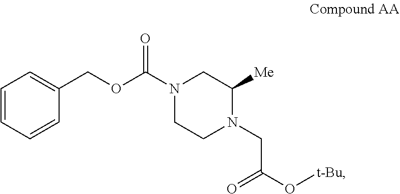 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

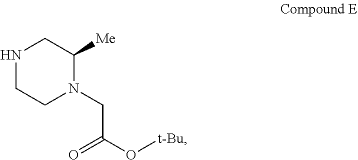 [SMILES: C[C@@H]1CNCCN1CC(=O)OC(C)(C)C]

3) contacting Compound E with Compound I

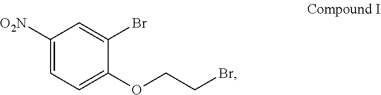 [SMILES: O=[N+]([O-])c1ccc(OCCBr)c(Br)c1]

in the presence of a base in a solvent under conditions suitable to provide Compound K

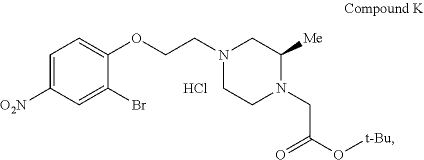 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

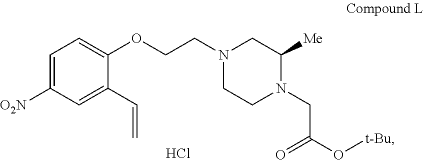 [SMILES: C=Cc1cc([N+](=O)[O-])ccc1OCCN1CCN(CC(=O)OC(C)(C)C)[C@H](C)C1 ; Cl]

5) contacting Compound L with a palladium catalyst in a solvent under conditions suitable to provide Compound N

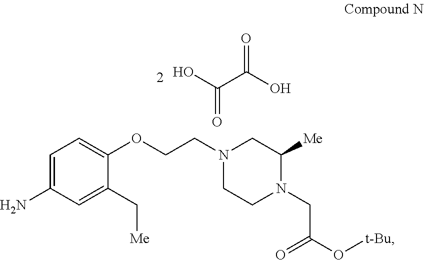

6) contacting Compound N with methyl 2-bromo-2-methylpropionate under conditions suitable to provide Compound P

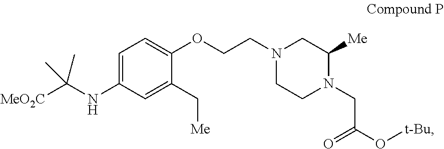 [SMILES: CCc1cc(NC(C)(C)C(=O)OC)ccc1OCCN1CCN(CC(=O)OC(C)(C)C)[C@H](C)C1]

7) contacting Compound P with Compound S

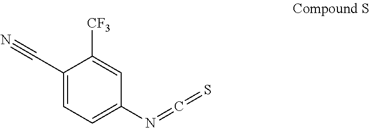 [SMILES: N#Cc1ccc(N=C=S)cc1C(F)(F)F]

under conditions suitable to provide Compound U

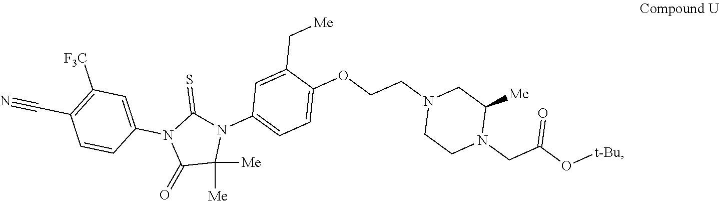 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

8) contacting Compound U with an acid to deprotect the tert-butyl ester group under conditions suitable to provide Compound W

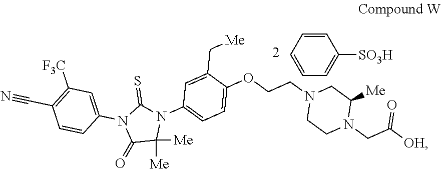

9) contacting Compound W with Compound (R)-J

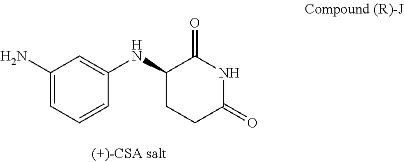 [SMILES: Nc1cccc(N[C@@H]2CCC(=O)NC2=O)c1]

under conditions suitable to provide suitable to provide BSA salt of Compound 1-1

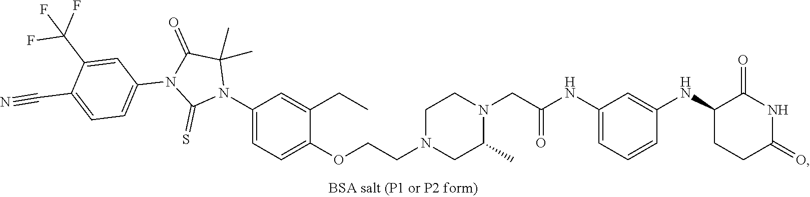 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

10) contacting the BSA salt of Compound 1-1 with a buffer in a solvent to obtain a solution comprising Compound 1-1, and
11) contacting the solution with water/acetone mixture to obtain Compound 1 1, wherein Compound (R)-J is prepared by
i) contacting piperidine-2,6-dione (Compound A)

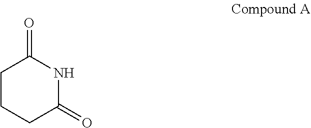

with oxalyl chloride under conditions suitable to provide Compound C

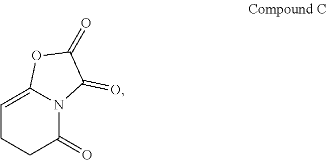 [SMILES: O=C1OC2=CCCC(=O)N2C1=O]

ii) contacting Compound C with n-bromosuccinimide (NBS) under conditions suitable to provide Compound D

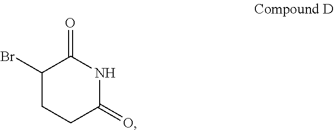

iii) contacting Compound D with tert-butyl (3-aminophenyl) carbamate

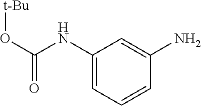 [SMILES: CC(C)(C)OC(=O)Nc1cccc(N)c1]

under conditions suitable to provide Compound H

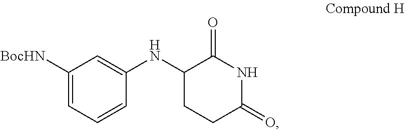 [SMILES: CC(C)(C)OC(=O)Nc1cccc(NC2CCC(=O)NC2=O)c1]

iv) contacting Compound H with p-toluenesulfonic acid monohydrate under conditions suitable to provide Compound J

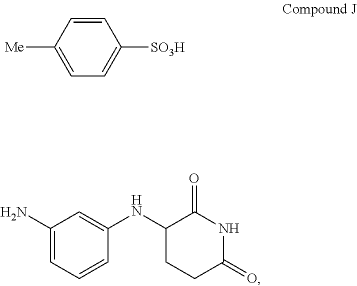 [SMILES: Cc1ccc(S(=O)(=O)O)cc1 ; Nc1cccc(NC2CCC(=O)NC2=O)c1]

and v) contacting Compound J with (+) camphor-10-sulphonic acid ((+) CSA) and a base under conditions suitable to provide Compound (R)-J

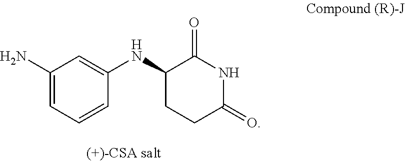 [SMILES: Nc1cccc(N[C@@H]2CCC(=O)NC2=O)c1]

Embodiment 62. A process for preparing Compound N

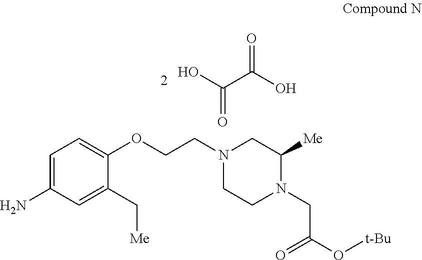

comprising contacting Compound L

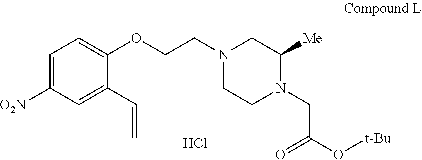 [SMILES: C=Cc1cc([N+](=O)[O-])ccc1OCCN1CCN(CC(=O)OC(C)(C)C)[C@H](C)C1 ; Cl]

with a palladium catalyst in a solvent under conditions suitable to provide Compound N.

Embodiment 63. The process of embodiment 62 comprising
1) contacting Compound A′

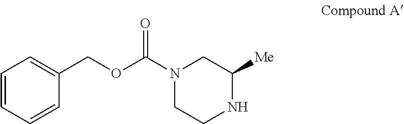 [SMILES: C[C@@H]1CN(C(=O)OCc2ccccc2)CCN1]

with tert-butyl 2-bromoacetate in a solvent under conditions suitable to provide Compound AA

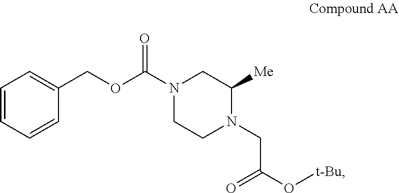 [SMILES: C[C@@H]1CN(C(=O)OCc2ccccc2)CCN1CC(=O)OC(C)(C)C]

2) contacting Compound AA with a palladium catalyst in a solvent under conditions suitable to provide Compound E

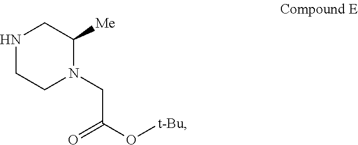 [SMILES: C[C@@H]1CNCCN1CC(=O)OC(C)(C)C]

3) contacting Compound E with Compound I

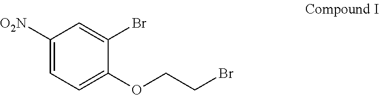 [SMILES: O=[N+]([O-])c1ccc(OCCBr)c(Br)c1]

in the presence of a base in a solvent under conditions suitable to provide Compound K

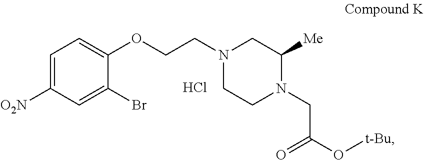 [SMILES: C[C@@H]1CN(CCOc2ccc([N+](=O)[O-])cc2Br)CCN1CC(=O)OC(C)(C)C ; Cl]

4) contacting Compound K with vinyl dioxaborolane in the presence of a palladium catalyst and a base in a solvent under conditions suitable to provide Compound L

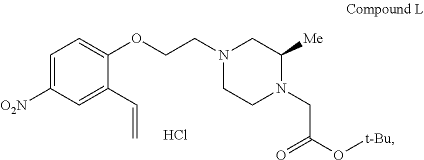 [SMILES: C=Cc1cc([N+](=O)[O-])ccc1OCCN1CCN(CC(=O)OC(C)(C)C)[C@H](C)C1 ; Cl]

and 5) contacting Compound L with a palladium catalyst in a solvent under conditions suitable to provide Compound N.

Embodiment 64. A process for preparing Compound U

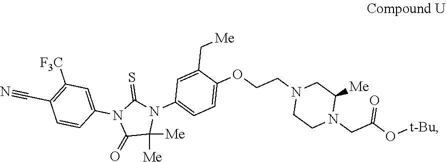 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

comprising contacting Compound P

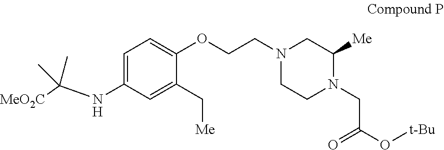 [SMILES: CCc1cc(NC(C)(C)C(=O)OC)ccc1OCCN1CCN(CC(=O)OC(C)(C)C)[C@H](C)C1]

with Compound S

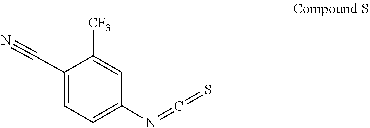 [SMILES: N#Cc1ccc(N=C=S)cc1C(F)(F)F]

in a solvent under conditions suitable to provide Compound U.

Embodiment 65. A process for preparing Compound P

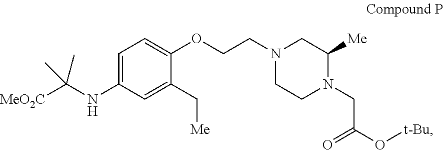 [SMILES: CCc1cc(NC(C)(C)C(=O)OC)ccc1OCCN1CCN(CC(=O)OC(C)(C)C)[C@H](C)C1]

comprising contacting Compound N

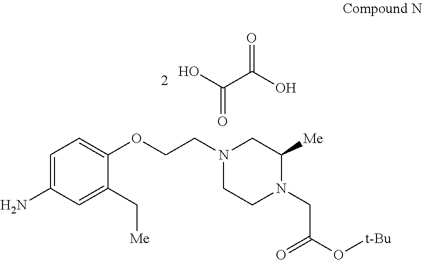

with methyl 2-bromo-2-methylpropionate in a solvent in the presence of a base under conditions suitable to provide Compound P.

Embodiment 66. A process for preparing Compound W

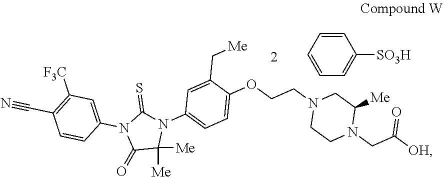

comprising contacting Compound U

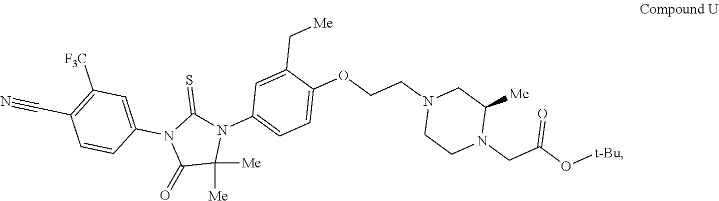 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

with BSA in a solvent under conditions suitable to provide Compound W.

Embodiment 67. A process for preparing Compound viii

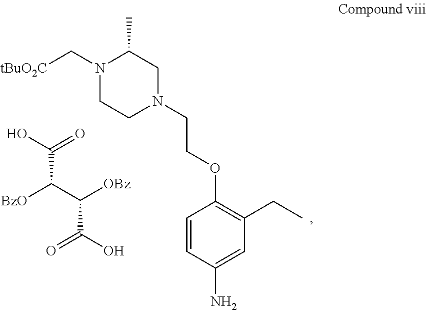

wherein the process comprises contacting Compound vii

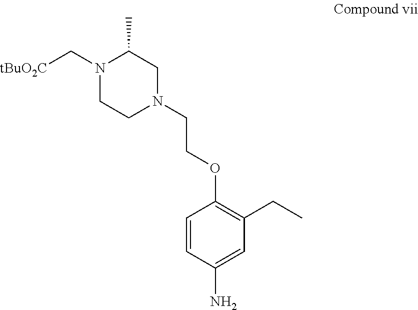 [SMILES: CCc1cc(N)ccc1OCCN1CCN(CC(=O)OC(C)(C)C)[C@H](C)C1]

with (+)-dibenzoyl-D-tartaric acid to provide Compound viii.

Embodiment 68. The process of embodiment 67, further comprising contacting Compound vi

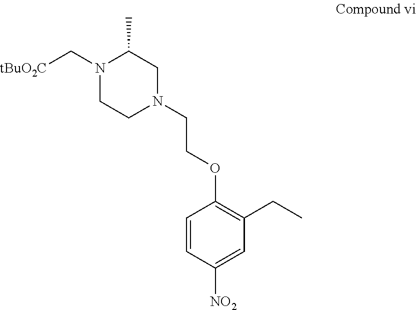 [SMILES: CCc1cc([N+](=O)[O-])ccc1OCCN1CCN(CC(=O)OC(C)(C)C)[C@H](C)C1]

with a palladium catalyst in a solvent under conditions suitable to provide Compound vii.

Embodiment 69. The process of embodiment 68, wherein the solvent comprises 2-methyltetrahydrofuran and ethanol.

Embodiment 70. The process of embodiment 68, wherein the process comprises contacting Compound vi with a palladium catalyst in a solvent comprising 2-methyltetrahydrofuran and ethanol mixture at about 20° C. to provide Compound vii.

Embodiment 71. The process of embodiment 68, further comprising contacting Compound v

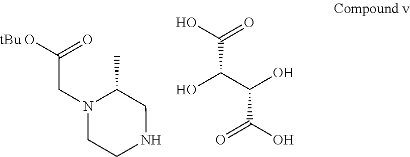

with Compound iv

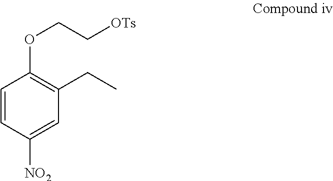 [SMILES: CCc1cc([N+](=O)[O-])ccc1OCCOS(=O)(=O)c1ccc(C)cc1]

in a solvent under conditions suitable to provide Compound vi.

Embodiment 72. The process of embodiment 71, wherein the solvent comprises 2-methyltetrahydrofuran and ethanol.

Embodiment 73. The process of embodiment 71, wherein the process comprises contacting Compound v with Compound iv in 2-methyltetrahydrofuran and water mixture in presence of sodium carbonate at about 71° C. to provide Compound vi.

Embodiment 74. The process of embodiment 71, further comprising contacting contacting (R)-(−)-2-methylpiperazine, Compound ix

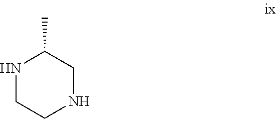

with a)ethyl trifluoroacetate in a solvent in presence of a base; b) tert-butyl bromoacetate; c) 25% tetramethylammonium hydroxide (Me₄NOH) in methanol; and d) D-tartaric acid under conditions suitable to provide Compound v.

Embodiment 75. The process of embodiment 74, wherein the process comprises contacting Compound ix with a)ethyl trifluoroacetate in 2-methyltetrahydrofuran in presence of DIEPA; b) tert-butyl bromoacetate; c) 25% tetramethylammonium hydroxide (Me₄NOH) in methanol; and d) D-tartaric acid under conditions suitable to provide Compound v.

Embodiment 76. The process of embodiment 71, further comprising contacting Compound iii

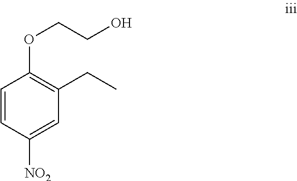

with 4-toluenesulfonyl chloride (TsC1) and triethylamine (TEA) in a solvent under conditions suitable to provide Compound iv.

Embodiment 77. The process of embodiment 76 comprising contacting Compound iii with TsCl and TEA in 2-methyltetrahydrofuran at about 20° C. to provide Compound iv.

Embodiment 78. The process of embodiment 76, further comprises contacting Compound ii

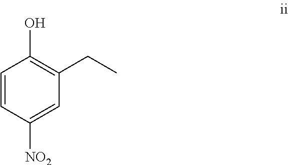

with sodium carbonate and ethylene carbonate in a solvent under conditions suitable to provide Compound iii.

Embodiment 79. The process of embodiment 78 comprising contacting Compound ii with sodium carbonate and ethylene carbonate in N-methyl-2-pyrrolidone at about 100° C. to provide Compound iii.

Embodiment 80. The process of embodiment 78, further comprising contacting 2-hydroxy-5-nitroacetophenone, Compound i

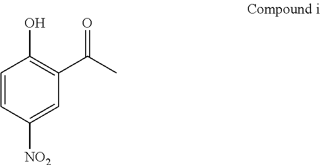

with trifluoroacetic acid and triethylsilane in a solvent under conditions suitable to provide Compound ii.

Embodiment 81. The process of embodiment 80 comprising contacting

Compound i with trifluoroacetic acid and triethylsilane in a mixture of toluene and heptane to provide Compound ii.

Embodiment 82. Compound K having structure:

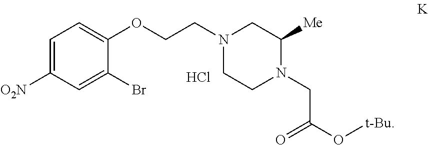 [SMILES: C[C@@H]1CN(CCOc2ccc([N+](=O)[O-])cc2Br)CCN1CC(=O)OC(C)(C)C ; Cl]

Embodiment 83. Compound L having structure:

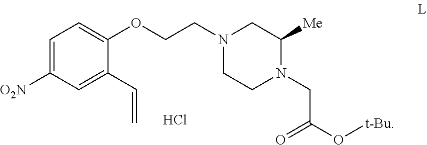 [SMILES: C=Cc1cc([N+](=O)[O-])ccc1OCCN1CCN(CC(=O)OC(C)(C)C)[C@H](C)C1 ; Cl]

Embodiment 84. Compound N having structure:

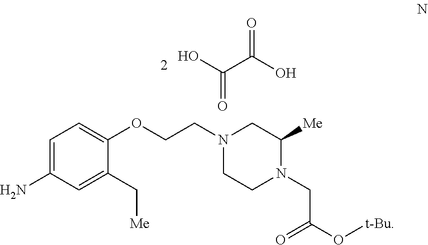

Embodiment 85. Compound U having structure:

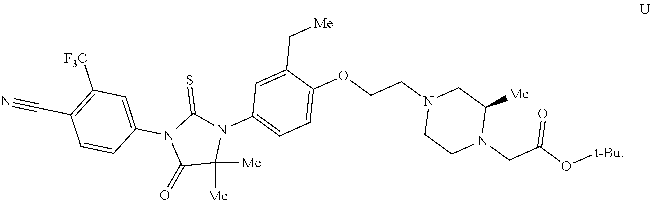 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]

Embodiment 86. Compound P having structure:

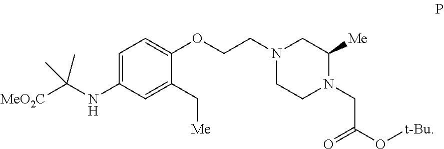 [SMILES: CCc1cc(NC(C)(C)C(=O)OC)ccc1OCCN1CCN(CC(=O)OC(C)(C)C)[C@H](C)C1]

Embodiment 87. Compound W having structure:

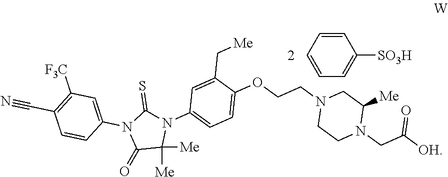

### EXAMPLES

The following abbreviations were used in descriptions and examples:
ACN or MeCN: Acetonitrile
AcOH: Acetic acid
BSA: Benzenesulfonic acid
CSA: 10-Camphorsulfonic acid
COMU: (1-Cyano-2-ethoxy-2-oxoethylidenaminooxy) dimethylamino-morpholino-carbenium hexafluorophosphate
DBU: 1,8-Diazabicyclo [5.4.0] undec-7-ene
DIEA or DIPEA or iPr2NEt: N,N-Diisopropylethylamine
DMA or DMAc: Dimethylacetamide
DMF: Dimethylformamide
DMSO: Dimethyl sulfoxide
DPPF: 1,1′ Bis (diphenylphosphino) ferrocene
EDC: 1-Ethyl-3-(3-dimethylaminopropyl) carbodiimide
eTFA: Ethyl trifluoroacetate
EtOAc: Ethyl acetate
HOBt: Hydroxybenzotriazole
HATU: Hexafluorophosphate Azabenzotriazole Tetramethyl Uronium
HPLC: High performance liquid chromatography
Hr: Hour
IPA or i-PrOH: Isopropyl alcohol
IPC: In process control
KF: Karl Fischer Titration
MeOH: Methanol
Me₂SO₄: Dimethylsulfate
MeTHF: 2-Methyltetrahydrofuran
MsCl: Methanesulfonyl chloride
Ms₂O: Methanesulfonic anhydride
MsOH: Methanesulfonic acid
MTBE: Methyl tert-Butyl Ether
NBS: N-Bromosuccinimide
NLT: Not less than
NMM: N-Methyl morpholine
NMP: N-Methyl-2-pyrrolidone
NMT: Not more than
Pd₂(dba)₃: Tris (dibenzylideneacetone) dipalladium (0)
1-PrOH: 1-Propyl alcohol, n-propanol
PyBop: Benzotriazol-1-yloxy)tripyrrolidinophosphonium hexafluorophosphate
TAA: Tert-amyl alcohol
T3P: Propylphosphonic anhydride
TBAB: Tetrabutylammonium Bromide
TBAI: Tetra-n-butylammonium iodide
TEA: Triethylamine
TsCl: 4-Toluenesulfonyl chloride
UPLC: Ultra Performance Liquid Chromatography
RT: Retention time
RRT: Relative retention time

The following Examples are presented by way of illustration, not limitation.

### Example 1: Synthesis of 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-((2,6-dioxopiperidin-3-yl)amino)phenyl) acetamide (Compound 1)

The title compound was prepared according to the following reaction scheme:

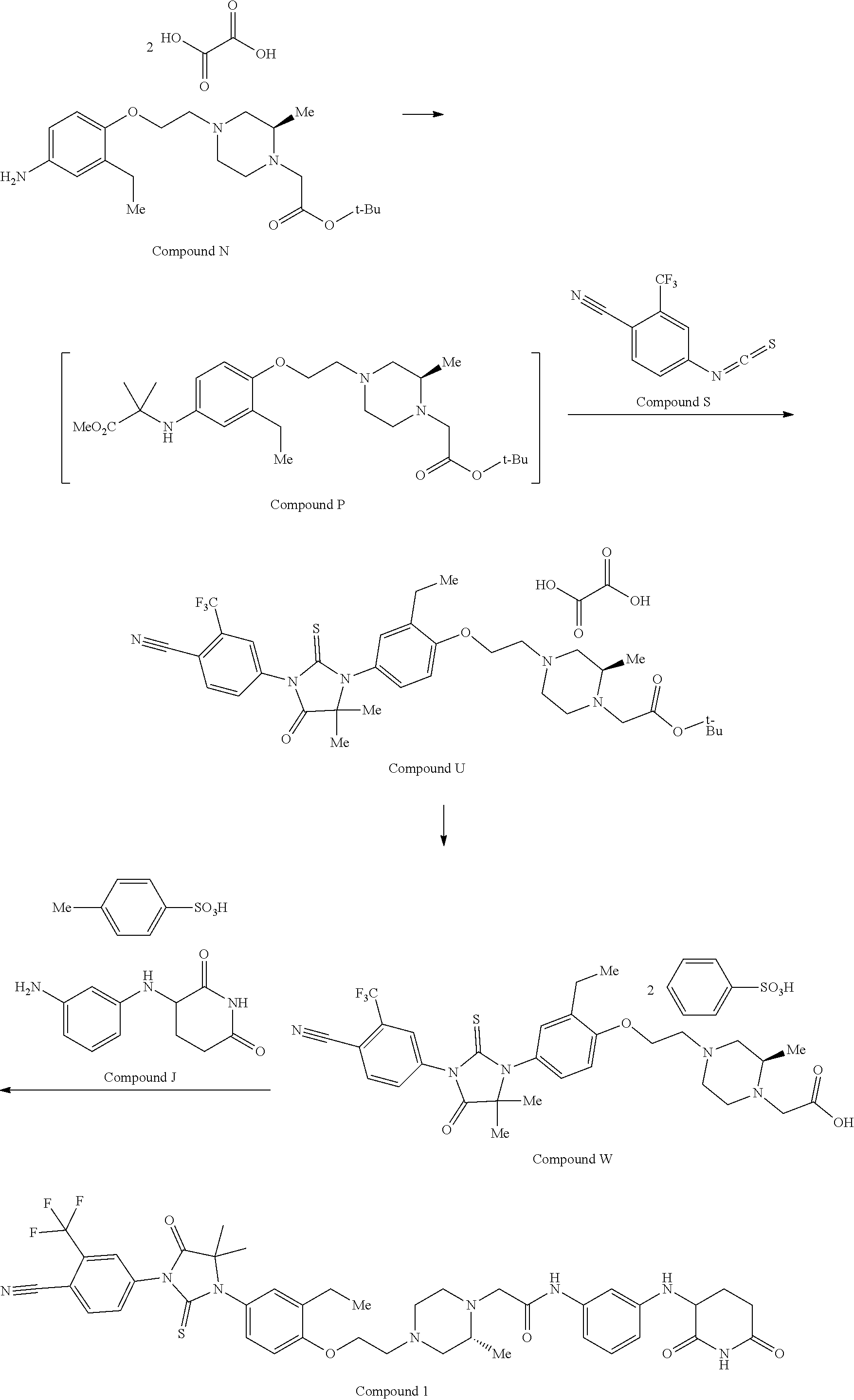

### Step 1: Synthesis of tert-butyl (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetate (Compound U)

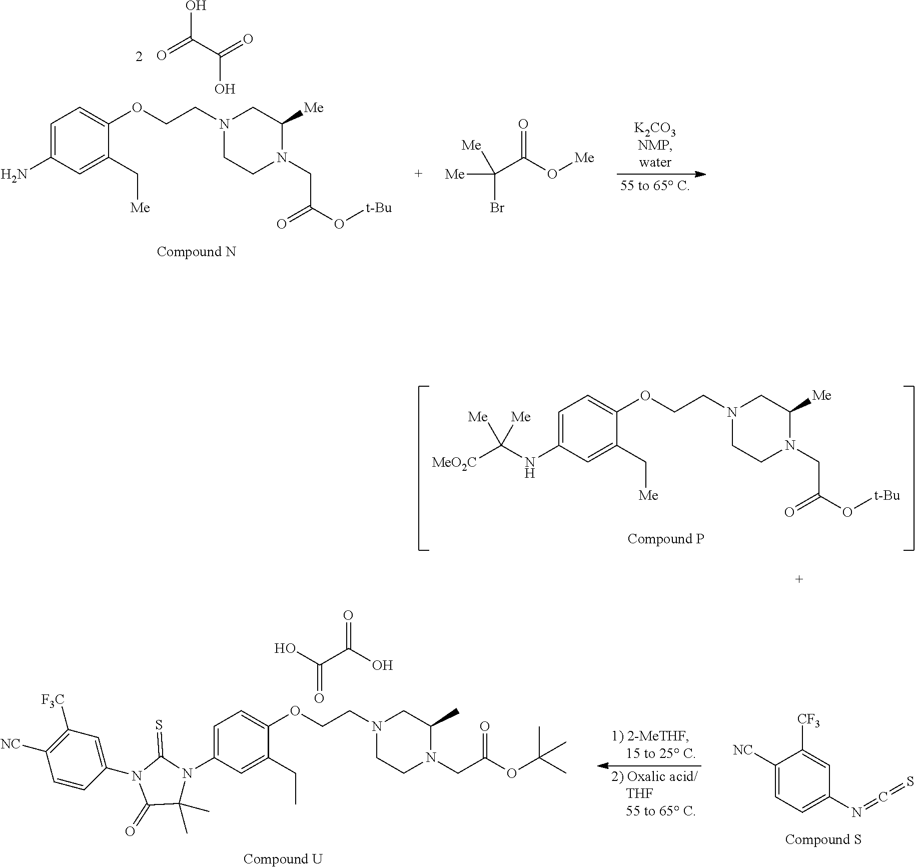

### Step 1a: Synthesis of methyl (R)-2-((4-(2-(4-(2-(tert-butoxy)-2-oxoethyl)-3-methylpiperazin-1-yl) ethoxy)-3-ethylphenyl)amino)-2-methylpropanoate (Compound P)

The following steps were undertaken to prepare Compound P:
1. Charge water (2×vol), K₂CO₃ (0.62×wt) and water (1×vol) sequentially and agitate to form a solution
2. Charge NMP (4.5×vol)
3. Charge Compound N (1.00×wt) in 8 portions at 15 to 20° C. over 2 hrs
4. Charge NMP (2.0×vol) and and agitate for 2 hrs
5. Charge methyl 2-bromo-2-methylpropionate (0.97×wt)
6. Heat the batch at 55 to 65° C. for NLT 38 hrs
7. Cool batch to 20 to 30° C.
8. Charge 2-Me-THF (10×vol) and water (15×vol)
9. Agitate batch for ˜ 30 mins
10. Stop agitation, settle for NLT 1 hr and remove bottom aq layer
11. Charge 5% wt aqueous NaCl (5.0×vol)
12. Agitate batch for ˜30 mins
13. Stop agitation, settle for NLT 1 hr and remove bottom aq layer
14. Charge 5% wt aqueous NaCl (5.0×vol)
15. Agitate batch for ˜30 mins
16. Stop agitation, settle for NLT 1 hr and remove bottom aq layer
17. Distill batch to 5.5 ×vol under vacuum at 45 to 55° C.
18. Distill batch at 5.5 ×vol while charging 2-Me-THF (6×vol) continuously under vacuum at 45 to 55° C.
19. Cool batch to 15 to 25° C.

### Step 1b: Synthesis of tert-butyl (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetate (Compound U)

The following steps were undertaken to prepare Compound U:
20. Charge Compound S (0.51×wt) and 2-Me-THF (0.5×vol)
21. Agitate batch at 15 to 25° C. for 10 to 12 hrs
22. Charge 2-Me-THF (4.0×vol) and 5% aqueous NaCl (4.0×vol)
23. Agitate batch for ˜30 mins
24. Stop agitation, settle for NLT 1 hr and remove bottom aq layer
25. Charge 5% aqueous NaCl (4.0×vol)
26. Agitate batch for ˜30 mins
27. Stop agitation, settle for NLT 1 hr and remove bottom aq layer
28. Distill batch to 4.5×vol under vacuum at 45 to 55° C.
29. Distill batch at 4.5×vol while charging 2-Me-THF (3×vol) continuously under vacuum at 45 to 55° C.
30. Charge THF (4.5×vol)
31. Heat batch to 55 to 65° C., targeting 60° C.
32. Prepare oxalic acid (0.22×wt) in THF (1.5×vol) at ambient temperature
33. Charge prepared oxalic acid in THE solution (20% wt, ˜0.32×vol)
34. Charge seed (Compound U, 1% wt)
35. Age batch for 2 hrs
36. Charge prepared oxalic acid in THE solution (0.3×vol, 0.2 mole eq) over 8 hrs
37. Cool batch to 20 to 30° C. targeting 25° C. over 14 hrs
38. Hold batch at 20 to 30° C. targeting 25° C. for at least 2 hrs
39. Filter and wash twice with 2-Me-THF (1.5×vol)/THF (0.75×vol)
40. Dry cake in vacuum oven at 45 to 50° C.

Expected yield: 73 to 85% by molar; Actual yield: 83% by molar.
Step 1a′ and Step 1b′ below describe alternative reaction conditions to prepare Compound P and Compound U:
   Step 1a′: Compound P Preparation
1. Charge water (3×vol) and K₂CO₃ (0.62×wt) and agitate to form a solution
   a. 0 to 5×vol water can be used
   b. 0.5 to 1.24 ×wt K₂CO₃ can be used
   c. TEA, DIEA, DBU, Na₂CO₃, Cs₂CO₃, Na₃PO₄ and K₃PO₄ can be used as base
   d. TBAI and TBAB can be used as additive
2. Charge NMP (4.5×vol)
   a. 4 to 10×vol NMP can be used
   b. CH₃CN, MeTHF, DMF, DMSO and DMAc can be used
3. Charge Compound N (1.00×wt) in 8 portions at 15 to 20° C. over 2 hrs
   a. 1 to 20 portions of charge can be used
   b. A time of 0 to 10 hrs can be used
   c. A temperature of 10 to 30° C. can be used
4. Charge NMP (2.0×vol) and agitate for 2 hrs
   a. A time of 0 to 4×vol can be used
   b. 0 to 10 hrs can be used
5 Charge methyl 2-bromo-2-methylpropionate (0.97×wt)
   a. 0.31 to 2 ×wt Methyl 2-bromo-2-methylpropionate can be used
6 Heat the batch at 55 to 65° C. for NLT 38 hrs
   a. A temperature of 50 to 75° C. can be used
   b. A time of 16 to 60 hrs can be used
7. Cool batch to 20 to 30° C.
   a. A temperature of 15 to 35° C. can be used
8. Charge 2-Me-THF (10×vol) and water (15×vol)
   a. 6 to 25×vol 2-Me-THF can be used
   b. 10 to 25×vol water can be used
9. Agitate batch for ˜ 30 mins
   a. A time of 0.5 to 4 hrs can be used
10 Stop agitation, settle for NLT 1 hr and remove bottom aq layer
   a. A time of 0.1 to 16 hrs can be used
11 Charge 5% wt aqueous NaCl (5.0×vol)
   a. 2 to 15×vol 5% wt aqueous NaCl can be used
   b. 2.5 to 25% aqueous NaCl can be used
12. Agitate batch for ˜30 mins
   a. A time of 0.5 to 4 hrs can be used
13. Stop agitation, settle for NLT 1 hr and remove bottom aq layer
   a. A time of 0.1 to 16 hrs can be used
14. Charge 5% wt aqueous NaCl (5.0×vol)
   a. 2 to 15×vol 5% wt aqueous NaCl can be used
   b. 2.5 to 25% aqueous NaCl can be used
15. Agitate batch for ˜30 mins
   a. A time of 0.5 to 4 hrs can be used
16. Stop agitation, settle for NLT 1 hr and remove bottom aq layer
   a. A time of 0.1 to 16 hrs can be used
17. Distill batch to 5.5×vol under vacuum at 45 to 55° C.
   a. A batch volume of 3 to 8×vol can be used
   b. A temperature of 25 to 65° C. can be used
18. Distill batch at 5.5×vol while charging 2-Me-THF (6×vol) continuously under vacuum at 45 to 55° C.
   a. 4 to 12×vol 2-Me-THF can be used
   b. A temperature of 25 to 65° C. can be used
19. Cool batch to 15 to 25° C.
   a. A temperature of 10 to 30° C. can be used
      Step 1b′: Compound U Preparation
1. Charge Compound S (0.51×wt) and 2-Me-THF (0.5×vol)
   a. 0.38 to 0.76 ×wt Compound S can be used
   b. 0 to 2×vol 2-Me-THF can be used
2. Agitate batch at 15 to 25° C. for 10 to 12 hrs
   a. A temperature of 10 to 30° C. can be used
   b. 4 to 30 hrs can be used
3. Charge 2-Me-THF (4.0×vol) and 5% aqueous NaCl (4.0×vol)
   a. 2 to 10×vol 2-Me-THF can be used
   b. 2 to 10×vol 5% aqueous NaCl can be used
   c. 2.5 to 25% aqueous NaCl can be used
4. Agitate batch for ˜30 mins
   a. A time of 0.1 to 4 hrs can be used
5. Stop agitation, settle for NLT 1 hr and remove bottom aq layer
   a. A time of 0.1 to 16 hrs can be used
6. Charge 5% aqueous NaCl (4.0×vol)
   a. 2 to 10×vol 5% aqueous NaCl can be used
   b. 2.5 to 25% aqueous NaCl can be used
7. Agitate batch for ˜30 mins
   a. A time of 0.1 to 4 hrs can be used
8. Stop agitation, settle for NLT 1 hr and remove bottom aq layer
   a. A time of 0.1 to 72 hrs can be used
9. Distill batch to 4.5×vol under vacuum at 45 to 55° C.
   a. A batch volume of 3 to 8×vol can be used
   b. A temperature of 25 to 65° C. can be used
10. Distill batch at 4.5×vol while charging 2-Me-THF (3×vol) continuously under vacuum at 45 to 55° C.
   a. A batch volume of 3 to 8×vol can be used
   b. A temperature of 25 to 65° C. can be used
11. Charge THF (4.5×vol)
   a. 3 to 10×vol THF can be used
12. Heat batch to 55 to 65° C., targeting 60° C.
   a. A temperature of 40 to 65° C. can be used
13 Prepare oxalic acid (0.22×wt) in THE solution (1.5×vol, 1.34×wt) at ambient temperature
   a. 0.16 to 0.35 ×wt oxalic acid can be used
   b. 1 to 4×vol THF can be used
14. Charge prepared solution of oxalic acid in THF (0.2×wt)
   a. 0.1 to 0.4 ×wt oxalic acid in THE solution can be used
15. Charge seed (Compound U, 1% wt)
   a. 0.1 to 5% wt seed can be used
16. Age batch for 2 hrs
   a. 0.5 to 6 hrs aging can be used
17. Charge the remainder of prepared oxalic acid in THE solution over 8 hrs
   a. A charging time of 4 to 24 hrs can be used
18. Cool batch to 20 to 30° C. targeting 25° C. over 14 hrs
   a. A temperature of 10 to 35° C. can be used
   b. A time of 4 to 20 hrs can be used
19. Hold batch at 20 to 30° C. targeting 25° C. for at least 2 hrs
   a. A temperature of 10 to 35° C. can be used
   b. A time of 4 to 20 hrs can be used
20. Filter and wash twice with 2-Me-THF (1.5×vol)/THF (0.75×vol)
   a. 1 to 4×vol 2-Me-THF can be used
   b. 0.5 to 2×vol THF can be used
21. Dry cake in vacuum oven at 45 to 50° C.
   a. A temperature of 35 to 55° C. can be used

Expected yield: 73 to 85% by molar; Actual yield: 83% by molar.

Analytical data for Compound U:

The HPLC purity of Compound U was 98.26% using the below HPLC method:

| Mobile Phase | Mobile Phase A: 0.1% Trifluoroacetic Acid | | |
|---|---|---|---|
| | Mobile Phase B: 0.1% TFA in Acetonitrile | | |
| Column | Waters CORTECS C18+, 3.0 × 100 mm, 1.6 μm | | |
| Flow Rate | 0.8 mL/min | | |
| Column Temperature | 45° C. | | |
| Auto-sampler | 5° C. | | |
| Temperature | | | |
| Detector Wavelength | UV @ 230 nm | | |
| Injection Volume | 1 μL | | |
| Sample Concentration | 0.4 mg/mL in amber glassware | | |
| LOQ | 0.2 mg/mL in amber glassware | | |
| | (0.05% of target cocn.) | | |
| Sample Diluent | 0.01% TFA:Acetonitrile (50:50) | | |
| | Time | A | B |
| | (minutes) | (%) | (%) |
| Gradient | 0.0 | 95 | 5 |
| | 2.0 | 95 | 5 |
| | 15.0 | 10 | 90 |
| | 17.0 | 10 | 90 |
| | 17.5 | 95 | 5 |
| | 20.0 | 95 | 5 |

| | |
|---|---|
| Run Time | 20.0 mins |

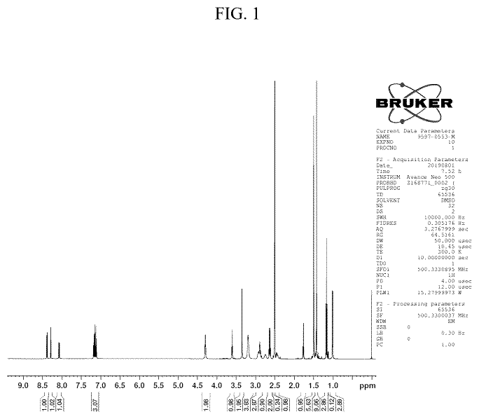

¹H NMR (500 MHZ, DMSO-d₆) δ ppm 1.01 (d, J=1.00 Hz, 3H) 1.16 (t, J=7.48 Hz, 3H) 1.42 (s, 9H) 1.49 (s, 6H) 2.40-2.47 (m, 1H) 2.63 (q, J=1.00 Hz, 2H) 2.69-2.79 (m, 1H) 2.82-2.97 (m, 3H) 3.11-3.26 (m, 4H) 3.34 (s, 2H) 4.29 (br t, J=5.11 Hz, 2H) 7.07-7.22 (m, 3H) 0.00 (dd, J=8.24, 1.68 Hz, 1H) 8.29 (d, J=1.00 Hz, 1H) 8.39 (d, J=1.00 Hz, 1H). FIG. 1 provides a ¹H NMR spectrum of Compound U.

MS Analysis: Calculated: [M+1]=674.3; Found: [M+1]=674.4

### Step 2: Synthesis of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid--benzenesulfonic acid (1/2) (Compound W)

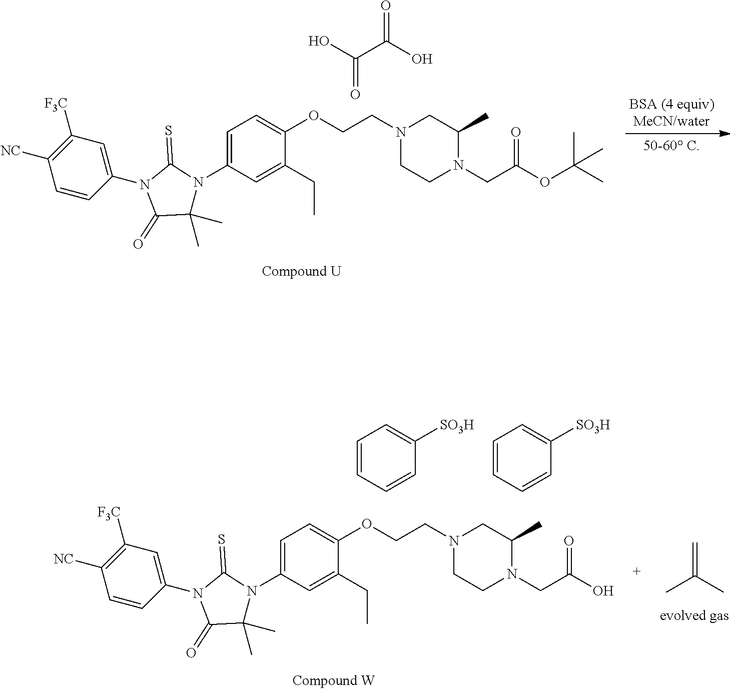

The following steps were undertaken to prepare Compound W:

### Reaction Setup:

1) Charge Compound U (1.00×wt).
2) Charge MeCN (5.0×vol).
3) Heat batch to 50 C.
4) Charge water (5.0×vol)
5) Charge BSA (0.94×wt, 4.0 equiv) in MeCN (4×vol) over NMT 30 mins.
6) Heat batch at 50 C for 30 hours.
7) IPC batch for reaction completion (acceptance is NMT 0.5 area % of Compound U normalized against Compound W). If not met, continue stirring for 4 hours and resample.
8) Charge 1.6×volumes of MeCN/water (85:15 mixture)
9) IPC batch for concentration (acceptance is between 60-70 mg/g). If not met (low), continue distilling. If not met (high), adjust batch concentration by addition of MeCN/water (85:15 ratio). If met, continue.

### Crystallization Procedure:

10) Heat batch between 50-60° C. (target 55° C.).
11) Charge deionized water (10.0×vol) over 30 mins while maintaining reaction temperature between 50-60° C. (target 55° C.)
12) Cool the batch slowly from 55° C. to 45° C. over NLT 4 hours.
13) Seed the batch with Compound W seed (0.01×wt) at ˜40° C.
14) Stir for NLT 15 mins.
15 IPC for visual seed present.
16) If met, proceed to step 15, if not, cool by 2° C. and seed again.
17) Allow reaction to cool slowly to 20 C over NLT 10 hours.
18) Reheat batch to seed temperature over 40 minutes.
19) Hold batch at seed temperature for NLT 2 hours.
20) Cool batch from seed temperature to 20 C over 6 hours.
21) Filter the batch.
22) Displacement wash the cake with water: MeCN (4:1, 5×volumes)
23) Sample the cake for purity (acceptance is NLT 98% area purity of Compound W).
24) Displacement wash the cake with water: MeCN (4:1, 5×volumes)
25) Sample the cake for purity (acceptance is NLT 98% area purity of Compound W).
26) Dry the cake at 40° C. under nitrogen flow overnight until loss on drying is less than 0.5%.
27) IPC reaction for residual MeCN (acceptance is <5000 ppm MeCN)
28) Remove dry cake and store protected from light and moisture Yield of Compound W: 87% molar.
   Analytical data for Compound W:

The HPLC purity of Compound W on NH₄OAc method was 99.47%. The HPLC method was as below:

| Mobile Phase | Mobile Phase A: Ammonium Acetate aqueous | | |
|---|---|---|---|
| | buffer (5 mM, pH 5.00):acetonitrile (95:5 v:v) | | |
| | Mobile Phase B: Ammonium Acetate aqueous | | |
| | buffer (5 mM, pH 5.00):acetonitrile (20:80 v:v) | | |
| Column | Acquity UPLC BEH phenyl hexyl, | | |
| | 3.0 × 100 mm, 1.7 μm particle size | | |
| Flow Rate | 0.8 mL/min | | |
| Column Temperature | 45° C. | | |
| Auto-sampler | 5° C. | | |
| Temperature | | | |
| Detector Wavelength | UV @ 230 nm | | |
| Injection Volume | 3 μL | | |
| Sample Concentration | 0.2 mg/mL in amber glassware | | |
| LOQ | 0.2 μg/mL in amber glassware | | |
| | (0.05% of target cocn.) | | |
| Sample Diluent | [1] Water:Acetonitrile (50:50) | | |
| | [2] Time | A | B |
| | (minutes) | (%) | (%) |
| Gradient | 0.0 | 100 | 0 |
| | 16.0 | 0 | 100 |
| | 16.1 | 100 | 0 |
| | 20.0 | 100 | 0 |

| | |
|---|---|
| Run Time | 20.0 minutes |

The HPLC purity of Compound W on TFA method was 98.85%. The HPLC method was as below:

| Mobile Phase | Mobile Phase A: 0.1% Trifluoroacetic Acid | | |
|---|---|---|---|
| | Mobile Phase B: 0.1% TFA in Acetonitrile | | |
| Column | Waters CORTECS C18+, 3.0 × 100 mm, | | |
| | 1.6 μm particle size | | |
| Flow Rate | 0.8 mL/min | | |
| Column Temperature | 45° C. | | |
| Auto-sampler | 5° C. | | |
| Temperature | | | |
| Detector Wavelength | UV @ 230 nm | | |
| Injection Volume | 1 μL | | |
| Sample Concentration | 0.4 mg/mL in amber glassware | | |
| LOQ | 0.2 μg/mL in amber glassware | | |
| | (0.05% of target cocn.) | | |
| Sample Diluent | 0.01% TFA:Acetonitrile (50:50) | | |
| | Time | A | B |
| | (minutes) | (%) | (%) |
| Gradient | 0.0 | 95 | 5 |
| | 2.0 | 95 | 5 |
| | 15.1 | 10 | 90 |
| | 17.0 | 10 | 90 |
| | 17.5 | 95 | 5 |
| | 20.0 | 95 | 5 |

| | |
|---|---|
| Run Time | 20.0 minutes |

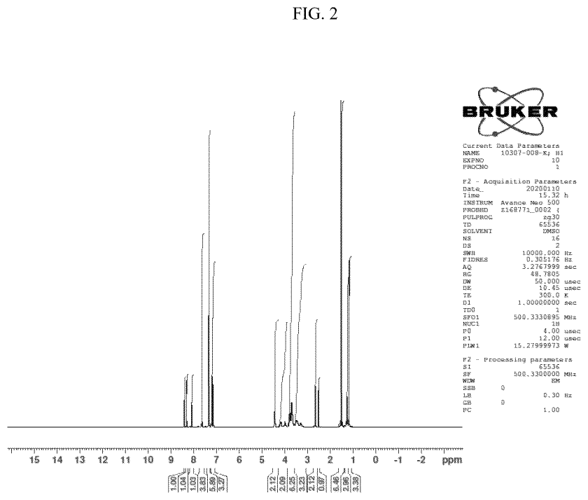

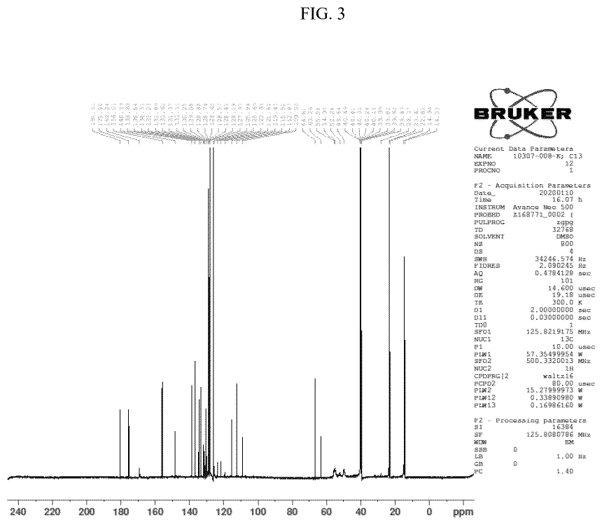

¹H NMR (500 MHZ, DMSO-d₆) δ ppm 1.01 (d, J=1.00 Hz, 3H) 1.16 (t, J=7.48 Hz, 3H) 1.42 (s, 9H) 1.49 (s, 6H) 2.40-2.47 (m, 1H) 2.63 (q, J=1.00 Hz, 2H) 2.69-2.79 (m, 1H) 2.82-2.97 (m, 3H) 3.11-3.26 (m, 4H) 3.34 (s, 2H) 4.29 (br t, J=5.11 Hz, 2H) 7.07-7.22 (m, 3H) 0.00 (dd, J=8.24, 1.68 Hz, 1H) 8.29 (d, J=1.00 Hz, 1H) 8.39 (d, J=1.00 Hz, 1H). FIG. 2 depicts a ¹H NMR spectrum of Compound W. FIG. 3 depicts a 13C NMR spectrum of Compound W.

MS Analysis: Calculated: [M+1]=618.24; Found: [M+1]=618.31

### Step 3: Synthesis of 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(2,6-dioxopiperidin-3-ylamino)phenyl) acetamide (Compound 1)

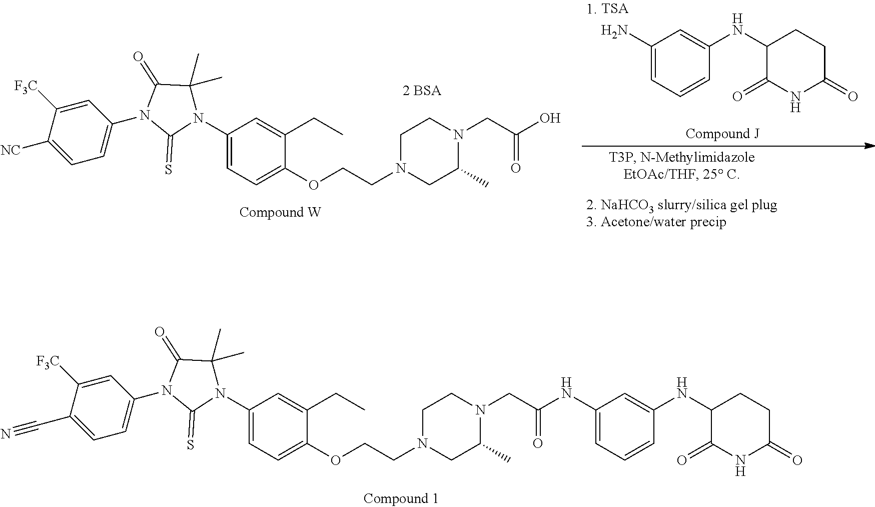 [SMILES: "TSA" ; CCc1cc(N2C(=S)N(c3ccc(C#N)c(C(F)(F)F)c3)C(=O)C2(C)C)ccc1OCCN1CCN(CC(=O)O)[C@H](C)C1 ; Nc1cccc(NC2CCC(=O)NC2=O)c1 ; CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1]

In Step 3, amide coupling of Compound W and Compound J was used to form Compound 1. The following steps were undertaken to prepare Compound 1, and the compound was isolated as an amorphous solid via precipitation from cold water.

### Reaction:

1. Charge Compound W (1.0×wt, 1.0 eq), Compound J (0.45×wt, 1.08 eq), THF (8 ×vol), and N-Methylimidazole (0.38×vol, 4.5 equiv).
2. Agitate batch at 25° C. for NLT 15 mins
3. IPC batch for KF (Acceptance criterion: NMT 0.3% water)
4. Charge T3P 50% in EtOAc (1.6×vol, 2.5 equiv) dropwise over NLT 4 h
5. Agitate batch at 25° C. for NLT 8 h,
6. IPC for reaction completion (NMT 1.0 A % Compound W remaining)

### Work-up:

7. Charge 10×vol EtOAc
8. Charge 2.5×vol water and 2.5×vol 1 M K₂HPO₄ pH=7.0 buffer
9. Heat batch to 30 to 40° C. for 15 mins
10. Agitate/settle/split
11. Charge and 5×vol 1M K₂HPO₄ pH=7.0 buffer.
12. Heat batch to 30 to 40° C. for 15 mins
13. Agitate/settle/split
14 Charge 2.5× vol 1 M K₂HPO₄ pH=7.0 and 2.5×vol water
15. Heat batch to 30 to 40° C. for 15 mins
16. Agitate/settle/split
17. Assay for solution yield and loss to aqueous splits (>90% solution yield)
   Solvent swap:
18. Concentrate batch to 3×vol at 35° C. under vacuum
19. Charge 8×vol Acetone
20 Vacuum distill to 3×vol at 35° C.
21. Charge 8×vol Acetone
22. Vacuum distill to 3×vol at 35° C.
23. Charge 7×vol Acetone
24 Vacuum distill to 3×vol at 35° C.
25. Charge 2×vol of Acetone
26 Vacuum distill to 3×vol at 35° C.
27. Cool to 25° C.

### Isolation:

28. Charge 0.2×wt NaHCO₃
29 Agitate batch at 25° C. for NLT 3 h
30. Filter batch over 0.5×wt silica gel
31 Wash silica gel pad with 14×vol Acetone
32. Concentrate filtrates to 3×vol
33 Charge 0.2×wt Acticarbone HPX-5 activated carbon
34 Agitate NLT 2 h
35 Filter followed by a polish filter
36 Wash filter pad with 10×vol Acetone
37 Vacuum distill to 3×vol at 35° C.
38. To a second vessel charge 27×vol water and cool to 5° C.
39. Charge Acetone solution to 27×vol of 5° C. water
40. Agitate at 5° C. for NMT 4 h
41. Filter batch at 0 to 10° C.
42. Wash cake two times with 5×vol water
43. Dry cake in a vacuum oven at 40 C with an N2 sweep
44. IPC for residual solvent and KF

Expected yield: 70-75% molar; Actual yield: 73% molar.

The following steps describe alternative reaction conditions to prepare Compound 1:
1. Charge Compound W (1.0×wt, 1.0 eq), Compound J (0.45×wt, 1.08 eq), THF (8 ×vol), and N-methylimidazole (0.38×vol, 4.5 equiv).
   a. Alternative solvents such as DMF, EtOAc, and mixtures of EtOAc/THF are viable. (MeCN, DMAc and DMSO will also provide product,
   b. Alternatives to N-methyl imidazole include, Et₃N and pyridine
   c. The equivalents of Compound J can be increased to 1.5 equiv, but ability to purge unreacted Compound J during isolation is difficult
2. Agitate batch at 25° C. for NLT 15 mins
   a. The batch can be agitated for up to 24 hrs
3. IPC batch for KF (Acceptance criterion: NMT 0.3% water)
   a. If batch fails KF spec, water can be azetotroped off with THF
4. Charge T3P 50% in EtOAc (1.6×vol, 2.5 equiv) dropwise over NLT 4 h
   a. T3P can be added as a solution in solvents that include EtOAc, THF and DMF
      b. Alternative amide coupling reagents that can be used include HATU, EDC, COMU, PyBop provide varying amounts of desired produt
      c. Addition of coupling reagent can be at once or dropwise over times of up to 12 h
5. Agitate batch at 25° C. for NLT 4 hrs,
   a. Batch can be agitated between 1 min and 48 hrs
   b. Temperatures can range between 20° C. and 45° C.
6. IPC for reaction completion (NMT 0.5 A % Compound W remaining)

### Work-up:

7. Charge 10× vol EtOAc
a. Increased volumes of EtOAc may be used
8 Charge 2.5× vol water and 2.5×vol 1 M K₂HPO₄ pH=7.0 buffer
9. Heat batch to 30 to 40 C for 15 mins
   a. Batch temperature can vary between 20° C. and 50° C.
10. Agitate/settle/split
11. Charge and 5× vol 1M K₂HPO₄ pH=7.0 buffer.
12. Heat batch to 30 to 40 C for 15 mins
   a. Batch temperature can vary between 20° C. and 50° C.
13. Agitate/settle/split.
14 Charge 2.5×vol 1 M K₂HPO₄ PH=7.0 and 2.5×vol water
   a. Varying ratio's of 1M K₂HPO₄ PH=7 and water can be used
15. Heat batch to 30 to 40 C for 15 mins
   a. Batch temperature can vary between 20° C. and 50° C.
16. Agitate/settle/split
17 Assay for solution yield and loss to aqueous splits (>90% solution yield)

### Solvent Swap:

18. Concentrate batch to 3×vol at 35° C. under vacuum
   a. Batch can also be concentrated to dryness
19. Charge 8×vol Acetone
   a. Solvent swap can be done in shots as outlined in steps 20-4
   b. Solvent swap can be done via continous distillation maintaining a batch volume of 3× and continously adding 24×vol acetone to eliminate steps 21 through 24
20. Vacuum distill to 3× vol at 35° C.
   a. Distillation temperature can range between 2° and 40° C.
21. Charge 8×vol Acetone
22 Vacuum distill to 3× vol at 35° C.
a. Distillation temperature can range between 2° and 40° C.
23. Charge 7×vol Acetone
24 Vacuum distill to 3×vol at 35° C.
   a. Distillation temperature can range between 2° and 40° C.
25 Charge 2×vol of Acetone
26 Vacuum distill to 3×vol at 35° C.
   a. Distillation temperature can range between 2° and 40° C.
27. Cool to 25° C.

### Isolation:

28 Charge 0.2× wt NaHCO₃
   a. Alternative bases such as KHCO₃ may be used
   b. Base loadings may vary between 0×wt and 0.5× wt
   c. Basic resins can be used
29. Agitate batch at 25 C for NLT 3 hrs
   a. Agitation times can vary between 1 hr and 16 h
30 Filter batch over 0.5×wt silica gel
   a. Silica gel loading can vary between 0.1×wt and 1×wt
31. Wash silica gel pad with 14×vol Acetone
   a. Volumes greater than 14× acetone can be used
32. Concentrate filtrates to 3×vol at 35° C.
   a. Distillation temperature can range between 2° and 40° C.
33. Charge 0.2× wt Acticarbone HPX-5 activated carbon
   a. Activated carbon loadings can vary between 0.05× and 0.5×wt
   b. Alternative activated carbons can be used that give varying degrees of decolorization and impurity purging
34 Agitate NLT 2 h
   a. Agitation times can be altered between 1 h and 24 h
35 Filter followed by a polish filter
36 Wash filter pad with 10×vol Acetone
   a. Volumes greater than 10×vol acetone can be used
37 Solution assay post carbon
38. Vacuum distill down to 3×vol at 35° C.
39 To a second vessel charge 27×vol water and cool to 5° C.
   a. Water temperatures can vary between 0 and 20° C.
   b. Increased volumes of water are tolerated
40 Charge Acetone solution to vessel two containing 27×vol of 5° C. water over NLT 1 h
   a. Addition of water to the acetone solution will resulting in gumming
   b. Alternative modes of addition of acetone to water may be used other than above suface addition. These include subsurface addition, simultaneous addition and mixing tee operations
41. Agitate at 5° C. for NMT 4 h
42 Filter batch at 0 to 10 C
   a. Gravity filtration or vacuum filtration with a pressure up to 50 psi is acceptable
43 Wash cake two times with 5×vol 5° C. water
   a. Additional water washes may be used
44 Dry cake in a vacuum oven at 40 C with an N2 sweep
   a. Temperatures up to 45 C are acceptable
   b. An agitated filter dryer is also acceptable
      With use of a filter dryer, agitation is not acceptable until the water content is NMT 15%
      Periodic agitation is acceptable (once KF NMT 15 wt. %) until LOD NMT 1% IPC for residual solvent and KF

### Analytical Data:

The HPLC purity of Compound 1 was 98.96%. The HPLC method was as below:

| Mobile Phase | Mobile Phase A: Ammonium Acetate aqueous |
|---|---|
| | buffer (5 mM, pH 5.00):acetonitrile (95:5 v:v) |
| | Mobile Phase B: Ammonium Acetate aqueous |
| | buffer (5 mM, pH 5.00):acetonitrile (20:80 v:v) |
| Column | Acquity UPLC BEH phenyl hexyl, |
| | 3.0 × 100 mm, 1.7 μm particle size |
| Flow Rate | 0.8 mL/min |
| Column Temperature | 45° C. |
| Auto-sampler | 5° C. |
| Temperature | |
| Detector Wavelength | UV @ 250 nm |
| Injection Volume | 3 μL |
| Sample Concentration | 0.2 mg/mL in amber glassware |
| LOQ | 0.2 g/mL in amber glassware |
| | (0.05% of target cocn.) |
| Sample Diluent | Water:Acetonitrile (50:50) |
| | Time (minutes) |
| Gradient | 0.0 |
| | 16.0 |
| | 16.1 |
| | 20.0 |
| Run Time | 20.0 mins |

¹H NMR (400 MHZ, DMSO-d₆) δ ppm 8 10.79 (s, 1H); 9.37 (s, 1H), 8.39 (d, J=8.4 Hz, 1H); 8.30 (s, 1H); 8.08 (dd, J=8.00 Hz, 1.2 Hz, 1H); 7.17-7.11 (m, 2H); 7.03-6.98 (m, 2H); 6.81 (d, J=8.00 Hz, 1H), 6.40 (d, J=8.4 Hz, 1H); 5.81 (d, J-8.00 Hz, 1H), 4.31-4.27 (m, 1H), 4.16 (t, J-5.6 Hz, 1H); 2.95 (d, 1H), 2.81-2.76 (m, 6H), 2.61-2.58 (m, 4H); 2.4-2.3 (m, 1H); 2.11-2.09 (m, 2H), 1.90-1.85 (m, 1H); 1.5 (s, 6H), 1.68 (t, J-7.6 Hz 3 H); 1.0 (d, J=6.0 Hz, 3H)

CHN Analysis: Calculated: C: 60.13%, H: 5.54%, N: 13.68% Found: C: 59.53%; H: 5.54%; N: 13.54%

### Example 2: Synthesis of 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(((R)-2,6-dioxopiperidin-3-yl)amino)phenyl) acetamide (Compound 1-1)

The title compound was prepared according to the following reaction scheme:

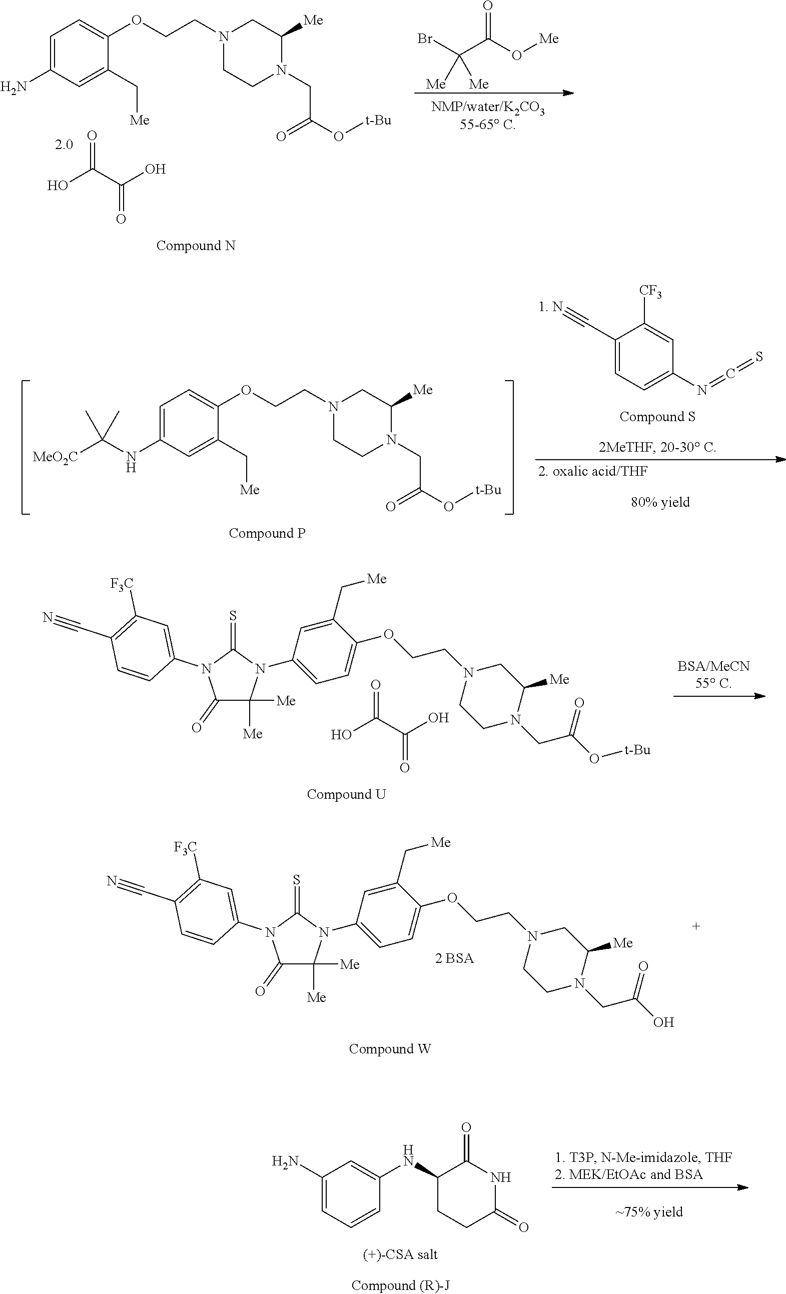

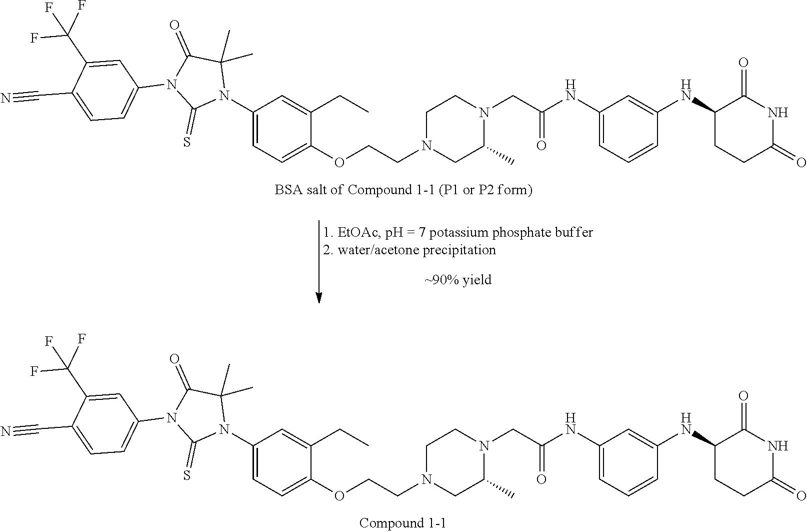 [SMILES: CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1 ; CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

The following steps were undertaken to prepare Compound 1-1: (trifluoromethyl)phenyl)-5,5-dimethyl-4-oxo-2-thioxoimidazolidin-1-yl)-2-ethylphenoxy)ethyl)-2-methylpiperazin-1-yl) acetate (Compound U)

Compound U was prepared as described in Example 1.

Step 2: Synthesis of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid--benzenesulfonic acid (1/2) (Compound W)

Compound W was prepared as described in Example 1.

Step 3a: Synthesis of BSA salt of 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(((R)-2,6-dioxopiperidin-3-yl)amino)phenyl) acetamide (BSA salt of Compound 1-1, P2 form)

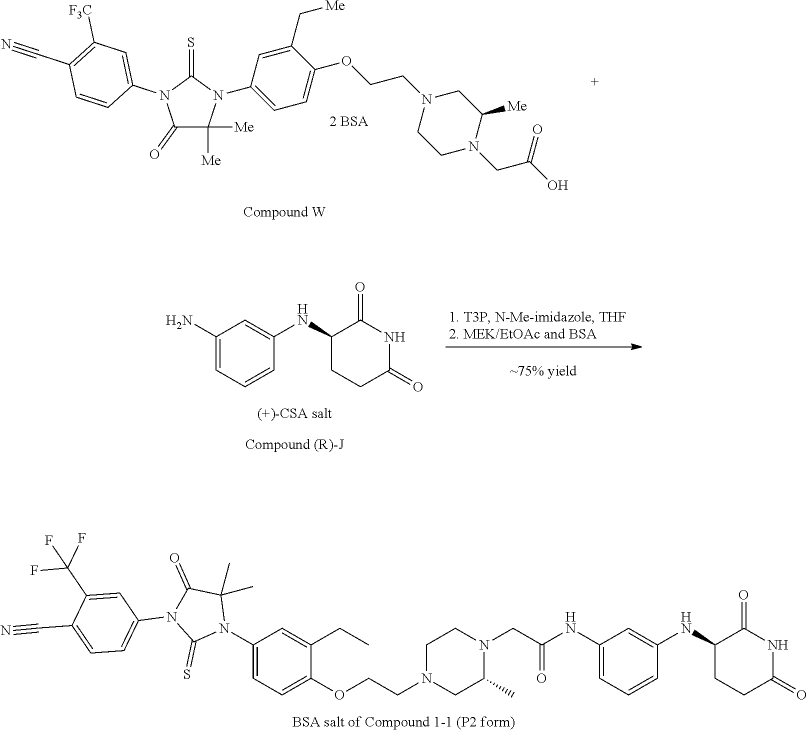 [SMILES: CCc1cc(N2C(=S)N(c3ccc(C#N)c(C(F)(F)F)c3)C(=O)C2(C)C)ccc1OCCN1CCN(CC(=O)O)[C@H](C)C1 ; Nc1cccc(N[C@@H]2CCC(=O)NC2=O)c1 ; CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

In this step, amide coupling of Compound W and Compound (R)-J was carried out as follows to prepare a crystalline BSA salt of Compound 1-1 (P2 form).

The reaction was conducted as described below:
[1] Adjust batch to 25° C.
[2] Charge
   Charge to Reactor 1: Compound W (LR, 1 kg, 1 L)
[3] Charge
   Charge to Reactor 1: Tetrahydrofuran (6 L/kg, 5.334 kg, 6 L) [4] Charge

Charge to Reactor 1:1-Methylimidazole (3 equiv., 0.2539 kg, 0.2465 L) [5] Collect IPC Sample IPC-1:
Sample Description: Residual water
Technique: KF/Coulametric (Compendial)
Range: 0 to 0.3 wt %
[6] Concentrate to ˜3 L/kg under reduced pressure Note: Adjust pressure to maintain reflux temp <35° C.
[7] Charge
Charge to Reactor 1: Tetrahydrofuran (6 L/kg, 5.34 kg, 6 L) [8] Concentrate to ˜3 L/kg under reduced pressure
Note: Adjust pressure to maintain reflux temp <35° C. [9] Charge
Charge to Reactor 1: Tetrahydrofuran (3 L/kg, 2.67 kg, 3 L)

Collect IPC

Sample IPC-1:

Sample Description: Residual water
Technique: KF/Coulametric (Compendial)
Range: 0 to 0.3 wt %

### Charge

Charge to Reactor 1: Compound (R)-J (1.08 equiv., 0.5027 kg, 0.5027 L) Charge

Charge to Reactor 1:1-Methylimidazole (1.5 equiv., 0.127 kg, 0.1233 L)

Charge to rinse solids and reagent charge lines

Charge to Reactor 1: Tetrahydrofuran (2 L/kg, 1.78 kg, 2 L)

Charge over NLT 1 hr

Charge to Reactor 1: T3P 50% in EtOAc (50 wt %) (2.5 equiv., 1.64 kg, 1.534 L)

Age for NLT 8 hrs at 25° C.

Collect IPC Sample IPC-2:
Sample Description: End of Reaction
Technique: UPLC/UV
Range: 0 to 0.5 RAP

### IPC Fail Path

Charge

Charge to Reactor 1: T3P 50% in EtOAc (50 wt %) (0.25 equiv., 0.164 kg, 0.1534 L)

Continue to age at 25° C. for NLT 4 hrs

Collect IPC

### Sample IPC-2:

Sample Description: End of Reaction
Technique: UPLC/UV
Range: 0 to 0.5 RAP

### Unit Op: Solution Prep 1-Prep Tank 1

Charge

Charge to Prep Tank 1: Potassium phosphate dibasic (1.12 kg/kg, 1.12 kg)

### Charge

Charge to Prep Tank 1: Potassium phosphate monobasic (0.757 kg/kg, 0.757 kg)

### Charge

Charge to Prep Tank 1: Water (12 L/kg, 12 kg, 12 L)

Agitate to dissolve to achieve full dissolution

Discharge for later use

### Unit Op: Extraction 1

Charge

Charge to Reactor 1: Ethyl acetate (10 L/kg, 9.02 kg, 10 L)

### Charge

Charge to Reactor 1: Water (2 L/kg, 2 kg, 2 L)

### Charge

Charge to Reactor 1: 1M K₂HPO₄/KH²PO⁴ (pH 7) (3 L/kg, 3.33 kg, 3 L)

Age at 25° C. for NLT 30 mins

Stop agitation and settle for NLT 1 hr

Discharge lower aqueous to water

### Unit Op: Extraction 2

### Charge

Charge to Reactor 1: 1M K₂HPO₄/KH₂PO₄ (pH 7) (5 L/kg, 5.55 kg, 5 L) Age at 25° C. for NLT 30 mins

Stop agitation and settle for NLT 1 hr

Discharge lower aqueous to water

### Unit Op: Extraction 3

Charge

Charge to Reactor 1: Water (2 L/kg, 2 kg, 2 L)

### Charge

Charge to Reactor 1: 1M K₂HPO₄/KH₂PO₄ (pH 7) (3 L/kg, 3.33 kg, 3 L)

Stop agitation and settle for NLT 1 hr

Discharge lower aqueous to water

Unit Op: Distillation 1

Note: Target pressure: 200 mbar

Max reflux temp: 35° C.

Max jacket temp: 45° C.

Concentrate to 4 L/kg Charge

Charge to Reactor 1: Methyl Ethyl Ketone (10 L/kg, 8.05 kg, 10 L)

Concentrate to 4 L/kg

### Charge

Charge to Reactor 1: Methyl Ethyl Ketone (10 L/kg, 8.05 kg, 10 L) Collect IPC

### Sample IPC-3:

Sample Description: Residual EtOAc
Technique: GC/FID
Range: 0 to 3 wt %

### IPC Fail Path

Charge

Charge to Reactor 1: Methyl Ethyl Ketone (4 L/kg, 3.22 kg, 4 L)

Concentrate to 4 L/kg

Collect IPC Sample IPC-3:
Sample Description: Residual EtOAc
Technique: GC/FID
Range: 0 to 3 wt %

### Collect IPC

Sample IPC-4:
Sample Description: Residual Water
Technique: KF/Coulametric (Compendial)
Range: 1.7 to 2.7 wt %

### IPC Fail Path

Fail path if water content is lower than specification Calculated water charge per IPC

### Charge to Reactor 1: Water

Note: Water charge should be calculated to achieve water concentration between 1.7-2.7 wt %.

### Collect IPC

Sample IPC-4:
Sample Description: Residual Water
Technique: KF/Coulametric (Compendial)
Range: 1.7 to 2.7 wt %

### IPC Fail Path

Fail path if water content is higher than specification

### Charge

Charge to Reactor 1: Methyl Ethyl Ketone (4 L/kg, 3.208 kg, 4 L)

Concentrate to 4 L/kg
Output: Distillate

### Collect IPC

Sample IPC-4:
Sample Description: Residual Water
Technique: KF/Coulametric (Compendial)
Range: 1.7 to 2.7 wt %

### Unit Op: Polish Filtration-Polish Filter

Transfer from Reactor 1 to Reactor 2 via Polish Filter

### Charge

Charge to Polish Filter: Methyl Ethyl Ketone (2 L/kg, 1.61 kg, 2 L)

Transfer from Reactor 1 to Reactor 2 via Polish Filter

### Unit Op: Solution Prep 2-Prep Tank 2

Charge

Charge to Prep Tank 2: Benzenesulfonic acid (0.96 equiv., 0.1565 kg, 0.1186 L)

### Charge

Charge to Prep Tank 2: Ethyl Acetate (1.7 L/kg, 1.533 kg, 1.7 L)

### Unit Op: Crystallization-Reactor 2

Adjust batch to 25° C.

Charge above surface over NLT 30 mins

Charge to Reactor 2: BSA in EtOAc (0.727 L/kg, 0.6754 kg, 0.727 L) Charge

Charge to Reactor 2: BSA salt of Compound 1-1, P2 form (0.01 kg/kg, 0.01 kg, 0.01 L)

Age for NLT 8 hr Charge above surface over NLT 4 hrs

Charge to Reactor 2: BSA in EtOAc (1.0916 L/kg, 1.014 kg, 1.091 L)

Charge over NLT 4 hrs

Charge to Reactor 2: Ethyl acetate (4.2 L/kg, 3.788 kg, 4.2 L)

Age for NLT 4 hrs

### Unit Op: Isolation-Filter 1

Filter slurry on Filter 1

### Charge

Charge to Filter 1:6:7 MEK/EtOAc (2.5 L/kg, 2.013 kg, 2.501 L)

### Charge

Charge to Filter 1:6:7 MEK/EtOAc (2.5 L/kg, 2.013 kg, 2.501 L)

### Unit Op: Drying-Dryer 1

Dry at 50° C. under vacuum for NLT 8 hrs

Collect IPC Sample IPC-5:
Sample Description: Residual EtOAc
Technique: GC/FID
Range: 0 to 0.4 wt %

Continue drying at 50° C. under vacuum for NLT 4 hrs Collect IPC

### Sample IPC-5:

Sample Description: Residual EtOAc
Technique: GC/FID
Range: 0 to 0.4 wt %

### Collect IPC

Sample IPC-6:
Sample Description: Residual MEK
Technique: GC/FID
Range: 0 to 0.4 wt %

### IPC Fail Path

Continue drying at 50° C. under vacuum for NLT 4 hrs

Collect IPC Sample IPC-6:
Sample Description: Residual MEK
Technique: GC/FID
Range: 0 to 0.4 wt %

### Collect IPC

Sample IPC-7:
Sample Description: Crystal Form
Technique: XRD

### IPC Fail Path

Continue drying at 50° C. under vacuum for NLT 4 hrs Collect IPC

### Sample IPC-7:

Sample Description: Crystal Form
Technique: XRD

Discharge product

Note: Delumping via co-mill is required.

Step 3b: Synthesis of BSA salt of 2-((R)-4-(2-(4-(3-(4-Cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(((R)-2,6-dioxopiperidin-3-yl)amino)phenyl) acetamide (BSA salt of Compound 1-1, P1 form)

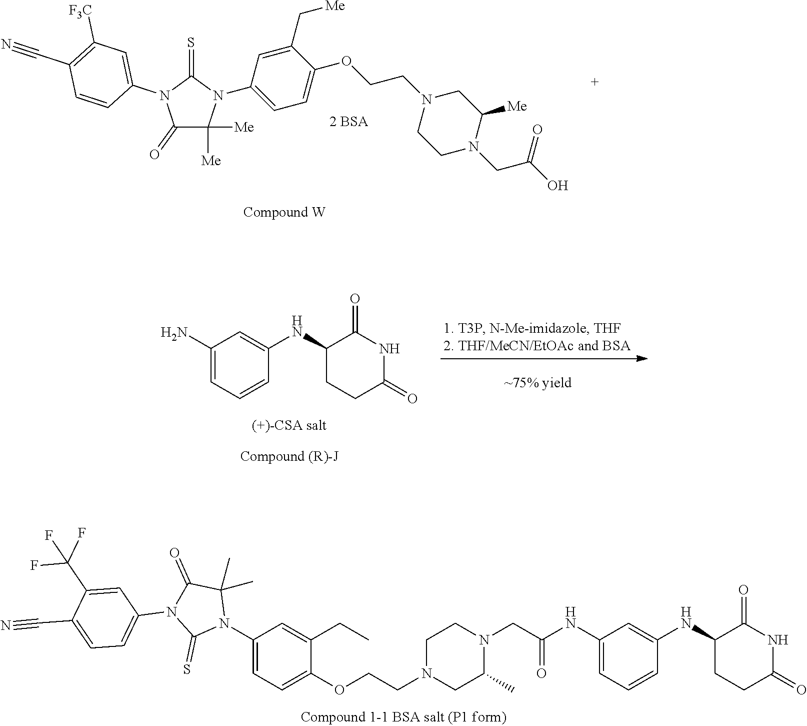 [SMILES: CCc1cc(N2C(=S)N(c3ccc(C#N)c(C(F)(F)F)c3)C(=O)C2(C)C)ccc1OCCN1CCN(CC(=O)O)[C@H](C)C1 ; Nc1cccc(N[C@@H]2CCC(=O)NC2=O)c1 ; CCc1cc(N2C(=S)N(c3ccc(C#N)c(C(F)(F)F)c3)C(=O)C2(C)C)ccc1OCCN1CCN(CC(=O)Nc2cccc(N[C@@H]3CCC(=O)NC3=O)c2)[C@H](C)C1]

In this step, amide coupling of Compound W and Compound (R)-J was carried out as follows to prepare BSA salt of Compound 1-1, P1 form.

### Unit Op: Reaction-Reactor 1

Note: Reaction is sensitive to moisture and protic solvents (MeOH, EtOH, etc.)

A boil-out and IPC is required to ensure that equipment is free of water and alcohols. [1] Adjust batch to 25° C. [2] Charge

Charge to Reactor 1: Tetrahydrofuran (4 L/kg, 3.5 kg, 4 L) [3] Charge

Charge to Reactor 1: Compound W (LR, 1 kg, 1 L) [4] Charge

Charge to Reactor 1: Tetrahydrofuran (2 L/kg, 1.8 kg, 2 L) [5] Charge

Charge to Reactor 1:1-Methylimidazole (2.8 equiv., 0.25 kg, 0.24 L) [6] Collect IPC

### Sample IPC-1:

Sample Description: Residual water
Technique: KF/Coulametric (Compendial)
Range: 0 to 0.6 wt %

IPC Fail Path [7] Concentrate to ˜3 L/kg under reduced pressure
Output: Distillate

Note: Adjust pressure to maintain reflux temp <35° C. [8] Charge

Charge to Reactor 1: Tetrahydrofuran (6 L/kg, 5.34 kg, 6 L) [9] Concentrate to ˜3 L/kg under reduced pressure

Note: Adjust pressure to maintain reflux temp <35° C.

### Charge

Charge to Reactor 1: Tetrahydrofuran (3 L/kg, 2.67 kg, 3 L)

Collect IPC Sample IPC-1:
Sample Description: Residual water
Technique: KF/Coulametric (Compendial)
Range: 0 to 0.6 wt %

### Charge

Charge to Reactor 1: Compound (R)-J (1.03 equiv., 0.50 kg, 0.50 L)

### Charge

Charge to Reactor 1:1-Methylimidazole (2.02 equiv., 0.17 kg, 0.17 L)

Charge to rinse solids and reagent charge lines Charge to Reactor 1: Tetrahydrofuran (2 L/kg, 1.78 kg, 2 L) Charge over NLT 1 hr

Charge to Reactor 1: T3P 50% in EtOAc (50 wt %) (2.88 equiv., 1.96 kg, 1.84 L)

Age for NLT 8 hrs at 25° C.

### Collect IPC

Sample IPC-2:
Sample Description: End of Reaction
Technique: UPLC/UV
Range: 0 to 0.5 RAP

### IPC Fail Path-1

Continue to age at 25° C. for NLT 8 hrs Collect IPC

### Sample IPC-2:

Sample Description: End of Reaction
Technique: UPLC/UV
Range: 0 to 0.5 RAP

### Unit Op: Solution Prep Thank

Charge

Charge to Reactor 1:1-Methylimidazole (0.48 equiv., 0.042 kg, 0.041 L) Charge

Charge to Reactor 1: T3P 50% in EtOAc (50 wt %) (0.96 equiv., 0.65 kg, 0.61 L)

Continue to age at 25° C. for NLT 4 hrs

Collect IPC Sample IPC-2:
Sample Description: End of Reaction
Technique: UPLC/UV
Range: 0 to 0.5 RAP

### Unit Op: Solution Prep 1-Prep Tank 1

Charge

Charge to Prep Tank 1: Potassium phosphate dibasic (1.12 kg/kg, 1.12 kg)

### Charge

Charge to Prep Tank 1: Potassium phosphate monobasic (0.757 kg/kg, 0.757 kg)

### Charge

Charge to Prep Tank 1: Water (12 L/kg, 12 kg, 12 L)
Output: 1M K₂HPO₄/KH₂PO₄ (pH 7)

Agitate to dissolve to achieve full dissolution

Discharge for later use

### Unit Op: Extraction 1-Reactor 1

Charge

Charge to Reactor 1: Ethyl acetate (10 L/kg, 9.02 kg, 10 L)

### Charge

Charge to Reactor 1: Water (2 L/kg, 2 kg, 2 L)

### Charge

Charge to Reactor 1: 1M K₂HPO₄/KH₂PO₄ (pH 7) (3 L/kg, 3.33 kg, 3 L)

Age at 25° C. for NLT 30 mins

Stop agitation and settle for NLT 1 hr

Discharge lower aqueous to water

### Unit Op: Extraction 2-Reactor 1

### Charge

Charge to Reactor 1: 1M K₂HPO₄/KH₂PO₄ (pH 7) (5 L/kg, 5.55 kg, 5 L)

Age at 25° C. for NLT 30 mins

Stop agitation and settle for NLT 1 hr

Discharge lower aqueous to water Unit Op: Extraction 3-Reactor 1 Charge

Charge to Reactor 1: Water (2 L/kg, 2 kg, 2 L) Charge

Charge to Reactor 1: 1M K₂HPO₄/KH₂PO₄ (pH 7) (3 L/kg, 3.33 kg, 3 L)

Age at 25° C. for NLT 30 mins

Stop agitation and settle for NLT 1 hr

Discharge lower aqueous to water Unit Op: Distillation 1-Reactor 1

Note: Target pressure: 200 mbar

Max reflux temp: 35° C.

Max jacket temp: 45° C.

Concentrate to 4 L/kg Charge

Charge to Reactor 1: Ethyl acetate (10 L/kg, 9.02 kg, 10 L)

Concentrate to 4 L/kg Charge

Charge to Reactor 1: Ethyl acetate (10 L/kg, 9.02 kg, 10 L) Collect IPC

### Sample IPC-3:

Sample Description: Residual Water
Technique: KF/Coulametric (Compendial)
Range: 0 to 0.5 wt %

### IPC Fail Path

Fail path if water content is higher than specification Charge

Charge to Reactor 1: Ethyl acetate (4 L/kg, 3.608 kg, 4 L)

Concentrate to 4 L/kg

Collect IPC Sample IPC-3:
Sample Description: Residual Water
Technique: KF/Coulametric (Compendial)
Range: 0 to 0.5 wt %

### Unit Op: Polish Filtration-Polish Filter

Transfer from Reactor 1 to Reactor 2 via Polish Filter

### Charge

Charge to Polish Filter: Tetrahydrofuran (5.5 L/kg, 4.89 kg, 5.5 L)

Transfer from Reactor 1 to Reactor 2 via Polish Filter

### Unit Op: Solution Prep 2-Prep Tank 2

Charge

Charge to Prep Tank 2: Benzenesulfonic acid (0.92 equiv., 0.15 kg, 0.11 L)

### Charge

Charge to Prep Tank 2: Ethyl Acetate (2 L/kg, 1.80 kg, 2 L)

Unit Op: Crystallization-Reactor 2 Adjust batch to 25° C.

Charge above surface over NLT 30 mins

Charge to Reactor 2: Acetonitrile (1.1 L/kg, 0.86 kg, 1.1 L)

Charge above surface over NLT 30 mins

Charge to Reactor 2: BSA in EtOAc (1.021 kg/kg, 1.02 kg, 1.10 L) Charge

Charge to Reactor 2: BSA salt of Compound 1-1, P1 form (0.01 kg/kg, 0.01 kg, 0.01 L) Age for NLT 16 hr

Charge above surface over NLT 3 hrs

Charge to Reactor 2: BSA in EtOAc (0.94 kg/kg, 0.94 kg, 1.016 L)

Age for NLT 3 hrs

Charge over NLT 4 hrs

Charge to Reactor 2: Ethyl acetate (8.2 L/kg, 7.396 kg, 8.2 L)

Age for NLT 8 hrs

### Unit Op: Isolation-Filter 1

Filter slurry on Filter 1

### Charge

Charge to Filter 1:27.5:5.5:67 MeCN: THF: EtOAc (3 L/kg, 2.6 kg, 3 L) Charge

Charge to Filter 1: Ethyl Acetate (3 L/kg, 2.7 kg, 3 L)

### Unit Op: Drying-Dryer 1

Dry at 50° C. under vacuum for NLT 8 hrs

### Collect IPC

Sample IPC-4:
Sample Description: Residual EtOAc
Technique: GC/FID
Range: 0 to 5 wt %

### IPC Fail Path

Continue drying at 50° C. under vacuum for NLT 4 hrs

Collect IPC Sample IPC-4:
Sample Description: Residual EtOAc
Technique: GC/FID
Range: 0 to 5 wt %

### Collect IPC

Sample IPC-5:
Sample Description: Residual THF
Technique: GC/FID
Range: 0 to 720 ppm

### Collect IPC

Sample IPC-6:
Sample Description: Residual MeCN
Technique: GC/FID
Range: 0 to 410 ppm

Discharge product

Note: Delumping via co-mill is required.

The HPLC purity of BSA salt of Compound 1-1 was determined using the below

HPLC method:

| Mobile Phase | Mobile Phase A: Ammonium Acetate aqueous buffer |
|---|---|
| | (5 mM, pH 5.00):acetonitrile (95:5 v:v) |
| | Mobile Phase B: Ammonium Acetate aqueous buffer |
| | (5 mM, pH 5.00):acetonitrile (20:80 v:v) |
| Column | Acquity UPLC BEH phenyl hexyl, 3.0 × 100 mm, 1.7 |
| | μm particle size |
| Flow Rate | 0.8 mL/min |
| Column | 45° C. |
| Temperature | |
| Auto-sampler | 5° C. |
| Temperature | |
| Detector | UV @ 250 nm |
| Wavelength | |
| Injection | 3 μL |
| Volume | |
| Sample | 0.2 mg/mL in amber glassware |
| Concentration | |
| LOQ | 0.2 μg/mL in amber glassware (0.05% of target cocn.) |
| Sample Diluent | Water:Acetonitrile (50:50) |
| Gradient | Time (minutes) |
| | 0.0 |
| | 16.0 |
| | 16.1 |
| | 20.0 |
| Run Time | 20.0 mins |

Step 4: Synthesis of 2-((R)-4-(2-(4-(3-(4-Cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-(((R)-2,6-dioxopiperidin-3-yl)amino)phenyl) acetamide (Compound 1-1)

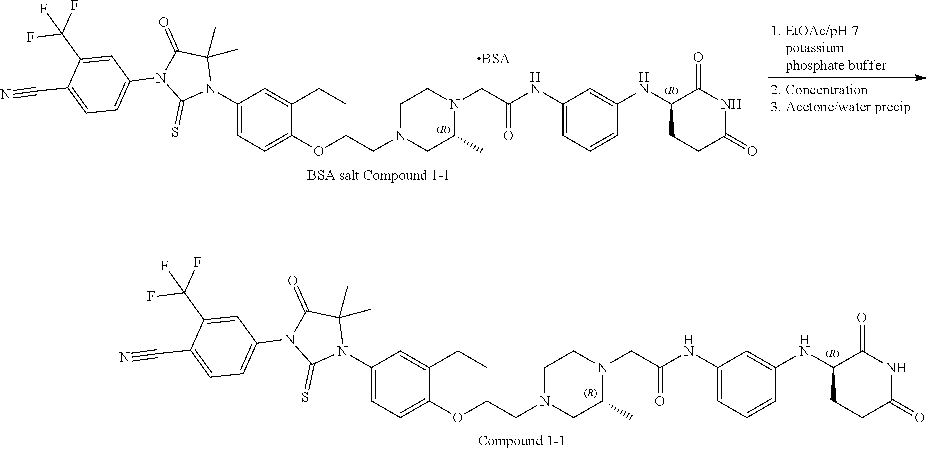

In this step, the BSA salt of Compound 1-1 was converted to the amorphous freebase, Compound 1-1, via phosphate buffer (pH 7) washes, a solvent swap to acetone and precipitation into water. The reaction was conducted as described below:

Unit Op: Solution Prep-Prep Tank 1 [1] Charge to Prep Tank 1: Water (6 L/kg, 6 kg, 6 L) [2] Charge to Prep Tank 1: Potassium phosphate dibasic (3.8 kg/kg, 3.8 kg, 1.67 L) [3] Charge to Prep Tank 1: Potassium phosphate monobasic (1.08 kg/kg, 1.08 kg, 0.4619 L) [4] Charge to Prep Tank 1: Water (1.5 L/kg, 1.5 kg, 1.5 L) [5] Agitate to achieve full dissolution [6] Discharge for later use
Output: 4M K₂HPO₄/KH₂PO₄ (pH 7.5)

Unit Op: Solution Prep-Prep Tank 1 [7] Charge to Prep Tank 1: Water (6 L/kg, 6 kg, 6 L) [8] Charge to Prep Tank 1: Potassium phosphate dibasic (2.8 kg/kg, 2.8 kg, 1.2 L) [9] Charge to Prep Tank 1: Potassium phosphate monobasic (1.9 kg/kg, 1.9 kg, 0.81 L)

Charge to Prep Tank 1: Water (1.5 L/kg, 1.5 kg, 1.5 L)

Agitate to achieve full dissolution

Discharge for later use
Output: 4M K₂HPO₄/KH₂PO₄ (pH 7.0)

### Unit Op: Extraction-Reactor 1

Adjust batch to 20° C.

Charge to Reactor 1: Ethyl acetate (8 L/kg, 7.21 kg, 8 L)

Charge to Reactor 1: BSA salt of Compound 1-1 (LR, 1 kg, 0.83 L)

Charge to Reactor 1: Ethyl acetate (2 L/kg, 1.80 kg, 2 L)

Charge to Reactor 1: 4M K₂HPO₄/KH₂PO₄ (pH 7.5) (5 L/kg, 6.76 kg, 5 L)

Agitate for NLT 1 hr to achieve full dissolution

Stop agitation and settle for NLT 1 hr.

Discharge lower aqueous

### Unit Op: Extraction-Reactor 1

Adjust batch to 20° C.

Charge to Reactor 1: 4M K₂HPO₄/KH₂PO₄ (pH 7.0) (5 L/kg, 6.76 kg, 5 L)

Agitate for NLT 1 hr

Stop agitation and settle for NLT 1 hr.

Discharge lower aqueous

Note: Expected aqueous pH: ˜ 7.0

Unit Op: Distillation (Constant Volume)-Reactor 1 Note: Target Pressure: 250 mbar

Max batch temp: 40° C., Max Jacket temp: 50C Typical reflux temp: 30-40° C. @ 250 mbar

Distill under vacuum to a target volume of 3 L/kg

Distill under vacuum while charging Solvent to maintain 3 L/kg Charge to Reactor 1: Acetone (25 L/kg, 19.8 kg, 25 L)

### Collect IPC

Sample IPC-1:
Sample Description: Residual EtOAC
Technique: GC/FID
Range: 0 to 1.0 wt %

### IPC Fail Path

Distill under vacuum while charge solvent to maintain 3 L/kg Charge to Reactor 1: Acetone (5 L/kg, 3.96 kg, 5 L) Collect IPC

### Sample IPC-1:

Sample Description: Residual EtOAC
Technique: GC/FID
Range: 0 to 1.0 wt %

### Unit Op: Precipitation-Reactor 2

Charge to Reactor 2: Water (27 L/kg, 27 kg, 27 L) Adjust T=10° C.

Transfer contents of Reactor 1 to Reactor 2 over NLT 90 mins

Charge to rinse vessel

Charge to Reactor 2: Acetone (1 L/kg, kg)

Transfer rinse to precipitation vessel Age for NLT 60 mins

### Unit Op: Isolation-Reactor 2

Transfer slurry to filter

Filter slurry and discharge filtrate to waste Charge via sprayball to flush crystallizer

Charge to Reactor 2: Water (5 L/kg, 5 kg, 5 L)

Perform displacment wash with crystallizer flush Discharge filtrate to waste

Charge via sprayball to flush crystallizer

Charge to Reactor 2: Water (5 L/kg, 5 kg, 5 L)

Perform displacment wash with crystallizer flush Discharge filtrate to waste

### Unit Op: Drying-Dryer 1

Dry at 40° C. under full vacuum for NLT 12 hrs

Collect IPC

### Sample IPC-2:

Sample Description: Residual Acetone
Technique: GC/FID
Range: 0 to 10000 ppm

### IPC Fail Path

Continue Drying at 40° C. for NLT 12 hrs

Collect IPC Sample IPC-2:
Sample Description: Residual Acetone
Technique: GC/FID
Range: 0 to 10000 ppm

### Collect IPC

IPC Fail Path
Continue Drying at 40° C. for NLT 12 hrs
Collect IPC Sample IPC-3:

Sample Description: Residual Water
Technique: KF/Coulametric (Compendial)
Range: 0 to 3.0 wt %

### Discharge Product

Output: Comound 1-1 (90% yield)

### Analytical Data:

The HPLC purity of Compound 1-1 was determined using the below method:

| | |
|---|---|
| Mobile Phase | Mobile Phase A: Ammonium |
| | Acetate aqueous buffer (5 mM, |
| | pH 5.00):acetonitrile (95:5 v:v) |
| | Mobile Phase B: Ammonium |
| | Acetate aqueous buffer (5 mM, |
| | pH 5.00):acetonitrile (20:80 v:v) |
| Column | Acquity UPLC BEH phenyl hexyl, |
| | 3.0 × 100 mm, 1.7 μm particle size |
| Flow Rate | 0.8 mL/min |
| Column Temperature | 45° C. |
| Auto-sampler | 5° C. |
| Temperature | |
| Detector Wavelength | UV @ 250 nm |
| Injection Volume | 3 μL |
| Sample Concentration | 0.2 mg/mL in amber glassware |
| LOQ | 0.2 μg/mL in amber glassware |
| | (0.05% of target cocn.) |
| Sample Diluent | Water: Acetonitrile (50:50) |
| Gradient | Time (minutes) |
| | 0.0 |
| | 16.0 |
| | 16.1 |
| | 20.0 |
| Run Time | 20.0 mins |

¹H NMR (400 MHZ, DMSO-d₆) δ ppm 8 10.79 (s, 1H); 9.37 (s, 1H), 8.39 (d, J=8.4 Hz, 1H); 8.30 (s, 1H); 8.08 (dd, J-8.00 Hz, 1.2 Hz, 1H); 7.17-7.11 (m, 2H); 7.03-6.98 (m, 2H); 6.81 (d, J=8.00 Hz, 1H), 6.40 (d, J=8.4 Hz, 1H); 5.81 (d, J-8.00 Hz, 1H), 4.31-4.27 (m, 1H), 4.16 (t, J-5.6 Hz, 1H); 2.95 (d, 1H), 2.81-2.76 (m, 6H), 2.61-2.58 (m, 4H); 2.4-2.3 (m, 1H); 2.11-2.09 (m, 2H), 1.90-1.85 (m, 1H); 1.5 (s, 6H), 1.68 (t, J-7.6 Hz 3 H); 1.0 (d, J-6.0 Hz, 3H)

CHN Analysis: Calculated: C: 60.13%, H: 5.54%, N: 13.68% Found: C: 59.53%; H: 5.54%; N: 13.54%

### Example 3: Synthesis of (R)-3-((3-aminophenyl)amino) piperidine-2,6-dione, (+)-CSA salt (Compound (R)-J)

The title compound was prepared according to the following reaction scheme:

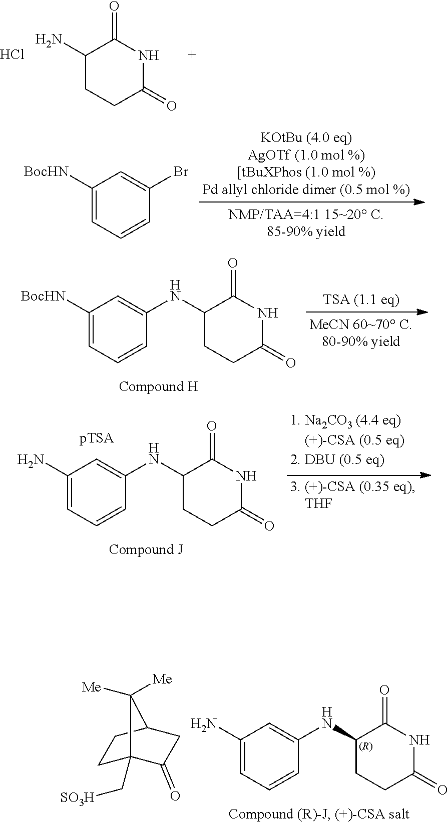

The following synthetic steps were undertaken to prepare Compound (R)-J:

### Step 1: Synthesis of tert-butyl (3-((2,6-dioxopiperidin-3-yl)amino)phenyl) carbamate (Compound H)

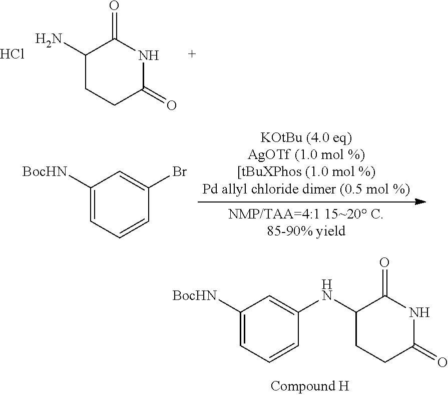 [SMILES: Cl ; NC1CCC(=O)NC1=O ; CC(C)(C)OC(=O)Nc1cccc(Br)c1 ; CC(C)(C)OC(=O)Nc1cccc(NC2CCC(=O)NC2=O)c1]

Two reactions routes (Step 1, Route 1 and Step 1, Route 2) described below were used to prepare Compound H: Step 1, Route 1: DMAC/TAA route
1) Charge DMAC (2.4 vol) and tert-amyl alcohol (0.6 vol) to R-1. Sample the mixture for KF analysis (target: KF≤0.05%). Continue to charge DMAC (9.5 vol) and tert-amyl alcohol (2.4 vol) to R-1. Adjust the mixture to 15-25° C. Bubble nitrogen to the mixture from the bottom for 0.5-1 h until oxygen content≤0.1% 2) Charge tert-butyl (3-bromophenyl) carbamate (1.0 eq) and 3-aminopiperidine-2,6-dione hydrochloride (1.5 eq) to R-1 through plastic-lined solid addition funnel at 15-25° C. Rinse the addition funnel with DMAC (0.1vol). Stir the mixture for 10-20 mins, and bubble nitrogen to the mixture from the bottom at 10-20 mins until oxygen content≤0.1%
3) Charge KOtBu (4.0 eq) portion-wise into the mixture through solid addition funnel at 15-25° C.
4) Adjust the mixture to 15-20° C.

### Catalyst Preparation

5) Charge THF (0.25 vol) to R-2, and bubble nitrogen to the mixture at 10˜20 mins. Add allylpalladium chloride dimer (0.08 eq) and AgOTf (0.16 eq). Adjust to 15-25° C., and stir for NLT 0.5h. Bubble nitrogen to the mixture at 10-20 mins until oxygen content≤0.1%.
6) Charge tBuXPhos (0.016 eq) and THF (0.25 vol) to R-2. Bubble with nitrogen at 10-20 mins until oxygen content≤0.1%. Stir for 0.5 h. Sample for 31P-NMR (FIO).
7) Transfer the catalyst in R-2 through a polish filter into R-1.
8) Charge THF (0.25 vol) to R-2 bubble with nitrogen at 10-20 mins until oxygen content≤0.1%. Transfer the THF solution in R-2 into R-1 through polish filter.

### Reaction

9) React at 15-20° C. After 3 hrs, sample the mixture every 2-5 hrs for HPLC analysis, until area % of Tert-Butyl N-(3-bromophenyl) carbamate is ≤0.5 area %. If tert-butyl N-(3-bromophenyl) carbamate content of two consecutive samples both >0.5 area % and the difference is ≤0.5%, add additional complexation catalyst. And then continue to react until qualified.

### Workup

10) Quench the reaction mixture with acetic acid (3.0 eq) at 15˜25° C. N-Ac solution preparation 11) Add purified water (5V) and N-acetyl-cysteine (0.5 eq.) into a plastic drum. Stir until solid dissolves completely by visual check and store for later use. Bubble with nitrogen for 10-20 mins.

### Crystallization

12) Charge N-acetyl-cysteine solution (1 vol) into R-1.
13) Charge Compound H seed crystal into the mixture, and stir for 1 h at 15-25° C. Confirm seed is not dissolved.
14) Charge remaining N-acetyl-cysteine solution (4 vol) into R-1.
15) Stir the mixture at 15-25° C. for crystallization. After 6 hrs, sample the mixture every 1-4 hrs for mother liquor Compound H wt % analysis until it is ≤0.5 wt %.
16) Filter, and rinse the wet cake with the solution of water/DMA=1:1 (5 vol).
17) Rinse with the wet cake with water (5 vol). Sample the filter cake for purity analysis, Pd residue and Ag residue (FIO).

### Recrystallization:

18) Charge DMAC (7 vol) and the wet cake at 15-25° C. Charge water (7 vol) slowly.

19) Stir the mixture at 15-25° C. for 3 hrs, and sample the mixture every 1-4 hrs for mother liquor Compound H wt % analysis until it is ≤0.2%.

20) Filter, and rise the wet cake with the solution of water/DMAC=1:1 (2 vol).

21) Rinse with the wet cake with water (5 vol). Sample for purity analysis (Purity≥98.5% and single impurity≤0.15%, RRT=1.24 impurity≤0.4%, RRT=1.43 impurity≤0.2%, 3-aminopiperidine-2,6-dione hydrochloride residual≤0.7%). If fail, repeat the recrystallization process. If the purity meets the specification, sample the filter cake for DMAC residue (NMT 1.0%). If fail, continue to rinse with water (5 vol), until DMAC residue NMT 1.0%.

### Drying:

22) Dry the product at 30° C.-45° C. until KF≤1.0%.

Step 1, Route 2: NMP/TAA route:

1) Charge NMP (2.4 vol) and tert-amyl alcohol (0.6 vol) to R-1. Sample the mixture for KF analysis (target: KF<0.05%). Continue to NMP (9.5 vol) and tert-amyl alcohol (2.4 vol) to R-1. Adjust the mixture to 15-25° C. Bubble nitrogen to the mixture from the bottom for 0.5-1 h until oxygen content≤0.1%
2) Charge tert-butyl (3-bromophenyl) carbamate (1.0 eq) and 3-aminopiperidine-2,6-dione hydrochloride (1.5 eq) to R-1 through plastic-lined solid addition funnel at 15-25° C. Rinse the addition funnel with NMP (0.1 vol). Stir the mixture for 10-20 mins, and bubble nitrogen to the mixture from the bottom at 10-20 mins until oxygen content≤0.1%
3) Adjust the mixture to 0-10° C. (target: 5° C.) and charge KOtBu (4.0 eq) portion-wise into the mixture through solid addition funnel at 0-10° C. (target: 5° C.). 4) Keep the mixture at 0-10° C. (target: 5° C.) before charging of catalyst.

Catalyst Preparation
5) Charge THF (0.25 vol) to R-2, and bubble nitrogen to the mixture at 10-20 mins. Add allylpalladium chloride dimer (0.005 eq) and AgOTf (0.01 eq). Adjust to 15-25° C., and stir for NLT 0.5h. Bubble nitrogen to the mixture at 10-20 mins until oxygen content≤0.1%.

6) Charge tBuXPhos (0.01 eq) and THF (0.25 vol) to R-2. Bubble with nitrogen at 10˜20 mins until oxygen content≤0.1%. Stir for 0.5 h. Sample for 31P-NMR (FIO).

7) Transfer the catalyst in R-2 through a polish filter into R-1 at 0-10° C.

8) Charge THF (0.25 vol) to R-2 bubble with nitrogen at 10-20 mins until oxygen content≤0.1%. Transfer the THE solution in R-2 into R-1 through polish filter.

Reaction
9) Adjust the mixture in R-1 to 15˜20° C. and react at 15˜20° C. After 3 h, sample the mixture every 2˜5h for HPLC analysis, until area % of tert-butyl N-(3-bromophenyl) carbamate is ≤0.5 area %. If tert-butyl N-(3-bromophenyl) carbamate content of two consecutive samples both >0.5 area % and the difference is ≤0.5%, add additional complexation catalyst. And then continue to react until qualified.

Workup 10) Quench the reaction mixture with acetic acid (3.0 eq) 15-20° C.

N-acetyl-cysteine solution preparation 11) Add purified water (10 vol) and N-acetyl-cysteine (1.0 eq.) into a plastic drum. Stir until solid dissolves completely by visual check and store for later use. Bubble with nitrogen for 10-20 mins.

Crystallization 12) Charge N-acetyl-cysteine solution (10 vol) into R-1 over 6-8 h at 15˜25° C.

13) After charging, stir the mixture at 15-25° C. for crystallization. After 6 h, sample the mixture every 1-4 h for mother liquor Compound H wt % analysis until NMT 0.2 wt %.

14) Filter, and rinse the wet cake with the solution of water/NMP=1:1 (3 vol).

15) Rinse with the wet cake with water (5 vol).
16) Sample for purity analysis (Purity>98.5% and single impurity≤0.15%, RRT=1.24 impurity≤0.4%, RRT=1.43 impurity≤0.2%, 3-aminopiperidine-2,6-dione hydrochloride residual≤0.7%). Sample for Pd residue and Ag residue (FIO) analysis.

17) Soaking the wet cake with water (5 vol) for 1-2 h and filter, repeat rinsing and filtration process again. Sample the filter cake for NMP residue (NMT 1.0%). If fail, continue to rinsing with water (5 vol), until NMP residue NMT 1.0%.

### Drying:

18) Dry the product at T_{jacket}≤45° C. until KF≤1

### Results and Discussion

Investigation of Reaction Parameters
1. Exp. 1: Optimize the crystallization condition with different volume water for DMAC/TAA as solvent condition. After charge water (7Vol), the purity was consistent with original condition. The yield loss will be decreased from 10% to 5%. After kicker charge water to 8Vol, RRT=0.96 impurity is increased from ND to 0.29%. So 7Vol water can be as the crystallization condition.
2. Evaluate the catalyst loading with DMAC/TAA as solvent (allylpalladium chloride dimer: 0.5 mol %, 0.6 mol %, 0.7 mol % and 0.8 mol %). 0.7 mol % is the optimal catalyst loading with DMAC/TAA as solvent.
1) Exp. 2: Evaluate the catalyst loading (allylpalladium chloride dimer (0.007 eq), tBuXPhos (0.014 eq) and AgOTf (0.014 eq)). 30g reaction was conducted, 3h IPC 98.9%, SM: ND. After crystallization with N-acetyl-cysteine solution (5Vol), the solid product is 99.95%, assay is 97.6% and the corrected yield is 83%. The result is normal.
2) Exp. 3: Evaluate the catalyst loading (allylpalladium chloride dimer (0.006 eq), tBuXPhos (0.012 eq) and AgOTf (0.012 eq)). 30 g scale reaction was conducted, 25 h IPC 97.61%, SM: 1.10% (spec. NMT0.5%). After crystallization with N-acetyl-cysteine solution (7Vol). 31.07g solid was obtained with 100% purity and 88.2% uncorrected yield. 1.1% SM residue can be purged in the crystallization stage.
3) Exp. 4: Evaluate the catalyst loading (allylpalladium chloride dimer (0.005 eq), tBuXPhos (0.010 eq) and AgOTf (0.010 eq)). 30 g scale reaction was conducted, 41 h IPC 96.98%, SM: 0.75% (spec. NMT0.5%). After crystallization with N-acetyl-cysteine solution (7Vol). 30.98g solid was obtained with 100% purity and 88% uncorrected yield.
3. Exp. 5: for the DMAC/TAA strategy, 100 g scale-up reaction was conducted with (allylpalladium chloride dimer (0.007 eq), tBuXPhos (0.014 eq) and AgOTf (0.014 eq)) to confirm the optimized condition. 3 h IPC 97.40%, SM: ND. After crystallization with N-acetyl-cysteine solution (7Vol). 103g solid was obtained with 100% purity, 95.08% assay and 84.3% corrected yield. The result is normal.
4. Try crystallization with different volume of N-acetyl-cysteine. 10Vol N-acetyl-cysteine solution is suitable for the crystallization. The yield loss can be controlled in ˜4%, the solid purity >99.8% and the other results are normal.

| Water | Stirred | Wet cake | | |
|---|---|---|---|---|
| volume | time | Purity | Filtrate wt % | Yield loss |
| 6 V | 3 h | 100% | 0.90 wt % | ~18 yield |
| | | | | loss. |
| 7 V | 1 h | 100% | 0.72 wt % | ~14 yield |
| | | | | loss |
| | 3 h | 100% | 0.63 wt % | ~12% yield |
| | | | | loss |
| 9 V | 3 h | 100% | 0.30 wt % | ~6% yield |
| | | | | loss. |
| 10 V | 3 h | — | 0.16% | ~4% yield |
| | | | | loss |
| 12 V | 20 h | N/A | 0.14 wt % | ~3% yield |
| | | | | loss. |

The wet cake washed with 7 V water.

| | | | | |
|---|---|---|---|---|
| Wash | 3 h | 99.83% | 97.68 wt % | 89% |
| with | | | (N-acetyl-cysteine | |
| water | | | residue <LOQ (LOQ = | |
| (7 Vol) | | | 0.05%)). | |
| | | | BMT-336549: 0.30%. | |
| | | | NMP residue: 0.54%. | |
| | | | THF residue: 0.08%. | |
| | | | TAA residue: 0.16%. | |
| | | | HOAc residue <LOQ | |
| | | | (LOQ = 0.05%) | |
| | | | Pd 6 ppm and Ag 5 ppm | |

5. Exp. 6 scale up 30 g to confirm the process with NMP/TAA as the solvent. 3h IPC 99.27%, SM: ND. After crystallization with 10Vol N-acetyl-cysteine solution and wash with water (7Vol), then drying at RT for 20 h, 32.97g product was obtained with 99.92% purity, 96.44% assay and 90.3% corrected yield. Other test result as table below.

| HOAc | NMP | THF | TAA | NMT- | N-acetyl- | | Pd | Ag |
|---|---|---|---|---|---|---|---|---|
| residue | residue | residue | residue | 336549 | cysteine | KF | residue | residue |
| <LOQ | 0.63% | 0.21% | 0.23% | 0.12% | <LOQ | 2.58% | 34 ppm | 6 ppm |
| (LOQ = | | | | | (LOQ = | | | |
| 0.05%) | | | | | 0.05%) | | | |

6. Evaluate the catalyst loading with NMP/TAA as reaction solvent. 0.5 mol % catalyst is suitable for the reaction.
   1) Exp. 7: Reduce the catalyst loading (allylpalladium chloride dimer (0.5 mol %), tBuXPhos (1.0 mol %) and AgOTf (1.0 mol %)) with NMP/TAA=12:3 as the solvent. 3 h IPC 96.91%, SM: 2.87, 5ah IPC 99.23%, SM: ND. The result is normal.
   2) Exp. 8: Reduce the catalyst loading (allylpalladium chloride dimer (0.35 mol %), tBuXPhos (0.7 mol %) and AgOTf (0.7 mol %)) with NMP/TAA=12:3 as the solvent. Extend the reaction time for 21 h, IPC 39.71%, SM: 57.51%. The reaction looks stalled.
   3) Exp. 9: Reduce the catalyst loading (allylpalladium chloride dimer (0.4 mol %), tBuXPhos (0.8 mol %) and AgOTf (0.8 mol %)) with NMP/TAA=12:3 as the solvent. Extend the reaction time for 19 h, IPC 99.36%, SM: ND. The result is normal
7. Exp. 10 and 11 and 12 and 13. Evaluate the impact of reaction temperature (10° C., 15° C., 25° C., 30° C.). The reactions are normal at 15-30° C., the reaction rate is slow at 10° C.
   Stress test:

### IPC Methods Developed

The reaction was monitored by UPLC (The analysis method is AM-129023-07)

| | |
|---|---|
| Column | Infinitylab Poroshell 120 EC-C18 2.1*50 mm*1.9 |
| | um P/N: 699675-902 |
| Mobile phase A | 10 mM NH₄OAC in 95water:5ACN |
| Mobile phase B | 10 mM NH₄OAC in 5water:95ACN |
| Gradient program | T (min): 0.0 4.0 4.5 4.51 6.0 |
| | B (%): 0 100 100 0 0 |
| Column temperature | 40° C. |
| Flow rate | 0.65 mL/min |
| Injection volume | 1 μL |
| Detector | UV at 225 nm |
| Needle wash solvent | MeCN |
| Run Time | 6.0 min |

### Step 2: Synthesis of 3-((3-aminophenyl)amino) piperidine-2,6-dione, p-toluenesulfonic acid salt (Compound J)

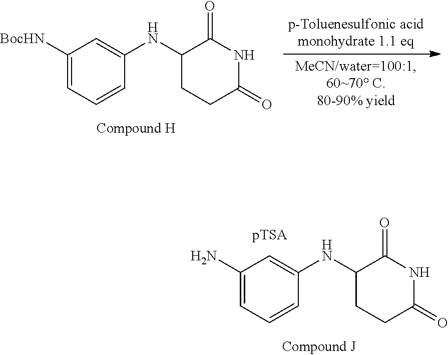 [SMILES: CC(C)(C)OC(=O)Nc1cccc(NC2CCC(=O)NC2=O)c1 ; Nc1cccc(NC2CCC(=O)NC2=O)c1]

The reaction was conducted as described below:
1) Charge acetonitrile (19 vol) into R-1. Sample the mixture for KF analysis (FIO).
2) Charge purified water (0.3 vol) into R-1.
3) Charge Compound H (1.0 eq) into R-1 through solid addition funnel at 15-25° C.
4) Charge Compound J seeds (0.02 kg/kg) into R-1 through solid addition funnel at 15-25° C. Rinse the funnel with acetonitrile (1 vol).
5) Heat the mixture to 60-70° C., at a reference rate of 10˜20° C./h.
6) Charge acetonitrile (9 vol) into R-2.
7) Charge p-toluenesulfonic acid monohydrate (1.leq) into R-2 through a solid addition funnel. Rinse the funnel with acetonitrile (1 vol).
8) Charge the P-TSA solution through a peristaltic pump at 60-70° C.
9) React at 60-70° C. After 12 h, sample the mixture every 2˜5h for HPLC analysis, until area % of Compound His≤1.8 area %. If Compound HI content of two consecutive samples both >1.8 area % and the difference is ≤0.5%, add additional p-Toluenesulfonic acid monohydrate solution. Then continue to react until qualified.
10) Cool the mixture to 15-25° C., at a reference rate of 10-15° C./h.
11) Stir the mixture at 15-25° C. for crystallization. After 2 h, sample the mixture every 1-4 h for mother liquor wt % analysis until it is ≤0.2 wt %.
12) Filter, and rinse the wet cake with acetonitrile (3 vol). Sample the filter cake for purity analysis (FIO).
13) Dry the solid at ≤40° C. until acetonitrile residual≤1.0%.

### Step 3: Part 1: Synthesis of (R)-3-((3-aminophenyl)amino) piperidine-2,6-dione (+) CSA salt (Compound (R)-J)

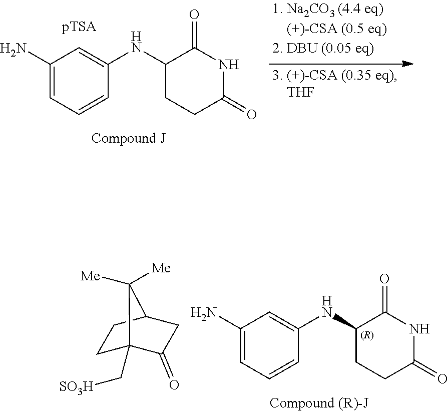

The reaction was conducted as described below:
1) Freebasing: Compound J and 4.40 eq. Na₂CO₃ were added to 20V THF and stirred for NLT 6 hrs at RT.
2) Waste filtration: The byproducts (TsONa, sodium bicarbonate) and unreacted Na₂CO₃ were filtered off and the solids were washed with 6 vol THF.
3) Distillation & Chiral Resolution: The filtered batch was then distilled down to 10 vol THF and subjected to a classical resolution by slowly adding 0.45 eq. (1S)-(+)-10-camphorsulfonic acid (CSA) in 4.5 vol THF. The reaction was stirred for NLT 10 h.
4) 1^{st} Isolation: Once this crystallization was complete the crystallized product (Compound (R)-J) was filtered off, washed with 6 vol THF, and dried under vacuum at 40° C. for NLT 12 hrs (IPC-2) to yield ˜43% isolated (Compound (R)-J) with 97:3 (R) to(S) enantiopurity.
5) Recycling of the Mother Liquor (ML)-Racemization: The ML, enriched with the undesired(S) enantiomer, was then distilled down to 5 vol and 5 mol % 1,8-Diazabicyclo [5.4.0] undec-7-ene (DBU) was added to initiate the racemization process. Once IPC-3 was met (racemic (Compound J was regenerated) a similar crystallization protocol to the one mentioned above commenced.
6) Quench & Crystallization: 0.35 eq. CSA in 2.2 vol THF was slowly added, and the reaction was stirred for NLT 10 h.
7) 2^{nd} Isolation: After this second drop of product is collected by filtration, washed with 6 vol THE, and dried under vacuum at 40° C. for 12 hrs to yield ˜23% isolated (Compound (R)-J) with 98:2 (R) to(S) enantiopurity. Both batches of (Compound (R)-J) were combined to give ˜67% yield over all yield with excellent enantiopurity.

### Optimized Conditions:

Stage 1: free-basing.
1. Charge THF (18 vol) into the reactor 1, Adjust the mixture to 15-25° C.
2. At 15-25° C., add Compound J (1.0 eq) into the glass-lined reactor1 through solid addition equipment. Rinse with THF (1 vol).
3. At 15-25° C., add sodium carbonate (4.4 eq, 1.19 g/g) into the glass-lined reactor1 through solid addition equipment. Rinse with THF (1 vol).
4. The mixture is allowed to react at T=15-25° C. for at least 6 h,
5. Filter the mixture with nutsche filter at 15˜25° C. Rinse the filter cake with THE (3 vol).
6. Concentrate the mixture at T≤40° C. (T_{jacket}≤50° C.) under reduced pressure (reference P≤-0.08 MPa) until 10˜11 vol left. Sample the mixture for KF analysis (Criterion: KF≤0.3 wt %). If KF>0.3 wt %, continue swap with THF (5 vol) until KF NMT 0.3 wt %.

### Stage 2: 1^{st} Chiral Resolution.

7. Transfer the mixture of glass-lined reactor 1 to glass-lined reactor 2 by polish filter. Rinse the reactor 1 with THF (1 vol) and transfer the mixture to glass-lined reactor 2 by polish filter. Adjust the mixture in glass-lined reactor 2 to 15-25° C. 8. At 15-25° C., add Compound-(R) J seed (0.01 g/g) into the glass-lined reactor and stir for NLT 2 h.
9. At 15-25° C., add (1S)-(+)-Camphor-10-sulfonic acid solution (0.5 eq dissolved in 5 vol THF) into the mixture in glass-lined reactor 2 by Halar liquid material filter at rate of 15-23 Kg/h and control the drop time NLT 10 h.
10. Stir the mixture at 15-25° C. for crystallization. After 6 h, sample the mixture for filter cake chiral purity (FIO) and filtrate chiral purity (FIO) and wt % analysis.
11. Filter the mixture with a halar-lined nutsche filter at 15-25° C.
12. Rinse the filter cake with THF (3 vol), combine the filtrate and rinsing, and sample the combined filtrate for chiral purity, Compound J wt % and camphor sulfonic acid residue analysis.
13. Rinse the filter cake with acetonitrile (3 vol). Sample the acetonitrile rinse liquid for Compound J wt % analysis.
14. Sample the filter cake for purity, chiral purity, Pd residue (FIO) and silver residue (FIO) analysis.

### Stage 2: Purification Process.

15. Add acetonitrile (2 vol) and purified water (0.15 g/g) into the glass-lined reactor 2. Adjust the mixture to 15-25° C. Stir for 5 mins at least.
16. At 15-25° C., add filter cake in step 14 into the glass-lined reactor 2 through solid addition equipment. Rinse with acetonitrile (0.5 vol).
17. Heat the mixture to 50-60° C., at a reference rate of 10-20° C./h.
18. Maintain temperature and stir for at least 2 h at 50-60° C.,
19. Filter the mixture with a halar-lined nutsche filter. Rinse with acetonitrile (1 vol). Sample the filtrate for Compound J wt % analysis. Sample the filter cake for purity, Chiral purity, Pd residue (FIO) and silver residue (FIO) analysis.

### Stage 2: Drying

20. Sweep with nitrogen for NLT 2 h in halar-lined nutsche filter, then dry the solid at T_{jacket}≤40° C., 12 h later, sample the solid for GC analysis every 4-8 h until Total residual (THF residue+acetonitrile residue)≤1.8 wt %. During drying, turn over the solid every 4-8 h. After drying, cool to T≤30° C.

### Stage 3: 2^{nd} Chiral Resolution.

21. Transfer the filtrate in step 12 into glass-lined reactor. Concentrate the mixture at T<40° C. (reference T_{jacket}≤50° C.) under reduced pressure (reference P<-0.08 MPa) until 5-6 V left.

22 At 15-25° C., add DBU (0.05 eq) into the mixture in glass-lined reactor. The mixture is allowed to react at T=15-25° C. After 3 h, sample the mixture every 1-5 h for HPLC analysis, until Compound J Chiral purity>49%. If Compound J Chiral purity of two consecutive samples both <49% and the difference is≤0.5%, add additional DBU. And then continue to react until qualified (Each time of charging additional amount of DBU is 0.01 eq).
23. Transfer the mixture into glass-lined reactor 5 through polish filter.
24 At 15-25° C., add Compound (R)-J seed crystal (0.05 g/g) into the glass-lined reactor through a halar-lined solid addition equipment, stir for NLT 2h.
25 At 15-25° C., add (1S)-(+)-Camphor-10-sulfonic acid solution (0.35 eq dissolved in 3.5 vol THF) into glass-lined reactor 5 by liquid material filter at a reference rate of 10-15 Kg/h.
26. Stir the mixture at 15-25° C. for crystallization. After 6 h, sample the mixture for filter cake chiral purity (FIO) and filtrate chiral purity (FIO) and wt % (FIO) analysis.
27. Filter the mixture with a halar-lined nutsche filter at 15-25° C.
28. Rinse the filter cake with THF (1.5 vol).
29. Rinse the filter cake with acetonitrile (1.5 vol).
30. Sample the filter cake for purity, chiral purity, Pd residue (FIO) and silver residue (FIO) analysis.

Stage 3: Optional purification process.
31. Add acetonitrile (1 vol) and purified water (0.075 g/g) into the glass-lined reactor 2. Adjust the mixture to 15-25° C. Stir for 5 mins at least.
32. At 15˜25° C., add filter cake in step 30 into the glass-lined reactor 2 through solid addition equipment. Rinse with acetonitrile (0.25 vol).
33 Heat the mixture to 50-60° C., at a reference rate of 10-20° C./h.
34. Maintain temperature and stir for 2 h at least at 50-60° C.
35. Filter the mixture with a halar-lined nutsche filter. Rinse with acetonitrile (1 vol).
36. Sample the filter cake for purity, chiral purity, Pd residue (FIO) and silver residue (FIO) analysis.

Stage 3: Drying
37. Sweep with nitrogen for NLT 2 h in halar-lined nutsche filter, then dry the solid at T_{jacket}≤40° C., 12 h later, sample the solid for GC analysis every 4-8 h until Total residual (THF residue+acetonitrile residue)≤1.8 wt %. During drying, turn over the solid every 4-8 h. After drying, cool to T≤30° C.

The final condition of 3^{rd} chiral resolution:
1. Add the mother liquor (Compound J, 1.0 eq) into reactor 1.
2. Adjust to 15-25° C., stir for NLT 0.5h, sample the mother liquor Compound J wt % (FIO) analysis.
3. At 15-25° C., adjust to pH=10.0-11.0 with DBU (0.26-0.44 Kg/Kg). After NLT 3h, sample the mixture every 1-5 h for analysis, until area % of Compound (R)-J Chiral purity >49%. If Chiral purity content of two consecutive samples both <49% and the difference is ≤0.5%, then add additional DBU and continue to react until qualified.
4. Transfer the mixture into reactor 2 by a polish filter.
5. Add THF into glass-lined reactor 3, add (1S)-(+)-Camphor-10-sulfonic acid through Halar lined or PTFE lining solid addition equipment. Rinse with THF. Stir until solid dissolves completely by visual check and store for later use.
   1) Charging Qty of CSA=(Mixture in step 1 charging Qty * Compound J wt % * 232.3/219.24*0.5)+ (Mixture in step 1 charging Qty * DBU residue * 232.3/152.24)
   2) Charging Qty of THF=CSA charging Qty * 6.4
   3) Rinse Charging Qty of THF=CSA charging Qty * 0.7
6. At 15-25° C., add 1% seed, stir for 1-2 h and continue add the CSA solution prepared in step 5 to reactor 2. After 6 h, take samples every 1-4 hours until the chiral purity of the supernatant≤11.0%. Sample the solid for chiral purity analysis (FIO). If the result of two consecutive samples>11.0% and the difference is ≤1.0%, continue to add 0.leq (1S)-(+)-Camphor-10-sulfonic acid solution until the chiral purity≤11.0%.
7. Filter the mixture, rinse filter cake with THF (2.65V) and acetonitrile (2.65V). If unqualified, sweep with nitrogen for NLT 2 h in Nutsche filter/Agitating filter dryer, then dry the solid at T_{jacket}≤40° C. 12 h later, sample the solid for GC analysis every reference 4-8h until Total residual (THF residue+acetonitrile residue)≤5 wt %. During drying, every 4-8 h turn over the solid in Nutsche filter/stir the solid for reference 2-5 mins in agitating filter dryer. After drying, cool to T ≤30° C. Proceed to step 8 Operation 8-Operation 11, the charging volume is based on the operation of the filter cake: B (operation 7 cake weight).
8. Add acetonitrile (4.5V), purified water (0.3V), adjust to15-25° C., and stir for NLT 5 mins.
9. Add filter cake in step 7, rinse with acetonitrile (0.5V).
10 Heat to 50-60° C., maintain and stir for at least 2 h at 50-60° C. and sample for the purity and chiral purity. If unqualified, add purified water until qualified (0.1V each time, upper limit: 0.3V), cool to 15-25° C., stir NLT 2 h.
11. Filter the mixture, rinse filter cake with (3V) acetonitrile. If unqualified, repeat steps 8-11.
12. Sweep with nitrogen for NLT 2 h in Nutsche filter/Agitating filter dryer, then dry the solid at T_{jacket}≤40° C. 12 h later, sample the solid for GC analysis every reference 4˜8h until Total residual solvent (THF residue+acetonitrile residue) ≤1.8 wt % and KF≤1.0 wt %. During drying, every 4-8 h turn over the solid in Nutsche filter or stir the solid for reference 2-5 mins in agitating filter dryer. After drying, cool to T≤30° C.

### Investigation of Reaction Parameters

1. Screen the base (Cs₂CO₃, K₂CO₃, NEt₂, DIPEA, Pyridine, DMAP) for the 1^{st} racemization.

| | | Chiral purity of |
|---|---|---|
| Condition | Purity | Compound (R)-J |
| The 1^{st} chiral resolution filtrate | 94.9% | 6.60% |
| 0.3eq Cs₂CO₃ was added into | 92.6% | 46.70% |
| the 1^{st} chiral resolution filtrate. Stir | | |
| at RT for 15 h. | | |
| Stir at RT for 89 h | — | 50.7% |
| The 1^{st} chiral resolution filtrate | 94.9% | 6.60% |
| 0.5eq Cs₂CO₃ was added into | 91.17% | 49.50% |
| the 1^{st} chiral resolution filtrate. Stir | | |
| at RT for 15 h. | | |

2. Purity upgrade: The solid chiral purity can be increased from 96.17% to 98.93% and the yield loss is 2.5%.

| Condition | Purity | Chiral purity |
|---|---|---|
| Before purification | 99.88% | 96.17% |
| 7 g solid was slurry with ACN (5 Vol, | 99.95% | 97.87% |
| 35 mL)/water (0.3 Vol, 2.1 g) at 50- | | |
| 60° C. for 3 h. | | |
| Stir at 50-60° C. for 16 h. | 100% | 99.88% |
| Cool to RT and stir for 2 h | 100% | 98.93% |
| Mother liquor | Loss yield in the | 52.47% |
| | M.L. is 2.5%. | |

3. Exp. 32 Purified the solid (7g, purity 99.88%, chiral purity: 96.17%) with ACN (5 vol)/water (0.6 vol). The chiral purity can be increased from 96.17% to 99.80%. But the yield loss is high (7%).

| Condition | Purity | Chiral purity |
|---|---|---|
| Before purification | 99.88% | 96.17% |
| 7 g solid was slurry with ACN | 99.95% | 99.29% |
| (5 Vol, 35 mL)/water (0.3 Vol, | | |
| 2.1 g) at 50-60° C. for 3 h. | | |
| Stir at 50-60° C. for 16 h. | 100% | 99.52% |
| Cool to RT and stir for 2 h | 100% | 99.80% |
| Mother liquor | Loss yield in the M.L. | 62.29% |
| | is 7% | |

4. For the 2^{nd} racemization, The result is similar with concentration and no-concentration.
   1) Exp. 39A: Head to head comparison for concentration and no-concentration. 33.5 g (free-base) scale reaction was conducted, the filtrate was concentrated to 1/2 of the initial, then added DBU to pH=˜ 9.5. The racemization was completed in 20 h. Then run the 3^{rd} chiral resolution with CSA to pH-3-3.5, 20.1g solid was obtained with 99.66% purity, 98.34% chiral purity and 29% uncorrected yield.
   2) Exp. 39B: Head to head comparison for concentration and no-concentration. 33.5g (free-base) scale reaction was conducted, the filtrate was directly racemic with DBU to pH=˜ 9.5. 20h IPC 48.0% chiral purity. Then run the 3^{rd} chiral resolution with CSA to pH=3-3.5, 20.08g solid was obtained with 99.84% purity, 98.34% chiral purity and 29% uncorrected yield.
5 Evaluate the pH range for the 2^{nd} racemization. The mixture purity and assay is dropped with extending the racemization time. After balance racemization rate and purity drop, pH=10.0-11.0 (target: 10.5) should be the optimum pH range.

| | | | Chiral | Product:biphenyl | |
|---|---|---|---|---|---|
| pH | Sample time | Purity | purity | ratio | Assay |
| 9.56 | 0 h | 95.89% | 8.73% | 50.30:49.70 | 4.35 wt % |
| | 2 h IPC | 96.27% | 12.85% | 50.80:49.20 | 4.35 wt % |
| | 4 h IPC | 96.10% | 15.02% | 50.72:49.28 | 4.26 wt % |
| | 20 h IPC | 96.33% | 31.14% | 50.43:49.57 | 4.29 wt % |
| | 25 h IPC | 96.30% | 32.04% | 50.52:49.48 | 4.23 wt % |
| | 44 h IPC | 94.72% | 37.24% | 49.92:50.08 | 4.25 wt % |

| | | | Chiral | Product:biphenyl | |
|---|---|---|---|---|---|
| pH | Sample time | Purity | purity | ratio | Assay |
| 9.86 | 0 h | 96.44% | 8.28% | 49.72:50.28 | 4.55 wt % |
| | 2 h IPC | 96.58% | 25.37% | 49.32:50.68 | 4.25 wt % |
| | 4 h IPC | 96.42% | 30.59% | 49.58:50.42 | 4.30 wt % |
| | 6 h IPC | 96.36% | 34.42% | 49.53:50.47 | 4.17 wt % |
| | 22 h IPC | 88.82% | 47.40% | 47.44:52.56 | 3.92 wt % |
| | 28 h IPC | 92.15% | 49.38% | 46.94:53.06 | 3.94 wt % |
| | 46 h IPC | 89.29% | — | 45.03:54.97 | 3.64 wt % |

| | | | Chiral | Product:biphenyl | |
|---|---|---|---|---|---|
| pH | Sample time | Purity | purity | ratio | Assay |
| 10.08 | 0 h IPC | 96.06% | 7.47% | 49.87:50.13 | 4.48 wt % |
| | 2 h IPC | 95.62% | 30.01% | 49.54:50.46 | 4.38 wt % |
| | 4 h IPC | 96.59% | 34.57% | 49.76:50.24 | 4.59 wt % |
| | 6 h IPC | 96.83% | 39.58% | 49.69:50.31 | 4.18 wt % |
| | 22 h IPC | 90.39% | 48.89% | 47.84:52.16 | 4.15 wt % |
| | 28 h IPC | 92.71% | 49.89% | 47.26:52.74 | 4.01 wt % |
| | 46 h IPC | 89.84% | — | 45.53:54.47 | 3.70 wt % |

| | | | Chiral | Product:biphenyl | |
|---|---|---|---|---|---|
| pH | Sample time | Purity | purity | ratio | Assay |
| 10.52 | 0 h IPC | 95.10% | 8.22% | 49.83:50.17 | 4.49 wt % |
| | 2 h IPC | 96.01% | 41.23% | 49.48:50.52 | 4.21 wt % |
| | 4 h IPC | 96.34% | 45.45% | 49.46:50.54 | 4.23 wt % |
| | 6 h IPC | 95.96% | 47.35% | 48.95:51.05 | 4.08 wt % |
| | 22 h IPC | 92.05% | 49.99% | 47.74:52.26 | 3.91 wt % |
| | 28 h IPC | 92.66% | 49.98% | 46.50:53.50 | 3.80 wt % |
| | 46 h IPC | 88.30% | — | 43.71:56.29 | 3.36 wt % |

| | | | Chiral | Product:biphenyl | |
|---|---|---|---|---|---|
| pH | Sample time | Purity | purity | ratio | Assay |
| 11.02 | 0 h IPC | 96.44% | 29.89% | 48.71:51.29 | 3.99 wt % |
| | 2 h IPC | 96.22% | 45.05% | 49.19:50.81 | 4.14 wt % |
| | 4 h IPC | 96.18% | 49.18% | 49.35:50.65 | 4.06 wt % |
| | 20 h IPC | 89.70% | 49.77% | 45.03:54.97 | 3.43 wt % |
| | 25 h IPC | 89.88% | 49.96% | 43.08:56.92 | 3.26 wt % |
| | 44 h IPC | 87.21% | — | 39.86:69.14 | 2.94 wt % |

### Exemplary Batch

1. 100 g scale to confirm the finalized process for Compound (R)-J. The result is normal.
   1) After 1^{st} chiral resolution, 51.0 g Compound(S)-J was obtained with 99.75% purity, 98.18% chiral purity and 44.3% yield.
   2) After 2^{nd} chiral resolution, 24.8g product was obtained with 99.94% purity, 98.08% chiral purity and 21.6% yield.
   3) After 3^{rd} chiral resolution, after purification, 8.7g product was obtained with 7.5% yield.
4) IPC Methods Developed

UPLC method: Compound (R)-J-15.M (For IPC purity and assay of Compound (R)-J)

| | |
|---|---|
| Column | Infinitylab Poroshell 120 EC-C18 |
| | 2.1*50 mm*1.9 um P/N: 699675-902 |
| Mobile phase A | 10 mM NH₄OAC in 95water:5ACN |
| Mobile phase B | 10 mM NH₄OAC in 5water:95ACN |
| Gradient program | T (min): 0.0 4.0 4.5 4.51 6.0 |
| | B (%): 0 100 100 0 0 |
| Column temperature | 40° C. |
| Flow rate | 0.65 mL/min |
| Injection volume | 0.5 μL |
| Detector | UV at 225 nm |
| Needle wash solvent | 65ACN:35WATER |
| Run Time | 6.0 min |

HPLC method: Compound (R)-J-14.M (For IPC chiral purity of Compound (R)-J)

| | |
|---|---|
| Column | CHIRALCEL AD-H 5 um 4.6 × 250 mm |
| | (P/N: 19325) |
| Column Temperature | 40° C. |
| Flow Rate | 1.0 mL/min |
| Injection Volume | 5 μL |
| Mobile Phase | 0.1% DEA in MeOH |
| Run time | 15 min |
| Needle Wash Solvent | EtOH |
| Detection | UV at 225 nm |

### Step 3: Part 2: Synthesis of(S)-3-((3-aminophenyl)amino) piperidine-2,6-dione (−) CSA salt (Compound(S)-J)

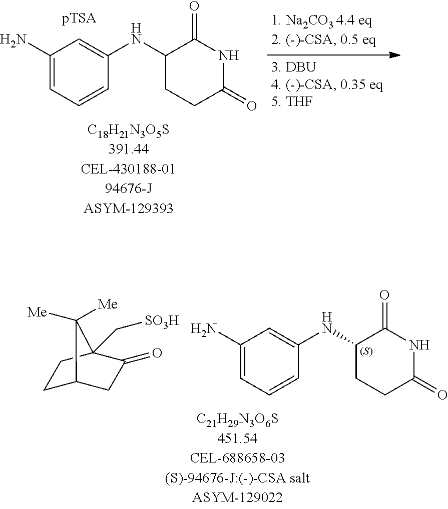

The reaction was conducted as described below:

### Stage 1: Free-basing.

1. Charge THF (18 vol) into the reactor 1, Adjust the mixture to 15˜25° C.
2. At 15-25° C., add Compound J (1.0 eq) into the glass-lined reactor 1 through solid addition equipment. Rinse with THF (1 vol).
3. At 15-25° C., add sodium carbonate (4.4 eq, 1.19 g/g) into the glass-lined reactor 1 through solid addition equipment. Rinse with THF (1 vol).
4. The mixture is allowed to react at T=15-25° C. for at least 6 h
5. Filter the mixture with nutsche filter at 15-25° C. Rinse the filter cake with THF (3 vol).
6. Concentrate the mixture at T≤40° C. (T_{jacket}≤50° C.) under reduced pressure (reference P≤-0.08 MPa) until 10-11V left. Sample the mixture for KF analysis (Criterion: KF≤0.3 wt %). If KF>0.3 wt %, continue swap with THF (5 vol) until KF NMT0.3 wt %.
   Stage 2: 1^{st} Chiral resolution.
7. Transfer the mixture of glass-lined reactor 1 to glass-lined reactor 2 by polish filter. Rinse the reactor1 with THF (1 vol) and transfer the mixture to glass-lined reactor 2 by polish filter. Adjust the mixture in glass-lined reactor 2 to 15-25° C.
8. At 15-25° C., add Compound(S)-J seed (0.01 g/g) into the glass-lined reactor and stir for NLT 2 h.
9. At 15-25° C., add (1R)-(−)-Camphor-10-sulfonic acid solution (0.5 eq dissolved in 5 vol THF) into the mixture in glass-lined reactor 2 by Halar liquid material filter at rate of 15-23 Kg/h and control the drop time NLT 10h.
10. Stir the mixture at 15-25° C. for crystallization. After 6 h, sample the mixture for filter cake chiral purity (FIO) and filtrate chiral purity (FIO) and wt % analysis.
11. Filter the mixture with a halar-lined nutsche filter at 15-25° C.
12. Rinse the filter cake with THF (3vol).
13. Rinse the filter cake with acetonitrile (3 vol).
14. Sample the filter cake for purity, Chiral purity, Pd residue (FIO) and silver residue (FIO) analysis. If fail, run the purification process.

### Stage 2: Purification Process.

15. Add acetonitrile (2 vol) and purified water (0.15 g/g) into the glass-lined reactor 3. Adjust the mixture to 15˜25° C. Stir for 5 mins at least.

16. At 15-25° C., add filter cake in step 14 into the glass-lined reactor 3 through solid addition equipment. Rinse with acetonitrile (0.5 vol).
17. Heat the mixture to 50-60° C., at a reference rate of 10˜20° C./h.
18. Maintain temperature and stir for at least 2 h at 50-60° C., the samples should be taken every 1-4 h to detect the purity and chiral purity. If fail, continue kicker charge water (0.05 g/g), until meet the specification. The Maximum amount of water kicker charge is 0.15 g/g.
19. Filter the mixture with a halar-lined nutsche filter. Rinse with acetonitrile (1 vol). If fail, repeat the purification process.

### Stage 2: Drying

20. Sweep with nitrogen for NLT 2 h in halar-lined nutsche filter, then dry the solid at T_{jacket}≤40° C., 12 h later, sample the solid for GC analysis every 4-8 h until Total residual (THF residue+acetonitrile residue)≤1.8 wt %. During drying, turn over the solid every 4-8 h. After drying, cool to T≤30° C.

### Stage 3: 2^{nd} Chiral Resolution.

21. Transfer the filtrate in step 12 into glass-lined reactor 1. Concentrate the mixture at T≤40° C. (reference T_{jacket}≤50° C.) under reduced pressure (reference P≤-0.08 MPa) until 5-6 V left.
22 At 15-25° C., adjust to pH=10.0-11.0 with DBU (the reference of charging amount is 0.05 eq˜0.06 eq). After NLT 3h, sample the mixture every 1˜5h for HPLC analysis, until Compound(S)-J Chiral purity>49%. If Compound(S)-J Chiral purity of two consecutive samples both <49% and the difference is≤0.5%, add additional DBU. And then continue to react until qualified.
23. Transfer the mixture into glass-lined reactor 2 through polish filter.
24 At 15-25° C., add Compound(S)-J seed crystal (0.05 g/g) into the glass-lined reactor 2 through a halar-lined solid addition equipment, stir for NLT 2 h.
25. At 15-25° C., add (1R)-(−)-Camphor-10-sulfonic acid solution (0.35 eq dissolved in 3 vol THF) into glass-lined reactor 2 by liquid material filter at a reference rate of 10-15 Kg/h.
26. Stir the mixture at 15-25° C. for crystallization. After 6 h, sample the mixture for filter cake chiral purity (FIO) and filtrate chiral purity (FIO) and wt % (FIO) analysis.
27. Filter the mixture with a halar-lined nutsche filter at 15-25° C.
28 Rinse the filter cake with THF (1.5 vol).
29. Rinse the filter cake with acetonitrile (1.5 vol).
30 Sample the filter cake for purity, Chiral purity, Pd residue (FIO) and silver residue (FIO) analysis. If fail, run the purification process.
Stage 3: Purification process.
31. Add acetonitrile (1 vol) and purified water (0.075 g/g) into the glass-lined reactor 3. Adjust the mixture to 15-25° C. Stir for 5 mins at least.
32. At 15-25° C., add filter cake in step 30 into the glass-lined reactor 2 through solid addition equipment. Rinse with acetonitrile (0.25 vol).
33. Heat the mixture to 50-60° C., at a reference rate of 10-20° C./h.
34 Maintain temperature and stir for 2 h at least at 50-60° C., the samples were taken every 1-4 h to detect the purity and chiral purity. If fail, continue kicker charge water (0.025 g/g), until meet the specification. The Maximum amount of water kicker charge is 0.075 g/g.
35 Filter the mixture with a halar-lined nutsche filter. Rinse with acetonitrile (1 vol).
36 Sample the filter cake for purity, Chiral purity, Pd residue (FIO) and silver residue (FIO) analysis. If fail, repeat the purification process.

### Stage 3: Drying

37 Sweep with nitrogen for NLT 2 h in halar-lined nutsche filter, then dry the solid at T_{jacket}≤40° C., 12 h later, sample the solid for GC analysis every 4˜8h until Total residual (THF residue+acetonitrile residue)≤1.8 wt %. During drying, turn over the solid every 4˜8 h. After drying, cool to T≤30° C.

The final condition of 3^{rd} chiral resolution:
1. Add the mother liquor (Compound(S)-J, 1.0 eq) into reactor 1.
2. Adjust to 15-25° C., stir for NLT 0.5h, sample the mother liquor Compound(S)-J wt % (FIO) analysis.
3. At 15-25° C., adjust to pH=10.0-11.0 with DBU (0.26˜0.44 Kg/Kg). After NLT 3h, sample the mixture every 1-5 h for analysis, until area % of Compound(S)-J Chiral purity >49%. If Chiral purity content of two consecutive samples both <49% and the difference is ≤0.5%, then add additional DBU and continue to react until qualified.
4. Transfer the mixture into reactor 2 by a polish filter, sample the Compound(S)-J wt % (FIO) and DBU residue analysis.
5. Add THE into glass-lined reactor 3, add ((1S)-(+)-Camphor-10-sulfonic acid through Halar lined or PTFE lining solid addition equipment. Rinse with THF. Stir until solid dissolves completely by visual check and store for later use.
   1) Charging Qty of CSA=(Mixture in step 1 charging Qty * Compound(S)-J wt % * 232.3/219.24*0.5)+ (Mixture in step 1 charging Qty * DBU residue * 232.3/152.24)
   2) Charging Qty of THF=CSA charging Qty * 6.4
   3) Rinse Charging Qty of THF=CSA charging Qty * 0.7
6. At 15-25° C., add 1% seed, stir for 1-2 h and continue add the CSA solution prepared in step 5 to reactor 2. After 6 h, take samples every 1-4 hours until the chiral purity of the supernatant≤11.0%. Sample the solid for chiral purity analysis (FIO). If the result of two consecutive samples >11.0% and the difference is ≤1.0%, continue to add 0.leq (1S)-(+)-Camphor-10-sulfonic acid solution until the chiral purity≤11.0%.
7. Filter the mixture, rinse filter cake with THF (2.65V) and acetonitrile (2.65V), sweep with nitrogen for NLT 2 h in Nutsche filter/Agitating filter dryer, then dry the solid at T_{jacket} <40° C. 12 h later, sample the solid for GC analysis every reference 4˜8h until Total residual (THF residue+acetonitrile residue)≤5 wt %. During drying, every 4-8 h turn over the solid in Nutsche filter/stir the solid for reference 2-5 mins in agitating filter dryer. After drying, cool to T≤30° C. Proceed to step 8

Operation 8-Operation 11, the charging volume is based on the operation of the filter cake: B (operation 7 cake weight).
8. Add acetonitrile (4.5V), purified water (0.3V), adjust to15-25° C., and stir for NLT 5 mins.
9. Add filter cake in step 7, rinse with acetonitrile (0.5V).
10. Heat to 50-60° C., maintain and stir for at least 2 h at 50-60° C. and sample for the purity and chiral purity. If unqualified, add purified water until qualified (0.1V each time, upper limit: 0.3V), cool to 15-25° C., stir NLT 2 h.
11. Filter the mixture, rinse filter cake with (3V) acetonitrile.
12. Sweep with nitrogen for NLT 2 h in Nutsche filter/Agitating filter dryer, then dry the solid at T_{jacket}≤40° C. 12 h later, sample the solid for GC analysis every reference 4-8h until Total residual solvent (THF residue+acetonitrile residue) <1.8 wt % and KF≤1.0 wt %. During drying, every 4-8 h turn over the solid in Nutsche filter or stir the solid for reference 2-5 mins in agitating filter dryer. After drying, cool to T≤30° C.

Chiral resolution for Compound(S)-J
1. The process for the chiral resolution for Compound(S)-J is the same as Compound (R)-J except the CSA configuration ((−)-CSA used for the resolution of Compound J, 50 g scale reaction was conducted to confirm the process. The result is normal.
   1) After 1^{st} chiral resolution the product was obtained with 99.85% purity, 97.51% chiral purity and 45% corrected yield.
   2) After purification with MeCN/H₂O (5V/0.3V), 42.17g product was obtained with 99.96% purity, 98.23% chiral purity and 42% corrected yield.
   3) After 2^{nd} chiral resolution, 21g product was obtained with 99.84% purity, 98.16% chiral purity and 21% yield.

### IPC Methods Developed

UPLC method: Compound (R)-J-17.M (For IPC purity and assay of Compound(S)-J)

| | |
|---|---|
| Column | Infinitylab Poroshell 120 EC-C18 |
| | 2.1*50 mm*1.9 um P/N: 699675-902 |
| Mobile phase A | 10 mM NH₄OAC in 95water:5ACN |
| Mobile phase B | 10 mM NH₄OAC in 5water:95ACN |
| Gradient program | T (min): 0.0 4.0 4.5 4.51 6.0 |
| | B (%): 0 100 100 0 0 |
| Column temperature | 40° C. |
| Flow rate | 0.65 mL/min |
| Injection volume | 0.5 μL |
| Detector | UV at 225 nm |
| Needle wash solvent | 65ACN:35WATER |
| Run Time | 6.0 min |

HPLC method: Compound (R)-J-16.M (For IPC chiral purity of Compound(S)-J)

| | |
|---|---|
| Column | CHIRALCEL AD-H 5 um 4.6 × 250 mm (P/N: 19325) |
| Column | 40° C. |
| Temperature | |
| Flow Rate | 1.0 mL/min |
| Injection Volume | 5 μL |
| Mobile Phase | 0.1% DEA in MeOH |
| Run time | 15 min |
| Needle Wash | EtOH |
| Solvent | |
| Detection | UV at 225 nm |

8) Overall Conclusions for Step 3
   1. Qualified products (Compound (R)-J or Compound(S)-J) can be obtained with yields of 67˜72% with three times of chiral resolution.

### Example 4: Synthesis of tert-butyl (R)-2-(4-(2-(4-amino-2-ethylphenoxy)ethyl)-2-methylpiperazin-1-yl)acetate (2S,3S)-2,3-bis (benzoyloxy) succinate (Compound viii)

The title compound was prepared according to reaction scheme 5.

### A. Step 1: Preparation of Compound ii

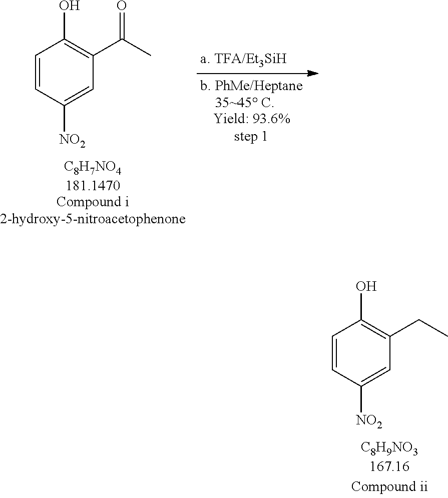

The reaction was conducted in accordance with the steps described below:
1. TFA (trifluoroacetic acid) (462.2 kg) was charged into the glass-lined reactor 1.
2. 2-Hydroxy-5-nitroacetophenone (113.0 kg) was added into the glass-lined reactor 1 through solid addition equipment, stirred for at least 30 min until all the solids were dissolved.
3. The mixture temperature was adjusted to 35˜ 45° C. (target temperature was 40° C.).
4. Triethylsilane (217.0 kg) was added into reactor 1 at 35˜45° C., the adding time was controlled for at least 10 h, at the reference rate of 18.1 kg/h˜21.8 kg/h, the stirring speed was 80˜95 rpm.
5. The mixture was allowed to react at 35˜ 45° C. (target temperature was 40° C.). The mixture was sampled (˜5 ml mixture was submitted for analysis testing) every 4˜12 h for HPLC analysis until area % of 2-hydroxy-5-nitroacetophenone≤1.0%. The total reaction time was 15 hours and 17 minutes.
6. The mixture was adjusted to 20˜ 30° C., target temperature being 25° C.
7. Acetone (144.8 kg) was added into reactor 1 at 20˜ 30° C., controlling the adding time for at least 2 h, at the reference rate of 36 Kg/h˜73 Kg/h. After addition, the reaction mixture was maintained and stirred for not less than 30 min, sampling and testing until the triethyl silane residue was less than 0.1%.
8. Anhydrous ethanol (201.2 kg) was added into reactor 1 at 20˜ 30° C., controlling the adding time for at least 10 h, at the reference rate of 16 kg/h˜20 kg/h. After addition, the mixture was maintained and stirred for not less than 6 h.
9. The mixture was settled. The lower product phase was transferred into 3000L glass-lined reactor 2 . . .
10. The mixture was concentrated at T≤40° C. (T_{jacket}≤50° C.) (T hot water≤50° C.) under reduced pressure (reference P≤-0.08Mpa) until (226L˜452L) 2˜4V (target 3V) was left. Toluene (291.8 kg+295.0 kg) was added into the glass-lined reactor 2 at T<40° C. The mixture was sampled for anhydrous ethanol residual and ethyl trifluoroacetate residual analysis until anhydrous ethanol residual≤0.1%, and ethyl trifluoroacetate residual≤0.1%.
11. The mixture was heated to 45˜55° C. (target 50° C.).
12. n-Heptane (771.8 kg) was added into reactor 2 at 45˜55° C. (target 50° C.), controlling the adding time for at least 6 h. After addition, the reaction mixture was maintained at 45˜55° C. (target 50° C.), and stirred for at least 1 h. The reference agitation speed was not less than 80 rpm.
13. The mixture was cooled to 15˜25° C. (target 20° C.), controlling the cooling time for not less than 2 h.
14. The mixture was stirred at 15˜25° C. (target 20° C.) for crystallization.
15. The mixture was filtered. . . . N-Heptane (309.3 kg+309.1 kg) was added into reactor 1, and then the reactor wall was rinsed, stirred for 0.5˜1 h and then the filter cake was rinsed. The filter cake was sampled for purity and hexaethyldisiloxane residue analysis by HPLC until hexaethyldisiloxane residue≤0.1%.
16. The filter cake was swept with nitrogen for not less than 2 h until dry. The mixture was cooled to 20˜30° C.
17. The product had the following characteristics:
Weight: 97.6 kg
Purity: 99%
Potency (wt %): 100%
Yield: 93.6%
Appearance: Off-white solid

### B. Step 2 and 3: Preparation of Compound iv

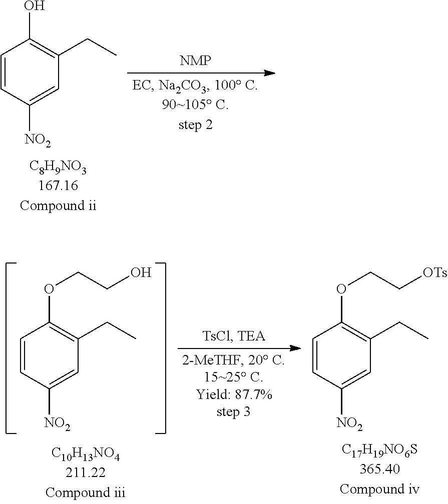

The reaction was conducted in accordance with the steps described below:
1. NMP (499.2 kg) was added into a reactor 1. The stirrer was started.
2. Compound ii (97.4 kg) was added into the glass-lined reactor 1 through solid addition funnel. The mixture was stirred for NLT 20 min. Sodium carbonate (67.6 kg) and ethylene carbonate (102.2 kg) were added into the mixture, at a reference agitation rate of NLT 80 rpm. Then the pipeline was rinsed with NMP (100.0 kg)
3. The mixture was heated to 90˜105° C. (target: 95˜100° C.).
4. The mixture was allowed to react at 90˜105° C. (target: 95˜ 100° C.), at a reference rate of NLT 80 rpm. After 18 h, the mixture was sampled every 2˜15 h for HPLC analysis until area % of Compound ii was ≤2.0%.
5. The mixture was cooled to 20˜30° C. (target: 25° C.).
6. Purified water (1940.0 kg) was added into the glass-lined reactor1 at 20˜30° C. After a clear mixture was obtained, the mixture was transferred into 8000L glass-lined reactor 2. Reactor 1 was rinsed with 2-Me-THF (828.6 kg), then the rinsing liquor was transferred into reactor 2.
7. The mixture was stirred for NLT 20 min at 20˜30° C. and then settled.
8. The aqueous phase was extracted with 2-Me-THF (414.2 kg) at 20˜30° C. The mixture was stirred for NLT 20 min at 20˜30° C. and then settled.
9. The aqueous phase was extracted with 2-Me-THF (414.6 kg) at 20˜30° C. The mixture stirred for NLT 20 min at 20˜30° C. and then settled. The organic phase of steps 7, 8, and 9 was combined and sampled for dimer impurity to confirm it was ≤0.07%.

If dimer impurity was unqualified, step 10 was conducted.
10. The organic phase was washed with methanol (236.0 kg) and sodium hydroxide solution (348.0 kg, prepared with purified water (290.0 kg) and sodium hydroxide (58.0 kg)) at 20˜30° C., stirred for NLT 15 min. Organic phase was tested for dimer impurity)≤0.07%. After qualified, the layers were settled and separated.
11. The mixture was adjusted to 15˜25° C. (target: 20° C.).
12. Sodium chloride solution (455.0 kg, prepared with purified water (367.0 kg) and sodium chloride (89.2 kg)) was added into glass-lined reactor 2 at 15˜25° C. Hydrochloric acid solution (15.0 kg) was added into the mixture to adjust pH to 6 ˜7.
13. The mixture was stirred for NLT 15 min at 15˜25° C. and then settled.
14. The organic phase was washed with sodium chloride solution (558.0 kg, prepared with purified water (1338.0 kg) and sodium chloride (334.8 kg)) at 15˜25° C., stirred for NLT 15 min and settled.
15. The organic phase was washed with sodium chloride solution (558.0 kg) which was prepared in step 14, at 15˜25° C., stirred for NLT 15 min and settled. The organic phase was sampled for NMP residual, ethylene carbonate residual and ethylene glycol residual analysis until NMP residual≤2%, ethylene glycol residual≤0.05%. ethylene carbonate residual≤0.2%.
16. The organic phase was concentrated at T≤45° C. (T_{jacket}≤60° C.) under reduced pressure (reference P≤-0.08 MPa) until (˜487L) ˜5V left. 2-Me-THF (417.6 kg) was added into the mixture at T≤45° C. The mixture was sampled for KF, methanol residual analysis until KF≤0.2%, methanol residual≤0.2%.

If KF and methanol residual was unqualified, proceed to step 17.
17. The organic phase was concentrated at T≤45° C. (T_{jacket}≤60° C.) under reduced pressure (reference P≤-0.08 MPa) until (˜487L) ˜5V left. 2-Me-THF (417.0 kg) was added into the mixture at T≤45° C. The mixture was sampled for KF, methanol residual analysis until KF≤0.2%, methanol residual≤0.2%

Note: KF=0.5% and methanol residual=0.0% (0.04%) was unqualified, step 17 was repeated until qualified.
18. The mixture was adjusted to 15˜25° C. Triethylamine (117.4 kg) was added into the mixture. DMAP (7.2 kg) was added into the mixture though Halar-lined Solid addition funnel at 15˜25° C.
19. 4-Methylbenzene sulphonyl chloride (176.8 kg) was added into the mixture (reference 28˜34 Kg in each portion) though Halar-lined solid addition funnel at 15˜25° C. The reference adding rate was NMT 8.5 kg/15 min, and the actual adding rate was mainly determined by temperature control.
20. The mixture was allowed to react at 15˜25° C. After 10 h, the mixture was sampled every 2˜15h for HPLC analysis, until area % of Compound iii was ≤1.0%.
21. The mixture was adjusted to 20˜30° C. Purified water (486.0 kg) and 2-Me-THF (417.6 kg) was added into the mixture at 20˜30° C., stirred for NLT 15 min and settled until the mixture layered before separation.
22. The organic phase was washed with sodium chloride solution, prepared in step 14, (557.8 kg) at 20˜30° C., stirred for NLT 20 min and settled until the mixture layered before separation.
23. The mixture was concentrated at T≤25° C. (T_{jacket}≤35° C.) under reduced pressure (P≤-0.08 MPa) until (485L) ˜5 V left.
24. 2-Me-THF (418.6 kg) was added into the mixture at T≤25° C. The mixture was concentrated at T≤25° C. (T_{jacket}≤35° C.) under reduced pressure (P≤-0.08 MPa) until (485L) ˜5 V left. The mixture was sampled for KF analysis until KF≤0.1%.
25. The mixture was heated to 50˜60° C. (target: 55° C.).
26. The mixture was cooled to 15 ˜ 25° C. (target: 20° C.), and stirred for NLT 1h until solid precipitated.
27. n-Heptane (861.4 kg) was added into the mixture at 15 ˜ 25° C. The adding time was at least 8 h.
28. The mixture was stirred at 15˜25° C. (target: 20° C.) for crystallization.
29. The mixture was filtered with an agitated filter dryer. 2-Me-THF (119.2 kg) and n-heptane (172.8 kg) were added into a reactor, stirred for 0.5˜1 h and discharged. The reactor (where the crystallization took place) was rinsed with it, and then the filter cake was rinsed. The filter cake was sampled for purity.

30. The filter cake was swept with nitrogen for NLT 2 h, then the solid was dried at reference T_{jacket}≤50° C., 8h later, the solid was sampled for analysis every 4˜12 h until 2-MeTHF residual≤1.0% and n-heptane residual≤1.0%. After drying, cooled to ≤30° C.
31. The product had the following characteristics:
Weight: 188.6 kg, 186.7 kg corrected
Purity: 100%
Potency (wt %): 99 wt %
Yield: 87.9%
Appearance: Yellow solid

### C. Step 3a: Preparation of Compound v

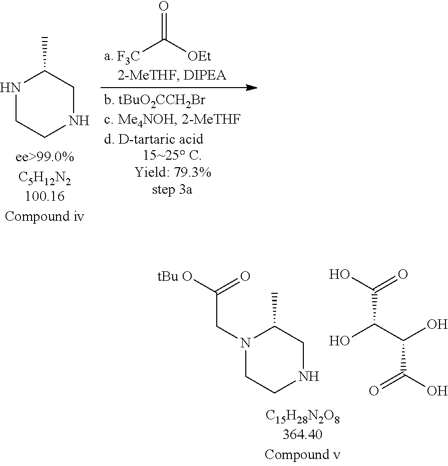

The reaction was conducted in accordance with the steps described below:
1. 2-Me-THF (638.6 kg) was added into 3000L glass-lined reactor 2. The mixture was stirred for NLT 10 min. The mixture was sampled for KF analysis until KF≤0.1%.
2. The mixture was adjusted to 15˜25° C. (R)-(−)-2-Methylpiperazine (72.4 kg) was added into the 3000 L glass-lined reactor 2 though solid addition funnel at 15˜25° C. Nitrogen was bubbled into the mixture from the bottom valve. After the mixture was dissolved, the reactor wall was rinsed with 2-Me-THF (40.0 kg).
3. DIPEA (232.6 kg) was added into the mixture at 15˜25° C.
4. Ethyl trifluoroacetate (112.2 kg) was added into the 3000L reactor 2 at 15˜25° C., at a reference rate of 9.4 ˜11.2 Kg/h. The drop-adding time was controlled for at least 10 h.
5. The mixture was allowed to react at 15˜25° C. After 12 h, the mixture was sampled every 6˜12 h for GC analysis, until area % of (R)-(−)-2-Methylpiperazine was ≤1.5 area %.
6. The mixture was transferred into 5000L glass-lined reactor 3 at 15˜25° C.
7. Tert-butyl bromoacetate (150.2 kg) was added into the 5000L glass-lined reactor 3 at 15˜25° C.
8. The mixture was heated to 55˜65° C.
9. The mixture was allowed to react at 55˜65° C. After 10 h, the mixture was sampled every 4˜10 h for GC analysis, until area % of Product A was ≤1.0 area %.
10. Triethylamine (145.40 kg) was added into the 5000L glass-lined reactor 3 at 55˜65° C.
11. The mixture was allowed to react at 55˜65° C. After 2 h, the mixture was sampled every 2˜ 8 h for UPLC analysis, until area % of tert-Butyl bromoacetate was ≤0.5 area %.
12. The mixture was cooled to 0˜10° C.
13. Citric acid monohydrate solution (975.0 kg) which was prepared with purified water (790.0 kg) and citric acid monohydrate (200.0 kg) were added into the mixture to adjust pH to 6˜7 at 0˜10° C. The mixture was stirred for 15˜30 min at 0˜10° C., and settled for NLT 20 min before separation.
14. The upper organic phase in 5000L reactor 3 was adjusted to 15˜25° C.
15. The organic phase was washed with 10% potassium dihydrogen phosphate solution which was prepared with purified water (720.0 kg) and potassium dihydrogen phosphate (80.0 kg) at 15˜25° C., the mixture was stirred for 15˜30 min and settled for NLT 20 min before separation. The upper organic phase was left in reactor 3 for temporary storage and waited for washing. The organic phase was sampled for DIPEA residue and triethylamine residue analysis until DIPEA residue≤0.2%, triethylamine residue≤0.2%.
16. The organic phase was washed with 13% sodium chloride solution which was prepared with purified water (720.0 kg) and sodium chloride (119.0 kg) at 15˜25° C. The mixture was stirred for 15˜30 min and settled for NLT 20 min before separation.
17. 25% Me₄NOH in methanol (261.8 kg) for over NLT 10 h was added into the 5000L glass-lined reactor 3 though a pump at 15˜25° C. The reference adding rate: 21.8˜26.2 Kg/h.
18. The mixture was allowed to react at 15˜25° C. After 2 h, the mixture was sampled every 1˜5 h for GC analysis, until area % of Product B was ≤1.0 area %.
19. 25% Me₄NOH in methanol (9.6 kg) was added into the 5000L glass-lined reactor 3 at 15˜25° C.
20. The mixture as allowed to react at 15˜25° C. After 1 h, the mixture was sampled every 1˜2 h for GC analysis, until area % of Product B was ≤1.0 area %.
21. The mixture was transferred into 3000L glass-lined reactor 4.
22. The mixture was concentrated at T≤50° C. (T_{jacket}≤55° C.) under reduced pressure (P≤-0.08 MPa) until ˜5V (˜360L) left, 2-Me-THF (932.4 kg) was added into the mixture at T≤50° C.
23. The mixture was concentrated at T≤50° C. (T_{jacket}≤55° C.) under reduced pressure (P≤-0.08 MPa) until ˜5V (˜360L) left., 2-Me-THF (942.4 kg) was added into the mixture at T≤50° C.
24. The mixture was concentrated at T≤50° C. (T_{jacket}≤55° C.) under reduced pressure (P≤-0.08 MPa) until ˜5V (˜360 L) left. The mixture was sampled for KF and methanol residual analysis until KF<0.6%, methanol residual≤2%.
25. 2-Me-THF (317.2 kg) was added into the mixture at T≤50° C.
26. The mixture was cooled to 15˜25° C.
27. The mixture was filtered . . . 2-Me-THF (312.0 kg) was added into glass-lined reactor 4, the mixture was stirred for 5˜10 min, then rinsed filter cake. The filter cake was sampled for Compound v wt % analysis until wt %≤1.5%.
28. The filtrate and rinse liquid in step 27 were transferred into 3000L glass-lined reactor 5. The mixture was concentrated at T≤50° C. (T_{jacket}≤55° C.) under reduced pressure (P≤-0.08 MPa) until ˜5V (˜360L) left.
29. The mixture was adjusted to 15˜25° C.
30. The solution was added into the mixture in reactor 5 at 15˜25° C. over at least 8 h, the reference agitation speed was NLT 80 rpm. The reference charging speed NMT 157.5 kg/h. The solution was prepared with acetone (855.4 kg) and purified water (72.0 kg) and D-(−)-tartaric acid (108.0 kg).
31. The mixture was stirred at 15˜25° C. for crystallization . . .
32. The mixture in the reactor was filtered. Reactor 5 was rinsed with the solution which was prepared with 2-Me-THF (156.2 kg) and acetone (429.4 kg) and purified water (36.0 kg). The mixture was stirred for 5˜10 min, and then rinsed the filter cake. Reactor 5 was rinsed with acetone (571.2 kg). The mixture was stirred for 5˜10 min, and then rinsed the filter cake.
33. Swept with nitrogen for NLT 2 h, then the solid was dried at T_{jacket}=40˜50° C., reference P≤-0.08 MPa, 8h later, the solid was sampled for analysis every 4˜12 h until acetone residue≤0.5%, 2-Me-THF residue≤0.5%, KF: 5.7%. After drying, the mixture was cooled to 20˜30° C.
34. Product had the following characteristics:
Weight: 223.0 kg, 208.1 kg, corrected
Purity: 100%
Yield: 79.3%
Appearance: Off white solid
   D. Steps 4 and 5: Preparation of Compound viii

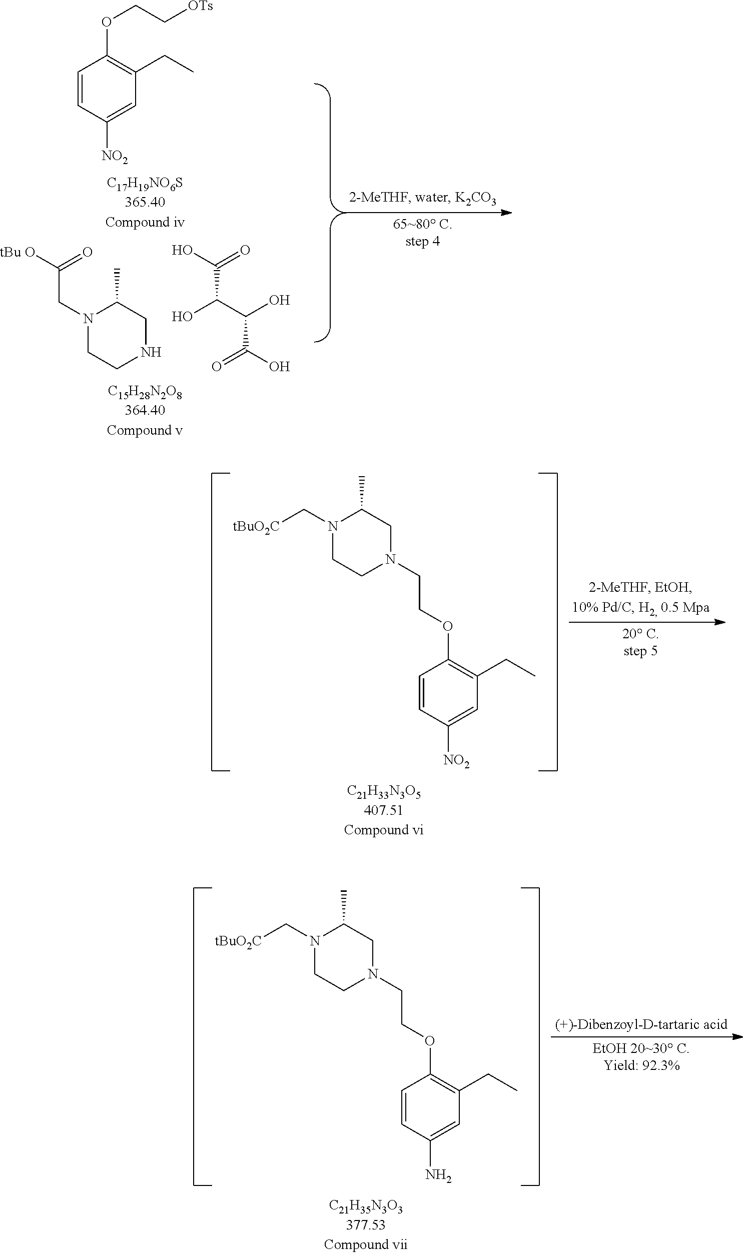

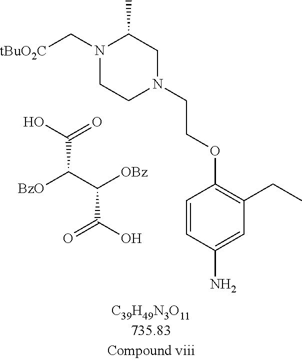

The reaction was conducted in accordance with the steps described below:
1. Purified water (1642.0 kg) and sodium carbonate (285.4 kg) were charged into a 8000L glass-lined reactor 1. And the mixture was stirred until solid dissolved completely by visual check and stored for later use.
2. 2-Me-THF (1400.8 kg) was added into the 8000L glass-lined reactor 1. Then the mixture was adjusted to 20˜30° C.
3. Compound iv (165.8 kg, 164.1 kg) and and Compound v (215.8 kg, 201.3 kg corrected) were added into the 8000L glass-lined reactor 1 through solid addition box at 20˜30° C.
4. The mixture was heated to 65˜80° C.
5. The mixture was allowed to react at 65˜80° C. After 16 h, the mixture was sampled every 2˜10h for UPLC analysis until area % of Compound iv was ≤1.0%. The samplling was done by taking ˜5 ml mixture and submitting for analysis testing.
6. The mixture was cooled to 20˜30° C.
7. The mixture was settled for NLT 0.5 h before separation at 20˜30° C.
8. The organic phase was washed with purified water (1644.0 kg) at 20˜30° C., the mixture was stirred for NLT 20 min and settled for NLT 20 min before separation.
9. The organic phase was washed with sodium chloride solution which was prepared with sodium chloride (86.0 kg) and purified water (1616.0 kg) at 20˜30° C., the mixture was stirred for NLT 20 min and settled for NLT 20 min before separation.
10. The organic phase was washed with purified water (1640.0 kg) at 20˜30° C., the mixture was stirred for NLT 20 min and settled for NLT 20 min before separation. The organic phase was sampled for Compound v free base (Product C) residue analysis until Compound v free base (Product C) residue≤0.10%.

Note: If Compound v free base (Product C) residue was unqualified, proceeded to Step 11.
11. The organic phase was washed with purified water (1641.0 kg) at 20˜30° C., the mixture was stirred for NLT 20 min and settled for NLT 20 min before separation. The organic phase was sampled for Compound v free base (Product C) residue analysis until Compound v free base (Product C) residue≤0.10%.
12. The organic phase was diluted with 2-Me-THF (566.0 kg) to 9.5 V. Then the mixture was discharged into drums, and the 8000L reactor 1 was rinsed with 2-Me-THF (70.6 kg). The rinsing liquor was transferred into drums.
13. The organic phase was transferred into 3000L autoclave reactor 3 through liquid material filter. Then the stirrer was started.
14. The 3000L autoclave reactor 3 was purged with nitrogen via subsurface pipe to 0.2˜0.3 MPa at 10˜30° C., then vented to 0.02˜0.05 MPa, repeating the purge and vent operation for 5˜6 times until oxygen content≤0.1% after exchanging. Filled with nitrogen to normal pressure the last time.
15. 10% Pd/C (8.20 kg) was added into the 3000L reactor 3 through stainless steel solid addition funnel at 20˜30° C., reactor wall was rinsed with anhydrous ethanol (258.8 kg)
16. The mixture was purged with nitrogen via subsurface pipe to 0.2˜0.3 MPa at 20˜30° C., then vented to 0.02˜0.05 MPa, repeated the purge and vent operation for 5˜6 times until oxygen content≤0.1% after exchanging.
17. The mixture was purged with hydrogen via subsurface pipe to 0.2˜0.3 MPa, then vented to 0.02˜0.05 MPa at 20˜30° C., repeated the purge and vent operation for 5˜6times. Then the mixture pressure was increased to 0.4˜0.6 MPa.
18. The mixture was allowed to react at 20˜30° C., P=0.4˜0.6 MPa (target: 25° C., 0.5 MPa). After 3 h, the mixture was sampled every 2˜8h for UPLC analysis until reaction reached completion.
19. The mixture was purged with nitrogen via subsurface pipe to 0.2˜0.3 MPa at 20˜30° C., then vented to 0.02˜0.05 MPa, repeating the purge and vent operation for 8 times. Hydrogen content was detected≤0.1% after exchanging. If unqualified, continued to exchange until≤0.1%, then filled with nitrogen to normal pressure.
20. The mixture was filtered with the 1000L stainless steel Nutsche filter. The filter cake was rinsed with anhydrous ethanol (64.6 kg+64.8 kg+65.0 kg+65.2 kg) until Compound viii wt % in rinsing liquor≤0.1 wt %. The filtrate was sampled for Pd residue analysis Pd residual≤5 ppm.
21. The filtrate was transferred into 5000L reactor 4 through stainless steel liquid material filter. The mixture was concentrated at T_{jacket}≤35° C. under reduced pressure (P≤-0.08 MPa) until ˜5V (820L˜830 L) left.
22. Ethanol (648.9 kg) was added into the 5000L glass-lined reactor 4 through a liquid material filter, until the total volume to 10V.
23. The mixture was adjusted to 15˜25° C.
24. (+)-Dibenzoyl-D-tartaric acid solution prepared with anhydrous ethanol (1297.1 kg) and (+)-dibenzoyl-D-tartaric acid (169.0 kg) was added into the mixture at a reference rate of 74˜112 Kg/h at 15˜25° C.
25. The mixture was stirred at 15˜25° C. for crystallization. After 3 h, the mixture was sampled every 1˜3h for mother liquor wt % analysis until it was ≤0.3 wt %.
26. The mixture was filtered. The filter cake was rinsed with anhydrous ethanol (649.2 kg). The filter cake was rinsed with MTBE (608.7 kg).
27. Solid was transferred into oven and swept with nitrogen for NLT 2 h, then the solid was dried at T_{jacket}≤30° C., until ethanol residual≤14%. Then the oven was heated to T_{jacket} =40˜45° C., then the mixture was dried at T_{jacket}≤45° C. until ethanol residual≤0.5%, MTBE residual≤0.5%, 2-Me-THF residual≤0.5%. During drying, the solid was turned over every 6˜12 h. After drying, the mixture was cooled to ≤30° C.
28. The solid was sieved until the appearance of the product was uniform and without blocking.
30. Product had the following characteristics:
Weight: 305.0 kg
Purity: 99.59%
Assay: 100.7%
Appearance: Off white solid
Yield: 92.3.

### Example 5: Synthesis of Amorphous 2-((R)-4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl)-N-(3-((2,6-dioxopiperidin-3-yl)amino)phenyl) acetamide (Compound 1)

The title compound was prepared according to reaction scheme 6.

### Step 1: Preparation of Compound U

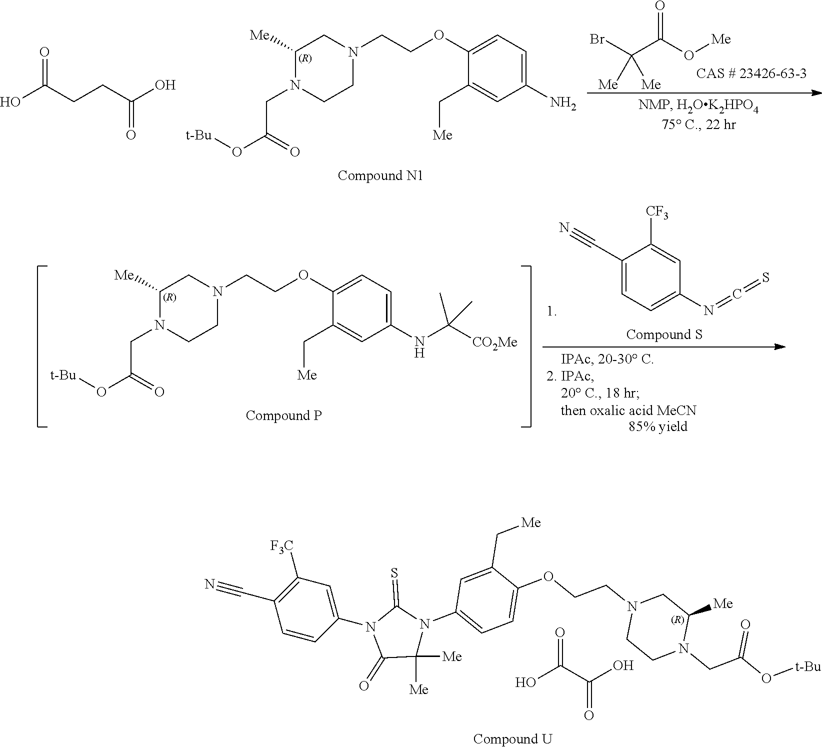

Compound N1 (LR, 1 kg) was charged to a reactor with NMP (5.25L/kg, 5.408 kg). A solution of potassium tribasic (2.0 eq, 0.856 kg) in water (1.75L/kg, 1.75 L) and methyl-2-bromo-2-methylpropionate (4.5 eq, 1.64 kg) were charged at once. The reactor was sparged with N2 for 30 min and then heated to 70° C. and aged for 18 h. Once the reaction was complete, the batch was cooled to 35° C. and diluted with isopropyl acetate (7.5 L/kg, 6.525 kg). A solution of potassium carbonate (1.5 eq, 0.418 kg) in water (10.5 L/kg, 10.5 kg) was added to the reaction and the mixture was stirred for 1 h and the layers were separated. The temperature of the batch was cooled to 25° C. and the organic layer was washed with water (7.5 L/kg, 7.5 kg) and the layers separated. Isopropyl acetate was added (1.5 L/kg, 1.305 kg) to the organic layer and the batch was distilled under vacuum to a total volume of 7.5 L/kg, targeting a final KF of ˜ 0.5-1.5% water. Compound S (1.6 eq, 0.736 kg) was charged followed by isopropyl acetate (0.75 L/kg, 0.6525 kg) and the resulting solution was allowed to stir for 4 h. Once the reaction was complete, acetonitrile (0.82 L/kg, 0.644 kg) was added to the reactor. Next an oxalic acid (1.2 eq, 0.218 kg) solution in isopropyl acetate (5.4 L/kg, 4.698 kg) and acetonitrile (0.6 L/kg, 0.471 kg) was added dropwise to the reactor over 10 h. The resulting slurry was aged for 1 h and then filtered. The filter cake was washed twice with a 9:1 IPrOAc/MeCN (7.5L/kg) solution. The resulting solids were dried under vacuum to afford Compound U in ˜85-90% isolated yield.

¹H NMR (500 MHZ, DMSO-d₆) δ ppm 1.01 (d, J=1.00 Hz, 3H) 1.16 (t, J=7.48 Hz, 3H) 1.42 (s, 9H) 1.49 (s, 6H) 2.40-2.47 (m, 1H) 2.63 (q, J=1.00 Hz, 2H) 2.69-2.79 (m, 1H) 2.82-2.97 (m, 3H) 3.11-3.26 (m, 4H) 3.34 (s, 2H) 4.29 (br t, J=5.11 Hz, 2H) 7.07-7.22 (m, 3H) 8.00 (dd, J=8.24, 1.68 Hz, 1H) 8.29 (d, J=1.00 Hz, 1H) 8.39 (d, J=1.00 Hz, 1H)

¹³C NMR (101 MHZ, DMSO-d₆) δ 180.5, 175.5, 169.9, 163.6, 156.5, 138.7, 136.6, 134.5, 133.2, 131.7, 130.3, 128.7, 128.3, 124.1, 121.4, 115.5, 112.4, 109.0, 81.0, 66.6, 64.8, 59.2, 56.0, 54.9, 52.9, 52.5, 28.3, 23.4, 23.1, 16.7, 14.4

MS Analysis: Calculated: [M+1]=674.3; Found: [M+1]=674.4

### Step 2: Compound W

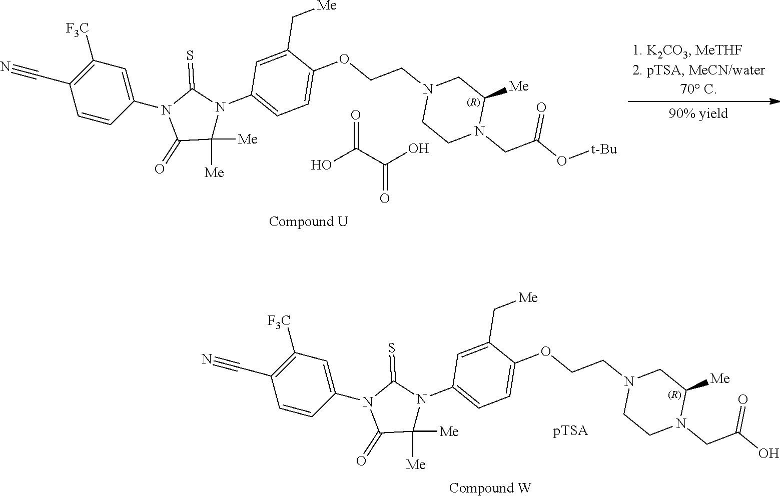

To a reactor was charged Compound U (LR, 1 kg) followed by MeTHF (5L/kg, 4.27 kg). Next, a solution of potassium carbonate (2.5 eq, 0.4524 kg) in water (2.5L/kg, 2.kg) was added. The mixture was allowed to stir for 30 min and the layers separated. The resulting organic layer was washed with water (2.5L/kg, 2.5 kg) and the layers separated. The organic stream was concentrated to a volume of 3 L and solvent swapped to acetonitrile under vacuum distillation. Once the solvent swap was complete p-toluenesulfonic acid monohydrate (4 eq, 0.996 kg) was added followed by acetonitrile (1 L/kg, 0.786 kg) and water (3 L/kg, 3 kg). The resulting solution was heated to 65 C and allowed to react for at least 10 h. Once the reaction was deemed complete, the batch was cooled to 60 C and the pH was adjusted to ˜pH=2.4 by charging NMI dropwise (˜3 eq, 0.322 kg, 0.313L). The batch was crystallized by adding water (6L/kg, 6 kg) and Compound W seeds. Additional water (6 L/kg, 6 kg) was charged over 5 h for further desaturation and then the batch was cooled to 20 C over 3 h. The resulting white slurry was filtered, and the cake washed with 85:15 water/acetonitrile (5 L/kg, 4.84 kg) followed by water (5 L/kg, 5 kg). The resulting wet cake was dried under vacuum to afford Compound W as a white solid in ˜95% yield.

¹H NMR (400 MHZ, DMSO-d₆) δ 8.40 (d, J-8.3 Hz, 1H), 8.30 (d, J=1.6 Hz, 1H), 8.08 (dd, J=8.3, 1.6 Hz, 1H), 7.49 (d, J=8.0 Hz, 2H), 7.22-7.10 (m, 5H), 4.38 (br s, 2H), 3.65 (br s, 1H), 3.50 (br s, 4H), 3.20 (br s, 2H), 3.09 (br s, 3H), 2.82 (br s, 1H), 2.73-2.53 (m, 2H), 2.29 (s, 3H), 1.50 (s, 6H), 1.17 (t, J=7.5 Hz, 3H), 1.08 (br s, 3H)

¹³C NMR (101 MHz, DMSO-d₆) δ 180.5, 175.5, 156.2, 146.2, 138.7, 138.1, 136.6, 134.5, 133.2, 131.7, 131.3, 130.3, 128.7, 128.5, 126.0, 124.1, 121.4, 115.5, 112.4, 109.0, 66.6, 55.8, 53.1, 23.4, 22.9, 21.2, 14.4

MS Analysis: Calculated: [M+1]=618.24; Found: [M+1]=618.31

### Step 3: Compound 1 butyl acetate solvate

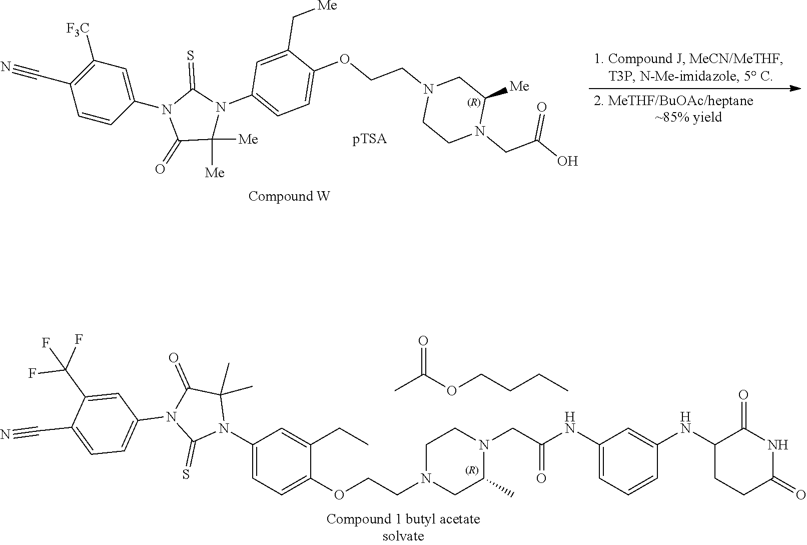

To a reactor was charged acetonitrile (2.0 L/kg, 30.1 equiv.) and 2-MeTHF (1.5 L/kg, 11.8 equiv.) followed by 1-methylmidazole (0.52 kg/kg, 5.0equiv), Compound W (1.00 kg/kg, 1.00 equiv.) and Compound J (0.575 kg/kg, 1.16 equiv.). The solid charges were rinsed with acetonitrile (1.0 L/kg, 15.1 equiv.) and the temperature of the reaction was adjusted 15˜25° C. The mixture was allowed to stir until a homogenous solution formed. Next, the batch was cooled to 0˜10° C. (target 5° C.) at which point a 50 wt % T3P in 2-MeTHF solution (2.01 kg/kg, 2.50 equiv.) was added slowly, maintaining a temperature of 0˜10° C. The reactor was then rinsed with 2-MeTHF (0.5 L/kg, 3.9 equiv.) and the reaction was allowed to age for at least 2 h at 0˜10° C. until deemed complete. Aq. Na₂HPO₄ (6L/kg, 2.leq, 6.0 wt %) was slowly added into the reaction mixture, followed by 2-MeTHF (2.0 L/kg) and BuOAc (2.0 L/kg). The mixture was heated to 40˜50° C. with vigorous mixing. The layers were settled and then split. The organic phase was washed a second time with aq. Na₂HPO₄ (6L/kg, 2.leq, 6.0 wt %) followed once more with 6L/kg aq. Na₂HPO₄ (1.69 eq, 4.8 wt %) & NaH₂PO₄ (0.50 eq, 1.2 wt %). The layers were separated, and the solution was cooled to 15-25° C. The organic phase was distilled at 40° C. to a final volume of 3.7˜4.3 L/kg (target: 4.0L/kg). 2-MeTHF (6 L/kg) was added and the solution filtered though activated carbon filter at 15-30° C. The activated carbon was rinsed with 2 L/kg 2-MeTHF at 15˜30° C. The filtrates were combined and the resulting solution was distilled at 40° C. to a final volume of 3.8˜4.3 L/kg (target: 4.0L/kg). The batch was cooled to 25˜35° C. and 0.6 L/kg BuOAc and 1.3 L/kg 2-MeTHF were added to the concentrated stream. Next, a seed solution of 1% Compound 1 in 0.1 L/kg BuOAc was charged to form a slurry which was allowed to age for 12 h. Heptane (1 L/kg) was added slowly over to 4˜6 h and aged once again aged for 12 h. The mixture was then cooled to −5˜5° C. and aged for another 3 h. The resulting slurry was filtered and the cake washed with 2 L/kg Hep: BuOAc: 2-MeTHF (1:2:4, adjusted to −5˜5° C.). Next, the cake was washed with 2 L/kg BuOAc (adjusted to −5˜5° C.). Finally the cake was washed twice with 3 L/kg Heptane (adjusted to 15˜25° C.). The resulting solids were dried under vacuum to afford Compound 1 butyl acetate solvate as an off-white to green to blue solid in 85% yield.

### Step 4: Compound 1 (free base)

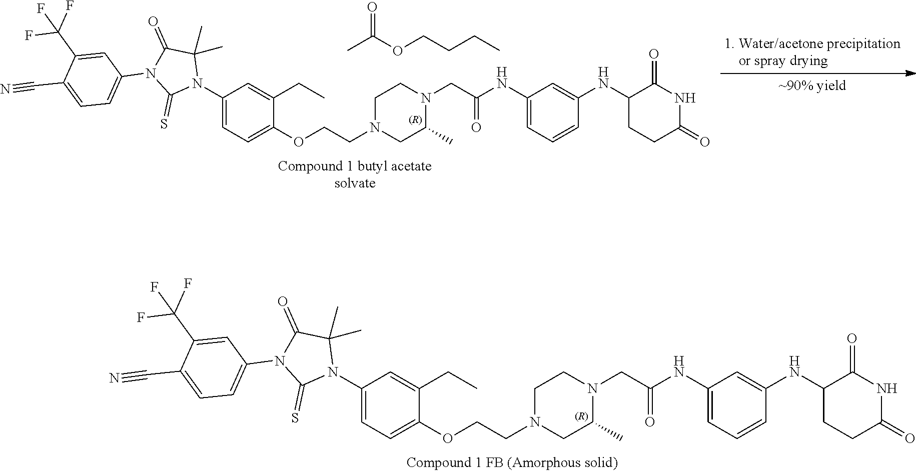

Compound 1 butyl acetate solvate (1 kg, LR) was dissolved in 10 kg/kg acetone at 20C in an agitated temperature-controlled vessel. After full dissolution was achieved, the solution was passed through a polish filter and then charged into the spray dryer at ˜50 kg/hr feed rate. The desired outlet temperature 60-80C (target 70C) was achieved by setting the nitrogen inlet to 85-100C (target 95C). Spray drying was run at 0.20-0.3 MPa (target 0.22 MPa). Material accumulation around the nozzle was checked every 8 h visually to prevent significant material accumulation. Once spray drying was complete, Compound 1 (free base API) was then collected from the cyclone separator and then taken to secondary drier for drying under vacuum at Tj 70C with N2 sweep for at least 15 h.

Yield is dependent on the equipment and batch sizing (mostly physical hold up) and has been around 90% based on recent scale up experience at 30 kg and 50 kg scale.

¹H NMR (400 MHZ, DMSO-d₆) δ ppm 8 10.79 (s, 1H); 9.37 (s, 1H), 8.39 (d, J=8.4 Hz, 1H); 8.30 (s, 1H); 8.08 (dd, J=8.00 Hz, 1.2 Hz, 1H); 7.17-7.11 (m, 2H); 7.03-6.98 (m, 2H); 6.81 (d, J=8.00 Hz, 1H), 6.40 (d, J=8.4 Hz, 1H); 5.81 (d, J-8.00 Hz, 1H), 4.31-4.27 (m, 1H), 4.16 (t, J-5.6 Hz, 1H); 2.95 (d, 1H), 2.81-2.76 (m, 6H), 2.61-2.58 (m, 4H); 2.4-2.3 (m, 1H); 2.11-2.09 (m, 2H), 1.90-1.85 (m, 1H); 1.5 (s, 6H), 1.68 (t, J=7.6 Hz 3 H); 1.0 (d, J=6.0 Hz, 3H)

¹³C NMR (101 MHz, DMSO-d₆) δ 179.9, 175.1, 173.5, 173.0, 168.77, 156.4, 148.4, 138.9, 138.2, 136.1, 134.0, 132.7, 131.0, 129.7, 129.0, 128.2, 128.1, 122.2, 115.0, 111.9, 108.5, 107.9, 104.01, 66.12, 66.1, 60.5, 57.7, 56.4, 54.9, 53.2, 52.4, 30.7, 24.6, 22.9, 22.7, 16.4, 13.9

CHN Analysis: Calculated: C: 60.13%, H: 5.54%, N: 13.68% Found: C: 59.53%; H: 5.54%; N: 13.54%

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed:

1. A process for preparing Compound 1 or a stereoisomer or mixture of stereoisomers thereof 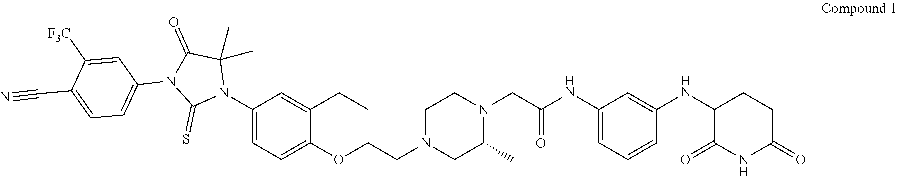 [SMILES: CCc1cc(N2C(=S)N(c3ccc(C#N)c(C(F)(F)F)c3)C(=O)C2(C)C)ccc1OCCN1CCN(CC(=O)Nc2cccc(NC3CCC(=O)NC3=O)c2)[C@H](C)C1]
comprising contacting an acid salt of (R)-2-(4-(2-(4-(3-(4-cyano-3-(trifluoromethyl)phenyl)-5,5-dimethyl-4-oxo-2-thioxoimidazolidin-1-yl)-2-ethylphenoxy)ethyl)-2-methylpiperazin-1-yl) acetic acid with an acid salt of 3-((3-aminophenyl)amino) piperidine-2,6-dione in the presence of a base in a solvent under conditions suitable to provide Compound 1 or a stereoisomer or mixture of stereoisomers thereof,
optionally the base is N-methyl imidazole, triethyl amine or pyridine,
optionally the solvent comprises THF, DMF, EtOAc, MeCN, DMAc, DMSO or a mixture thereof.

2. The process of claim 1, wherein the process comprises contacting Compound W 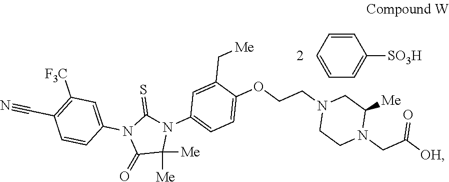
with Compound J 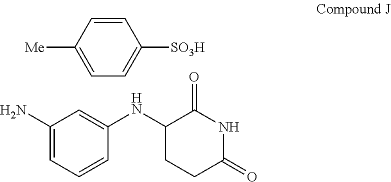
in the presence of a base in a solvent under conditions suitable to provide Compound 1 or a stereoisomer or mixture of stereoisomers thereof,
optionally the solvent comprises THF.

3. The process of claim 1, wherein the process further comprises contacting Compound U 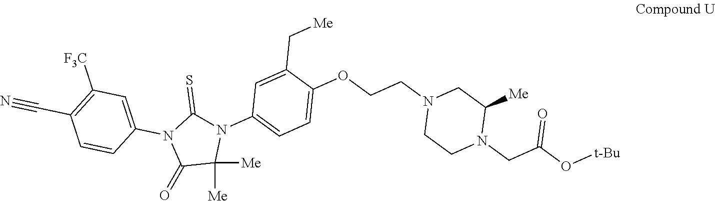 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
with BSA in a solvent under conditions suitable to provide Compound W,
optionally the solvent comprises acetonitrile.

4. The process of claim 3, wherein the process further comprises contacting Compound P 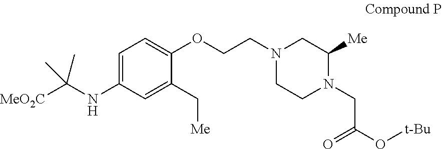 [SMILES: CCc1cc(NC(C)(C)C(=O)OC)ccc1OCCN1CCN(CC(=O)OC(C)(C)C)[C@H](C)C1]
with Compound S 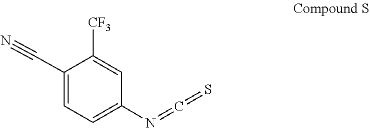 [SMILES: N#Cc1ccc(N=C=S)cc1C(F)(F)F]
in a solvent under conditions suitable to provide Compound U,
optionally the solvent comprises 2-methyltetrahydrofuran, IPrOAc or MeCN.

5. The process of claim 4, wherein the process further comprises contacting Compound N 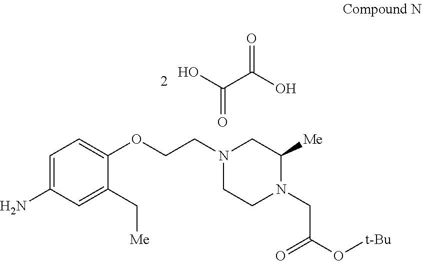
with methyl 2-bromo-2-methylpropionate in a solvent in the presence of a base under conditions suitable to provide Compound P,
optionally the base comprises K₂CO₃, TEA, DIEA, DBU, Na₂CO₃, Cs₂CO₃, Na₃PO₄, K₃PO₄, K₂HPO₄ or NazHPO₄,
optionally the solvent comprises NMP, CH₃CN, MeTHF, DMF, DMSO, DMAc or MeOH.

6. The process of claim 5, wherein the process further comprises contacting Compound L 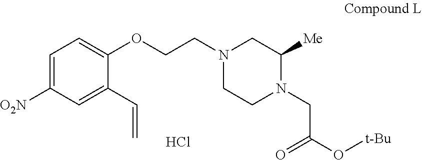 [SMILES: C=Cc1cc([N+](=O)[O-])ccc1OCCN1CCN(CC(=O)OC(C)(C)C)[C@H](C)C1 ; Cl]
with a palladium catalyst in a solvent under conditions suitable to provide Compound N,
optionally the solvent comprises 2-methyltetrahydrofuran.

7. The process of claim 6, wherein the process further comprises contacting Compound K 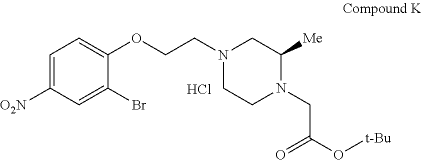 [SMILES: C[C@@H]1CN(CCOc2ccc([N+](=O)[O-])cc2Br)CCN1CC(=O)OC(C)(C)C ; Cl]
with vinyl dioxaborolane in the presence of a palladium catalyst and a base in a solvent under conditions suitable to provide Compound L,
optionally the solvent comprises a mixture of tetrahydrofuran and water.

8. The process of claim 7, wherein the process further comprises contacting Compound E 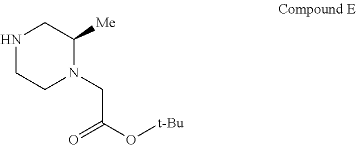 [SMILES: C[C@@H]1CNCCN1CC(=O)OC(C)(C)C]
with Compound I 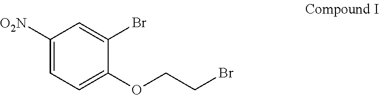 [SMILES: O=[N+]([O-])c1ccc(OCCBr)c(Br)c1]
in the presence of a base in a solvent under conditions suitable to provide Compound K,
optionally the solvent comprises a mixture of tetrahydrofuran and water,
optionally the base is potassium carbonate.

9. The process of claim 8, wherein the process further comprises contacting Compound AA 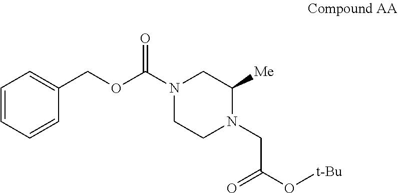 [SMILES: C[C@@H]1CN(C(=O)OCc2ccccc2)CCN1CC(=O)OC(C)(C)C]
with a palladium catalyst in a solvent under conditions suitable to provide Compound E,
optionally the process comprises contacting Compound A′ 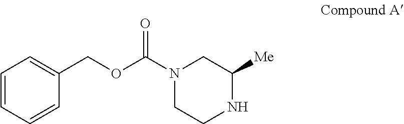 [SMILES: C[C@@H]1CN(C(=O)OCc2ccccc2)CCN1]
with tert-butyl 2-bromoacetate in a solvent under conditions suitable to provide Compound AA,
optionally the process comprises contacting Compound H 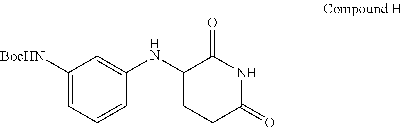 [SMILES: CC(C)(C)OC(=O)Nc1cccc(NC2CCC(=O)NC2=O)c1]
with p-toluenesulfonic acid monohydrate under conditions suitable to provide Compound J,
optionally the process comprises contacting an acid salt of 3-aminopiperidine-2,6-dione 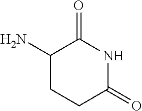
with tert-butyl (3-bromophenyl) carbamate 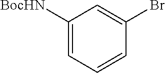 [SMILES: CC(C)(C)OC(=O)Nc1cccc(Br)c1]
under conditions suitable to provide Compound H,
optionally the process comprises contacting 3-bromopiperidine-2,6-dione (Compound D) 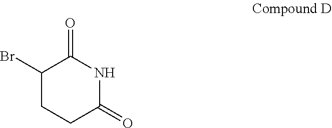
with tert-butyl (3-aminophenyl) carbamate 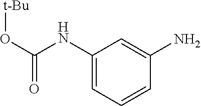 [SMILES: CC(C)(C)OC(=O)Nc1cccc(N)c1]
under conditions suitable to provide Compound H,
optionally the process comprises contacting 6,7-dihydro-5H-oxazolo[3,2-a]pyridine-2,3,5-trione (Compound C) 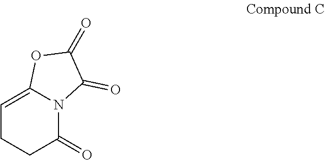 [SMILES: O=C1OC2=CCCC(=O)N2C1=O]
with n-bromosuccinimide (NBS) under conditions suitable to provide Compound D,
optionally the process comprises contacting piperidine-2,6-dione (Compound A) 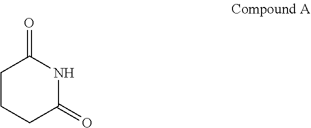
with oxalyl chloride under conditions suitable to provide Compound C.

10. The process of claim 1, wherein the process comprises
a) contacting Compound N 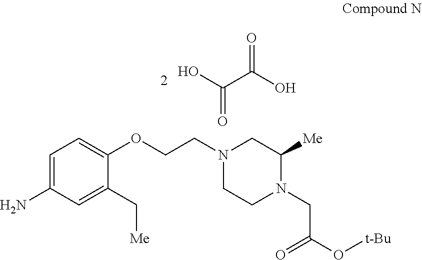
with methyl 2-bromo-2-methylpropionate under conditions suitable to provide Compound P 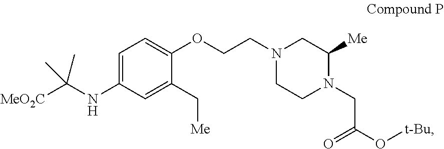 [SMILES: CCc1cc(NC(C)(C)C(=O)OC)ccc1OCCN1CCN(CC(=O)OC(C)(C)C)[C@H](C)C1]
b) contacting Compound P with Compound S 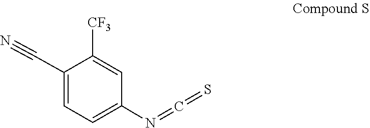 [SMILES: N#Cc1ccc(N=C=S)cc1C(F)(F)F]
under conditions suitable to provide Compound U 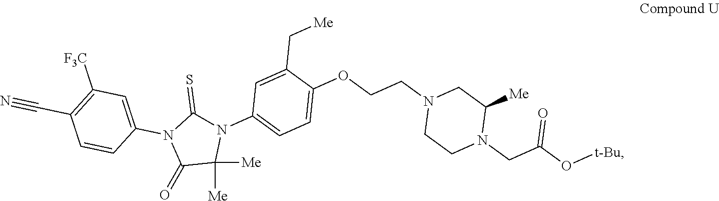 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
c) contacting Compound U with an acid under conditions suitable to provide Compound W 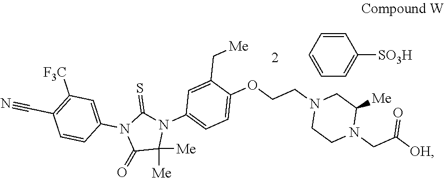
and d) contacting Compound W with Compound J 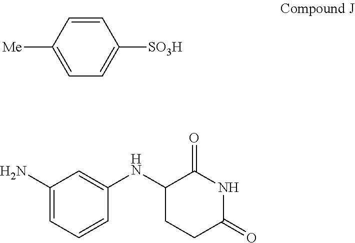 [SMILES: Cc1ccc(S(=O)(=O)O)cc1 ; Nc1cccc(NC2CCC(=O)NC2=O)c1]
under conditions suitable to provide Compound 1.

11. The process of claim 10, wherein the process further comprises
i) contacting an acid salt of 3-aminopiperidine-2,6-dione 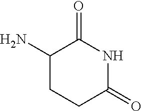
with tert-butyl (3-bromophenyl) carbamate 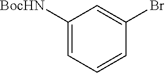 [SMILES: CC(C)(C)OC(=O)Nc1cccc(Br)c1]
under conditions suitable to provide Compound H 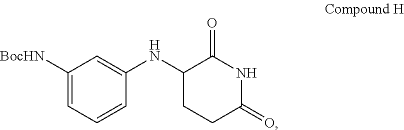 [SMILES: CC(C)(C)OC(=O)Nc1cccc(NC2CCC(=O)NC2=O)c1]
and ii) contacting Compound H with p-toluenesulfonic acid monohydrate under conditions suitable to provide Compound J

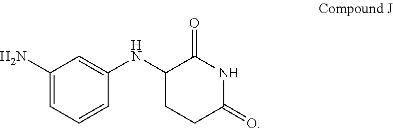 [SMILES: Nc1cccc(NC2CCC(=O)NC2=O)c1]

12. The process of claim 11 further comprising
i) contacting piperidine-2,6-dione (Compound A) 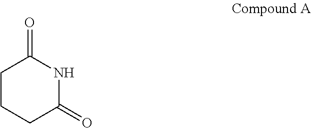
with oxalyl chloride under conditions suitable to provide Compound C 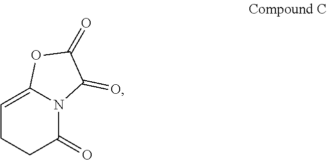 [SMILES: O=C1OC2=CCCC(=O)N2C1=O]
ii) contacting Compound C with n-bromosuccinimide (NBS) under conditions suitable to provide Compound D 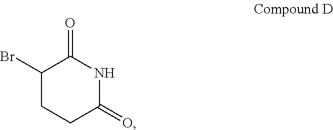
iii) contacting Compound D with tert-butyl (3-aminophenyl) carbamate 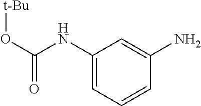 [SMILES: CC(C)(C)OC(=O)Nc1cccc(N)c1]
under conditions suitable to provide Compound H 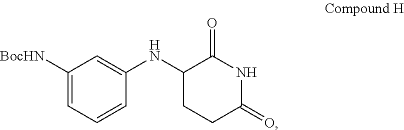 [SMILES: CC(C)(C)OC(=O)Nc1cccc(NC2CCC(=O)NC2=O)c1]
and
iv) contacting Compound H with p-toluenesulfonic acid monohydrate under conditions suitable to provide Compound J.

13. The process of claim 10, where Compound N is prepared by
1) contacting Compound A′ 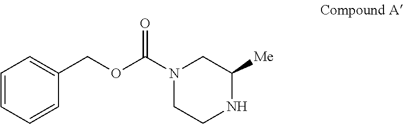 [SMILES: C[C@@H]1CN(C(=O)OCc2ccccc2)CCN1]
with tert-butyl 2-bromoacetate in a solvent under conditions suitable to provide Compound AA 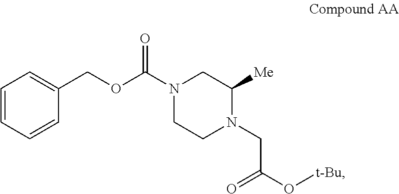 [SMILES: C[C@@H]1CN(C(=O)OCc2ccccc2)CCN1CC(=O)OC(C)(C)C]
2) contacting Compound AA with a palladium catalyst in a solvent under conditions suitable to provide Compound E 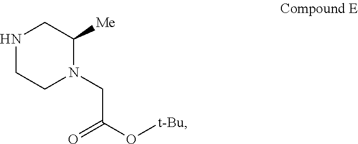 [SMILES: C[C@@H]1CNCCN1CC(=O)OC(C)(C)C]
3) contacting Compound E with Compound I 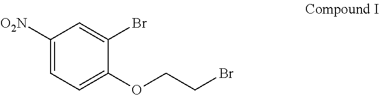 [SMILES: O=[N+]([O-])c1ccc(OCCBr)c(Br)c1]
in the presence of a base in a solvent under conditions suitable to provide Compound K 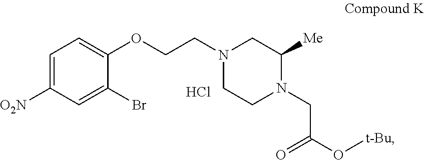 [SMILES: C[C@@H]1CN(CCOc2ccc([N+](=O)[O-])cc2Br)CCN1CC(=O)OC(C)(C)C ; Cl]
4) contacting Compound K with vinyl dioxaborolane in the presence of a palladium catalyst and a base in a solvent under conditions suitable to provide Compound L 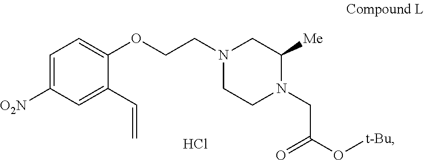 [SMILES: C=Cc1cc([N+](=O)[O-])ccc1OCCN1CCN(CC(=O)OC(C)(C)C)[C@H](C)C1 ; Cl]
and 5) contacting Compound L with a palladium catalyst in a solvent under conditions suitable to provide Compound N.

14. The process of claim 1, wherein the process comprises
1) contacting Compound A′ 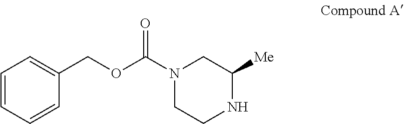 [SMILES: C[C@@H]1CN(C(=O)OCc2ccccc2)CCN1] 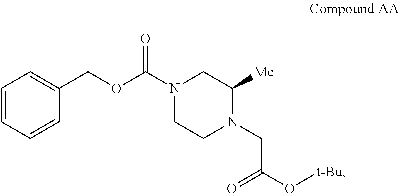 [SMILES: C[C@@H]1CN(C(=O)OCc2ccccc2)CCN1CC(=O)OC(C)(C)C]
2) contacting Compound AA with a palladium catalyst in a solvent under conditions suitable to provide Compound E 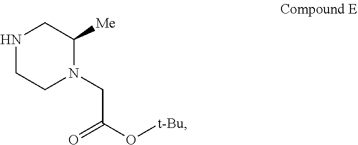 [SMILES: C[C@@H]1CNCCN1CC(=O)OC(C)(C)C]
3) contacting Compound E with Compound I 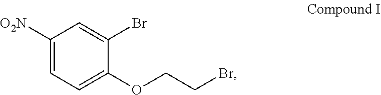 [SMILES: O=[N+]([O-])c1ccc(OCCBr)c(Br)c1]
in the presence of a base in a solvent under conditions suitable to provide Compound K 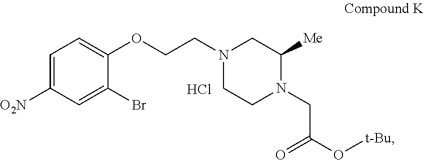 [SMILES: C[C@@H]1CN(CCOc2ccc([N+](=O)[O-])cc2Br)CCN1CC(=O)OC(C)(C)C ; Cl]
4) contacting Compound K with vinyl dioxaborolane in the presence of a palladium catalyst and a base in a solvent under conditions suitable to provide Compound L 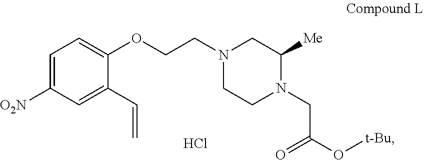 [SMILES: C=Cc1cc([N+](=O)[O-])ccc1OCCN1CCN(CC(=O)OC(C)(C)C)[C@H](C)C1 ; Cl]
5) contacting Compound L with a palladium catalyst in a solvent under conditions suitable to provide Compound N 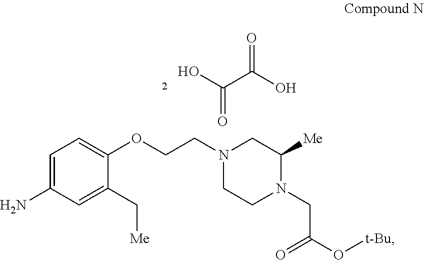
6) contacting Compound N with methyl 2-bromo-2-methylpropionate under conditions suitable to provide Compound P 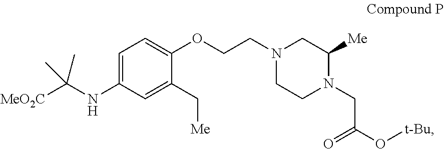 [SMILES: CCc1cc(NC(C)(C)C(=O)OC)ccc1OCCN1CCN(CC(=O)OC(C)(C)C)[C@H](C)C1]
7) contacting Compound P with Compound S 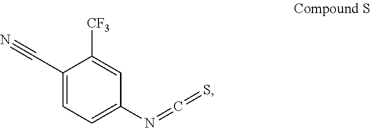 [SMILES: N#Cc1ccc(N=C=S)cc1C(F)(F)F]
under conditions suitable to provide Compound U 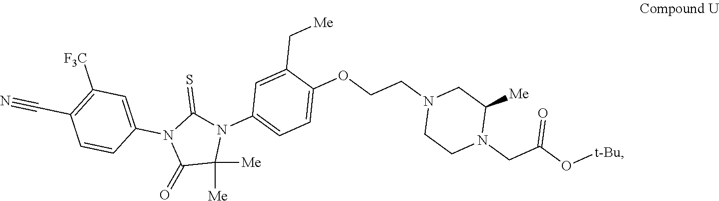 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
8) contacting Compound U with an acid to deprotect the tert-butyl ester group under conditions suitable to provide Compound W 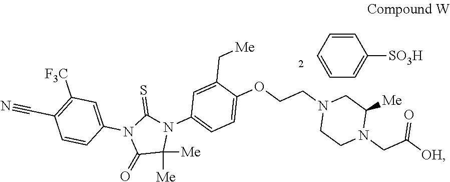
and 9) contacting Compound W with Compound J 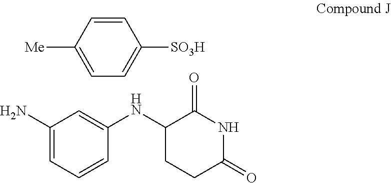
under conditions suitable to provide Compound 1,
wherein Compound J is prepared by
i) contacting an acid salt of 3-aminopiperidine-2,6-dione 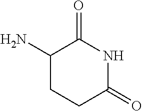
with tert-butyl (3-bromophenyl) carbamate 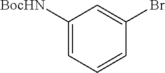 [SMILES: CC(C)(C)OC(=O)Nc1cccc(Br)c1]
under conditions suitable to provide Compound H 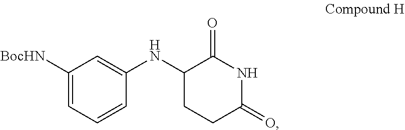 [SMILES: CC(C)(C)OC(=O)Nc1cccc(NC2CCC(=O)NC2=O)c1]
and ii) contacting Compound H with p-toluenesulfonic acid monohydrate under conditions suitable to provide Compound J.

15. The process of claim 1, wherein the process comprises
1) contacting Compound A′ 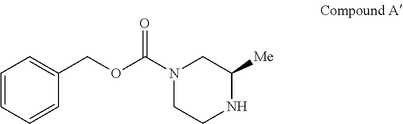 [SMILES: C[C@@H]1CN(C(=O)OCc2ccccc2)CCN1]
with tert-butyl 2-bromoacetate in a solvent under conditions suitable to provide Compound AA 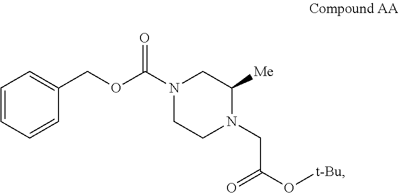 [SMILES: C[C@@H]1CN(C(=O)OCc2ccccc2)CCN1CC(=O)OC(C)(C)C]
2) contacting Compound AA with a palladium catalyst in a solvent under conditions suitable to provide Compound E 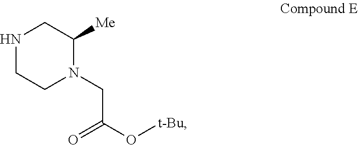 [SMILES: C[C@@H]1CNCCN1CC(=O)OC(C)(C)C]
3) contacting Compound E with Compound I 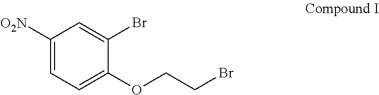 [SMILES: O=[N+]([O-])c1ccc(OCCBr)c(Br)c1]
in the presence of a base in a solvent under conditions suitable to provide Compound K 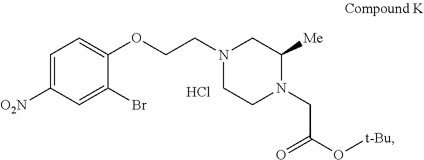 [SMILES: C[C@@H]1CN(CCOc2ccc([N+](=O)[O-])cc2Br)CCN1CC(=O)OC(C)(C)C ; Cl]
4) contacting Compound K with vinyl dioxaborolane in the presence of a palladium catalyst and a base in a solvent under conditions suitable to provide Compound L 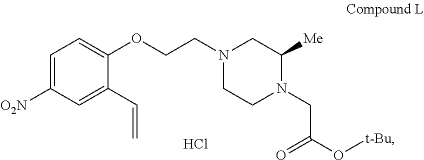 [SMILES: C=Cc1cc([N+](=O)[O-])ccc1OCCN1CCN(CC(=O)OC(C)(C)C)[C@H](C)C1 ; Cl]
5) contacting Compound L with a palladium catalyst in a solvent under conditions suitable to provide Compound N 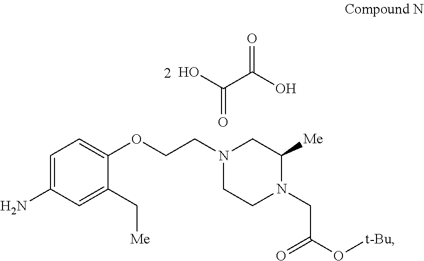
6) contacting Compound N with methyl 2-bromo-2-methylpropionate under conditions suitable to provide Compound P 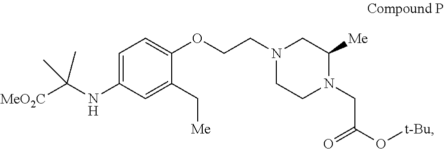 [SMILES: CCc1cc(NC(C)(C)C(=O)OC)ccc1OCCN1CCN(CC(=O)OC(C)(C)C)[C@H](C)C1]
7) contacting Compound P with Compound S 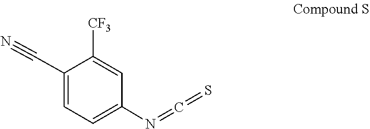 [SMILES: N#Cc1ccc(N=C=S)cc1C(F)(F)F]
under conditions suitable to provide Compound U 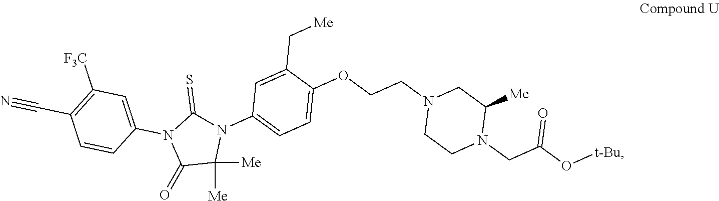 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
8) contacting Compound U with an acid to deprotect the tert-butyl ester group under conditions suitable to provide Compound W 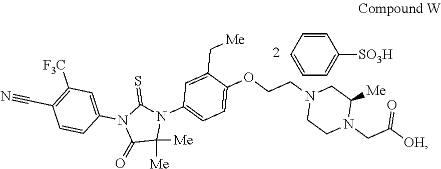
and 9 contacting Compound W with Compound J 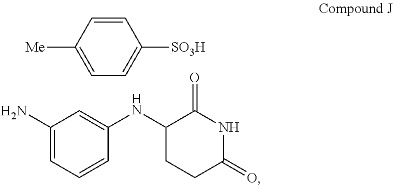
under conditions suitable to provide Compound 1,
wherein Compound J is prepared by
i) contacting piperidine-2,6-dione (Compound A) 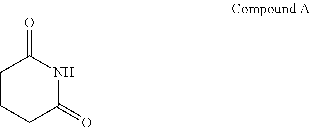
with oxalyl chloride under conditions suitable to provide Compound C 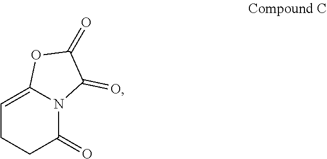 [SMILES: O=C1OC2=CCCC(=O)N2C1=O]
ii) contacting Compound C with n-bromosuccinimide (NBS) under conditions suitable to provide Compound D 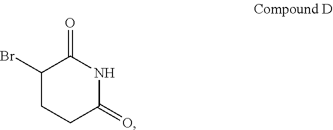
iii) contacting Compound D with tert-butyl (3-aminophenyl) carbamate 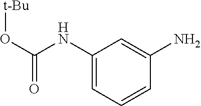 [SMILES: CC(C)(C)OC(=O)Nc1cccc(N)c1]
under conditions suitable to provide Compound H 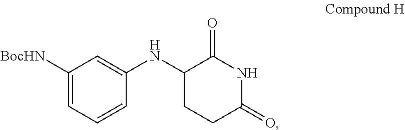 [SMILES: CC(C)(C)OC(=O)Nc1cccc(NC2CCC(=O)NC2=O)c1]
and iv) contacting Compound H with p-toluenesulfonic acid monohydrate under conditions suitable to provide Compound J.

16. A process for preparing Compound 1, comprising
a) contacting 2-hydroxy-5-nitroacetophenone, Compound i 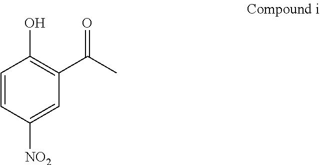
with trifluoroacetic acid and triethylsilane in a solvent under conditions suitable to provide Compound ii 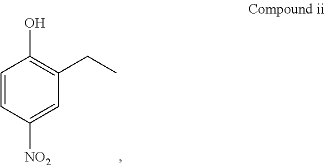
b) contacting Compound ii with ethylene carbonate in a solvent under conditions suitable to provide Compound iii 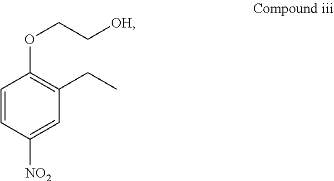
c) contacting Compound iii with 4-toluenesulfonyl chloride (TsCl) and triethylamine (TEA) in a solvent under conditions suitable to provide Compound iv in solution 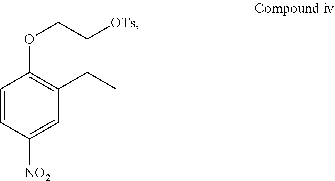 [SMILES: CCc1cc([N+](=O)[O-])ccc1OCCOS(=O)(=O)c1ccc(C)cc1]
d) contacting Compound iv in solution with Compound E in the presence of a base in a solvent, followed by oxalic acid under conditions suitable to provide Compound T 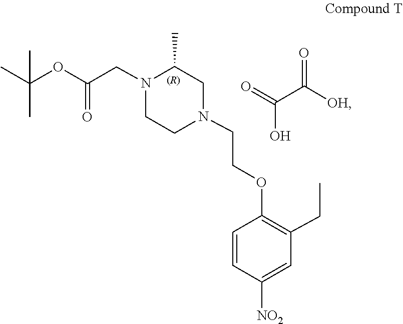
e) contacting Compound T with a platinum catalyst in a solvent under conditions suitable to provide Compound N1 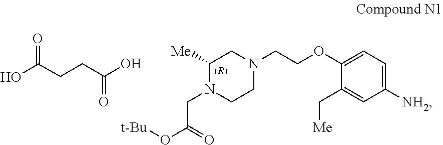
f) contacting Compound N1 with methyl 2-bromo-2-methylpropionate under conditions suitable to provide Compound P 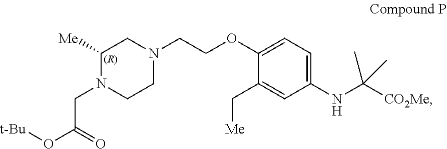
g) contacting Compound P with Compound S 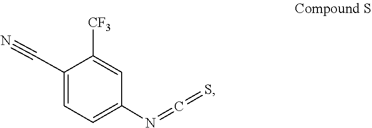 [SMILES: N#Cc1ccc(N=C=S)cc1C(F)(F)F]
h) followed by oxalic acid in a solvent under conditions suitable to provide Compound U 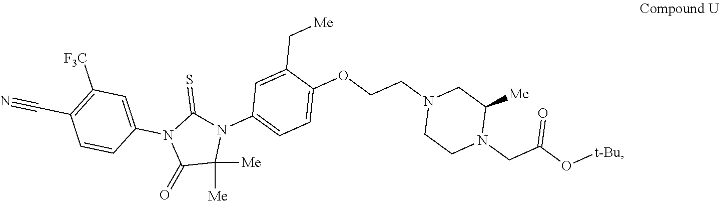 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
i) contacting Compound U with potassium carbonate followed by p-tosyl sulfonic acid in a solvent under conditions suitable to provide Compound W 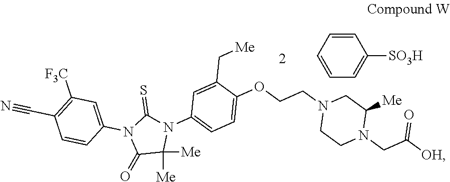
j) contacting Compound W with Compound J 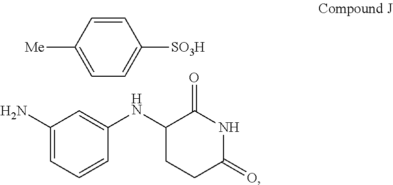
followed by butyl acetate under conditions suitable to provide Compound 1, butyl acetate solvate 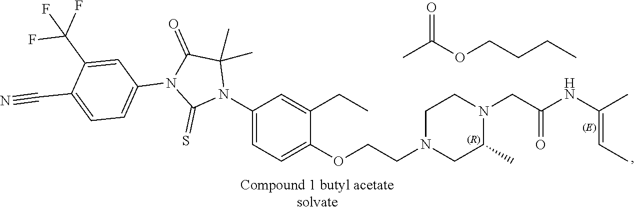
k) dissolving Compound 1, butyl acetate solvate to obtain a solution, and
l) spray drying the solution to obtain Compound 1

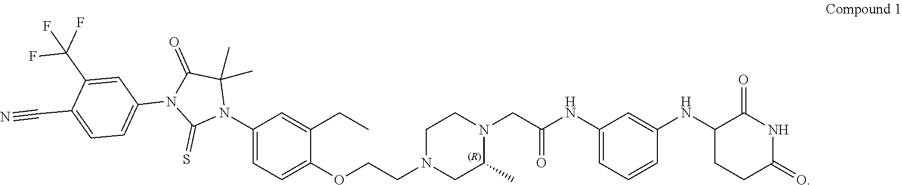

17. A process for preparing Compound N 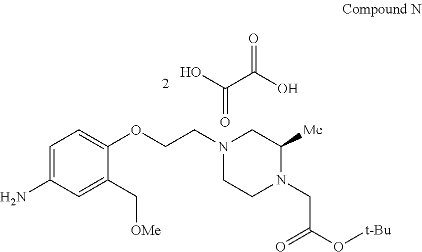
comprising contacting Compound L 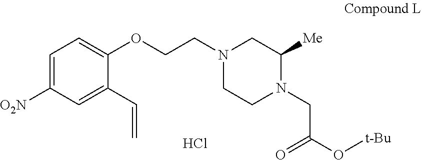 [SMILES: C=Cc1cc([N+](=O)[O-])ccc1OCCN1CCN(CC(=O)OC(C)(C)C)[C@H](C)C1 ; Cl]
with a palladium catalyst in a solvent under conditions suitable to provide Compound N, optionally the process comprises
1) contacting Compound A′ 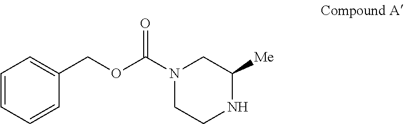 [SMILES: C[C@@H]1CN(C(=O)OCc2ccccc2)CCN1]
with tert-butyl 2-bromoacetate in a solvent under conditions suitable to provide Compound AA 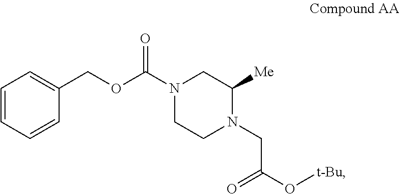 [SMILES: C[C@@H]1CN(C(=O)OCc2ccccc2)CCN1CC(=O)OC(C)(C)C]
2) contacting Compound AA with a palladium catalyst in a solvent under conditions suitable to provide Compound E 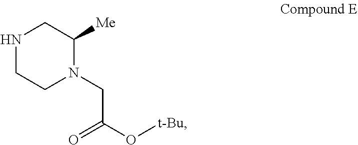 [SMILES: C[C@@H]1CNCCN1CC(=O)OC(C)(C)C]
3) contacting Compound E with Compound I 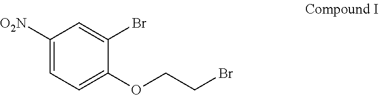 [SMILES: O=[N+]([O-])c1ccc(OCCBr)c(Br)c1]
in the presence of a base in a solvent under conditions suitable to provide Compound K 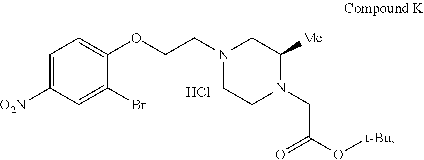 [SMILES: C[C@@H]1CN(CCOc2ccc([N+](=O)[O-])cc2Br)CCN1CC(=O)OC(C)(C)C ; Cl]
4) contacting Compound K with vinyl dioxaborolane in the presence of a palladium catalyst and a base in a solvent under conditions suitable to provide Compound L 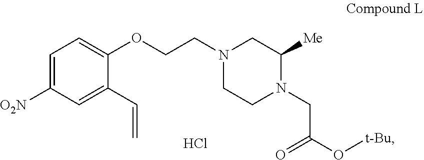 [SMILES: C=Cc1cc([N+](=O)[O-])ccc1OCCN1CCN(CC(=O)OC(C)(C)C)[C@H](C)C1 ; Cl]
and 5) contacting Compound L with a palladium catalyst in a solvent under conditions suitable to provide Compound N.

18. A process for preparing Compound U 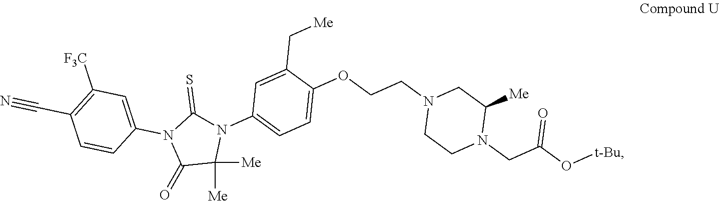 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
comprising contacting Compound P 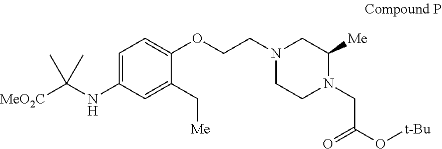 [SMILES: CCc1cc(NC(C)(C)C(=O)OC)ccc1OCCN1CCN(CC(=O)OC(C)(C)C)[C@H](C)C1]
with Compound 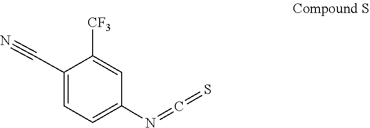 [SMILES: N#Cc1ccc(N=C=S)cc1C(F)(F)F]
in a solvent under conditions suitable to provide Compound U.

19. A process for preparing Compound P 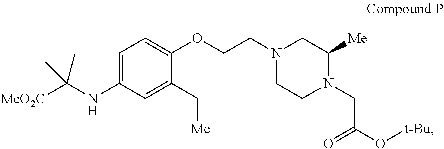 [SMILES: CCc1cc(NC(C)(C)C(=O)OC)ccc1OCCN1CCN(CC(=O)OC(C)(C)C)[C@H](C)C1]
comprising contacting Compound N 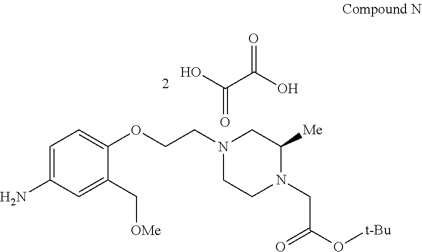
with methyl 2-bromo-2-methylpropionate in a solvent in the presence of a base under conditions suitable to provide Compound P.

20. A process for preparing Compound W 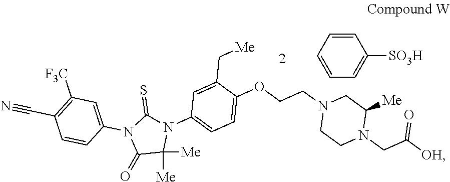
comprising contacting Compound U 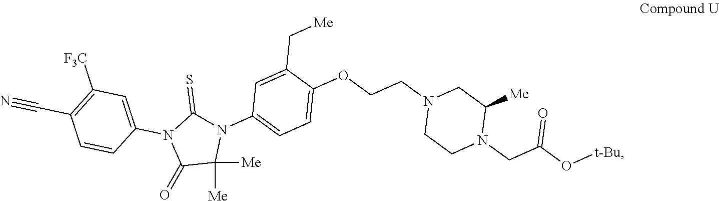 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
with BSA in a solvent under conditions suitable to provide Compound W.

21. A process for preparing Compound viii 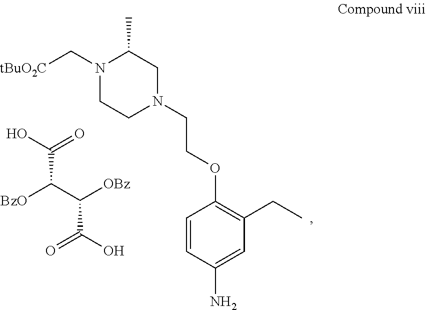
wherein the process comprises contacting Compound vii 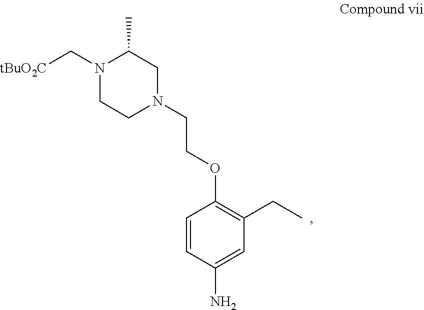 [SMILES: CCc1cc(N)ccc1OCCN1CCN(CC(=O)OC(C)(C)C)[C@H](C)C1]
with (+)-dibenzoyl-D-tartaric acid to provide Compound viii, optionally the process further comprises contacting Compound vi 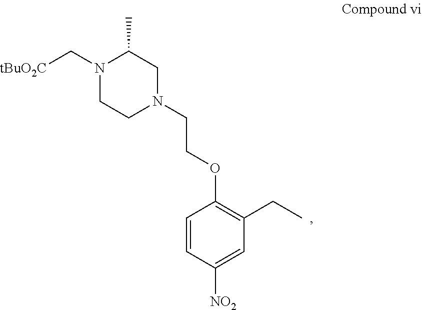 [SMILES: CCc1cc([N+](=O)[O-])ccc1OCCN1CCN(CC(=O)OC(C)(C)C)[C@H](C)C1]
with a palladium catalyst in a solvent under conditions suitable to provide Compound vii, optioanlly the process further comprises contacting Compound v 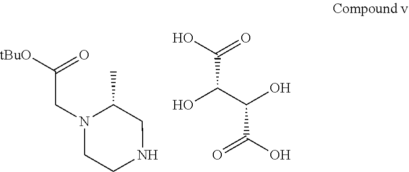
with Compound iv 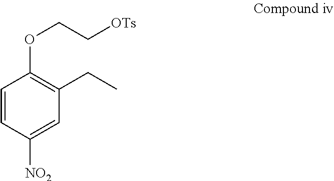 [SMILES: CCc1cc([N+](=O)[O-])ccc1OCCOS(=O)(=O)c1ccc(C)cc1]
in a solvent under conditions suitable to provide Compound vi,
optionally the process further comprises contacting contacting (R)-(−)-2-methylpiperazine, Compound ix 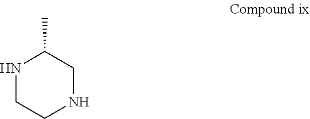
with a)ethyl trifluoroacetate in a solvent in presence of a base; b) tert-butyl bromoacetate;
c) 25% tetramethylammonium hydroxide (Me₄NOH) in methanol; and d) D-tartaric acid under conditions suitable to provide Compound v,
optionally the process comprises contacting Compound iii 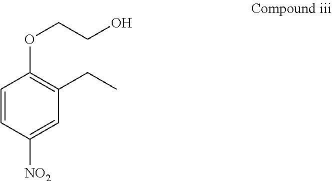
with 4-toluenesulfonyl chloride (TsC1) and triethylamine (TEA) in a solvent under conditions suitable to provide Compound iv,
optionally the process comprises contacting Compound ii 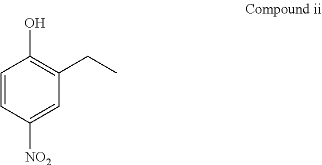
with sodium carbonate and ethylene carbonate in a solvent under conditions suitable to provide Compound iii,
optionally the process comprises contacting 2-hydroxy-5-nitroacetophenone, Compound i 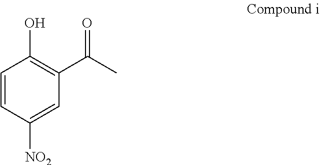
with trifluoroacetic acid and triethylsilane in a solvent under conditions suitable to provide Compound ii.

22. A compound selected from
Compound K having structure: 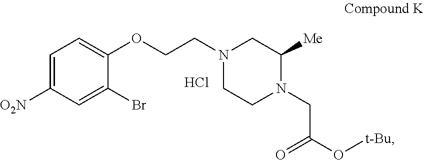 [SMILES: C[C@@H]1CN(CCOc2ccc([N+](=O)[O-])cc2Br)CCN1CC(=O)OC(C)(C)C ; Cl]
Compound L having structure: 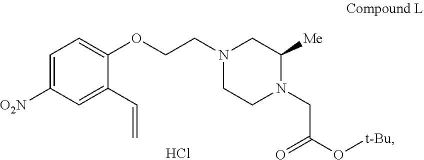 [SMILES: C=Cc1cc([N+](=O)[O-])ccc1OCCN1CCN(CC(=O)OC(C)(C)C)[C@H](C)C1 ; Cl]
Compound N having structure: 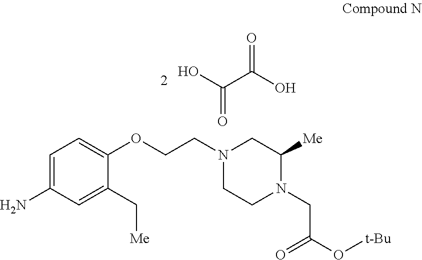
Compound U having structure: 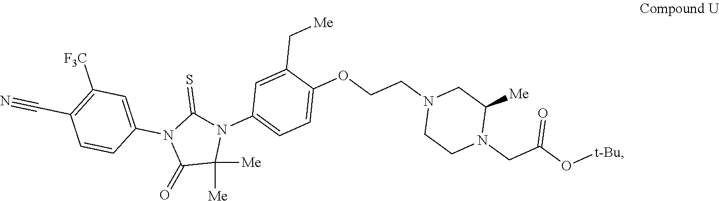 [SMILES: CCc1cc(N2C(=S)N(c3ccc(C#N)c(C(F)(F)F)c3)C(=O)C2(C)C)ccc1OCCN1CCN(CC(=O)OC(C)(C)C)[C@H](C)C1]
Compound P having structure: 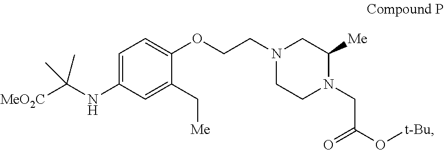 [SMILES: CCc1cc(NC(C)(C)C(=O)OC)ccc1OCCN1CCN(CC(=O)OC(C)(C)C)[C@H](C)C1]
and
Compound W having structure: [chemical structure]
